SEQUENCE LISTING (311 sequences)

SEQ ID NO: 1 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 662 aa, synthetic construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 6 - protein, 522 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 7 - protein, 862 aa, synthetic construct
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 8 - protein, 599 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 9 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 12 - protein, 231 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 231 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 14 - protein, 5 aa, synthetic construct
GGGGS

SEQ ID NO: 15 - protein, 10 aa, synthetic construct
GGGGSGGGGS

SEQ ID NO: 16 - protein, 15 aa, synthetic construct
GGGGSGGGGSGGGGS

SEQ ID NO: 17 - protein, 17 aa, synthetic construct
TRGGGGSGGGGSGGGGS

SEQ ID NO: 18 - protein, 20 aa, synthetic construct
GGGGSGGGGSGGGGSGGGGS

SEQ ID NO: 19 - protein, 25 aa, synthetic construct
GGGGSGGGGSGGGGSGGGGSGGGGS

SEQ ID NO: 20 - protein, 30 aa, synthetic construct
GGGGSGGGGSGGGGSGGGGSGGGGSGGGGS

SEQ ID NO: 21 - protein, 35 aa, synthetic construct
GGGGSGGGGSGGGGSGGGGSGGGGSGGGGSGGGGS

SEQ ID NO: 22 - protein, 40 aa, synthetic construct
GGGGSGGGGSGGGGSGGGGSGGGGSGGGGSGGGGSGGGGS

SEQ ID NO: 23 - protein, 18 aa, synthetic construct
GSTSGSGKPGSGEGSTKG

SEQ ID NO: 24 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 26 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNAS

SEQ ID NO: 27 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNAS

SEQ ID NO: 28 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 29 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 30 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNAS

SEQ ID NO: 32 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNAS

SEQ ID NO: 33 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 34 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 35 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNAS

SEQ ID NO: 36 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNAS

SEQ ID NO: 37 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 38 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 39 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNAS

SEQ ID NO: 40 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTKIKLCILLHAFRIRAVTIDRVMSYLNAS

SEQ ID NO: 41 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATKIKLCILLHAFRIRAVTIDRVMSYLNAS

SEQ ID NO: 42 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTKIKLCILLHAFRIRAVTIDRVMSYLNAS

SEQ ID NO: 43 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATKIKLCILLHAFRIRAVTIDRVMSYLNAS

SEQ ID NO: 44 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATKIKLCILLHAFRIRAVTIDRVMSYLNAS

SEQ ID NO: 45 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNAS

SEQ ID NO: 46 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNAS

SEQ ID NO: 47 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 48 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 49 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNAS

SEQ ID NO: 50 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTKIKLCILLHAFRIRAVTIDRVMSYLNAS

SEQ ID NO: 51 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATKIKLCILLHAFRIRAVTIDRVMSYLNAS

SEQ ID NO: 52 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTKIKLCILLHAFRIRAVTIDRVMSYLNAS

SEQ ID NO: 53 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATKIKLCILLHAFRIRAVTIDRVMSYLNAS

SEQ ID NO: 54 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATKIKLCILLHAFRIRAVTIDRVMSYLNAS

SEQ ID NO: 55 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNAS

SEQ ID NO: 56 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTKIKLCILLHAFRIRAVTIDRVMSYLNAS

SEQ ID NO: 57 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATKIKLCILLHAFRIRAVTIDRVMSYLNAS

SEQ ID NO: 58 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTKIKLCILLHAFRIRAVTIDRVMSYLNAS

SEQ ID NO: 59 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATKIKLCILLHAFRIRAVTIDRVMSYLNAS

SEQ ID NO: 60 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATKIKLCILLHAFRIRAVTIDRVMSYLNAS

SEQ ID NO: 61 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTKIKLCILLHAFRIRAVTIDRVMSYLNAS

SEQ ID NO: 62 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATKIKLCILLHAFRIRAVTIDRVMSYLNAS

SEQ ID NO: 63 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATKIKLCILLHAFRIRAVTIDRVMSYLNAS

SEQ ID NO: 64 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATKIKLCILLHAFRIRAVTIDRVMSYLNAS

SEQ ID NO: 65 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNAS

SEQ ID NO: 66 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTKIKLCILLHAFRIRAVTIDRVMSYLNAS

SEQ ID NO: 67 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATKIKLCILLHAFRIRAVTIDRVMSYLNAS

SEQ ID NO: 68 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTKIKLCILLHAFRIRAVTIDRVMSYLNAS

SEQ ID NO: 69 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATKIKLCILLHAFRIRAVTIDRVMSYLNAS

SEQ ID NO: 70 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATKIKLCILLHAFRIRAVTIDRVMSYLNAS

SEQ ID NO: 71 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTKIKLCILLHAFRIRAVTIDRVMSYLNAS

SEQ ID NO: 72 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATKIKLCILLHAFRIRAVTIDRVMSYLNAS

SEQ ID NO: 73 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATKIKLCILLHAFRIRAVTIDRVMSYLNAS

SEQ ID NO: 74 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATKIKLCILLHAFRIRAVTIDRVMSYLNAS

SEQ ID NO: 75 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTKIKLCILLHAFRIRAVTIDRVMSYLNAS

SEQ ID NO: 76 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATKIKLCILLHAFRIRAVTIDRVMSYLNAS

SEQ ID NO: 77 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATKIKLCILLHAFRIRAVTIDRVMSYLNAS

SEQ ID NO: 78 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATKIKLCILLHAFRIRAVTIDRVMSYLNAS

SEQ ID NO: 79 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATKIKLCILLHAFRIRAVTIDRVMSYLNAS

SEQ ID NO: 80 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTKIKLCILLHAFRIRAVTIDRVMSYLNAS

SEQ ID NO: 81 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATKIKLCILLHAFRIRAVTIDRVMSYLNAS

SEQ ID NO: 82 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATKIKLCILLHAFRIRAVTIDRVMSYLNAS

SEQ ID NO: 83 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATKIKLCILLHAFRIRAVTIDRVMSYLNAS

SEQ ID NO: 84 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATKIKLCILLHAFRIRAVTIDRVMSYLNAS

SEQ ID NO: 85 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATKIKLCILLHAFRIRAVTIDRVMSYLNAS

SEQ ID NO: 86 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATKIKLCILLHAFRIRAVTIDRVMSYLNAS

SEQ ID NO: 87 - protein, 197 aa, synthetic construct
RNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 88 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 89 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 90 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 91 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 547 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 438 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 438 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 438 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 438 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 438 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 438 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 438 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 104 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 105 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 106 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTAIKLCILLHAFRIRAVTIDRVMSYLNAS

SEQ ID NO: 107 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTAIKLCILLHAFRIRAVTIDRVMSYLNAS

SEQ ID NO: 108 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTAIKLCILLHAFRIRAVTIDRVMSYLNAS

SEQ ID NO: 109 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATAIKLCILLHAFRIRAVTIDRVMSYLNAS

SEQ ID NO: 110 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 111 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTAIKLCILLHAFRIRAVTIDRVMSYLNAS

SEQ ID NO: 112 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTAIKLCILLHAFRIRAVTIDRVMSYLNAS

SEQ ID NO: 113 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTAIKLCILLHAFRIRAVTIDRVMSYLNAS

SEQ ID NO: 114 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATAIKLCILLHAFRIRAVTIDRVMSYLNAS

SEQ ID NO: 115 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTAIKLCILLHAFRIRAVTIDRVMSYLNAS

SEQ ID NO: 116 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTAIKLCILLHAFRIRAVTIDRVMSYLNAS

SEQ ID NO: 117 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTAIKLCILLHAFRIRAVTIDRVMSYLNAS

SEQ ID NO: 118 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATAIKLCILLHAFRIRAVTIDRVMSYLNAS

SEQ ID NO: 119 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTAIKLCILLHAFRIRAVTIDRVMSYLNAS

SEQ ID NO: 120 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTAIKLCILLHAFRIRAVTIDRVMSYLNAS

SEQ ID NO: 121 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATAIKLCILLHAFRIRAVTIDRVMSYLNAS

SEQ ID NO: 122 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTAIKLCILLHAFRIRAVTIDRVMSYLNAS

SEQ ID NO: 123 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATAIKLCILLHAFRIRAVTIDRVMSYLNAS

SEQ ID NO: 124 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATAIKLCILLHAFRIRAVTIDRVMSYLNAS

SEQ ID NO: 125 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYKTAIKLCILLHAFRIRAVTIDRVMSYLNAS

SEQ ID NO: 126 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYKTAIKLCILLHAFRIRAVTIDRVMSYLNAS

SEQ ID NO: 127 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTAIKLCILLHAFRIRAVTIDRVMSYLNAS

SEQ ID NO: 128 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATAIKLCILLHAFRIRAVTIDRVMSYLNAS

SEQ ID NO: 129 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTAIKLCILLHAFRIRAVTIDRVMSYLNAS

SEQ ID NO: 130 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTAIKLCILLHAFRIRAVTIDRVMSYLNAS

SEQ ID NO: 131 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATAIKLCILLHAFRIRAVTIDRVMSYLNAS

SEQ ID NO: 132 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTAIKLCILLHAFRIRAVTIDRVMSYLNAS

SEQ ID NO: 133 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATAIKLCILLHAFRIRAVTIDRVMSYLNAS

SEQ ID NO: 134 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATAIKLCILLHAFRIRAVTIDRVMSYLNAS

SEQ ID NO: 135 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTAIKLCILLHAFRIRAVTIDRVMSYLNAS

SEQ ID NO: 136 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTAIKLCILLHAFRIRAVTIDRVMSYLNAS

SEQ ID NO: 137 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATAIKLCILLHAFRIRAVTIDRVMSYLNAS

SEQ ID NO: 138 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTAIKLCILLHAFRIRAVTIDRVMSYLNAS

SEQ ID NO: 139 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATAIKLCILLHAFRIRAVTIDRVMSYLNAS

SEQ ID NO: 140 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATAIKLCILLHAFRIRAVTIDRVMSYLNAS

SEQ ID NO: 141 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTAIKLCILLHAFRIRAVTIDRVMSYLNAS

SEQ ID NO: 142 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATAIKLCILLHAFRIRAVTIDRVMSYLNAS

SEQ ID NO: 143 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATAIKLCILLHAFRIRAVTIDRVMSYLNAS

SEQ ID NO: 144 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATAIKLCILLHAFRIRAVTIDRVMSYLNAS

SEQ ID NO: 145 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYKTAIKLCILLHAFRIRAVTIDRVMSYLNAS

SEQ ID NO: 146 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYKTAIKLCILLHAFRIRAVTIDRVMSYLNAS

SEQ ID NO: 147 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKRQIFLDQNMLAVIDELMQALNFNSETVPQKSSLEEPDFYATAIKLCILLHAFRIRAVTIDRVMSYLNAS

SEQ ID NO: 148 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYKTAIKLCILLHAFRIRAVTIDRVMSYLNAS

SEQ ID NO: 149 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATAIKLCILLHAFRIRAVTIDRVMSYLNAS

SEQ ID NO: 150 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYATAIKLCILLHAFRIRAVTIDRVMSYLNAS

SEQ ID NO: 151 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTAIKLCILLHAFRIRAVTIDRVMSYLNAS

SEQ ID NO: 152 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATAIKLCILLHAFRIRAVTIDRVMSYLNAS

SEQ ID NO: 153 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATAIKLCILLHAFRIRAVTIDRVMSYLNAS

SEQ ID NO: 154 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATAIKLCILLHAFRIRAVTIDRVMSYLNAS

SEQ ID NO: 155 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTAIKLCILLHAFRIRAVTIDRVMSYLNAS

SEQ ID NO: 156 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATAIKLCILLHAFRIRAVTIDRVMSYLNAS

SEQ ID NO: 157 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATAIKLCILLHAFRIRAVTIDRVMSYLNAS

SEQ ID NO: 158 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATAIKLCILLHAFRIRAVTIDRVMSYLNAS

SEQ ID NO: 159 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATAIKLCILLHAFRIRAVTIDRVMSYLNAS

SEQ ID NO: 160 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYKTAIKLCILLHAFRIRAVTIDRVMSYLNAS

SEQ ID NO: 161 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKRQIFLDQNMLAVIDELMQALNFNSETVPQKSSLEEPDFYATAIKLCILLHAFRIRAVTIDRVMSYLNAS

SEQ ID NO: 162 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATAIKLCILLHAFRIRAVTIDRVMSYLNAS

SEQ ID NO: 163 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMDAKAQIFLDQNMLAVIDELMQALNFNSETVPQKSSLEEPDFYATAIKLCILLHAFRIRAVTIDRVMSYLNAS

SEQ ID NO: 164 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAPKAQIFLDQNMLAVIDELMQALNFNSETVPQKSSLEEPDFYATAIKLCILLHAFRIRAVTIDRVMSYLNAS

SEQ ID NO: 165 - protein, 197 aa, synthetic construct
RNLPVATPDPGMFPCLHHSQNLLRAVSNMLQKARQTLEFYPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATAIKLCILLHAFRIRAVTIDRVMSYLNAS

SEQ ID NO: 166 - protein, 197 aa, synthetic construct
RNLPVATPDPGMFPCLHHSQNLLRAVSNMLQKARQTLEFAPCASEEIDHEDITKDKTSTVEACLPLELTKNESCLNSRETSFITNGSCLASRKTSFMMALCLSSIYEDLKMYQVEFKTMNAKLLMAAKAQIFLDQNMLAVIDELMQALNFNSETVPQKSSLEEPDFYATAIKLCILLHAFRIRAVTIDRVMSYLNAS

SEQ ID NO: 167 - protein, 438 aa, synthetic construct; identical to SEQ ID NO: 100

SEQ ID NO: 168 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDAKRQIFLDQNMLAVIDELMQALNFNSETVPQKSSLEEPDFYATKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 438 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 547 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 171 - protein, 438 aa, synthetic construct; identical to SEQ ID NO: 91

SEQ ID NO: 172 - protein, 438 aa, synthetic construct; identical to SEQ ID NO: 98

SEQ ID NO: 173 - protein, 438 aa, synthetic construct; identical to SEQ ID NO: 99

SEQ ID NO: 174 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 438 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 197 aa, synthetic construct
RNLPVATPDPGMFPCLHHSQNLLRAVSNMLQKARQTLEFCPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 178 - protein, 197 aa, synthetic construct
RNLPVATPDPGMFPCLHHSQNLLRAVSNMLQKARQTLEFDPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 179 - protein, 197 aa, synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 180 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 181 - protein, 197 aa, synthetic construct
RNLPVATPDPGMFPCLHHSQNLLRAVSNMLQKARQTLEFKPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 182 - protein, 197 aa, synthetic construct
RNLPVATPDPGMFPCLHHSQNLLRAVSNMLQKARQTLEFNPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 183 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 184 - protein, 197 aa, synthetic construct
RNLPVATPDPGMFPCLHHSQNLLRAVSNMLQKARQTLEFQPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 185 - protein, 197 aa, synthetic construct
RNLPVATPDPGMFPCLHHSQNLLRAVSNMLQKARQTLEFRPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 186 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 187 - protein, 197 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 188 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTCIKLCILLHAFRIRAVTIDRVMSYLNAS

SEQ ID NO: 189 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTDIKLCILLHAFRIRAVTIDRVMSYLNAS

SEQ ID NO: 190 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTEIKLCILLHAFRIRAVTIDRVMSYLNAS

SEQ ID NO: 191 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTGIKLCILLHAFRIRAVTIDRVMSYLNAS

SEQ ID NO: 192 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTIIKLCILLHAFRIRAVTIDRVMSYLNAS

SEQ ID NO: 193 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTMIKLCILLHAFRIRAVTIDRVMSYLNAS

SEQ ID NO: 194 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTPIKLCILLHAFRIRAVTIDRVMSYLNAS

SEQ ID NO: 195 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTSIKLCILLHAFRIRAVTIDRVMSYLNAS

SEQ ID NO: 196 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTTIKLCILLHAFRIRAVTIDRVMSYLNAS

SEQ ID NO: 197 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTVIKLCILLHAFRIRAVTIDRVMSYLNAS

SEQ ID NO: 198 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTWIKLCILLHAFRIRAVTIDRVMSYLNAS

SEQ ID NO: 199 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMCPKRQIFLDQNMLAVIDELMQALNFNSETVPQKSSLEEPDFYKTKIKLCILLHAFRIRAVTIDRVMSYLNAS

SEQ ID NO: 200 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMEPKRQIFLDQNMLAVIDELMQALNFNSETVPQKSSLEEPDFYKTKIKLCILLHAFRIRAVTIDRVMSYLNAS

SEQ ID NO: 201 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMFPKRQIFLDQNMLAVIDELMQALNFNSETVPQKSSLEEPDFYKTKIKLCILLHAFRIRAVTIDRVMSYLNAS

SEQ ID NO: 202 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMGPKRQIFLDQNMLAVIDELMQALNFNSETVPQKSSLEEPDFYKTKIKLCILLHAFRIRAVTIDRVMSYLNAS

SEQ ID NO: 203 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMIPKRQIFLDQNMLAVIDELMQALNFNSETVPQKSSLEEPDFYKTKIKLCILLHAFRIRAVTIDRVMSYLNAS

SEQ ID NO: 204 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMKPKRQIFLDQNMLAVIDELMQALNFNSETVPQKSSLEEPDFYKTKIKLCILLHAFRIRAVTIDRVMSYLNAS

SEQ ID NO: 205 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMLPKRQIFLDQNMLAVIDELMQALNFNSETVPQKSSLEEPDFYKTKIKLCILLHAFRIRAVTIDRVMSYLNAS

SEQ ID NO: 206 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMMPKRQIFLDQNMLAVIDELMQALNFNSETVPQKSSLEEPDFYKTKIKLCILLHAFRIRAVTIDRVMSYLNAS

SEQ ID NO: 207 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMNPKRQIFLDQNMLAVIDELMQALNFNSETVPQKSSLEEPDFYKTKIKLCILLHAFRIRAVTIDRVMSYLNAS

SEQ ID NO: 208 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMPPKRQIFLDQNMLAVIDELMQALNFNSETVPQKSSLEEPDFYKTKIKLCILLHAFRIRAVTIDRVMSYLNAS

SEQ ID NO: 209 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMQPKRQIFLDQNMLAVIDELMQALNFNSETVPQKSSLEEPDFYKTKIKLCILLHAFRIRAVTIDRVMSYLNAS

SEQ ID NO: 210 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMRPKRQIFLDQNMLAVIDELMQALNFNSETVPQKSSLEEPDFYKTKIKLCILLHAFRIRAVTIDRVMSYLNAS

SEQ ID NO: 211 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMSPKRQIFLDQNMLAVIDELMQALNFNSETVPQKSSLEEPDFYKTKIKLCILLHAFRIRAVTIDRVMSYLNAS

SEQ ID NO: 212 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMTPKRQIFLDQNMLAVIDELMQALNFNSETVPQKSSLEEPDFYKTKIKLCILLHAFRIRAVTIDRVMSYLNAS

SEQ ID NO: 213 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMVPKRQIFLDQNMLAVIDELMQALNFNSETVPQKSSLEEPDFYKTKIKLCILLHAFRIRAVTIDRVMSYLNAS

SEQ ID NO: 214 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMWPKRQIFLDQNMLAVIDELMQALNFNSETVPQKSSLEEPDFYKTKIKLCILLHAFRIRAVTIDRVMSYLNAS

SEQ ID NO: 215 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKCQIFLDQNMLAVIDELMQALNFNSETVPQKSSLEEPDFYKTKIKLCILLHAFRIRAVTIDRVMSYLNAS

SEQ ID NO: 216 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKDQIFLDQNMLAVIDELMQALNFNSETVPQKSSLEEPDFYKTKIKLCILLHAFRIRAVTIDRVMSYLNAS

SEQ ID NO: 217 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKEQIFLDQNMLAVIDELMQALNFNSETVPQKSSLEEPDFYKTKIKLCILLHAFRIRAVTIDRVMSYLNAS

SEQ ID NO: 218 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKFQIFLDQNMLAVIDELMQALNFNSETVPQKSSLEEPDFYKTKIKLCILLHAFRIRAVTIDRVMSYLNAS

SEQ ID NO: 219 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKGQIFLDQNMLAVIDELMQALNFNSETVPQKSSLEEPDFYKTKIKLCILLHAFRIRAVTIDRVMSYLNAS

SEQ ID NO: 220 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKHQIFLDQNMLAVIDELMQALNFNSETVPQKSSLEEPDFYKTKIKLCILLHAFRIRAVTIDRVMSYLNAS

SEQ ID NO: 221 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKIQIFLDQNMLAVIDELMQALNFNSETVPQKSSLEEPDFYKTKIKLCILLHAFRIRAVTIDRVMSYLNAS

SEQ ID NO: 222 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKKQIFLDQNMLAVIDELMQALNFNSETVPQKSSLEEPDFYKTKIKLCILLHAFRIRAVTIDRVMSYLNAS

SEQ ID NO: 223 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKLQIFLDQNMLAVIDELMQALNFNSETVPQKSSLEEPDFYKTKIKLCILLHAFRIRAVTIDRVMSYLNAS

SEQ ID NO: 224 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKMQIFLDQNMLAVIDELMQALNFNSETVPQKSSLEEPDFYKTKIKLCILLHAFRIRAVTIDRVMSYLNAS

SEQ ID NO: 225 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKNQIFLDQNMLAVIDELMQALNFNSETVPQKSSLEEPDFYKTKIKLCILLHAFRIRAVTIDRVMSYLNAS

SEQ ID NO: 226 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKPQIFLDQNMLAVIDELMQALNFNSETVPQKSSLEEPDFYKTKIKLCILLHAFRIRAVTIDRVMSYLNAS

SEQ ID NO: 227 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKSQIFLDQNMLAVIDELMQALNFNSETVPQKSSLEEPDFYKTKIKLCILLHAFRIRAVTIDRVMSYLNAS

SEQ ID NO: 228 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKTQIFLDQNMLAVIDELMQALNFNSETVPQKSSLEEPDFYKTKIKLCILLHAFRIRAVTIDRVMSYLNAS

SEQ ID NO: 229 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKVQIFLDQNMLAVIDELMQALNFNSETVPQKSSLEEPDFYKTKIKLCILLHAFRIRAVTIDRVMSYLNAS

SEQ ID NO: 230 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKWQIFLDQNMLAVIDELMQALNFNSETVPQKSSLEEPDFYKTKIKLCILLHAFRIRAVTIDRVMSYLNAS

SEQ ID NO: 231 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKYQIFLDQNMLAVIDELMQALNFNSETVPQKSSLEEPDFYKTKIKLCILLHAFRIRAVTIDRVMSYLNAS

SEQ ID NO: 232 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYCTKIKLCILLHAFRIRAVTIDRVMSYLNAS

SEQ ID NO: 233 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNAS

SEQ ID NO: 234 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNAS

SEQ ID NO: 235 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYFTKIKLCILLHAFRIRAVTIDRVMSYLNAS

SEQ ID NO: 236 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYGTKIKLCILLHAFRIRAVTIDRVMSYLNAS

SEQ ID NO: 237 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYHTKIKLCILLHAFRIRAVTIDRVMSYLNAS

SEQ ID NO: 238 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITKIKLCILLHAFRIRAVTIDRVMSYLNAS

SEQ ID NO: 239 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYLTKIKLCILLHAFRIRAVTIDRVMSYLNAS

SEQ ID NO: 240 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYMTKIKLCILLHAFRIRAVTIDRVMSYLNAS

SEQ ID NO: 241 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYNTKIKLCILLHAFRIRAVTIDRVMSYLNAS

SEQ ID NO: 242 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYPTKIKLCILLHAFRIRAVTIDRVMSYLNAS

SEQ ID NO: 243 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYQTKIKLCILLHAFRIRAVTIDRVMSYLNAS

SEQ ID NO: 244 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYSTKIKLCILLHAFRIRAVTIDRVMSYLNAS

SEQ ID NO: 245 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTKIKLCILLHAFRIRAVTIDRVMSYLNAS

SEQ ID NO: 246 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYWTKIKLCILLHAFRIRAVTIDRVMSYLNAS

SEQ ID NO: 247 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYYTKIKLCILLHAFRIRAVTIDRVMSYLNAS

SEQ ID NO: 248 - protein, 197 aa, Synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTLIKLCILLHAFRIRAVTIDRVMSYLNAS

SEQ ID NO: 249 - protein, 197 aa, Synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTTIKLCILLHAFRIRAVTIDRVMSYLNAS

SEQ ID NO: 250 - protein, 197 aa, Synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 251 - protein, 197 aa, Synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITKIKLCILLHAFRIRAVTIDRVMSYLNAS

SEQ ID NO: 252 - protein, 197 aa, Synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTKIKLCILLHAFRIRAVTIDRVMSYLNAS

SEQ ID NO: 253 - protein, 197 aa, Synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 254 - protein, 197 aa, Synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 255 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITKIKLCILLHAFRIRAVTIDRVMSYLNAS

SEQ ID NO: 256 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTKIKLCILLHAFRIRAVTIDRVMSYLNAS

SEQ ID NO: 257 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 258 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 259 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNAS

SEQ ID NO: 260 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITKIKLCILLHAFRIRAVTIDRVMSYLNAS

SEQ ID NO: 261 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTKIKLCILLHAFRIRAVTIDRVMSYLNAS

SEQ ID NO: 262 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITKIKLCILLHAFRIRAVTIDRVMSYLNAS

SEQ ID NO: 263 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTKIKLCILLHAFRIRAVTIDRVMSYLNAS

SEQ ID NO: 264 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 265 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTLIKLCILLHAFRIRAVTIDRVMSYLNAS

SEQ ID NO: 266 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTTIKLCILLHAFRIRAVTIDRVMSYLNAS

SEQ ID NO: 267 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKAQIFLDQNMLAVIDELMQALNFNSETVPQKSSLEEPDFYKTKIKLCILLHAFRIRAVTIDRVMSYLNAS

SEQ ID NO: 268 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITAIKLCILLHAFRIRAVTIDRVMSYLNAS

SEQ ID NO: 269 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTAIKLCILLHAFRIRAVTIDRVMSYLNAS

SEQ ID NO: 270 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKEQIFLDQNMLAVIDELMQALNFNSETVPQKSSLEEPDFYKTAIKLCILLHAFRIRAVTIDRVMSYLNAS

SEQ ID NO: 271 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATPIKLCILLHAFRIRAVTIDRVMSYLNAS

SEQ ID NO: 272 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITPIKLCILLHAFRIRAVTIDRVMSYLNAS

SEQ ID NO: 273 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTPIKLCILLHAFRIRAVTIDRVMSYLNAS

SEQ ID NO: 274 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKEQIFLDQNMLAVIDELMQALNFNSETVPQKSSLEEPDFYKTPIKLCILLHAFRIRAVTIDRVMSYLNAS

SEQ ID NO: 275 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATTIKLCILLHAFRIRAVTIDRVMSYLNAS

SEQ ID NO: 276 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYITTIKLCILLHAFRIRAVTIDRVMSYLNAS

SEQ ID NO: 277 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYTTTIKLCILLHAFRIRAVTIDRVMSYLNAS

SEQ ID NO: 278 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKEQIFLDQNMLAVIDELMQALNFNSETVPQKSSLEEPDFYKTTIKLCILLHAFRIRAVTIDRVMSYLNAS

SEQ ID NO: 279 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDCKRQIFLDQNMLAVIDELMQALNFNSETVPQKSSLEEPDFYKTKIKLCILLHAFRIRAVTIDRVMSYLNAS

SEQ ID NO: 280 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDDKRQIFLDQNMLAVIDELMQALNFNSETVPQKSSLEEPDFYKTKIKLCILLHAFRIRAVTIDRVMSYLNAS

SEQ ID NO: 281 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDEKRQIFLDQNMLAVIDELMQALNFNSETVPQKSSLEEPDFYKTKIKLCILLHAFRIRAVTIDRVMSYLNAS

SEQ ID NO: 282 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDFKRQIFLDQNMLAVIDELMQALNFNSETVPQKSSLEEPDFYKTKIKLCILLHAFRIRAVTIDRVMSYLNAS

SEQ ID NO: 283 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDGKRQIFLDQNMLAVIDELMQALNFNSETVPQKSSLEEPDFYKTKIKLCILLHAFRIRAVTIDRVMSYLNAS

SEQ ID NO: 284 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDHKRQIFLDQNMLAVIDELMQALNFNSETVPQKSSLEEPDFYKTKIKLCILLHAFRIRAVTIDRVMSYLNAS

SEQ ID NO: 285 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDKKRQIFLDQNMLAVIDELMQALNFNSETVPQKSSLEEPDFYKTKIKLCILLHAFRIRAVTIDRVMSYLNAS

SEQ ID NO: 286 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDMKRQIFLDQNMLAVIDELMQALNFNSETVPQKSSLEEPDFYKTKIKLCILLHAFRIRAVTIDRVMSYLNAS

SEQ ID NO: 287 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDNKRQIFLDQNMLAVIDELMQALNFNSETVPQKSSLEEPDFYKTKIKLCILLHAFRIRAVTIDRVMSYLNAS

SEQ ID NO: 288 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDQKRQIFLDQNMLAVIDELMQALNFNSETVPQKSSLEEPDFYKTKIKLCILLHAFRIRAVTIDRVMSYLNAS

SEQ ID NO: 289 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDRKRQIFLDQNMLAVIDELMQALNFNSETVPQKSSLEEPDFYKTKIKLCILLHAFRIRAVTIDRVMSYLNAS

SEQ ID NO: 290 - protein, 197 aa, synthetic construct
RNLPVATPDPGMFPCLHHSQNLLRAVSNMLQKARQTLEFAPCTSEEIDHEDITKDKTSTVEACLPLELTKNESCLNSRETSFITNGSCLASRKTSFMMALCLSSIYEDLKMYQVEFKTMNAKLLMDSKRQIFLDQNMLAVIDELMQALNFNSETVPQKSSLEEPDFYKTKIKLCILLHAFRIRAVTIDRVMSYLNAS

SEQ ID NO: 291 - protein, 513 aa, synthetic construct
MGILPSPGMPALLSLVSLLSVLLMGCVAESLLAWITQVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 292 - protein, 612 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 293 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDCKRQIFLDQNMLAVIDELMQALNFNSETVPQKSSLEEPDFYKTKIKLCILLHAFRIRAVTIDRVMSYLNAS

SEQ ID NO: 294 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDDKRQIFLDQNMLAVIDELMQALNFNSETVPQKSSLEEPDFYKTKIKLCILLHAFRIRAVTIDRVMSYLNAS

SEQ ID NO: 295 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDEKRQIFLDQNMLAVIDELMQALNFNSETVPQKSSLEEPDFYKTKIKLCILLHAFRIRAVTIDRVMSYLNAS

SEQ ID NO: 296 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDFKRQIFLDQNMLAVIDELMQALNFNSETVPQKSSLEEPDFYKTKIKLCILLHAFRIRAVTIDRVMSYLNAS

SEQ ID NO: 297 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDGKRQIFLDQNMLAVIDELMQALNFNSETVPQKSSLEEPDFYKTKIKLCILLHAFRIRAVTIDRVMSYLNAS

SEQ ID NO: 298 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDHKRQIFLDQNMLAVIDELMQALNFNSETVPQKSSLEEPDFYKTKIKLCILLHAFRIRAVTIDRVMSYLNAS

SEQ ID NO: 299 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDKKRQIFLDQNMLAVIDELMQALNFNSETVPQKSSLEEPDFYKTKIKLCILLHAFRIRAVTIDRVMSYLNAS

SEQ ID NO: 300 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDMKRQIFLDQNMLAVIDELMQALNFNSETVPQKSSLEEPDFYKTKIKLCILLHAFRIRAVTIDRVMSYLNAS

SEQ ID NO: 301 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDNKRQIFLDQNMLAVIDELMQALNFNSETVPQKSSLEEPDFYKTKIKLCILLHAFRIRAVTIDRVMSYLNAS

SEQ ID NO: 302 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDQKRQIFLDQNMLAVIDELMQALNFNSETVPQKSSLEEPDFYKTKIKLCILLHAFRIRAVTIDRVMSYLNAS

SEQ ID NO: 303 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDRKRQIFLDQNMLAVIDELMQALNFNSETVPQKSSLEEPDFYKTKIKLCILLHAFRIRAVTIDRVMSYLNAS

SEQ ID NO: 304 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDSKRQIFLDQNMLAVIDELMQALNFNSETVPQKSSLEEPDFYKTKIKLCILLHAFRIRAVTIDRVMSYLNAS

SEQ ID NO: 305 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKQQIFLDQNMLAVIDELMQALNFNSETVPQKSSLEEPDFYKTKIKLCILLHAFRIRAVTIDRVMSYLNAS

SEQ ID NO: 306 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTFIKLCILLHAFRIRAVTIDRVMSYLNAS

SEQ ID NO: 307 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTLIKLCILLHAFRIRAVTIDRVMSYLNAS

SEQ ID NO: 308 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTNIKLCILLHAFRIRAVTIDRVMSYLNAS

SEQ ID NO: 309 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 310 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 311 - protein, 197 aa, synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

(19) United States
(12) Patent Application Publication
(10) Pub. No.: US 2024/0150422 A1
(43) Pub. Date: May 9, 2024

(54) IL-12 FC FUSION PROTEINS AND USES THEREOF

(71) Applicant: oNKo-Innate Pty Ltd, Moonee Ponds (AU)

(72) Inventor: Richard BERRY, Mordialloc (AU)

(21) Appl. No.: 18/353,729

(22) Filed: Jul. 17, 2023

Related U.S. Application Data
(60) Provisional application No. 63/476,687, filed on Dec. 22, 2022.
(60) Provisional application No. 63/368,740, filed on Jul. 18, 2022.

(51) Int. Cl. C07K 14/54; A61K 38/00; A61P 35/00

(52) U.S. Cl. CPC C07K 14/5434; A61P 35/00; A61K 38/00; C07K 2319/30

(57) ABSTRACT

The present invention is directed to compositions of novel, non-naturally occurring IL-12 variants, homodimeric IL-12 Fc fusion proteins, and heterodimeric IL-12 Fc fusion proteins, as well as methods of making and using such compositions.

### CROSS-REFERENCE TO RELATED APPLICATIONS

This application claims priority to U.S. Provisional Application No. 63/476,687, filed Dec. 22, 2022, and to U.S. Provisional Application No. 63/368,740, filed Jul. 18, 2022, each of which is entirely incorporated herein by reference.

### SEQUENCE LISTING

The instant application contains a Sequence Listing which has been submitted electronically in XML format and is hereby incorporated by reference in its entirety. The XML copy, created on Jul. 17, 2023, is named 51400_4001.xml and is 336,708 bytes in size.

### BACKGROUND OF THE INVENTION

The present application generally relates to compositions and methods for modulating signal transduction mediated by interleukin 12 (IL-12). In particular, the disclosure provides novel, non-naturally occurring IL-12 variant polypeptides and fusion proteins, wherein the IL-12p35 subunit comprises novel amino acid substitutions that reduce binding affinity to interleukin 12 receptor beta 2 (IL-12Rβ2), as well as methods of making and using the non-naturally occurring IL-12 variant polypeptides and fusion proteins.

IL-12 is a potent, pro-inflammatory cytokine that is produced by antigen presenting cells, such as, for example, dendritic cells, macrophages, and neutrophils. IL-12 belongs to the IL-12 family of cytokines. The IL-12 family of cytokines is unique in that they comprise heterodimeric cytokines. IL-12 is comprised of an alpha-(α-) subunit (encoded by the IL12A gene; also referred to herein as an “IL-12p35 subunit”; Precursor Sequence—SEQ ID NO: 1 (as shown in FIG. 1); Mature Sequence—SEQ ID NO: 2 (as shown in FIG. 1)) and a beta-(β-) subunit (encoded by the IL12B gene; also referred to herein as an “IL-12p40 subunit”; Precursor Sequence—SEQ ID NO: 3 (as shown in FIG. 1); Mature Sequence—SEQ ID NO: 4 (as shown in FIG. 1) that assemble to form a 70,000 Dalton (70 kDa) disulfide-linked heterodimer. Upon assembly of the IL-12p35 and p40 subunits, a biologically active IL-12 heterodimer is formed. The receptor for IL-12, IL-12R, is a type I cytokine receptor comprising a beta-1 subunit (IL-12Rβ1—SEQ ID NO: 5 (as shown in FIG. 2); Extracellular Domain—SEQ ID NO: 6 (as shown in FIG. 2)) and a beta-2 subunit (IL-12Rβ2—SEQ ID NO: 7 (as shown in FIG. 2); Extracellular Domain—SEQ ID NO: 8 (as shown in FIG. 2)). The IL-12p40 subunit has a binding affinity for IL-12Rβ1, and the IL-12p35 subunit has a binding affinity for IL-12Rβ2.

The binding of IL-12 to IL-12R results in the phosphorylation of intracellular Signal Transducer And Activator Of Transcription 4 (STAT4) and triggers signaling pathways that: (i) induce TH1 cell differentiation, (ii) increase the activation and cytotoxic capacities of T- and natural killer (NK) cells, (iii) inhibit or reprogram immunosuppressive cells, such as, for example, tumor-associated macrophages (TAMs) and myeloid-derived suppressor cells (MDSCs), and (iv) induce the production of large amounts of interferon gamma (IFNγ), which is cytostatic/cytotoxic, anti-angiogenic, and can upregulate major histocompatibility complex (MHC) class 1 and MHC class 2 molecules on tumor cells to enable immune recognition.

Accordingly, IL-12 has demonstrated potent antitumor activity against a range of malignancies in preclinical studies. However, systemic administration of wild-type IL-12 in humans can result in severe toxicity, including in-trial fatalities, due to an over-activation of circulating immune cells. Moreover, the activated immune cells undergo cellular proliferation, contributing to a short serum half-life of administered IL-12 due to target-mediated drug disposition. The biologically active form of human IL-12 (i.e., the heterodimeric complex comprising the IL-12p35 subunit and the IL-12p40 subunit) has a reported in vivo half-life as low as 5 hours when administered as a therapeutic compound. In recent years, cytokine engineering has emerged as a promising strategy to tailor cytokines with desired activities and reduced toxicity. Hence, there is a need for additional approaches to improve properties of IL-12 for use a therapeutic agent. In particular, there is an unmet need for novel IL-12 variant polypeptides and/or fusion proteins that (i) bind to IL-12R with a modified binding efficiency or affinity, such that the therapeutic qualities of IL-12 are maintained (e.g., recognition and elimination of target cells, such as, for example, cancer cells) and the negative side-effects of IL-12 are reduced or eliminated, and (ii) increase serum half-life.

### BRIEF SUMMARY OF THE INVENTION

In one aspect, the present disclosure provides a non-naturally occurring IL-12 variant, comprising: a) a variant IL-12p35 subunit, wherein the variant IL-12p35 subunit comprises one or more amino acid substitutions selected from the group including: Y40A, T43A, D126A, P127A, R129A, K168A, and K170A; and b) an IL-12p40 subunit.

In a further embodiment and in accordance with the above, the variant IL-12p35 subunit comprises two or more amino acid substitutions selected from the group including: Y40A, T43A, D126A, P127A, R129A, K168A, and K170A.

In a further embodiment and in accordance with the above, the variant IL-12p35 subunit comprises three or more amino acid substitutions selected from the group including: Y40A, T43A, D126A, P127A, R129A, K168A, and K170A.

In a further embodiment and in accordance with the above, the variant IL-12p35 subunit comprises four or more amino acid substitutions selected from the group including: Y40A, T43A, D126A, P127A, R129A, K168A, and K170A.

In a further embodiment and in accordance with the above, the variant IL-12p35 subunit comprises five or more amino acid substitutions selected from the group including: Y40A, T43A, D126A, P127A, R129A, K168A, and K170A.

In a further embodiment and in accordance with the above, the variant IL-12p35 subunit comprises six or more amino acid substitutions selected from the group including: Y40A, T43A, D126A, P127A, R129A, K168A, and K170A.

In a further embodiment and in accordance with the above, the variant IL-12p35 subunit comprises seven or more amino acid substitutions selected from the group including: Y40A, T43A, D126A, P127A, R129A, K168A, and K170A.

In a further embodiment and in accordance with the above, the variant IL-12p35 subunit comprises amino acid substitutions Y40A and D126A.

In a further embodiment and in accordance with the above, the variant IL-12p35 subunit comprises amino acid substitutions Y40A and P127A.

In a further embodiment and in accordance with the above, the variant IL-12p35 subunit comprises amino acid substitutions Y40A and T43A.

In a further embodiment and in accordance with the above, the variant IL-12p35 subunit comprises amino acid substitutions Y40A, D126A, and P127A.

In a further embodiment and in accordance with the above, the variant IL-12p35 subunit comprises amino acid substitutions Y40A, T43A, D126A, and P127A.

In a further embodiment and in accordance with the above, the variant IL-12p35 subunit comprises amino acid substitutions Y40A and R129A.

In a further embodiment and in accordance with the above, the variant IL-12p35 subunit comprises amino acid substitutions Y40A and K168A.

In a further embodiment and in accordance with the above, the variant IL-12p35 subunit comprises amino acid substitutions Y40A and K170A.

In a further embodiment and in accordance with the above, the variant IL-12p35 subunit comprises amino acid substitutions Y40A, P127A, and R129A.

In a further embodiment and in accordance with the above, the variant IL-12p35 subunit comprises amino acid substitutions Y40A, P127A, and K168A.

In a further embodiment and in accordance with the above, the variant IL-12p35 subunit comprises amino acid substitutions Y40A, P127A, and K170A.

In a further embodiment and in accordance with any of the above, the variant IL-12p35 subunit comprises a substitution mutation at amino acid residue Y40. In some further embodiments, the substitution mutation at amino acid residue Y40 is selected from the group including: Y40C, Y40D, Y40E, Y40G, Y40K, Y40N, Y40P, Y40Q, Y40R, Y40S, and Y40T.

In a further embodiment and in accordance with the above, the variant IL-12p35 subunit comprises any one of SEQ ID NOs: 177-187.

In a further embodiment and in accordance with any of the above, the variant IL-12p35 subunit comprises a substitution mutation at amino acid residue D126. In some further embodiments, the substitution mutation at amino acid residue D126 is selected from the group including: D126C, D126E, D126F, D126G, D1261, D126K, D126L, D126M, D126N, D126P, D126Q, D126R, D126S, D126T, D126V, and D126W.

In a further embodiment and in accordance with any of the above, the variant IL-12p35 subunit comprises a substitution mutation at amino acid residue P127. In some further embodiments, the substitution mutation at amino acid residue P127 is selected from the group including: P127C, P127D, P127E, P127F, P127G, P127H, P127K, P127M, P127N, P127Q, P127R, and P127S.

In a further embodiment and in accordance with any of the above, the variant IL-12p35 subunit comprises a substitution mutation at amino acid residue R129. In some further embodiments, the substitution mutation at amino acid residue R129 is selected from the group including: R129C, R129D, R129E, R129F, R129G, R129H, R129I, R129K, R129L, R129M, R129N, R129P, R129Q, R129S, R129T, R129V, R129W, and R129Y.

In a further embodiment and in accordance with any of the above, the variant IL-12p35 subunit comprises a substitution mutation at amino acid residue K168. In some further embodiments, the substitution mutation at amino acid residue K168 is selected from the group including: K168C, K168D, K168E, K168F, K168G, K168H, K168I, K168L, K168M, K168N, K168P, K168Q, K168S, K168T, K168W, and K168Y.

In a further embodiment and in accordance with any of the above, the variant IL-12p35 subunit comprises a substitution mutation at amino acid residue K170. In some further embodiments, the substitution mutation at amino acid residue K170 is selected from the group including: K170C, K170D, K170E, K170G, K170I, K170M, K170P, K170S, K170T, K170V, and K170W.

In a further embodiment and in accordance with any of the above, the variant IL-12p35 subunit comprises a first substitution mutation selected from the group including: Y40A, Y40C, Y40D, Y40E, Y40G, Y40K, Y40N, Y40P, Y40Q, Y40R, Y40S, and Y40T.

In a further embodiment and in accordance with the above, the variant IL-12p35 subunit further comprises a second substitution mutation.

In a further embodiment and in accordance with the above, the second substitution mutation is selected from the group including: D126A, D126C, D126E, D126F, D126G, D1261, D126K, D126L, D126M, D126N, D126P, D126Q, D126R, D126S, D126T, D126V, D126W, P127A, P127C, P127D, P127E, P127F, P127G, P127H, P127K, P127M, P127N, P127Q, P127R, P127S, R129A, R129C, R129D, R129E, R129F, R129G, R129H, R129I, R129K, R129L, R129M, R129N, R129P, R129Q, R129S, R129T, R129V, R129W, R129Y, K168A, K168C, K168D, K168E, K168F, K168G, K168H, K168I, K168L, K168M, K168N, K168P, K168Q, K168S, K168T, K168W, K168Y, K170A, K170C, K170D, K170E, K170G, K170I, K170M, K170P, K170S, K170T, K170V, and K170W.

In a further embodiment and in accordance with any of the above, the variant IL-12p35 subunit comprises any one of SEQ ID NOs: 199-247 or SEQ ID NOs: 279-290.

In a further embodiment and in accordance with any of the above, the variant IL-12p35 subunit may further comprise a C74S substitution mutation.

In a further embodiment and in accordance with any of the above, the IL-12p40 subunit comprises a variant IL-12p40 subunit.

In a further embodiment and in accordance with the above, the variant IL-12p40 subunit comprises one or more amino acid substitutions selected from the group including: C177S, C252S, and C177S/C252S.

In a further embodiment and in accordance with any of the above, the variant IL-12p35 subunit comprises an amino acid sequence selected from the group including: SEQ ID NOs: 24-86 and SEQ ID NOs: 103-166, and the IL-12p40 subunit comprises an amino acid sequence selected from the group including: SEQ ID NO: 3, SEQ ID NO: 4, SEQ ID NO: 88, SEQ ID NO: 89, and SEQ ID NO: 90.

In a further embodiment and in accordance with the above, the variant IL-12p35 subunit comprises an amino acid sequence with at least 95% sequence identity to any one of SEQ ID NO: 1, SEQ ID NO: 2, SEQ ID NOs: 24-87, or SEQ ID NOs: 103-166, and the IL-12p40 subunit comprises an amino acid sequence with at least 95% sequence identity to SEQ ID NO: 3, SEQ ID NO: 4, SEQ ID NO: 88, SEQ ID NO: 89, or SEQ ID NO: 90.

In a further embodiment and in accordance with the above, the variant IL-12p35 subunit comprises an amino acid sequence with at least 96% sequence identity to any one of SEQ ID NO: 1, SEQ ID NO: 2, SEQ ID NOs: 24-87, or SEQ ID NOs: 103-166, and the IL-12p40 subunit comprises an amino acid sequence with at least 96% sequence identity to SEQ ID NO: 3, SEQ ID NO: 4, SEQ ID NO: 88, SEQ ID NO: 89, or SEQ ID NO: 90.

In a further embodiment and in accordance with the above, the variant IL-12p35 subunit comprises an amino acid sequence with at least 97% sequence identity to any one of SEQ ID NO: 1, SEQ ID NO: 2, SEQ ID NOs: 24-87, or SEQ ID NOs: 103-166, and the IL-12p40 subunit comprises an amino acid sequence with at least 97% sequence identity to SEQ ID NO: 3, SEQ ID NO: 4, SEQ ID NO: 88, SEQ ID NO: 89, or SEQ ID NO: 90.

In a further embodiment and in accordance with the above, the variant IL-12p35 subunit comprises an amino acid sequence with at least 98% sequence identity to any one of SEQ ID NO: 1, SEQ ID NO: 2, SEQ ID NOs: 24-87, or SEQ ID NOs: 103-166, and the IL-12p40 subunit comprises an amino acid sequence with at least 98% sequence identity to SEQ ID NO: 3, SEQ ID NO: 4, SEQ ID NO: 88, SEQ ID NO: 89, or SEQ ID NO: 90.

In a further embodiment and in accordance with the above, the variant IL-12p35 subunit comprises an amino acid sequence with at least 99% sequence identity to any one of SEQ ID NO: 1, SEQ ID NO: 2, SEQ ID NOs: 24-87, or SEQ ID NOs: 103-166, and the IL-12p40 subunit comprises an amino acid sequence with at least 99% sequence identity to SEQ ID NO: 3, SEQ ID NO: 4, SEQ ID NO: 88, SEQ ID NO: 89, or SEQ ID NO: 90.

In a further embodiment and in accordance with the above, the variant IL-12p35 subunit consists of an amino acid sequence selected from a group including: SEQ ID NOs: 24-86 and SEQ ID NOs: 103-166, and the IL-12p40 subunit consists of an amino acid sequence selected from a group including: SEQ ID NO: 3, SEQ ID NO: 4, SEQ ID NO: 88, SEQ ID NO: 89, and SEQ ID NO: 90.

In a further embodiment and in accordance with any of the above, the variant IL-12p35 subunit comprises SEQ ID NO: 87, and further comprises one, two, three, four, five, six, or all seven amino acid substitutions selected from the group including: Y40A, T43A, D126A, P127A, R129A, K168A, and K170A.

In a further embodiment and in accordance with any of the above, the one or more amino acid substitutions of the variant IL-12p35 subunit improves half-life, as compared to half-life of a reference IL-12, wherein the reference IL-12 comprises one or more of: a wild-type IL-12, a human wild-type IL-12, a commercially available IL-12 molecule, or an IL-12 Fc fusion protein.

In a further embodiment and in accordance with any of the above, the non-naturally occurring IL-12 variant further comprises one or more of the following fused to the variant IL-12p35 subunit and/or the IL-12p40 subunit: (i) a Fc domain, wherein the Fc domain comprises one or more amino acid sequences selected from the group including: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

In a further embodiment and in accordance with any of the above, wherein the C-terminus of the variant IL-12p35 subunit is covalently attached to the N-terminus of the IL-12p40 subunit.

In a further embodiment and in accordance with the above, the non-naturally occurring IL-12 variant further comprises a linker comprising an amino acid sequence selected from the group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the variant IL-12p35 subunit is covalently attached to the N-terminus of the linker domain and the C-terminus of the linker domain is covalently attached to the N-terminus of the IL-12p40 subunit.

In a further embodiment in accordance with any of the above, the C-terminus of the IL-12p40 subunit is covalently attached to the N-terminus of the variant IL-12p35 subunit.

In a further embodiment and in accordance with the above, the non-naturally occurring IL-12 variant further comprises a linker domain comprising an amino acid sequence selected from the group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the IL-12p40 subunit is covalently attached to the N-terminus of the linker domain and the C-terminus of the linker domain is covalently attached to the N-terminus of the variant IL-12p35 subunit.

In a further embodiment and in accordance with any of the above, the variant IL-12p35 subunit comprises additional amino acid substitutions.

In a further embodiment and in accordance with any of the above, the non-naturally occurring IL-12 variant is provided for use in treating cancer in a subject.

In another aspect, the present disclosure provides one or more nucleic acids encoding a non-naturally occurring IL-12 variant according to any of the aspects and embodiments above.

In another aspect, the present disclosure provides a host cell comprising the one or more nucleic acids encoding a non-naturally occurring IL-12 variant according to any of the aspects and embodiments above.

In another aspect, the present disclosure provides a method of producing a non-naturally occurring IL-12 variant, the method comprising: culturing a host cell with one or more nucleic acids or vectors under conditions whereby the non-naturally occurring IL-12 variant is produced, wherein the one or more nucleic acids or vectors comprises the one or more nucleic acids described in the aspects and embodiments above.

In a further embodiment and in accordance with the above, the method further comprises isolating and/or purifying the produced non-naturally occurring IL-12 variant.

In a further embodiment and in accordance with any of the above, the non-naturally occurring IL-12 variant further comprises one or more of the following fused to the variant IL-12p35 subunit and/or the IL-12p40 subunit: (i) a Fc domain, wherein the Fc domain comprises one or more amino acid sequences selected from the group including: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

In a further embodiment and in accordance with any of the above, the produced non-naturally occurring IL-12 variant has an altered binding affinity for Interleukin-12 Receptor Beta 2 (IL-12Rβ2) compared to binding affinity of a reference IL-12, wherein the reference IL-12 comprises one or more of: a wild-type IL-12, a human wild-type IL-12, a commercially available IL-12 molecule, or an IL-12 Fc fusion protein.

In a further embodiment and in accordance with the above, the produced non-naturally occurring IL-12 variant has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay.

In a further embodiment and in accordance with the above, the assay comprises an SPR assay.

In a further embodiment and in accordance with any of the above, the produced non-naturally occurring IL-12 variant has binding affinity for IL-12Rβ2 below the lower level of detectability of an assay, and binding affinity of a reference IL-12 is detectable, as determined by an assay.

In a further embodiment and in accordance with the above, the assay comprises an SPR assay.

In a further embodiment and in accordance with any of the above, the produced non-naturally occurring IL-12 variant has potency reduced by about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12, wherein the reference IL-12 comprises one or more of: a wild-type IL-12, a human wild-type IL-12, a commercially available IL-12 molecule, or an IL-12 Fc fusion protein, as determined by an assay.

In a further embodiment and in accordance with the above, the assay comprises an IL-12 HEK reporter assay.

In a further embodiment and in accordance with any of the above, the produced non-naturally occurring IL-12 variant has a reduced capability to stimulate IFNγ production by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to capability to stimulate IFNγ production of a reference IL-12, wherein the reference IL-12 comprises one or more of: a wild-type IL-12, a human wild-type IL-12, a commercially available IL-12 molecule, or an IL-12 Fc fusion protein, as determined by an assay.

In a further embodiment and in accordance with the above, the assay comprises one or more of the following: (i) an intracellular cytokine stain assay, (ii) a Luminex bead-based cytokine release assay, (iii) an ELISA, or (iv) an ELISpot assay.

In another aspect, the present disclosure provides a heterodimeric Fc fusion protein comprising: a) a first fusion construct, comprising: a variant IL-12p35 subunit domain and a first Fc domain, wherein the C-terminus of the variant IL-12p35 subunit domain is covalently attached to the N-terminus of the first Fc domain; and b) a second fusion construct, comprising: an IL-12p40 subunit domain and a second Fc domain, wherein the C-terminus of the IL-12p40 subunit domain is covalently attached to the N-terminus of the second Fc domain; optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain comprises one or more amino acid substitutions selected from the group including: Y40A, T43A, D126A, P127A, R129A, K168A, and K170A.

In a further embodiment and in accordance with the above, the variant IL-12p35 subunit domain comprises two or more amino acid substitutions selected from the group including: Y40A, T43A, D126A, P127A, R129A, K168A, and K170A.

In a further embodiment and in accordance with the above, the variant IL-12p35 subunit domain comprises three or more amino acid substitutions selected from the group including: Y40A, T43A, D126A, P127A, R129A, K168A, and K170A.

In a further embodiment and in accordance with the above, the variant IL-12p35 subunit domain comprises four or more amino acid substitutions selected from the group including: Y40A, T43A, D126A, P127A, R129A, K168A, and K170A.

In a further embodiment and in accordance with the above, the variant IL-12p35 subunit domain comprises five or more amino acid substitutions selected from the group including: Y40A, T43A, D126A, P127A, R129A, K168A, and K170A.

In a further embodiment and in accordance with the above, the variant IL-12p35 subunit domain comprises six or more amino acid substitutions selected from the group including: Y40A, T43A, D126A, P127A, R129A, K168A, and K170A.

In a further embodiment and in accordance with the above, the variant IL-12p35 subunit domain comprises seven or more amino acid substitutions selected from the group including: Y40A, T43A, D126A, P127A, R129A, K168A, and K170A.

In a further embodiment and in accordance with the above, the variant IL-12p35 subunit domain comprises amino acid substitutions Y40A and D126A.

In a further embodiment and in accordance with the above, the variant IL-12p35 subunit domain comprises amino acid substitutions Y40A and P127A.

In a further embodiment and in accordance with the above, the variant IL-12p35 subunit domain comprises amino acid substitutions Y40A and T43A.

In a further embodiment and in accordance with the above, the variant IL-12p35 subunit domain comprises amino acid substitutions Y40A, D126A, and P127A.

In a further embodiment and in accordance with the above, the variant IL-12p35 subunit domain comprises amino acid substitutions Y40A, T43A, D126A, and P127A.

In a further embodiment and in accordance with the above, the variant IL-12p35 subunit domain comprises amino acid substitutions Y40A and R129A.

In a further embodiment and in accordance with the above, the variant IL-12p35 subunit domain comprises amino acid substitutions Y40A and K168A.

In a further embodiment and in accordance with the above, the variant IL-12p35 subunit domain comprises amino acid substitutions Y40A and K170A.

In a further embodiment and in accordance with the above, the variant IL-12p35 subunit domain comprises amino acid substitutions Y40A, P127A, and R129A.

In a further embodiment and in accordance with the above, the variant IL-12p35 subunit domain comprises amino acid substitutions Y40A, P127A, and K168A.

In a further embodiment and in accordance with the above, the variant IL-12p35 subunit domain comprises amino acid substitutions Y40A, P127A, and K170A.

In a further embodiment and in accordance with any of the above, the variant IL-12p35 subunit domain comprises a substitution mutation at amino acid residue Y40. In some further embodiments, the substitution mutation at amino acid residue Y40 is selected from the group including: Y40C, Y40D, Y40E, Y40G, Y40K, Y40N, Y40P, Y40Q, Y40R, Y40S, and Y40T.

In a further embodiment and in accordance with the above, the variant IL-12p35 subunit domain comprises any one of SEQ ID NOs: 177-187.

In a further embodiment and in accordance with any of the above, the variant IL-12p35 subunit domain comprises a substitution mutation at amino acid residue D126. In some further embodiments, the substitution mutation at amino acid residue D126 is selected from the group including: D126C, D126E, D126F, D126G, D1261, D126K, D126L, D126M, D126N, D126P, D126Q, D126R, D126S, D126T, D126V, and D126W.

In a further embodiment and in accordance with any of the above, the variant IL-12p35 subunit domain comprises a substitution mutation at amino acid residue P127. In some further embodiments, the substitution mutation at amino acid residue P127 is selected from the group including: P127C, P127D, P127E, P127F, P127G, P127H, P127K, P127M, P127N, P127Q, P127R, and P127S.

In a further embodiment and in accordance with any of the above, the variant IL-12p35 subunit domain comprises a substitution mutation at amino acid residue R129. In some further embodiments, the substitution mutation at amino acid residue R129 is selected from the group including: R129C, R129D, R129E, R129F, R129G, R129H, R129I, R129K, R129L, R129M, R129N, R129P, R129Q, R129S, R129T, R129V, R129W, and R129Y.

In a further embodiment and in accordance with any of the above, the variant IL-12p35 subunit domain comprises a substitution mutation at amino acid residue K168. In some further embodiments, the substitution mutation at amino acid residue K168 is selected from the group including: K168C, K168D, K168E, K168F, K168G, K168H, K168I, K168L, K168M, K168N, K168P, K168Q, K168S, K168T, K168W, and K168Y.

In a further embodiment and in accordance with any of the above, the variant IL-12p35 subunit domain comprises a substitution mutation at amino acid residue K170. In some further embodiments, the substitution mutation at amino acid residue K170 is selected from the group including: K170C, K170D, K170E, K170G, K170I, K170M, K170P, K170S, K170T, K170V, and K170W.

In a further embodiment and in accordance with any of the above, the variant IL-12p35 subunit domain comprises a first substitution mutation selected from the group including: Y40A, Y40C, Y40D, Y40E, Y40G, Y40K, Y40N, Y40P, Y40Q, Y40R, Y40S, and Y40T.

In a further embodiment and in accordance with the above, the variant IL-12p35 subunit domain further comprises a second substitution mutation.

In a further embodiment and in accordance with the above, the second substitution mutation is selected from the group including: D126A, D126C, D126E, D126F, D126G, D1261, D126K, D126L, D126M, D126N, D126P, D126Q, D126R, D126S, D126T, D126V, D126W, P127A, P127C, P127D, P127E, P127F, P127G, P127H, P127K, P127M, P127N, P127Q, P127R, P127S, R129A, R129C, R129D, R129E, R129F, R129G, R129H, R129I, R129K, R129L, R129M, R129N, R129P, R129Q, R129S, R129T, R129V, R129W, R129Y, K168A, K168C, K168D, K168E, K168F, K168G, K168H, K168I, K168L, K168M, K168N, K168P, K168Q, K168S, K168T, K168W, K168Y, K170A, K170C, K170D, K170E, K170G, K170I, K170M, K170P, K170S, K170T, K170V, and K170W.

In a further embodiment and in accordance with any of the above, the variant IL-12p35 subunit domain comprises any one of SEQ ID NOs: 199-247 or SEQ ID NOs: 279-290.

In a further embodiment and in accordance with any of the above, the variant IL-12p35 subunit domain may further comprise a C74S substitution mutation.

In a further embodiment and in accordance with any of the above, the IL-12p40 subunit domain comprises a variant IL-12p40 subunit domain.

In a further embodiment and in accordance with the above, the variant IL-12p40 subunit domain comprises one or more amino acid substitutions selected from the group including: C177S, C252S, and C177S/C252S.

In a further embodiment and in accordance with any of the above, the first Fc domain comprises an amino acid selected from the group including: SEQ ID NO: 9, SEQ ID NO: 10, SEQ ID NO: 11, SEQ ID NO: 12, and SEQ ID NO: 13, and the second Fc domain comprises an amino acid selected from the group including: SEQ ID NO: 9, SEQ ID NO: 10, SEQ ID NO: 11, SEQ ID NO: 12, and SEQ ID NO: 13.

In a further embodiment and in accordance with any of the above, the variant IL-12p35 subunit domain comprises an amino acid sequence selected from the group including: SEQ ID NOs: 24-86 and SEQ ID NOs: 103-166; the first Fc domain comprises an amino acid sequence selected from the group including: SEQ ID NO: 9, SEQ ID NO: 10, SEQ ID NO: 11, SEQ ID NO: 12, and SEQ ID NO: 13; the IL-12p40 subunit domain comprises an amino acid sequence selected from the group including: SEQ ID NO: 3, SEQ ID NO: 4, SEQ ID NO: 88, SEQ ID NO: 89, and SEQ ID NO: 90; and the second Fc domain comprises an amino acid sequence selected from the group including: SEQ ID NO: 9, SEQ ID NO: 10, SEQ ID NO: 11, SEQ ID NO: 12, and SEQ ID NO: 13.

In a further embodiment and in accordance with the above, the variant IL-12p35 subunit domain comprises an amino acid sequence with at least 95% sequence identity to any one of SEQ ID NO: 1, SEQ ID NO: 2, SEQ ID NOs: 24-87, or SEQ ID NOs: 103-166; the first Fc domain comprises an amino acid sequence with at least 95% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 95% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 95% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 96% sequence identity to any one of SEQ ID NO: 1, SEQ ID NO: 2, SEQ ID NOs: 24-87, or SEQ ID NOs: 103-166; the first Fc domain comprises an amino acid sequence with at least 96% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 96% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 96% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 97% sequence identity to any one of SEQ ID NO: 1, SEQ ID NO: 2, SEQ ID NOs: 24-87, or SEQ ID NOs: 103-166; the first Fc domain comprises an amino acid sequence with at least 97% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 97% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 97% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 98% sequence identity to any one of SEQ ID NO: 1, SEQ ID NO: 2, SEQ ID NOs: 24-87, or SEQ ID NOs: 103-166; the first Fc domain comprises an amino acid sequence with at least 98% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 98% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 98% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 99% sequence identity to any one of SEQ ID NO: 1, SEQ ID NO: 2, SEQ ID NOs: 24-87, or SEQ ID NOs: 103-166; the first Fc domain comprises an amino acid sequence with at least 99% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 99% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 99% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain consists of an amino acid sequence selected from the group including: SEQ ID NOs: 24-86 and SEQ ID NOs: 103-166; the first Fc domain consists of an amino acid sequence selected from the group including: SEQ ID NO: 9, SEQ ID NO: 10, SEQ ID NO: 11, SEQ ID NO: 12, and SEQ ID NO: 13; the IL-12p40 subunit domain consists of an amino acid sequence selected from the group including: SEQ ID NO: 3, SEQ ID NO: 4, SEQ ID NO: 88, SEQ ID NO: 89, and SEQ ID NO: 90); and the second Fc domain consists of an amino acid sequence selected from the group including: SEQ ID NO: 9, SEQ ID NO: 10, SEQ ID NO: 11, SEQ ID NO: 12, and SEQ ID NO: 13.

In a further embodiment and in accordance with any of the above, the variant IL-12p35 subunit domain comprises SEQ ID NO: 87, and further comprises one, two, three, four, five, six, or all seven amino acid substitutions selected from the group including: Y40A, T43A, D126A, P127A, R129A, K168A, and K170A; the first Fc domain comprises an amino acid sequence with at least 99% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 99% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 99% sequence identity to SEQ ID NO: 9, SEQ ID NO: 10, SEQ ID NO: 11, SEQ ID NO: 12, or SEQ ID NO: 13.

In a further embodiment and in accordance with any of the above, the variant IL-12p35 subunit domain comprises additional amino acid substitutions.

In another aspect, the present disclosure provides a heterodimeric Fc fusion protein comprising: a) a first fusion construct, comprising: a variant IL-12p35 subunit domain and a first Fc domain, wherein the N-terminus of the variant IL-12p35 subunit domain is covalently attached to the C-terminus of the first Fc domain; and b) a second fusion construct, comprising: an IL-12p40 subunit domain and a second Fc domain, wherein the N-terminus of the IL-12p40 subunit domain is covalently attached to the C-terminus of the second Fc domain; optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain comprises one or more amino acid substitutions selected from the group including: Y40A, T43A, D126A, P127A, R129A, K168A, and K170A.

In a further embodiment and in accordance with the above, the variant IL-12p35 subunit domain comprises two or more amino acid substitutions selected from the group including: Y40A, T43A, D126A, P127A, R129A, K168A, and K170A.

In a further embodiment and in accordance with the above, the variant IL-12p35 subunit domain comprises three or more amino acid substitutions selected from the group including: Y40A, T43A, D126A, P127A, R129A, K168A, and K170A.

In a further embodiment and in accordance with the above, the variant IL-12p35 subunit domain comprises four or more amino acid substitutions selected from the group including: Y40A, T43A, D126A, P127A, R129A, K168A, and K170A.

In a further embodiment and in accordance with the above, the variant IL-12p35 subunit domain comprises five or more amino acid substitutions selected from the group including: Y40A, T43A, D126A, P127A, R129A, K168A, and K170A.

In a further embodiment and in accordance with the above, the variant IL-12p35 subunit domain comprises six or more amino acid substitutions selected from the group including: Y40A, T43A, D126A, P127A, R129A, K168A, and K170A.

In a further embodiment and in accordance with the above, the variant IL-12p35 subunit domain comprises seven or more amino acid substitutions selected from the group including: Y40A, T43A, D126A, P127A, R129A, K168A, and K170A.

In a further embodiment and in accordance with the above, the variant IL-12p35 subunit domain comprises amino acid substitutions Y40A and D126A.

In a further embodiment and in accordance with the above, the variant IL-12p35 subunit domain comprises amino acid substitutions Y40A and P127A.

In a further embodiment and in accordance with the above, the variant IL-12p35 subunit domain comprises amino acid substitutions Y40A and T43A.

In a further embodiment and in accordance with the above, the variant IL-12p35 subunit domain comprises amino acid substitutions Y40A, D126A, and P127A.

In a further embodiment and in accordance with the above, the variant IL-12p35 subunit domain comprises amino acid substitutions Y40A, T43A, D126A, and P127A.

In a further embodiment and in accordance with the above, the variant IL-12p35 subunit domain comprises amino acid substitutions Y40A and R129A.

In a further embodiment and in accordance with the above, the variant IL-12p35 subunit domain comprises amino acid substitutions Y40A and K168A.

In a further embodiment and in accordance with the above, the variant IL-12p35 subunit domain comprises amino acid substitutions Y40A and K170A.

In a further embodiment and in accordance with the above, the variant IL-12p35 subunit domain comprises amino acid substitutions Y40A, P127A, and R129A.

In a further embodiment and in accordance with the above, the variant IL-12p35 subunit domain comprises amino acid substitutions Y40A, P127A, and K168A.

In a further embodiment and in accordance with the above, the variant IL-12p35 subunit domain comprises amino acid substitutions Y40A, P127A, and K170A.

In a further embodiment and in accordance with any of the above, the variant IL-12p35 subunit domain comprises a substitution mutation at amino acid residue Y40. In some further embodiments, the substitution mutation at amino acid residue Y40 is selected from the group including: Y40C, Y40D, Y40E, Y40G, Y40K, Y40N, Y40P, Y40Q, Y40R, Y40S, and Y40T.

In a further embodiment and in accordance with the above, the variant IL-12p35 subunit domain comprises any one of SEQ ID NOs: 177-187.

In a further embodiment and in accordance with any of the above, the variant IL-12p35 subunit domain comprises a substitution mutation at amino acid residue D126. In some further embodiments, the substitution mutation at amino acid residue D126 is selected from the group including: D126C, D126E, D126F, D126G, D1261, D126K, D126L, D126M, D126N, D126P, D126Q, D126R, D126S, D126T, D126V, and D126W.

In a further embodiment and in accordance with any of the above, the variant IL-12p35 subunit domain comprises a substitution mutation at amino acid residue P127. In some further embodiments, the substitution mutation at amino acid residue P127 is selected from the group including: P127C, P127D, P127E, P127F, P127G, P127H, P127K, P127M, P127N, P127Q, P127R, and P127S.

In a further embodiment and in accordance with any of the above, the variant IL-12p35 subunit domain comprises a substitution mutation at amino acid residue R129. In some further embodiments, the substitution mutation at amino acid residue R129 is selected from the group including: R129C, R129D, R129E, R129F, R129G, R129H, R129I, R129K, R129L, R129M, R129N, R129P, R129Q, R129S, R129T, R129V, R129W, and R129Y.

In a further embodiment and in accordance with any of the above, the variant IL-12p35 subunit domain comprises a substitution mutation at amino acid residue K168. In some further embodiments, the substitution mutation at amino acid residue K168 is selected from the group including: K168C, K168D, K168E, K168F, K168G, K168H, K168I, K168L, K168M, K168N, K168P, K168Q, K168S, K168T, K168W, and K168Y.

In a further embodiment and in accordance with any of the above, the variant IL-12p35 subunit domain comprises a substitution mutation at amino acid residue K170. In some further embodiments, the substitution mutation at amino acid residue K170 is selected from the group including: K170C, K170D, K170E, K170G, K170I, K170M, K170P, K170S, K170T, K170V, and K170W.

In a further embodiment and in accordance with any of the above, the variant IL-12p35 subunit domain comprises a first substitution mutation selected from the group including: Y40A, Y40C, Y40D, Y40E, Y40G, Y40K, Y40N, Y40P, Y40Q, Y40R, Y40S, and Y40T.

In a further embodiment and in accordance with the above, the variant IL-12p35 subunit domain further comprises a second substitution mutation.

In a further embodiment and in accordance with the above, the second substitution mutation is selected from the group including: D126A, D126C, D126E, D126F, D126G, D1261, D126K, D126L, D126M, D126N, D126P, D126Q, D126R, D126S, D126T, D126V, D126W, P127A, P127C, P127D, P127E, P127F, P127G, P127H, P127K, P127M, P127N, P127Q, P127R, P127S, R129A, R129C, R129D, R129E, R129F, R129G, R129H, R129I, R129K, R129L, R129M, R129N, R129P, R129Q, R129S, R129T, R129V, R129W, R129Y, K168A, K168C, K168D, K168E, K168F, K168G, K168H, K168I, K168L, K168M, K168N, K168P, K168Q, K168S, K168T, K168W, K168Y, K170A, K170C, K170D, K170E, K170G, K170I, K170M, K170P, K170S, K170T, K170V, K170F, K170L, K170N, and K170W.

In a further embodiment and in accordance with any of the above, the variant IL-12p35 subunit domain comprises any one of SEQ ID NOs: 199-247 or SEQ ID NOs: 279-290.

In a further embodiment and in accordance with any of the above, the variant IL-12p35 subunit domain may further comprise a C74S substitution mutation.

In a further embodiment and in accordance with any of the above, the IL-12p40 subunit domain comprises a variant IL-12p40 subunit domain.

In a further embodiment and in accordance with the above, the variant IL-12p40 subunit domain comprises one or more amino acid substitutions selected from the group including: C177S, C252S, and C177S/C252S.

In a further embodiment and in accordance with any of the above, the first Fc domain comprises an amino acid selected from the group including: SEQ ID NO: 9, SEQ ID NO: 10, SEQ ID NO: 11, SEQ ID NO: 12, and SEQ ID NO: 13, and the second Fc domain comprises an amino acid selected from the group including: SEQ ID NO: 9, SEQ ID NO: 10, SEQ ID NO: 11, SEQ ID NO: 12, and SEQ ID NO: 13.

In a further embodiment and in accordance with any of the above, the variant IL-12p35 subunit domain comprises an amino acid sequence selected from the group including: SEQ ID NOs: 24-86 and SEQ ID NOs: 103-166; the first Fc domain comprises an amino acid sequence selected from the group including: SEQ ID NO: 9, SEQ ID NO: 10, SEQ ID NO: 11, SEQ ID NO: 12, and SEQ ID NO: 13; the IL-12p40 subunit domain comprises an amino acid sequence selected from the group including: SEQ ID NO: 3, SEQ ID NO: 4, SEQ ID NO: 88, SEQ ID NO: 89, and SEQ ID NO: 90; and the second Fc domain comprises an amino acid sequence selected from the group including: SEQ ID NO: 9, SEQ ID NO: 10, SEQ ID NO: 11, SEQ ID NO: 12, and SEQ ID NO: 13.

In a further embodiment and in accordance with the above, the variant IL-12p35 subunit domain comprises an amino acid sequence with at least 95% sequence identity to any one of SEQ ID NO: 1, SEQ ID NO: 2, SEQ ID NOs: 24-87, or SEQ ID NOs: 103-166; the first Fc domain comprises an amino acid sequence with at least 95% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 95% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 95% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 96% sequence identity to any one of SEQ ID NO: 1, SEQ ID NO: 2, SEQ ID NOs: 24-87, or SEQ ID NOs: 103-166; the first Fc domain comprises an amino acid sequence with at least 96% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 96% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 96% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 97% sequence identity to any one of SEQ ID NO: 1, SEQ ID NO: 2, SEQ ID NOs: 24-87, or SEQ ID NOs: 103-166; the first Fc domain comprises an amino acid sequence with at least 97% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 97% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 97% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 98% sequence identity to any one of SEQ ID NO: 1, SEQ ID NO: 2, SEQ ID NOs: 24-87, or SEQ ID NOs: 103-166; the first Fc domain comprises an amino acid sequence with at least 98% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 98% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 98% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain comprises an amino acid sequence with at least 99% sequence identity to any one of SEQ ID NO: 1, SEQ ID NO: 2, SEQ ID NOs: 24-87, or SEQ ID NOs: 103-166; the first Fc domain comprises an amino acid sequence with at least 99% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 99% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 99% sequence identity to SEQ ID NO: 9, SEQ ID NO: 10, SEQ ID NO: 11, SEQ ID NO: 12, or SEQ ID NO: 13.

In a further embodiment and in accordance with the above, the variant IL-12p35 subunit domain consists of an amino acid sequence selected from the group including: SEQ ID NOs: 24-86 and SEQ ID NOs: 103-166; the first Fc domain consists of an amino acid sequence selected from the group including: SEQ ID NO: 9, SEQ ID NO: 10, SEQ ID NO: 11, SEQ ID NO: 12, and SEQ ID NO: 13; the IL-12p40 subunit domain consists of an amino acid sequence selected from the group including: SEQ ID NO: 3, SEQ ID NO: 4, SEQ ID NO: 88, SEQ ID NO: 89, and SEQ ID NO: 90; and the second Fc domain consists of an amino acid sequence selected from the group including: SEQ ID NO: 9, SEQ ID NO: 10, SEQ ID NO: 11, SEQ ID NO: 12, and SEQ ID NO: 13.

In a further embodiment and in accordance with any of the above, the variant IL-12p35 subunit domain comprises SEQ ID NO: 87, and further comprises one, two, three, four, five, six, or all seven amino acid substitutions selected from the group including: Y40A, T43A, D126A, P127A, R129A, K168A, and K170A; the first Fc domain comprises an amino acid sequence with at least 99% sequence identity to SEQ ID NO: 9, SEQ ID NO: 10, SEQ ID NO: 11, SEQ ID NO: 12, or SEQ ID NO: 13; the IL-12p40 subunit domain comprises an amino acid sequence with at least 99% sequence identity to SEQ ID NO: 3, SEQ ID NO: 4, SEQ ID NO: 88, SEQ ID NO: 89, or SEQ ID NO: 90; and the second Fc domain comprises an amino acid sequence with at least 99% sequence identity to SEQ ID NO: 9, SEQ ID NO: 10, SEQ ID NO: 11, SEQ ID NO: 12, or SEQ ID NO: 13.

In a further embodiment and in accordance with any of the above, the variant IL-12p35 subunit domain comprises additional amino acid substitutions.

In a further embodiment and in accordance with any of the above, the heterodimeric Fc fusion protein is provided for use in treating cancer in a subject.

In another aspect, the present disclosure provides one or more nucleic acids encoding a heterodimeric Fc fusion protein according to any of the aspects and embodiments above.

In another aspect, the present disclosure provides a host cell comprising the one or more nucleic acids encoding a heterodimeric Fc fusion protein according to any of the aspects and embodiments above.

In another aspect, the present disclosure provides a host cell comprising the one or more nucleic acids encoding a heterodimeric Fc fusion protein according to any of the aspects and embodiments above.

In another aspect, the present disclosure provides a method of producing a heterodimeric Fc fusion protein, the method comprising: culturing a host cell with one or more nucleic acids or vectors under conditions whereby the heterodimeric Fc fusion protein is produced, wherein the one or more nucleic acids or vectors comprises the one or more nucleic acids described in the aspects and embodiments above, further wherein the produced heterodimeric Fc fusion protein has an increased half-life compared to half-life of a reference IL-12, wherein the IL-12 comprises one or more of: a wild-type IL-12, a human wild-type IL-12, a commercially available IL-12 molecule, or an IL-12 Fc fusion protein.

In a further embodiment and in accordance with the above, the method further comprises isolating and/or purifying the produced heterodimeric Fc fusion protein.

In a further embodiment and in accordance with any of the above, the produced heterodimeric Fc fusion protein has an altered binding affinity for Interleukin-12 Receptor Beta 2 (IL-12Rβ2) compared to binding affinity of a reference IL-12.

In a further embodiment and in accordance with the above, the produced heterodimeric Fc fusion protein has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay.

In a further embodiment and in accordance with the above, the assay comprises an SPR assay.

In a further embodiment and in accordance with any of the above, the produced heterodimeric Fc fusion protein has binding affinity for IL-12Rβ2 below the lower level of detectability of an assay, and binding affinity of a reference IL-12 is detectable, as determined by an assay.

In a further embodiment and in accordance with the above, the assay comprises an SPR assay.

In a further embodiment and in accordance with any of the above, the produced heterodimeric Fc fusion protein has potency reduced by about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12, wherein the reference IL-12 comprises one or more of: a wild-type IL-12, a human wild-type IL-12, a commercially available IL-12 molecule, or an IL-12 Fc fusion protein, as determined by an assay.

In a further embodiment and in accordance with the above, the assay comprises an IL-12 HEK reporter assay.

In a further embodiment and in accordance with any of the above, the produced heterodimeric Fc fusion protein has a reduced capability to stimulate IFNγ production by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to capability to stimulate IFNγ production of a reference IL-12, wherein the reference IL-12 comprises one or more of: a wild-type IL-12, a human wild-type IL-12, a commercially available IL-12 molecule, or an IL-12 Fc fusion protein, as determined by an assay.

In a further embodiment and in accordance with the above, the assay comprises one or more of the following: (i) an intracellular cytokine stain assay, (ii) a Luminex bead-based cytokine release assay, (iii) an ELISA, or (iv) an ELISpot assay.

In another aspect, the present disclosure provides a heterodimeric Fc fusion protein comprising: a) a first fusion construct, comprising: (i) a variant IL-12p35 subunit domain, wherein the variant IL-12p35 subunit domain is selected from a group including SEQ ID NOs: 24, 30-34, 49, 52, 53, 65, 103, 104, 112, 177-247, (ii) a first Fc domain, wherein the first Fc domain is selected from a group including SEQ ID NOs: 12 and 13, and (iii) a linker, wherein the linker comprises SEQ ID NO: 15, the C-terminus of the variant IL-12p35 subunit domain is covalently attached to the N-terminus of the linker, and the C-terminus of the linker is covalently attached to the N-terminus of the first Fc domain; and b) a second fusion construct, comprising: (i) a (variant) IL-12p40 subunit domain, wherein the (variant) IL-12p40 subunit is selected from a group including SEQ ID NOs: 4, 89, and 90, (ii) a second Fc domain, wherein the second Fc domain is selected from a group including SEQ ID NOs: 13 and 12, and (iii) a linker, wherein the linker comprises SEQ ID NO: 15, the C-terminus of the (variant) IL-12p40 subunit domain is covalently attached to the N-terminus of the linker, and the C-terminus of the linker is covalently attached to the N-terminus of the second Fc domain.

In a further embodiment and in accordance with the above, the variant IL-12p35 subunit may further comprise a C74S substitution mutation, and the (variant) IL-12p40 subunit may further comprise a C177S substitution mutation, such that the inter-chain disulfide bond between the variant IL-12p35 subunit domain and the (variant) IL-12p40 subunit domain is removed.

In one aspect, the present disclosure provides a non-naturally occurring IL-12 variant, comprising: a) a variant IL-12p35 subunit, wherein the variant IL-12p35 subunit comprises a first amino acid substitution mutation, wherein the first amino acid substitution is selected from a group including: Y40A, Y40E, Y40G, Y40P, Y40R, Y40S, K170A, K170P, K170T; and b) an IL-12p40 subunit.

In a further embodiment and in accordance with the above, i) the first amino acid substitution mutation is Y40A; ii) the variant IL-12p35 subunit further comprises a second substitution mutation; and iii) the second substitution mutation is selected from a group including: K168A, K168D, K168E, K168I, K168M, K168Q, K168T, K170A, K170L, and K170T.

In a further embodiment and in accordance with the above, i) the first amino acid substitution mutation is Y40E; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40G; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40P; ii) the variant IL-12p35 subunit further comprises a second substitution mutation; and iii) the second substitution mutation is selected from a group including: K170A, K168A, K168D, K168I, and K168T.

In a further embodiment and in accordance with the above, i) the first amino acid substitution mutation is Y40S; ii) the variant IL-12p35 subunit further comprises a second substitution mutation; and iii) the second substitution mutation is selected from a group including: K168I, K168T, K170A, K170L, K170T, and R129A.

In a further embodiment and in accordance with the above, i) the first amino acid substitution mutation is K170A; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170P; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170T; ii) the variant IL-12p35 subunit further comprises a second substitution mutation; and iii) the second substitution mutation is selected from a group including: K168A, K168I, K168T, and R129E.

In a further embodiment and in accordance with the above, the variant IL-12p35 subunit comprises any one of SEQ ID NOs: 24, 34, 103, 104, 109, 179, 180, 183, 185, 186, 194, 196, 233, 234, 238, 240, 243, 245, and 248-278.

In a further embodiment and in accordance with any of the above, the variant IL-12p35 subunit domain further comprises a C74S substitution mutation.

In a further embodiment and in accordance with any of the above, the IL-12p40 subunit comprises a variant IL-12p40 subunit, wherein the variant IL-12p40 subunit comprises one or more amino acid substitutions selected from a group including: C177S, C252S, and C177S/C252S.

In a further embodiment and in accordance with any of the above, the IL-12p40 subunit comprises any one of SEQ ID NOs: 4, 88, 89, and 90.

In a further embodiment and in accordance with any of the above, the non-naturally occurring IL-12 variant further comprises one or more of the following fused to the variant IL-12p35 subunit and/or the IL-12p40 subunit: (i) a Fc domain, wherein the Fc domain comprises one or more amino acid sequences selected from a group including: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

In a further embodiment and in accordance with any of the above, the C-terminus of the variant IL-12p35 subunit is covalently attached to the N-terminus of the IL-12p40 subunit.

In a further embodiment and in accordance with the above, the non-naturally occurring IL-12 variant further comprises a linker domain comprising an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the variant IL-12p35 subunit is covalently attached to the N-terminus of the linker domain and the C-terminus of the linker domain is covalently attached to the N-terminus of the IL-12p40 subunit.

In a further embodiment and in accordance with any of the above, the C-terminus of the IL-12p40 subunit is covalently attached to the N-terminus of the variant IL-12p35 subunit.

In a further embodiment and in accordance with the above, the non-naturally occurring IL-12 variant further comprises a linker domain comprising an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the IL-12p40 subunit is covalently attached to the N-terminus of the linker domain and the C-terminus of the linker domain is covalently attached to the N-terminus of the variant IL-12p35 subunit.

In a further embodiment and in accordance with any of the above, the variant 11-12p35 subunit comprises one or more additional amino acid substitutions.

In a further embodiment and in accordance with any of the above, the non-naturally occurring IL-12 variant is provided for use in treating cancer in a subject.

In another aspect, the present disclosure provides one or more nucleic acids encoding a non-naturally occurring IL-12 variant according to any of the aspects and embodiments above.

In another aspect, the present disclosure provides a host cell comprising the one or more nucleic acids encoding a non-naturally occurring IL-12 variant according to any of the aspects and embodiments above.

In another aspect, the present disclosure provides a method of producing a non-naturally occurring IL-12 variant, the method comprising: culturing a host cell with one or more nucleic acids or vectors under conditions whereby the non-naturally occurring variant is produced, wherein: i) the one or more nucleic acids or vectors comprises the one or more nucleic acids described in the aspects and embodiments above, and ii) at least one substitution mutation of the variant IL-12p35 subunit improves half-life as compared to half-life of a reference IL-12.

In a further embodiment and in accordance with the above, the method further comprises isolating and/or purifying the produced non-naturally occurring IL-12 variant.

In a further embodiment and in accordance with any of the above, the non-naturally occurring IL-12 variant further comprises one or more of the following fused to the variant IL-12p35 subunit and/or the IL-12p40 subunit: (i) a Fc domain, wherein the Fc domain comprises one or more amino acid sequences selected from the group including: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

In a further embodiment and in accordance with any of the above, the produced non-naturally occurring IL-12 variant has an altered binding affinity for Interleukin-12 Receptor Beta 2 (IL-12Rβ2) compared to binding affinity of a reference IL-12.

In a further embodiment and in accordance with the above, the produced non-naturally occurring IL-12 variant has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay.

In a further embodiment and in accordance with the above, the assay comprises an SPR assay.

In a further embodiment and in accordance with any of the above, the produced non-naturally occurring IL-12 variant has binding affinity for IL-12R32 below the lower level of detectability of an assay, and binding affinity of a reference IL-12 is detectable, as determined by an assay.

In a further embodiment and in accordance with the above, the assay comprises an SPR assay.

In a further embodiment and in accordance with any of the above, the produced non-naturally occurring IL-12 variant has potency reduced by about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12, as determined by an assay.

In a further embodiment and in accordance with the above, the assay comprises an IL-12 HEK reporter assay.

In a further embodiment and in accordance with any of the above, the produced non-naturally occurring IL-12 variant has a reduced capability to stimulate IFNγ production by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to capability to stimulate IFNγ production of a reference IL-12, as determined by an assay.

In a further embodiment and in accordance with the above, the assay comprises one or more of the following: (i) an intracellular cytokine stain assay, (ii) a Luminex bead-based cytokine release assay, (iii) an ELISA, or (iv) an ELISpot assay.

In a further embodiment and in accordance with any of the above, the reference IL-12 comprises one or more of: a wild-type IL-12, a human wild-type IL-12, a commercially available IL-12 molecule, or an IL-12 Fc fusion protein.

In one aspect, the present disclosure provides a heterodimeric Fc fusion protein, comprising: a) a first fusion construct, comprising: a variant IL-12p35 subunit domain and a first Fc domain, wherein: i) the C-terminus of the variant IL-12p35 subunit domain is covalently attached to the N-terminus of the first Fc domain, ii) the variant IL-12p35 subunit domain comprises a first amino acid substitution mutation, and iii) the first amino acid substitution mutation is selected from a group including: Y40A, Y40E, Y40G, Y40P, Y40R, Y40S, K170A, K170P, K170T; and b) a second fusion construct, comprising: an IL-12p40 subunit domain and a second Fc domain, wherein the C-terminus of the IL-12p40 subunit domain is covalently attached to the N-terminus of the second Fc domain, optionally wherein the first Fc domain and the second Fc domain comprise modifications that (i) promote heterodimerization of the first and second Fc domains and/or (ii) silence or inhibit effector function.

In a further embodiment and in accordance with the above, i) the first amino acid substitution mutation is Y40A; ii) the variant IL-12p35 subunit further comprises a second substitution mutation; and iii) the second substitution mutation is selected from a group including: K168A, K168D, K168E, K168I, K168M, K168Q, K168T, K170A, K170L, and K170T.

In a further embodiment and in accordance with the above, i) the first amino acid substitution mutation is Y40E; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40G; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40P; ii) the variant IL-12p35 subunit further comprises a second substitution mutation; and iii) the second substitution mutation is selected from a group including: K170A, K168A, K168D, K168I, and K168T.

In a further embodiment and in accordance with the above, i) the first amino acid substitution mutation is Y40S; ii) the variant IL-12p35 subunit further comprises a second substitution mutation; and iii) the second substitution mutation is selected from a group including: K168I, K168T, K170A, K170L, K170T, and R129A.

In a further embodiment and in accordance with the above, i) the first amino acid substitution mutation is K170A; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170P; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170T; ii) the variant IL-12p35 subunit further comprises a second substitution mutation; and iii) the second substitution mutation is selected from a group including: K168A, K168I, K168T, and R129E.

In a further embodiment and in accordance with any of the above, the variant IL-12p35 subunit domain comprises any one of SEQ ID NOs: 24, 34, 103, 104, 109, 179, 180, 183, 185, 186, 194, 196, 233, 234, 238, 240, 243, 245, and 248-278.

In a further embodiment and in accordance with any of the above, the variant IL-12p35 subunit domain further comprises a C74S substitution mutation.

In a further embodiment and in accordance with any of the above, the IL-12p40 subunit domain comprises a variant IL-12p40 subunit domain, wherein the variant IL-12p40 subunit domain comprises one or more amino acid substitutions selected from a group including: C177S, C252S, and C177S/C252S.

In a further embodiment and in accordance with any of the above, the IL-12p40 subunit domain comprises any one of SEQ ID NOs: 4, 88, 89, and 90.

In a further embodiment and in accordance with any of the above, i) the first Fc domain comprises an amino acid selected from a group including: SEQ ID NO: 9, SEQ ID NO: 10, SEQ ID NO: 11, SEQ ID NO: 12, and SEQ ID NO: 13; and ii) the second Fc domain comprises an amino acid selected from a group including: SEQ ID NO: 9, SEQ ID NO: 10, SEQ ID NO: 11, SEQ ID NO: 12, and SEQ ID NO: 13.

In a further embodiment and in accordance with any of the above, i) the first fusion construct further comprises a linker domain; ii) the linker domain comprises an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23; and iii) the C-terminus of the variant IL-12p35 subunit domain is covalently attached to the N-terminus of the linker domain and the C-terminus of the linker domain is covalently attached to the N-terminus of the first Fc domain.

In a further embodiment and in accordance with any of the above, i) the second fusion construct further comprises a linker domain; ii) the linker domain comprises an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23; and iii) the C-terminus of the IL-12p40 subunit domain is covalently attached to the N-terminus of the linker domain and the C-terminus of the linker domain is covalently attached to the N-terminus of the second Fc domain.

In a further embodiment and in accordance with any of the above, the variant IL-12p35 subunit domain comprises one or more additional amino acid substitutions.

In a further embodiment and in accordance with any of the above, the heterodimeric Fc fusion protein is provided for use in treating cancer in a subject.

In another aspect, the present disclosure provides one or more nucleic acids encoding a heterodimeric Fc fusion protein according to any of the aspects and embodiments above.

In another aspect, the present disclosure provides a host cell comprising the one or more nucleic acids encoding a heterodimeric Fc fusion protein according to any of the aspects and embodiments above.

In another aspect, the present disclosure provides a method of producing a heterodimeric Fc fusion protein, the method comprising: culturing a host cell with one or more nucleic acids or vectors under conditions whereby the heterodimeric Fc fusion protein is produced, wherein: i) the one or more nucleic acids or vectors comprises the one or more nucleic acids described in the aspects and embodiments above, and ii) at least one substitution mutation of the variant IL-12p35 subunit domain improves half-life as compared to half-life of a reference IL-12.

In a further embodiment and in accordance with the above, the method further comprises isolating and/or purifying the produced heterodimeric Fc fusion protein.

In a further embodiment and in accordance with any of the above, the produced heterodimeric Fc fusion protein has an altered binding affinity for Interleukin-12 Receptor Beta 2 (IL-12Rβ2) compared to binding affinity of a reference IL-12.

In a further embodiment and in accordance with the above, the produced heterodimeric Fc fusion protein has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay.

In a further embodiment and in accordance with the above, the assay comprises an SPR assay.

In a further embodiment and in accordance with any of the above, the produced heterodimeric Fc fusion protein has binding affinity for IL-12Rβ2 below the lower level of detectability of an assay, and binding affinity of a reference IL-12 is detectable, as determined by an assay.

In a further embodiment and in accordance with the above, the assay comprises an SPR assay.

In a further embodiment and in accordance with any of the above, the produced heterodimeric Fc fusion protein has potency reduced by about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12, as determined by an assay.

In a further embodiment and in accordance with the above, the assay comprises an IL-12 HEK reporter assay.

In a further embodiment and in accordance with any of the above, the produced heterodimeric Fc fusion protein has a reduced capability to stimulate IFNγ production by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to capability to stimulate IFNγ production of a reference IL-12, as determined by an assay.

In a further embodiment and in accordance with the above, the assay comprises one or more of the following: (i) an intracellular cytokine stain assay, (ii) a Luminex bead-based cytokine release assay, (iii) an ELISA, or (iv) an ELISpot assay.

In a further embodiment and in accordance with any of the above, the reference IL-12 comprises one or more of: a wild-type IL-12, a human wild-type IL-12, a commercially available IL-12 molecule, or an IL-12 Fc fusion protein.

In one aspect, the present disclosure provides a heterodimeric Fc fusion protein, comprising: a) a first fusion construct, comprising: a variant IL-12p35 subunit domain and a first Fc domain, wherein: i) the C-terminus of the first Fc domain is covalently attached to the N-terminus of the variant IL-12p35 subunit domain, ii) the variant IL-12p35 subunit domain comprises a first amino acid substitution mutation, and iii) the first amino acid substitution mutation is selected from a group including: Y40A, Y40E, Y40G, Y40P, Y40R, Y40S, K170A, K170P, K170T; and b) a second fusion construct, comprising: an IL-12p40 subunit domain and a second Fc domain, wherein the C-terminus of the second Fc domain is covalently attached to the N-terminus of the IL-12p40 subunit domain, optionally wherein the first Fc domain and the second Fc domain comprise modifications that (i) promote heterodimerization of the first and second Fc domains and/or (ii) silence or inhibit effector function.

In a further embodiment and in accordance with the above, i) the first amino acid substitution mutation is Y40A; ii) the variant IL-12p35 subunit further comprises a second substitution mutation; and iii) the second substitution mutation is selected from a group including: K168A, K168D, K168E, K168I, K168M, K168Q, K168T, K170A, K170L, and K170T.

In a further embodiment and in accordance with the above, i) the first amino acid substitution mutation is Y40E; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40G; ii) the variant IL-12p35 subunit further comprises a second substitution mutation; and iii) the second substitution mutation is selected from a group including: K170A, K168A, K168I, K168T, and R129A.

In a further embodiment and in accordance with the above, i) the first amino acid substitution mutation is Y40P; ii) the variant IL-12p35 subunit further comprises a second substitution mutation; and iii) the second substitution mutation is selected from a group including: K170A, K168A, K168D, K168I, and K168T.

In a further embodiment and in accordance with the above, i) the first amino acid substitution mutation is Y40S; ii) the variant IL-12p35 subunit further comprises a second substitution mutation; and iii) the second substitution mutation is selected from a group including: K168I, K168T, K170A, K170L, K170T, and R129A.

In a further embodiment and in accordance with the above, i) the first amino acid substitution mutation is K170A; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170P; ii) the variant IL-12p35 subunit further comprises a second substitution mutation; and iii) the second substitution mutation is selected from a group including: K168A, K168I, K168T, and R129E.

In a further embodiment and in accordance with the above, i) the first amino acid substitution mutation is K170T; ii) the variant IL-12p35 subunit further comprises a second substitution mutation; and iii) the second substitution mutation is selected from a group including: K168A, K168I, K168T, and R129E.

In a further embodiment and in accordance with any of the above, the variant IL-12p35 subunit domain comprises any one of SEQ ID NOs: 24, 34, 103, 104, 109, 179, 180, 183, 185, 186, 194, 196, 233, 234, 238, 240, 243, 245, and 248-278.

In a further embodiment and in accordance with any of the above, the variant IL-12p35 subunit domain further comprises a C74S substitution mutation.

In a further embodiment and in accordance with any of the above, the IL-12p40 subunit domain comprises a variant IL-12p40 subunit domain, wherein the variant IL-12p40 subunit domain comprises one or more amino acid substitutions selected from a group including: C177S, C252S, and C177S/C252S.

In a further embodiment and in accordance with any of the above, the IL-12p40 subunit domain comprises any one of SEQ ID NOs: 4, 88, 89, and 90.

In a further embodiment and in accordance with any of the above, i) the first Fc domain comprises an amino acid selected from a group including: SEQ ID NO: 9, SEQ ID NO: 10, SEQ ID NO: 11, SEQ ID NO: 12, and SEQ ID NO: 13; and ii) the second Fc domain comprises an amino acid selected from a group including: SEQ ID NO: 9, SEQ ID NO: 10, SEQ ID NO: 11, SEQ ID NO: 12, and SEQ ID NO: 13.

In a further embodiment and in accordance with any of the above, i) the first fusion construct further comprises a linker domain; ii) the linker domain comprises an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23; and iii) the C-terminus of the first Fc domain is covalently attached to the N-terminus of the linker domain and the C-terminus of the linker domain is covalently attached to the N-terminus of the variant IL-12p35 subunit domain.

In a further embodiment and in accordance with any of the above, i) the second fusion construct further comprises a linker domain; ii) the linker domain comprises an amino acid sequence selected from a group including: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23; and iii) the C-terminus of the second Fc domain is covalently attached to the N-terminus of the linker domain and the C-terminus of the linker domain is covalently attached to the N-terminus of the IL-12p40 subunit domain.

In a further embodiment and in accordance with any of the above, the variant IL-12p35 subunit domain comprises one or more additional amino acid substitutions.

In a further embodiment and in accordance with any of the above, the heterodimeric Fc fusion protein is provided for use in treating cancer in a subject.

In another aspect, the present disclosure provides one or more nucleic acids encoding a heterodimeric Fc fusion protein according to any of the aspects and embodiments above.

In another aspect, the present disclosure provides a host cell comprising the one or more nucleic acids encoding a heterodimeric Fc fusion protein according to any of the aspects and embodiments above.

In another aspect, the present disclosure provides a method of producing a heterodimeric Fc fusion protein, the method comprising: culturing a host cell with one or more nucleic acids or vectors under conditions whereby the heterodimeric Fc fusion protein is produced, wherein: i) the one or more nucleic acids or vectors comprises the one or more nucleic acids described in the aspects and embodiments above, and ii) at least one substitution mutation of the variant IL-12p35 subunit domain improves half-life as compared to half-life of a reference IL-12.

In a further embodiment and in accordance with the above, the method further comprises isolating and/or purifying the produced heterodimeric Fc fusion protein.

In a further embodiment and in accordance with any of the above, the produced heterodimeric Fc fusion protein has an altered binding affinity for Interleukin-12 Receptor Beta 2 (IL-12Rβ2) compared to binding affinity of a reference IL-12.

In a further embodiment and in accordance with the above, the produced heterodimeric Fc fusion protein has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay.

In a further embodiment and in accordance with the above, the assay comprises an SPR assay.

In a further embodiment and in accordance with any of the above, the produced heterodimeric Fc fusion protein has binding affinity for IL-12Rβ2 below the lower level of detectability of an assay, and binding affinity of a reference IL-12 is detectable, as determined by an assay.

In a further embodiment and in accordance with the above, the assay comprises an SPR assay.

In a further embodiment and in accordance with any of the above, the produced heterodimeric Fc fusion protein has potency reduced by about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12, as determined by an assay.

In a further embodiment and in accordance with the above, the assay comprises an IL-12 HEK reporter assay.

In a further embodiment and in accordance with any of the above, the produced heterodimeric Fc fusion protein has a reduced capability to stimulate IFNγ production by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to capability to stimulate IFNγ production of a reference IL-12, as determined by an assay.

In a further embodiment and in accordance with the above, the assay comprises one or more of the following: (i) an intracellular cytokine stain assay, (ii) a Luminex bead-based cytokine release assay, (iii) an ELISA, or (iv) an ELISpot assay.

In a further embodiment and in accordance with any of the above, the reference IL-12 comprises one or more of: a wild-type IL-12, a human wild-type IL-12, a commercially available IL-12 molecule, or an IL-12 Fc fusion protein.

In another aspect, the present disclosure provides a heterodimeric Fc fusion protein, comprising: a) a first fusion construct, comprising: a variant IL-12p35 subunit domain, a first linker domain, and a first Fc domain, wherein: i) the C-terminus of the variant IL-12p35 subunit domain is covalently attached to the N-terminus of the first linker domain and the C-terminus of the first linker domain is covalently attached to the N-terminus of the first Fc domain, ii) the first linker domain comprises SEQ ID NO: 15, iii) the first Fc domain comprises SEQ ID NO: 12, and iv) the variant IL-12p35 subunit domain comprises one or more amino acid substitutions selected from a group including: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E; and b) a second fusion construct, comprising: an IL-12p40 subunit domain, a second linker domain, and a second Fc domain, wherein: i) the C-terminus of the IL-12p40 subunit domain is covalently attached to the N-terminus of the second linker domain and the C-terminus of the second linker domain is covalently attached to the N-terminus of the second Fc domain, ii) the second linker domain comprises SEQ ID NO: 15, iii) the second Fc domain comprises SEQ ID NO: 13, and iv) the IL-12p40 subunit domain comprises SEQ ID NO: 89, optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain further comprises a C74S substitution mutation.

In a further embodiment and in accordance with any of the above, the IL-12p40 subunit domain further comprises a C177S substitution mutation.

In another aspect, the present disclosure provides a heterodimeric Fc fusion protein, comprising: a) a first fusion construct, comprising: a variant IL-12p35 subunit domain, a first linker domain, and a first Fc domain, wherein: i) the C-terminus of the variant IL-12p35 subunit domain is covalently attached to the N-terminus of the first linker domain and the C-terminus of the first linker domain is covalently attached to the N-terminus of the first Fc domain, ii) the first linker domain comprises SEQ ID NO: 15, iii) the first Fc domain comprises SEQ ID NO: 13, and iv) the variant IL-12p35 subunit domain comprises one or more amino acid substitutions selected from a group including: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E; and b) a second fusion construct, comprising: an IL-12p40 subunit domain, a second linker domain, and a second Fc domain, wherein: i) the C-terminus of the IL-12p40 subunit domain is covalently attached to the N-terminus of the second linker domain and the C-terminus of the second linker domain is covalently attached to the N-terminus of the second Fc domain, ii) the second linker domain comprises SEQ ID NO: 15, iii) the second Fc domain comprises SEQ ID NO: 12, and iv) the IL-12p40 subunit domain comprises SEQ ID NO: 89, optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain further comprises a C74S substitution mutation.

In a further embodiment and in accordance with any of the above, the IL-12p40 subunit domain further comprises a C177S substitution mutation.

In another aspect, the present disclosure provides a heterodimeric Fc fusion protein, comprising: a) a first fusion construct, comprising: a variant IL-12p35 subunit domain, a first linker domain, and a first Fc domain, wherein: i) the C-terminus of the first Fc domain is covalently attached to the N-terminus of the first linker domain and the C-terminus of the first linker domain is covalently attached to the N-terminus of the variant IL-12p35 subunit domain, ii) the first linker domain comprises SEQ ID NO: 15, iii) the first Fc domain comprises SEQ ID NO: 12, and iv) the variant IL-12p35 subunit domain comprises one or more amino acid substitutions selected from a group including: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E; and b) a second fusion construct, comprising: an IL-12p40 subunit domain, a second linker domain, and a second Fc domain, wherein: i) the C-terminus of the second Fc domain is covalently attached to the N-terminus of the second linker domain and the C-terminus of the second linker domain is covalently attached to the N-terminus of the IL-12p40 subunit domain, ii) the second linker domain comprises SEQ ID NO: 15, iii) the second Fc domain comprises SEQ ID NO: 13, and iv) the IL-12p40 subunit domain comprises SEQ ID NO: 89, optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain further comprises a C74S substitution mutation.

In a further embodiment and in accordance with any of the above, the IL-12p40 subunit domain further comprises a C177S substitution mutation.

In another aspect, the present disclosure provides a heterodimeric Fc fusion protein, comprising: a) a first fusion construct, comprising: a variant IL-12p35 subunit domain, a first linker domain, and a first Fc domain, wherein: i) the C-terminus of the first Fc domain is covalently attached to the N-terminus of the first linker domain and the C-terminus of the first linker domain is covalently attached to the N-terminus of the variant IL-12p35 subunit domain, ii) the first linker domain comprises SEQ ID NO: 15, iii) the first Fc domain comprises SEQ ID NO: 13, and iv) the variant IL-12p35 subunit domain comprises one or more amino acid substitutions selected from a group including: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E; and b) a second fusion construct, comprising: an IL-12p40 subunit domain, a second linker domain, and a second Fc domain, wherein: i) the C-terminus of the second Fc domain is covalently attached to the N-terminus of the second linker domain and the C-terminus of the second linker domain is covalently attached to the N-terminus of the IL-12p40 subunit domain, ii) the second linker domain comprises SEQ ID NO: 15, iii) the second Fc domain comprises SEQ ID NO: 12, and iv) the IL-12p40 subunit domain comprises SEQ ID NO: 89, optionally wherein the first Fc domain and the second Fc domain comprise modifications that (i) promote heterodimerization of the first and second Fc domains and/or (ii) silence or inhibit effector function.

In a further embodiment and in accordance with the above, the variant IL-12p35 subunit domain further comprises a C74S substitution mutation.

In a further embodiment and in accordance with any of the above, the IL-12p40 subunit domain further comprises a C177S substitution mutation.

### BRIEF DESCRIPTION OF THE DRAWINGS

The novel features of the invention are set forth with particularity in the appended claims.

A better understanding of the features and advantages of the present invention will be obtained by reference to the following detailed description that sets forth illustrative embodiments, in which the principles of the invention are utilized, and the accompanying drawings (also “Fig.,” “FIG.,” “Figure,” “Figures,” “Figs.,” and “FIGs.” herein) of which:

FIG. 1 shows a list of sequences for human, wild-type IL-12 subunits alpha and beta (precursor and mature form sequences).

FIG. 2 shows a list of sequences for human, wild-type IL-12Rβ1 (+ the sequence for the extracellular domain) and IL-12Rβ2 (+ the sequence for the extracellular domain).

FIG. 3 shows exemplary sequences for human IgG1 Fc domains (G1m allotype, as well as two sets of knob/hole mutants).

FIG. 4 shows a list of exemplary domain linker sequences.

FIG. 5 shows a list of variant IL-12p35 subunit sequences comprising one mutation.

FIGS. 6A and 6B show a list of variant IL-12p35 subunit sequences comprising two mutations.

FIGS. 7A and 7B show a list of variant IL-12p35 subunit sequences comprising three mutations.

FIGS. 8A and 8B show a list of variant IL-12p35 subunit sequences comprising four mutations.

FIG. 9 shows a list of variant IL-12p35 subunit sequences comprising: (i) five mutations, (ii) six mutations, and (iii) a C74S mutation.

FIG. 10 shows a list of variant IL-12p40 subunit sequences.

FIGS. 11A-11E show a list of exemplary heterodimeric Fc fusion proteins and associated sequences.

FIG. 12 shows a schematic representation of an exemplary IL-12 Fc fusion protein in a monovalent format.

FIG. 13 shows size exclusion (top panel) and cation exchange (bottom panel) chromatography profiles of a wild-type IL-12 heterodimeric Fc fusion protein (also referred to herein as “wild-type IL-12 Fc,” “IL-12 Fc wt,” or “IL-12 Fc w.t.,” or “WT”). Fractions that were pooled for further purification and/or analysis are indicated by rectangles.

FIG. 14 shows reducing (+DTT; left panel) and non-reducing (−DTT; right panel) SDS-PAGE analysis of purified wild-type IL-12 Fc.

FIG. 15 shows a model of the interaction between IL-12 and IL-12Rβ2. An IL-12p35 subunit, an IL-12p40 subunit, and IL-12Rβ2 are shown in cartoon representation, and the IL-12p35 subunit residues selected for mutational analysis are shown as sticks.

FIGS. 16A and 16B show reducing (+DTT; 16A) and non-reducing (−DTT; 16B) SDS-PAGE analysis of purified heterodimeric IL-12 Fc fusion proteins, wherein the IL-12p35 subunit domain comprises one mutation and the Fc domains comprise knob and hole mutations that promote heterodimerization of the Fc domains.

FIG. 17 shows SPR sensorgrams (i.e., a plot of SPR response in response units (RU) vs. time in seconds generated from an SPR instrument) representing the binding of 7.8, 15.6, 31.2, 62.5, 125, 250, 500, 1000 and 2000 nM IL-12Rβ2 to immobilized wild-type IL-12 Fc (“w.t.”; top row) or mutants (middle row and bottom row) as indicated.

FIG. 18 shows a summary of SPR data for IL-12Rβ2 binding to wild-type IL-12 Fc (“w.t.”) or heterodimeric IL-12 Fc fusion proteins, wherein the IL-12p35 subunit domain comprises one mutation. The amount of IL-12 Fc immobilized on the chip (Capture) and dissociation constant (K_{D}) are provided. As used herein, when referring to the results of an SPR assay, the term “weak” refers to a K_{D} value below the lower level of detectability for the assay employed (i.e., the K_{D} value could not be accurately determined with respect to the lower limits of detection of the assay).

FIG. 19 shows the activity of a commercially available IL-12 (Miltenyi; top row) or heterodimeric IL-12 Fc fusion proteins, wherein the IL-12p35 subunit domain comprises one mutation (middle row and bottom row), in a HEK-Blue IL-12 reporter assay compared to a wild-type IL-12 Fc (“w.t.”). Symbols and error bars indicate the mean and SD, respectively. With respect to the symbols, a filled-in circle shows the mean for the wild-type IL-12 Fc, and an empty square shows the mean for the commercially available IL-12 or the heterodimeric IL-12 Fc fusion proteins.

FIG. 20 shows a summary of EC₅₀ values and fold change (relative to w.t.) of a commercially available IL-12 (Miltenyi), wild-type IL-12 Fc (“w.t.”), and heterodimeric IL-12 Fc fusion proteins, wherein the IL-12p35 subunit domain comprises one mutation, using a HEK-Blue IL-12 assay.

FIG. 21 shows the activity of a commercially available IL-12 (Miltenyi; top row) and heterodimeric IL-12 Fc fusion proteins, wherein the IL-12p35 subunit domain comprises one mutation (middle row and bottom row), in a primary T cell IFNγ release assay compared to a wild-type IL-12 Fc (“w.t.”). Symbols and error bars indicate the mean and SD, respectively.

With respect to the symbols, a filled-in circle shows the mean for the wild-type IL-12 Fc, and an empty square shows the mean for the commercially available IL-12 or the heterodimeric IL-12 Fc fusion proteins.

FIG. 22 shows a summary of EC₅₀ values and fold change (relative to w.t.) of a commercially available IL-12 (Miltenyi), wild-type IL-12 Fc (“w.t.”), and heterodimeric IL-12 Fc fusion proteins, wherein the IL-12p35 subunit domain comprises one mutation, using a primary T cell IFNγ release assay.

FIG. 23 shows reducing (+DTT; left panel) and non-reducing (−DTT; right panel) SDS-PAGE analysis of purified heterodimeric IL-12 Fc fusion proteins, wherein the IL-12p35 subunit domain comprises two, three, or four mutations and the Fc domains comprise knob and hole mutations that promote heterodimerization of the Fc domains.

FIGS. 24A and 24B show the activity of a commercially available IL-12 (Miltenyi) or heterodimeric IL-12 Fc fusion proteins, wherein the IL-12p35 subunit domain comprises one, two, three, or four mutations, in a HEK-Blue IL-12 reporter assay compared to a wild-type IL-12 Fc (“w.t.”). Symbols and error bars indicate the mean and SD, respectively. With respect to the symbols, a filled-in circle shows the mean for the wild-type IL-12 Fc, and an empty square shows the mean for the commercially available IL-12 or the heterodimeric IL-12 Fc fusion proteins.

FIG. 25 shows a summary of EC₅₀ values and fold change (relative to w.t.) of a commercially available IL-12 (Miltenyi), wild-type IL-12 Fc (“w.t.”), and heterodimeric IL-12 Fc fusion proteins, wherein the IL-12p35 subunit domain comprises one, two, three, or four mutations, using a HEK-Blue IL-12 assay.

FIG. 26 shows the activity of heterodimeric IL-12 Fc fusion proteins, wherein the IL-12p35 subunit domain comprises two, three, or four mutations (top row and bottom row) in a primary T cell IFNγ release assay. Symbols and error bars indicate the mean and SD, respectively. With respect to the symbols, a filled-in circle shows the mean for the wild-type IL-12 Fc, and an empty square shows the mean for the heterodimeric IL-12 Fc fusion proteins.

FIG. 27 shows a summary of EC₅₀ values and fold change (relative to w.t.) of a wild-type IL-12 Fc (“w.t.”) and heterodimeric IL-12 Fc fusion proteins, wherein the IL-12p35 subunit comprises two, three, or four mutations, using a primary T cell IFNγ release assay.

FIG. 28 shows SPR sensorgrams (i.e., a plot of SPR response in response units (RU) vs. time in seconds generated from an SPR instrument) representing the binding of 0.7, 2.1, 6.2, 18.5, 55.6, 167, 500 nM IL-12Rβ2 to immobilized wild-type IL-12 Fc (“w.t.”; left) or IL-12 Fc K170A mutant (“K170A”; right) as indicated.

FIGS. 29A and 29B show the activity of heterodimeric IL-12 Fc fusion proteins, wherein the IL-12p35 subunit domain comprises one, two, or three mutations, in a HEK-Blue IL-12 reporter assay compared to a wild-type IL-12 Fc (“WT”). Symbols and error bars indicate the mean and SD, respectively. With respect to the symbols, a filled-in circle shows the mean for the wild-type IL-12 Fc, and an empty square shows the mean for the heterodimeric IL-12 Fc mutant fusion proteins.

FIG. 30 shows a summary of EC₅₀ values and fold change (relative to w.t.) of a commercially available IL-12 (Miltenyi), wild-type IL-12 Fc (“w.t.”), and heterodimeric IL-12 Fc fusion proteins, wherein the IL-12p35 subunit domain comprises one, two, or three mutations, using a HEK-Blue IL-12 assay.

FIG. 31 shows the activity of heterodimeric IL-12 Fc fusion proteins, wherein the IL-12p35 subunit domain comprises one, two, or three mutations, in a primary T cell IFNγ release assay. Symbols and error bars indicate the mean and SD, respectively. With respect to the symbols, a filled-in circle shows the mean for the wild-type IL-12 Fc, and an empty square shows the mean for the heterodimeric IL-12 Fc mutant fusion proteins.

FIG. 32 shows a summary of EC₅₀ values and fold change (relative to w.t.) of heterodimeric IL-12 Fc fusion proteins, wherein the IL-12p35 subunit domain comprises one, two, or three mutations, using a primary T cell IFNγ release assay.

FIG. 33 shows a schematic representation of an exemplary IL-12 Fc fusion protein in a monovalent format.

FIG. 34 shows cation exchange (top panel) and analytical size exclusion (bottom panel) chromatography profiles of a wild-type IL-12 heterodimeric Fc fusion protein (also referred to herein as “wild-type IL-12 Fc,” “IL-12 Fc wt,” or “IL-12 Fc w.t.,” or “WT”). Fractions that were pooled for functional assays or analysis are indicated by rectangles.

FIGS. 35A and 35B show reducing (+DTT; 35A) and non-reducing (−DTT; 35B) SDS-PAGE analysis of heterodimeric IL-12 Fc fusion proteins, wherein the IL-12p35 subunit domain comprises one, two, or three mutations.

FIG. 36 shows the activity of a commercially available IL-12 (Miltenyi) and heterodimeric IL-12 Fc fusion proteins, wherein the IL-12p35 subunit domain comprises one, two, or three mutations, in a HEK-Blue IL-12 reporter assay compared to a wild-type IL-12 Fc (“WT”). Symbols and error bars indicate the mean and SD, respectively. With respect to the symbols, a filled-in circle shows the mean for the wild-type IL-12 Fc, and an empty square shows the mean for commercially available IL-12 or the heterodimeric IL-12 Fc mutant fusion proteins.

FIG. 37 shows a summary of EC₅₀ values and fold change (relative to w.t.) of a commercially available IL-12 (Miltenyi), wild-type IL-12 Fc (“w.t.”), and heterodimeric IL-12 Fc fusion proteins, wherein the IL-12p35 subunit domain comprises one, two, or three mutations, using a HEK-Blue IL-12 assay.

FIG. 38 shows the activity of a commercially available IL-12 (Miltenyi) and heterodimeric IL-12 Fc fusion proteins, wherein the IL-12p35 subunit domain comprises one, two, or three mutations, in a primary T cell IFNγ release assay. Symbols and error bars indicate the mean and SD, respectively. With respect to the symbols, a filled-in circle shows the mean for the wild-type IL-12 Fc, and an empty square shows the mean for the commercially available IL-12 (Miltenyi) and the heterodimeric IL-12 Fc mutant fusion proteins.

FIG. 39 shows a summary of EC₅₀ values and fold change (relative to w.t.) of a commercially available IL-12 (Miltenyi) and heterodimeric IL-12 Fc fusion proteins, wherein the IL-12p35 subunit domain comprises one, two, or three mutations, using a primary T cell IFNγ release assay.

FIGS. 40A and 40B show the expression of Lag-3, PD-1, CD27, CD45RA/CD45RO, TCF1, CD62L, CD39, and Granzyme B on CD8+ T cells derived from a donor (“donor 245”) following culture in the presence or absence of either 1, 5, or 10 ng/ml of wild-type IL-12 Fc or 10 ng/ml of heterodimeric IL-12 Fc fusion proteins containing mutations within the IL-12p35 subunit domain as indicated. The horizontal line represents the mean value of two duplicate measurements (symbols).

FIGS. 41A and 41B show the expression of Lag-3, PD-1, CD27, CD45RA/CD45RO, TCF1, CD62L, CD39, and Granzyme B on CD8+ T cells derived from a donor (“donor 247”) following culture in the presence or absence of either 1, 5, or 10 ng/ml of wild-type IL-12 Fc or 10 ng/ml of heterodimeric IL-12 Fc fusion proteins containing mutations within the IL-12p35 subunit domain as indicated. The horizontal line represents the mean value of two duplicate measurements (symbols).

FIGS. 42A and 42B show the expression of Lag-3, PD-1, CD27, CD45RA/CD45RO, TCF1, CD62L, CD39, and Granzyme B on CD4+ T cells derived from a donor (“donor 245”) following culture in the presence or absence of either 1, 5, or 10 ng/ml of wild-type IL-12 Fc or 10 ng/ml of heterodimeric IL-12 Fc fusion proteins containing mutations within the IL-12p35 subunit domain as indicated. The horizontal line represents the mean value of two duplicate measurements (symbols).

FIGS. 43A and 43B show the expression of Lag-3, PD-1, CD27, CD45RA/CD45RO, TCF1, CD62L, CD39, and Granzyme B on CD4+ T cells derived from a donor (“donor 247”) following culture in the presence or absence of either 1, 5, or 10 ng/ml of wild-type IL-12 Fc or 10 ng/ml of heterodimeric IL-12 Fc fusion proteins containing mutations within the IL-12p35 subunit domain as indicated. The horizontal line represents the mean value of two duplicate measurements (symbols).

FIG. 44 shows principal component analysis (PCA) in CD8+ T cells based on the complete panel of phenotypic markers described in the main text. Donor batch effects were removed using limma “removeBatchEffect” function, and the corrected MFI values for each functional marker were analyzed using limma “plotMDS” function to obtain a PCA plot. PC1 component, which correlated the most with variant potency, was displayed as a box and whisker plot where the horizontal lines represent the median value derived from three donor samples, each performed in duplicate (symbols).

FIG. 45 shows principal component analysis (PCA) in CD4+ T cells based on the complete panel of phenotypic markers described in the main text. Donor batch effects were removed using limma “removeBatchEffect” function, and the corrected MFI values for each functional marker were analyzed using limma “plotMDS” function to obtain a PCA plot. PC2 component, which correlated the most with variant potency, was displayed as a box and whisker plot where the horizontal lines represent the median value derived from three donor samples, each performed in duplicate (symbols).

FIG. 46 shows the number of CD4+ cells (top panel), CD8+ cells (middle panel), and the CD4:CD8 ratio (defined as the number of CD4+ cells divided by the number of CD8+ cells; bottom panel) present in 1 μl of mouse plasma twelve days after injection of 10×10⁶ freshly isolated human PBMC and either wild-type IL-12 Fc or heterodimeric IL-12 Fc fusion proteins containing mutations within the IL-12p35 subunit domain as indicated. The horizontal line represents the mean value of measurements performed from three mice (symbols).

FIG. 47 shows the expression of PD-1, Lag-3, Tim-3, CD95, Ki67, Granzyme B, and CD45RA/CD45RO on CD4+ T cells derived from the plasma of mice twelve days after injection of 10×10⁶ freshly isolated human PBMC and either wild-type IL-12 Fc or heterodimeric IL-12 Fc fusion proteins containing mutations within the IL-12p35 subunit domain as indicated. The horizontal line represents the mean value of measurements performed from three mice (symbols).

FIG. 48 shows the expression of PD-1, Lag-3, Tim-3, CD95, Ki67, Granzyme B and CD45RA/CD45RO on CD8+ T cells derived from the plasma of mice twelve days after injection of 10×10⁶ freshly isolated human PBMC and either wild-type IL-12 Fc or heterodimeric IL-12 Fc fusion proteins containing mutations within the IL-12p35 subunit domain as indicated. The horizontal line represents the mean value of measurements performed from three mice (symbols).

FIG. 49 shows the concentration of IFNγ present in the plasma of mice that were injected with 10×10⁶ freshly isolated human PBMC at time 0 hours and either wild-type IL-12 Fc or heterodimeric IL-12 Fc fusion proteins containing mutations within the IL-12p35 subunit domain as indicated. IL-12 Fc was injected at either 0.01 mg/kg (top panel) or 0.1 mg/kg (bottom panel) at time 0 hours and at time 168 hours as indicated by the downward facing arrows. Data points were collected across multiple timepoints as indicated on the graphs.

FIG. 50 shows the concentration of IL-12 Fc present in the plasma of mice that were injected with 10×10⁶ freshly isolated human PBMC at time 0 hours and either wild-type IL-12 Fc or heterodimeric IL-12 Fc fusion proteins containing mutations within the IL-12p35 subunit domain as indicated. IL-12 Fc was injected at either 0.01 mg/kg (top panel) or 0.1 mg/kg (bottom panel) at time 0 hours and at time 168 hours as indicated by the downward facing arrows. Data points were collected across multiple timepoints as indicated on the graphs.

FIG. 51 shows the number of CD4+ cells and CD8+ cells present in 1 μl of mouse blood and the expression of Granzyme B, Lag-3, PD-1, T-bet, CD45RA+/CD45RO− and Ki-67, on CD8+ T cells seven days after injection of 2.5×10⁶ T Cell Receptor Alpha Constant (TRAC) knockout, 1G4 transduced primary T cells and either wild-type IL-12 Fc or heterodimeric IL-12 Fc fusion proteins containing mutations within the IL-12p35 subunit domain as indicated. The horizontal line represents the median value of measurements performed from five-six mice per group (symbols).

FIG. 52 shows the number of CD4+ cells and CD8+ cells present in 1 μl of mouse blood and the expression of Granzyme B, Lag-3, PD-1, T-bet, CD45RA+/CD45RO− and Ki67, on CD8+ T cells fourteen days after injection of 2.5×10⁶ TRAC knockout, 1G4 transduced primary T cells and either wild-type IL-12 Fc or heterodimeric IL-12 Fc fusion proteins containing mutations within the IL-12p35 subunit domain as indicated. The horizontal line represents the median value of measurements performed from five-six mice per group (symbols).

FIG. 53 shows the concentration of IL-12 Fc present in the plasma of mice that were injected with 2.5×10⁶ TRAC knockout, 1G4 transduced primary T cells and either wild-type IL-12 Fc or heterodimeric IL-12 Fc fusion proteins containing mutations within the IL-12p35 subunit domain as indicated at day 1, 7, 14, and 21. Symbols and error bars represent the mean and standard deviation, respectively, of measurements performed from plasma from five-six mice via Luminex assay.

FIG. 54 shows the concentration of IFNγ present in the plasma of mice that were injected with 2.5×10⁶ TRAC knockout, 1G4 transduced primary T cells and either wild-type IL-12 Fc or heterodimeric IL-12 Fc fusion proteins containing mutations within the IL-12p35 subunit domain as indicated at day 1, 7, 14, and 21. Symbols and error bars represent the mean and standard deviation, respectively, of measurements performed from plasma from five-six mice via Luminex assay.

FIG. 55 shows tumor growth curves for NSG mice (n=5-6 per treatment group) that were injected subcutaneously with 2×10⁵ HLA-A2/NY-ESO-1 A4 variant-expressing HCT116 human colorectal carcinoma cells on day −14 and then treated on day 0 with 2.5×10⁶ TRAC knockout, 1G4 transduced primary T cells and either wild-type IL-12 Fc or heterodimeric IL-12 Fc fusion proteins containing mutations within the IL-12p35 subunit domain as indicated on day 0, 7, and 14. Symbols and error bars represent the mean and standard error of the mean, respectively.

FIG. 56 shows tumor measurements on day 18 for NSG mice (n=5-6 per treatment group) that were injected subcutaneously with 2×10⁵ HLA-A2/NY-ESO-1 A4 variant-expressing HCT116 human colorectal carcinoma cells on day −14 and then treated on day 0 with 2.5×10⁶ TRAC knockout, 1G4 transduced primary T cells and either wild-type IL-12 Fc or heterodimeric IL-12 Fc fusion proteins containing mutations within the IL-12p35 subunit domain as indicated. Symbols represent tumor volumes for individual mice whereas the black lines represent the median within each treatment group. Statistics were calculated using one-way ANOVA. ns indicates not significant, ** indicates P<0.01.

FIGS. 57A and 57B show non-reducing SDS-PAGE analysis of protein A purified heterodimeric IL-12 Fc fusion proteins, wherein the IL-12p35 subunit domain contains either one or two mutations, as indicated.

FIGS. 58A and 58B show reducing SDS-PAGE analysis of protein A purified heterodimeric IL-12 Fc fusion proteins, wherein the IL-12p35 subunit domain contains either one or two mutations, as indicated.

FIG. 59 shows AlphaLISA measurements of IFNγ in the medium of activated T cells that were either unstimulated or incubated with either protein A purified expi-CHO media (“No DNA”) or with 20 pM wild-type IL-12 Fc (“WT”) or 20 pM heterodimeric IL-12 Fc fusion proteins, wherein the IL-12p35 subunit domain contained a mutation at amino acid position 40 as indicated on the x-axis. The horizontal line indicates the amount of IFNγ produced following treatment with IL-12 Fc Y40Y.

FIG. 60 shows AlphaLISA measurements of IFNγ in the medium of activated T cells that were either unstimulated or incubated with either protein A purified expi-CHO media (“No DNA”) or with 80 pM wild-type IL-12 Fc (“WT”) or 80 pM heterodimeric IL-12 Fc fusion proteins, wherein the IL-12p35 subunit domain contained a Y40A mutation and an additional mutation at amino acid position 126 as indicated on the x-axis. The horizontal line indicates the amount of IFNγ produced following treatment with IL-12 Fc Y40A/D126D.

FIG. 61 shows AlphaLISA measurements of IFNγ in the medium of activated T cells that were either unstimulated or incubated with either protein A purified expi-CHO media (“No DNA”) or with 80 pM wild-type IL-12 Fc (“WT”) or 80 pM heterodimeric IL-12 Fc fusion proteins, wherein the IL-12p35 subunit domain contained a Y40A mutation and an additional mutation at amino acid position 127 as indicated on the x-axis. The horizontal line indicates the amount of IFNγ produced following treatment with IL-12 Fc Y40A/P127P.

FIG. 62 shows AlphaLISA measurements of IFNγ in the medium of activated T cells that were either unstimulated or incubated with either protein A purified expi-CHO media (“No DNA”) or with 80 pM wild-type IL-12 Fc (“WT”) or 80 pM heterodimeric IL-12 Fc fusion proteins, wherein the IL-12p35 subunit domain contained a Y40A mutation and an additional mutation at amino acid position 129 as indicated on the x-axis. The horizontal line indicates the amount of IFNγ produced following treatment with IL-12 Fc Y40A/R129R.

FIG. 63 shows AlphaLISA measurements of IFNγ in the medium of activated T cells that were either unstimulated or incubated with either protein A purified expi-CHO media (“No DNA”) or with 80 pM wild-type IL-12 Fc (“WT”) or 80 pM heterodimeric IL-12 Fc fusion proteins, wherein the IL-12p35 subunit domain contained a Y40A mutation and an additional mutation at amino acid position 168 as indicated on the x-axis. The horizontal line indicates the amount of IFNγ produced following treatment with IL-12 Fc Y40A/K168K.

FIG. 64 shows AlphaLISA measurements of IFNγ in the medium of activated T cells that were either unstimulated or incubated with either protein A purified expi-CHO media (“No DNA”) or with 20 pM wild-type IL-12 Fc (“WT”) or 20 pM heterodimeric IL-12 Fc fusion proteins, wherein the IL-12p35 subunit contained a mutation at amino acid position 170 as indicated on the x-axis. The horizontal line indicates the amount of IFNγ produced following treatment with IL-12 Fc K170K.

FIG. 65 shows the activity of wild type IL-12 Fc and heterodimeric IL-12 Fc fusion proteins, wherein the IL-12p35 subunit domain comprises a Y40X single mutant (top panel) or a Y40A/K168X double mutant (bottom panel), in a primary T cell IFNγ release assay. Symbols and error bars indicate the mean and SD, respectively.

FIG. 66 shows a summary of EC₅₀ values and fold change (relative to w.t.) of heterodimeric IL-12 Fc fusion proteins, wherein the IL-12p35 subunit domain comprises one or two mutations, using a primary T cell IFNγ release assay.

FIG. 67 shows thermal unfolding profiles for WT IL-12 Fc and heterodimeric IL-12 Fc fusion proteins, wherein the IL-12p35 subunit domain comprises either Y40A, D126A, R129A, P127A, K168A or K170A as indicated. The solid line represents the mean of three independent replicates.

FIG. 68 shows the percentage activity of heterodimeric IL-12 Fc fusion proteins, wherein the IL-12p35 subunit domain comprises either one or two mutations as indicated, in a primary T cell IFNγ release assay. Percentage activity refers to activity of samples following incubation at a given temperature (indicated in the title of each panel) relative to the activity of untreated or 48° C. treated samples (as indicated on the y-axis of each panel).

FIG. 69 shows a summary of individual melting temperatures (TM₁ and TM₂) determined by differential scanning fluorimetry (DSF) for WT IL-12 Fc and heterodimeric IL-12 Fc fusion proteins, wherein the IL-12p35 subunit domain comprises either one or two mutations.

FIG. 70 shows the activity of three different batches of WT IL-12 Fc (WT1, WT2 and WT3) and heterodimeric IL-12 Fc fusion proteins, wherein the IL-12p35 subunit domain comprises one or two mutations, in a primary T cell IFNγ release assay. Symbols and error bars indicate the mean and SD, respectively.

FIG. 71 shows a summary of EC₅₀ values and fold change (relative to w.t.) of three different batches of WT IL-12 Fc (WT1, WT2 and WT3) and heterodimeric IL-12 Fc fusion proteins, wherein the IL-12p35 subunit domain comprises one or two mutations, using a primary T cell IFNγ release assay in four separate donors.

FIG. 72 shows a killing curve generated from incubation of TRAC knockout, 1G4 transduced primary T cells (effectors) with HLA-A2/NY-ESO A4 variant/GFP transduced HCT116 cells (targets) at a 2.5:1 effector to target cell ratio. Prior to assay setup, effectors were incubated overnight with 0-10 ng/ml WT IL-12 Fc as indicated by the symbols. The number of target cells remaining (depicted as confluence of GFP normalized to 0h) is plotted as a function of time.

FIG. 73 shows a summary of the results of a killing assay whereby TRAC knockout, 1G4 transduced primary T cells (effectors) were incubated with HLA-A2/NY-ESO A4 variant/GFP transduced HCT116 cells (targets) at a 2.5:1 effector to target cell ratio. Prior to assay setup, effectors were incubated overnight with either 0-10 ng/ml WT IL-12 Fc (clear bars) or a single defined concentration of a heterodimeric IL-12 Fc fusion proteins, wherein the IL-12p35 subunit domain comprised one or two mutations (shaded bars), as indicated. Bars and error bars represent the mean and standard deviation of the number of target cells remaining at the 8h timepoint (depicted as confluence of GFP). Horizontal dashed lines represent the number of remaining target cells following incubation with 0 and 10 ng/ml WT IL-12 Fc (upper and lower horizontal dashed lines, respectively).

FIG. 74 shows a summary of interpolated fold change in activity relative to WT IL-12 Fc of heterodimeric IL-12 Fc fusion proteins, wherein the IL-12p35 subunit domain comprises one or two mutations, as determined by a T cell killing assay.

FIG. 75 shows a summary of individual melting temperatures (TM₁ and TM₂) determined by DSF for WT IL-12 Fc and heterodimeric IL-12 Fc fusion proteins, wherein the IL-12p35 subunit domain comprises either one or two mutations.

FIG. 76 shows a summary of EC₅₀ values and fold change (relative to w.t.) of WT IL-12 Fc and heterodimeric IL-12 Fc fusion proteins, wherein the IL-12p35 subunit domain comprises one or two mutations, using a primary T cell IFNγ release assay.

FIG. 77 shows a schematic representation of an exemplary IL-12 Fc fusion protein in a monovalent format.

FIGS. 78A-78G show a list of additional variant IL-12p35 subunit sequences.

FIGS. 79A-79D show a list of additional exemplary heterodimeric Fc fusion proteins and associated sequences.

FIGS. 80A-80M show a list of additional variant IL-12p35 subunit sequences (alanine mutations, select non-alanine mutations, select alanine mutations in combination with select non-alanine mutations, and select non-alanine mutations in combination with additional non-alanine mutations).

FIG. 81 shows the sequences for “single chain trimer of HLA-A2, β2-microglobulin and NY-ESO-1 A4 variant peptide” and “1G4 T cell receptor beta chain-P2A-1G4 T cell receptor alpha chain.”

FIG. 82 shows the activity of two different configurations of WT IL-12 Fc (p40-Fc(knob)/p35-Fc(hole) and p40-Fc(hole)/p35-Fc(knob)) and heterodimeric IL-12 Fc fusion proteins, wherein the IL-12p35 subunit domain comprises two mutations, in a primary T cell IFNγ release assay. Symbols and error bars indicate the mean and SD, respectively.

FIG. 83 shows a summary of EC₅₀ values and fold change (relative to w.t.) of two different configurations of WT IL-12 Fc (p40-Fc(knob)/p35-Fc(hole) and p40-Fc(hole)/p35-Fc(knob)) and heterodimeric IL-12 Fc fusion proteins, wherein the IL-12p35 subunit domain comprises two mutations, using a primary T cell IFNγ release assay.

### DETAILED DESCRIPTION OF THE INVENTION

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety respective to the related technology.

### I. Definitions

Unless defined otherwise, technical, and scientific terms used herein have the same meaning as commonly understood by one of ordinary skill in the art to which the present disclosure belongs. For purposes of the present disclosure, the following terms are defined below. The definitions provided are intended to apply to a given term, as well as other derivative linguistic re-phrasings and grammatical equivalents of the term.

As used herein, the term “protein” refers to at least two, three, four, five, six, seven, eight, nine, 10, 11, 12, 13, 14, 15, 16, 17, 18, 19, 20, 30, 40, 50, 60, 70, 80, 90, 100 or more covalently attached amino acids, which includes proteins, polypeptides, oligopeptides, and peptides. When a “biologically functional molecule” comprises two or more proteins, such as, for example, IL-12 (which comprises an IL-12p35 polypeptide and an IL-12p40 polypeptide), the individual proteins comprising the two or more proteins may each be referred to as a “subunit,” “monomer,” or “domain,” and the biologically functional molecule may be referred to as a “complex.” In some embodiments, the two or more proteins of a functional complex are non-covalently attached. In some embodiments, the two or more proteins of a functional complex are covalently attached, such as, for example, through a disulfide bond.

As used herein, the term “cytokine” refers to a broad category of proteins, such as, for example, chemokines, interferons, interleukins, lymphokines, tumor necrosis factors, and the like, that are secreted by a first cell to cause an effect on one or more cells through a binding of the secreted cytokine to a receptor on the one or more cells. Cytokines may be involved in autocrine, paracrine, juxtacrine, and/or endocrine signaling.

As used herein, the term “wild-type” refers to an amino acid sequence or a nucleotide sequence that is found in nature, including allelic variations. A wild-type protein has an amino acid sequence (or a nucleotide sequence encoding the amino acid sequence) that has not been intentionally modified.

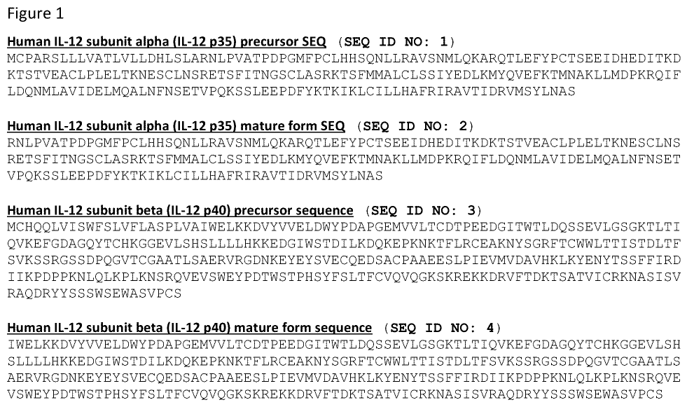

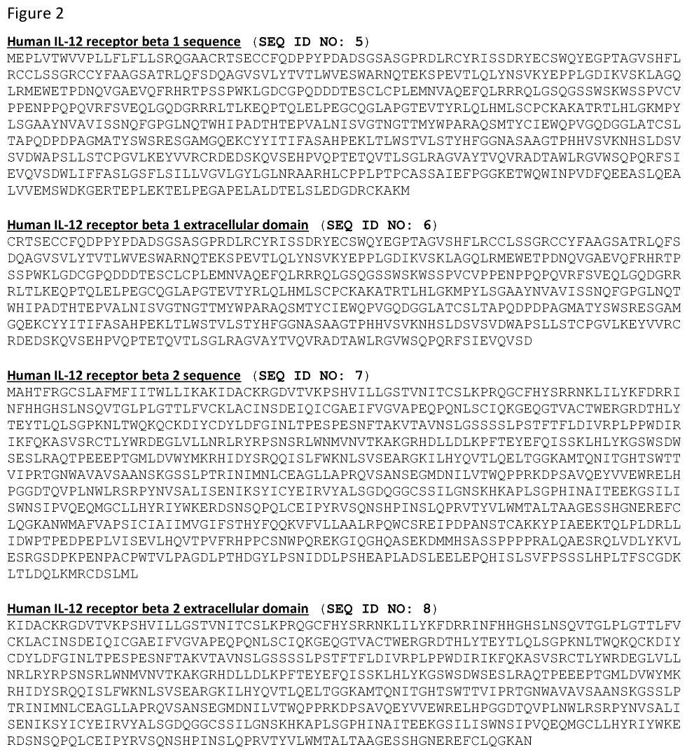

As used herein, the term “IL-12p35 subunit” refers to the human wild-type IL-12p35 polypeptide, whether native or recombinant. As such, an IL-12p35 subunit refers to recombinantly produced IL-12p35 polypeptide, synthetically produced IL-12p35 polypeptide, as well as IL-12p35 extracted from cells or tissues. An amino acid sequence of human wild-type IL-12p35 subunit is depicted in FIG. 1 (Precursor: SEQ ID NO: 1; Mature: SEQ ID NO: 2). In the context of a fusion protein, the IL-12p35 subunit may also be referred to as an “IL-12p35 subunit domain,” wherein the IL-12p35 subunit domain comprises at least part of the amino acid sequence encoding the IL-12p35 subunit.

As used herein, the term “IL-12p40 subunit” refers to the human wild-type IL-12p40 polypeptide, whether native or recombinant. As such, an IL-12p40 subunit refers to recombinantly produced IL-12p40 polypeptide, synthetically produced IL-12p40 polypeptide, as well as IL-12p40 extracted from cells or tissues. An amino acid sequence of human wild-type IL-12p40 subunit is depicted in FIG. 1 (Precursor: SEQ ID NO: 3; Mature: SEQ ID NO: 4). In the context of a fusion protein, the IL-12p40 subunit may also be referred to as an “IL-12p40 subunit domain,” wherein the IL-12p40 subunit domain comprises at least part of the amino acid sequence encoding the IL-12p40 subunit. As used herein, the phrase “(variant) IL-12p40 subunit” is used to disclose embodiments wherein the IL-12p40 subunit comprises a wildtype IL-12p40 polypeptide or a variant IL-12p40 subunit.

As used herein, the term “single-chain” refers to a molecule comprising two or more protein domains linearly linked by peptide bonds. In some embodiments, the biologically functional IL-12 is a single chain IL-12 complex (sc-IL-12) (i.e., the IL-12p35 subunit and the IL-12p40 subunit are fused to form a single peptide chain). In further embodiments, the C-terminus of the IL-12p35 subunit is connected to the N-terminus of the IL-12p40 subunit (sc-IL-12(p35/p40)). In yet further embodiments, the sc-IL-12(p35/p40) further comprises a linker, wherein the C-terminus of the IL-12p35 subunit is linked to the N-terminus of the linker and the C-terminus of the linker is linked to the N-terminus of the IL-12p40 subunit. In other embodiments, the C-terminus of the IL-12p40 subunit is connected to the N-terminus of the IL-12p35 subunit (sc-IL-12(p40/p35)). In yet further embodiments, the sc-IL-12(p40/p35) further comprises a linker, wherein the C-terminus of the IL-12p40 subunit is linked to the N-terminus of the linker and the C-terminus of the linker is linked to the N-terminus of the IL-12p35 subunit.

As used herein, the term “residue” refers to a position in a protein and its associated amino acid identity. For example, Cysteine 252 (also referred to as Cys252 or C252) is a residue at position 252.

As used herein, the term “parent protein” refers to a “reference” protein, the amino acid sequence that encodes the reference protein, or the DNA sequence that encodes the amino acid sequence that encodes the reference protein. In some embodiments, the reference protein comprises a wild-type protein, the amino acid sequence encoding the wild-type protein, and/or the nucleic acid sequence encoding the amino acid sequence encoding the wild-type protein. In some embodiments, the reference protein comprises a human wild-type protein, the amino acid sequence encoding the human wild-type protein, and/or the nucleic acid sequence encoding the amino acid sequence encoding the human wild-type protein. In some embodiments, the reference protein comprises a (human) wild-type protein conjugated to an Fc domain.

As used herein, the terms “variant protein,” “protein variant,” or “variant” refer to a protein that differs from that of a parent protein by virtue of at least one, two, three, four, five, six, seven, eight, nine, 10, 11, 12, 13, 14, 15, 16, 17, 18, 19, 20, 25, 30, 40, 50, 60, 70, 80, 90, 100 or more modifications. The terms may refer to the protein itself, a composition comprising the protein, the amino acid sequence that encodes it, or the DNA sequence that encodes it. In some embodiments, the parent protein refers to a wild-type sequence. In some embodiments, the parent protein refers to a human wild-type sequence. Thus, a “variant” of an IL-12p35 subunit (or “variant” of an IL-12p35 subunit domain) refers to a polypeptide in which one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions, deletions, and/or insertions are present as compared to the amino acid sequence of a reference IL-12p35 subunit, such as, for example, a (human) wild-type IL-12p35 subunit. Further, a “variant” of an IL-12p40 subunit (or “variant” of an IL-12p40 subunit domain) refers to a polypeptide in which one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions, deletions, and/or insertions are present as compared to the amino acid sequence of a reference IL-12p40 subunit, such as, for example, a (human) wild-type IL-12p40 subunit.

As used herein, the term “modification” refers to an amino acid substitution, insertion, and/or deletion in a polypeptide sequence or an alteration to a moiety chemically linked to a protein. As used herein, the term “amino acid modification” refers to an amino acid substitution, insertion, and/or deletion in a polypeptide sequence. The position(s) where the amino acid(s) are modified and the number of amino acid(s) that may be modified in the amino acid sequence are not particularly limited.

As will be appreciated the modifications to the IL-12p35 subunit (domain) described throughout refer to modifications made to the mature form of the sequence (SEQ ID NO: 2) and/or variants thereof, as opposed to the precursor sequence (SEQ ID NO: 1). The precursor sequence for the IL-12p35 subunit comprises an additional 22 amino acid residues on the N-terminus comprising the sequence: MCPARSLLLVATLVLLDHLSLA. However, the listed modifications to the mature form of the IL-12p35 subunit can be made to the precursor form of the IL-12p35 subunit after correcting the position of the stated modification in view of the additional leading amino acid residues in the precursor sequence. For example, the substitution modification Y40A is disclosed herein as a potential modification to the IL-12p35 subunit mature sequence but can also refer to a substitution modification of the IL-12p35 subunit precursor sequence comprising Y62A.

Similarly, modifications to the IL-12p40 subunit (domain) described throughout are intended to refer to modifications made to the mature form of the sequence (SEQ ID NO: 4) and/or variants thereof, as opposed to the precursor sequence (SEQ ID NO: 3). The precursor sequence for the IL-12p40 subunit comprises an additional 22 amino acid residues on the N-terminus comprising the sequence: MCHQQLVISWFSLVFLASPLVA. However, it will also be appreciated that the listed modifications to the mature form of the IL-12p40 subunit can be made to the precursor form of the IL-12p40 subunit after correcting the position of the stated modification in view of the additional leading amino acid residues in the precursor sequence. For example, the substitution modification C177S is disclosed herein as a potential modification to the IL-12p40 subunit mature sequence but can also refer to a substitution modification of the IL-12p40 subunit precursor sequence comprising C199S.

As used herein, the terms “amino acid substitution” or “substitution” refer to the replacement of an amino acid at a particular position in a parent polypeptide sequence with a different amino acid. For example, Y40A designates a substitution of tyrosine at position 40 with an alanine at the same position. In some embodiments, the substitution is to an amino acid that is not naturally occurring at the particular position, either not naturally occurring within the organism or in any organism. For clarity, a protein that has been engineered to change the nucleic acid coding sequence but not to change the resulting amino acid (for example, exchanging CCU (encoding proline) to CCC (still encoding proline)) is not an “amino acid substitution.” Phrased differently, despite the creation of a new gene encoding the same protein, if the protein has the same amino acid at the particular position that it started with, it is not an amino acid substitution.

As used herein, the terms “amino acid insertion” or “insertion” refer to the addition of an amino acid residue or sequence at a particular position in a parent polypeptide sequence. For example, −40A designates an insertion of alanine after position 40 and before position 41. As a separate example, D8EPKSS or −8EPKSS designates an insertion of the sequence Glu-Pro-Lys-Ser-Ser after position 8 and before position 9.

As used herein, the terms “amino acid deletion” or “deletion” refer to the removal of an amino acid or sequence at a particular position in a parent polypeptide sequence. For example, Y40-, Y40#, Y40( ), or Y40del designates a deletion of tyrosine at position 40. As a separate example, EPKSS8-, EPKSS8#, EPKSS8del designates a deletion of the sequence Glu-Pro-Lys-Ser-Ser that begins at position 8.

As used herein, the terms “non-naturally occurring protein” or “non-naturally occurring protein variant” refer to a variant protein that differs from that of a parent protein by virtue of at least one, two, three, four, five, six, seven, eight, nine, 10, 11, 12, 13, 14, 15, 16, 17, 18, 19, 20, 25, 30, 40, 50, 60, 70, 80, 90, 100 or more modifications that are not isotypic. For example, because the IL-12p35 subunit of IL-12 does not comprise an alanine at position 40, the substitution Y40A is considered a non-naturally occurring IL-12p35 variant (or, more generally, a non-naturally occurring IL-12 variant). Modifications to a protein that are not isotypic can be referred to as “non-naturally occurring modifications.”

As used herein, the terms “percent (%) identity” and “percent (%) sequence identity,” when used in the context of two or more proteins or nucleic acids, refer to a percentage of amino acid residues (or nucleic acids encoding the amino acid residues) in a candidate sequence that are identical with the amino acid residues (or nucleic acids encoding the amino acid residues) in a specific sequence, such as, for example, the amino acid sequence of a parent protein, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill of the art, such as, for example, using publicly available computer software (e.g., BLAST, BLAST-2, ALIGN, or Megalign (DNASTAR) software). Those skilled in the art can determine appropriate parameters for measuring alignment, including any algorithms needed to achieve maximal alignment over the full length of the sequences being compared. In some embodiments, two or more amino acid sequences are at least about 50%, 60%, 70%, 80%, 90%, 91%, 92%, 93%, 94%, 95%, 96%, 97%, 98%, 99%, or even 100% identical.

As used herein, the term “half-life” of an agent can refer to the time it takes for the agent to lose half of its pharmacologic, physiologic, or other activity, relative to such activity at the time of administration into the serum or tissue of an organism or subject, or relative to any other defined time-point. “Half-life” can also refer to the time it takes for the amount or concentration of an agent to be reduced by half of a starting amount administered into the serum or tissue of an organism or subject, relative to such amount or concentration at the time of administration into the serum or tissue of an organism or subject, or to any other defined time-point. The half-life can be measured in serum and/or any one or more selected tissues.

As used herein, the terms “host cell” and “recombinant cell” refer to an individual cell or a cell culture that can be or has been a recipient of any recombinant vector(s) or isolated polynucleotide(s). A host cell can be a transfected, transformed, transduced, or infected cell of any origin, including, but not limited to, prokaryotic, eukaryotic, mammalian, avian, insect, plant, or bacteria cells, or it can be of any origin that can be used to propagate a nucleic acid described herein. A host cell includes cells transfected or infected in vivo or in vitro with a recombinant vector or a polynucleotide of the invention. A host cell that comprises a recombinant vector of the invention may be called a “recombinant host cell.”

Host cells can include, without limitation, the cells of mammals, plants, insects, fungi, and bacteria. Bacterial cells include, without limitation, the cells of Gram-positive bacteria, such as, for example, species of the genus Bacillus, Streptomyces, and Staphylococcus, and cells of Gram-negative bacteria, such as, for example, cells of the genus Escherichia and Pseudomonas. Fungal cells can include, without limitation, yeast cells, such as, for example, Saccharomyces, Pichia pastoris, and Hansenula polymorpha. Insect cells can include, without limitation, cells of Drosophila and Sf9 cells. Plant cells include, without limitations, cells from crop plants, medicinal or ornamental plants or bulbs. Suitable mammal cells for the present invention include, but are not limited to, epithelial cell lines (e.g., porcine epithelial cells), osteosarcoma cell lines, neuroblastoma cell lines, epithelial carcinomas, glial cells, liver cell lines, Chinese hamster ovary (CHO) cells, COS cells, BHK cells, HeLa cells, D3 cells of the line of murine embryonic stem cells (mESCs), human embryonic stem cells (e.g., HS293 cells and BG01V cells), NIH 3T3 cells, human embryonic kidney (HEK) 293T cells, human mesenchymal stem cells (hMSCSs), and the like.

As used herein, the terms “cell,” “cell culture,” “cell line,” and “host cell” refer not only to the particular cell, cell culture, cell line, or host cell, but also to the progeny or potential progeny of such a cell, cell culture, cell line, or host cell, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the terms as used herein, so long as the progeny retain the same functionality or substantially the same functionality as that of the original cell, cell culture, cell line, or host cell.

As used herein, the terms “medium” or “culture medium” include any culture medium, solution, solid, semi-solid, or rigid support that may support or contain any host cells, including, but not limited to, bacterial host cells, yeast or fungal host cells, insect host cells, plant host cells, eukaryotic host cells, mammalian host cells, CHO cells, prokaryotic host cells, E. coli host cells, Pseudomonas host cells, and the like, and cell contents. Thus, the term may encompass medium in which the host cell has been grown, (e.g., medium into which a polypeptide has been secreted, including medium either before, during, or after a proliferation step). The term also may encompass buffers or other reagents that contain host cell lysates, such as, for example, in the case where a polynucleotide is produced intracellularly, and the host cells are lysed or disrupted to release the polypeptide.

As used herein, the term “fusion protein” refers to a covalent joining of at least two proteins or protein domains. Fusion proteins may comprise artificial sequences, such as, for example, a domain linker, variant Fc domains, a variant IL-12p35 subunit domain, a(n) (variant) IL-12p40 subunit domain, and the like, as described herein. As used herein, the term “Fc fusion protein” refers to a protein comprising an Fc domain, generally linked (optionally through a domain linker, as described herein) to one or more different protein domains. In some embodiments, the C-terminus of the Fc domain is linked to the N-terminus of one or more different protein domains (optionally through a domain linker, wherein the C-terminus of the Fc domain is linked to the N-terminus of the domain linker and the C-terminus of the domain linker is linked to the N-terminus of the one or more different protein domains). In some embodiments, the N-terminus of the Fc domain is linked to the C-terminus of one or more different protein domains (optionally through a domain linker, wherein the C-terminus of the one or more protein domains is linked to the N-terminus of the domain linker and the C-terminus of the domain linker is linked to the N-terminus of the Fc domain). Accordingly, an “IL-12 Fc fusion” comprises an Fc region linked (optionally through a domain linker) to a variant IL-12p35 subunit domain, an IL-12p40 subunit domain, a variant IL-12p40 subunit domain, a sc-IL-12, a sc-IL-12(p35/p40), and/or a sc-IL-12(p40/p35). A “Fc fusion protein” may refer to a “heterodimeric Fc fusion protein” or a “homodimeric Fc fusion protein.”

As used herein, the term “heterodimeric Fc fusion protein” refers to a complex comprising a first fusion construct and a second fusion construct, wherein the first fusion construct comprises a first Fc domain and a first IL-12 subunit domain, and wherein the second fusion construct comprises a second Fc domain and a second IL-12 subunit domain. In some embodiments, the first Fc domain and the second Fc domain comprise modifications that promote heterodimerization of the first and second Fc domains. In some embodiments, the first Fc domain and/or the second Fc domain comprises one or more modifications that alter Fc binding. In some embodiments, the first Fc domain and/or the second Fc domain comprises one or more modifications that alter Fc half-life. In some embodiments, the first Fc domain and/or the second Fc domain comprises one or more modifications that alter the binding of the first Fc domain and/or the second Fc domain to the neonatal Fc receptor (FcRn). In certain further embodiments, the first Fc domain and/or the second Fc domain comprise one or more modifications that increase half-life and/or binding to FcRn. In some embodiments, the first IL-12 subunit domain comprises a variant IL-12p35 subunit domain, and the second IL-12 subunit domain comprises an IL-12p40 subunit domain or a variant IL-12p40 subunit domain. In some embodiments, the first IL-12 subunit domain comprises an IL-12p40 subunit domain or a variant IL-12p40 subunit domain, and the second IL-12 subunit domain comprises a variant IL-12p35 subunit domain. In some embodiments, the C-terminus of the first IL-12 subunit domain is linked to the N-terminus of the first Fc domain (optionally through a domain linker), and the C-terminus of the second IL-12 subunit domain is linked to the N-terminus of the second Fc domain (optionally through a domain linker). In some embodiments, the C-terminus of the first Fc domain is linked to the N-terminus of the first IL-12 subunit domain (optionally through a domain linker), and the C-terminus of the second Fc domain is linked to the N-terminus of the second IL-12 subunit domain (optionally through a domain linker).

As used herein, the term “homodimeric fusion protein” refers to a complex comprising two identical instances of a fusion construct, wherein the individual instances of the fusion construct comprise a Fc domain and one or more protein domains (optionally linked through a domain linker). In some embodiments, the C-terminus of the Fc domain is linked to the N-terminus of the one or more protein domains (optionally through a domain linker) for both instances of the fusion construct. In some embodiments, the C-terminus of the one or more protein domains is linked to the N-terminus of the Fc domain (optionally through a domain linker) for both instances of the fusion construct. In some embodiments, the identical instances of the Fc domain comprise one or more modifications that alter Fc binding. In some embodiments, the identical instances of the Fc domain comprise one or more modifications that alter Fc half-life. In some embodiments, the identical instances of the Fc domain comprise one or more modifications that alter the binding of the identical instances of the Fc domain to the neonatal Fc receptor (FcRn). In certain further embodiments, the identical instances of the domain comprise one or more modifications that increase half-life and/or binding to FcRn.

As used herein, the term “isolated,” when used to describe the various polypeptides disclosed herein, refers to a polypeptide that has been identified and separated and/or recovered from a cell (e.g., a host cell and/or a cell line) or cell culture from which it was expressed. Generally, an isolated polypeptide will be prepared by at least one purification step. As used herein, the term “isolated protein” refers to a protein which is substantially free of other proteins from a cell culture, such as, for example, host cell proteins.

As used herein, the terms “Fc,” “Fc region,” or “Fc domain” refer to the polypeptide comprising the CH2-CH3 domains of an immunoglobulin G (IgG) molecule, and in some cases, inclusive of all or part of the hinge, as well as variants thereof. In EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge-CH2-CH3), or fragments thereof. A “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.). Herein, unless specifically outlined, a “Fc domain” generally refers to the CH2-CH3 domains (and optionally all or part of the hinge) of human IgG1. Described below are various human IgG1 Fc domains, at least some of which comprise one or more modifications. For clarity, while the modifications are discussed herein primarily in the context of human IgG1 Fc domains for the sake of brevity, it is expressly contemplated that mutations to other immunoglobulins, such as, for example, IgG2, IgG3, IgG4, IgA, IgM, and IgE, can be made at residue positions corresponding to the mutations in human IgG1 that are described herein. Those skilled in the relevant art will readily be able to determine residue locations in other immunoglobulins that correspond with the modifications to human IgG1 described herein.

As used herein, the term “vector” refers to a nucleic acid molecule or sequence capable of transferring or transporting another nucleic acid molecule. The transferred nucleic acid molecule is generally linked to (e.g., inserted into) the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments thereof in vitro and/or in vivo. A vector may include sequences that direct autonomous replication in a cell or may include sequences sufficient to allow integration into host cell DNA. Useful vectors include, but are not limited to, plasmids (e.g., DNA plasmids or RNA plasmids), transposons, cosmids, bacterial artificial chromosomes, and viral vectors. Useful viral vectors include, but are not limited to, replication defective retroviruses and lentiviruses. In some embodiments, a vector is a gene delivery vector. In some embodiments, a vector is used as a gene delivery vehicle to transfer a gene into a cell.

As used herein, the term “recombinant,” with respect to a nucleic acid molecule, refers to a polynucleotide of genomic, cDNA, viral, semisynthetic, and/or synthetic origin which, by virtue of its own origin or manipulation, is not associated with all or a portion of the polynucleotide with which it is associated in nature. The term “recombinant,” as used with respect to a protein or polypeptide, refers to a polypeptide produced by expression of a recombinant polynucleotide. The term “recombinant,” as used with respect to a host cell, refers to a host cell into which a recombinant polynucleotide or vector comprising a recombinant polynucleotide has been introduced.

As used herein, the term “operably linked” refers to a physical or functional linkage between two or more elements (e.g., polypeptide sequences or polynucleotide sequences) that permits them to operate in their intended fashion. For example, an operable linkage between a polynucleotide of interest and a regulatory sequence (such as, for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. Thus, a promoter is in an operable linkage with a nucleic acid sequence if it can mediate transcription of the nucleic acid sequence. It should be understood that elements that are operably linked may be contiguous or non-contiguous. In the context of a polypeptide, “operably linked” refers to a physical linkage (e.g., directly, or indirectly linked) between amino acid sequences (e.g., different segments, modules, or domains) to provide for a described activity of the polypeptide.

As used herein, the term “binding affinity” refers to the “strength” of binding of a given molecule (such as, for example, a non-naturally occurring IL-12 variant, a homodimeric IL-12 Fc fusion protein, and/or a heterodimeric IL-12 Fc fusion protein) to its ligand (such as, for example, IL-12Rβ2) and/or the rate at which the molecule associates with and/or dissociates from its ligand. Binding affinity is often expressed in terms of the dissociation constant (K_{D}). The binding activity of the non-naturally occurring IL-12 variants, homodimeric IL-12 Fc fusion proteins, and/or heterodimeric IL-12 Fc fusion proteins of the disclosure can be assayed by any suitable method known in the art, such as, for example, a surface plasmon resonance (SPR) assay, an enzyme-linked immunosorbent assay (ELISA), an ELISpot assay, Biacore assays, KinExA assays, and the like.

As used herein, the term “potency” refers to the ability of a given protein, cytokine, fusion protein, antibody, and the like (such as, for example, a non-naturally occurring IL-12 variant, a homodimeric IL-12 Fc fusion protein, and/or a heterodimeric IL-12 Fc fusion protein) to elicit a response at a certain dose or concentration in a given biological system or experimental setting. The potency of the non-naturally occurring IL-12 variants, homodimeric IL-12 Fc fusion proteins, and/or heterodimeric IL-12 Fc fusion proteins of the disclosure can be assayed by any suitable method known in the art, such as, for example, an IL-12 HEK reporter assay (e.g., InvivoGen's IL-12 HEK reporter assay (Catalog No. hkb-i112)), a ligand-binding assay (e.g., ELISA or flow cytometry), and/or a functional assay. Generally, changes in potency may be demonstrated graphically as a leftward or rightward shift in a response curve compared to a control. A rightward shift of the response curve is generally indicative of a reduction in potency, whereas as a leftward shift of the response curve is generally indicative of an increase in potency.

As used herein, the term “activity” refers to the specific response of a given protein, cytokine, fusion protein, antibody, and the like (such as, for example, a non-naturally occurring IL-12 variant, a homodimeric IL-12 Fc fusion protein, and/or a heterodimeric IL-12 Fc fusion protein) elicited in a particular biological system or experimental setting at a given dose or concentration. The activity of the non-naturally occurring IL-12 variants, homodimeric IL-12 Fc fusion proteins, and/or heterodimeric IL-12 Fc fusion proteins of the disclosure can be assayed by any suitable method known in the art, such as, for example, a ligand-binding assay and/or a functional assay. Generally, changes in activity may be demonstrated graphically as an upward or downward shift in a response curve compared to a control. An upward shift of the response curve is generally indicative of an increase in activity, whereas a downward shift of the response curve is generally indicative of a decrease in activity. As described in further detail below, a change in activity may or may not be related to and/or caused by a change in potency.

As used herein, the term “manufacturability” refers to any property that may impact the process of producing and/or storing a given protein, cytokine, fusion protein, antibody, and the like at a scale and quantity sufficient for administration to an individual. Examples of properties that impact manufacturability include, but are not limited to, the stability, purity, aggregation levels, and/or yield of expression of a given protein, cytokine, fusion protein, antibody, and the like (such as, for example, a non-naturally occurring IL-12 variant, a homodimeric IL-12 Fc fusion protein, and/or a heterodimeric IL-12 Fc fusion protein).

As used herein, the term “stability” refers to the ability of a given protein, cytokine, fusion protein, antibody, and the like (such as, for example, a non-naturally occurring IL-12 variant, a homodimeric IL-12 Fc fusion protein, and/or a heterodimeric IL-12 Fc fusion protein) to retain the same properties and characteristics that it possessed at the time of its manufacture within specified limits and/or storage and/or use parameters. The stability of the non-naturally occurring IL-12 variants, homodimeric IL-12 Fc fusion proteins, and/or heterodimeric IL-12 Fc fusion proteins of the disclosure can be assayed by any suitable method known in the art, such as, for example ELISA, Western blot, Biacore assay, SDS-PAGE, size exclusion chromatography, dynamic light scattering, differential scanning calorimetry, and differential scanning fluorimetry.

As used herein, the term “yield of expression” refers to the amount or quantity of a given protein, cytokine, fusion protein, antibody, and the like (such as, for example, a non-naturally occurring IL-12 variant, a homodimeric IL-12 Fc fusion protein, and/or a heterodimeric IL-12 Fc fusion protein) produced using a prokaryotic or eukaryotic host system. Recombinant expression of proteins, cytokines, fusion proteins, antibodies, and the like are well-known in relevant arts.

Any suitable method for quantitating or determining a yield of expression may be employed, such as, for example, UV absorption measurements, colorimetric assays (e.g., Bradford assay, BCA assay, and Lowry assay), and fluorometric assays.

As used herein, the terms “subject” or an “individual” for purposes of treatment refer to any animal classified as a mammal (including but not limited to humans, primates, and/or non-human primates), domestic animals, farm animals, zoo animals, research animals, sports animals, and/or pet animals, such as dogs, horses, cats, cows, etc.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone).”

As used herein, the term “about” a value (or parameter) refers to ±10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Overview

The present invention is directed to novel, non-naturally occurring IL-12 variants and fusion proteins, wherein the IL-12p35 subunit comprises novel amino acid substitutions that reduce binding affinity to IL-12Rβ2, as well as methods of making and using the non-naturally occurring IL-12 variants and fusion proteins.

As noted above, IL-12 is composed of an α-chain (the p35 subunit; IL-12p35 subunit) and a 3-chain (the p40 subunit; IL-12p40 subunit) covalently linked to form the biologically functional IL-12 complex. IL-12 exerts its cell signaling function by binding to the dimeric IL-12 receptor complex composed of IL-12Rβ1 and IL-12Rβ2, leading to the phosphorylation of STAT4 and the initiation of multiple down-stream signaling pathways, including, but not limited to, the induction of IFNγ secretion. Systemic administration of wild-type IL-12 can result in severe toxicity, including death, due to an over-activation of circulating immune cells. Moreover, the activated immune cells undergo cellular proliferation contributing to a short serum half-life of administered IL-12 due to target-mediated drug disposition.

In some embodiments, the compositions and methods described herein reduce the toxicity associated with IL-12 treatment by providing novel IL-12 variants with decreased binding affinity to IL-12Rβ2. In further embodiments, the compositions and methods described herein utilize IL-12 Fc fusion proteins that in still further embodiments comprise such novel IL-12 variants.

In some embodiments, the compositions and methods described herein addresses the short half-life of IL-12 by providing novel IL-12 variants with decreased binding affinity to IL-12Rβ2. In further embodiments, the half-life is further improved by a fusion of the novel IL-12 variants to one or more Fc domains (such as, for example, one or more of the Fc domains encoded by the amino acid sequences SEQ ID NOs: 9-13, Fc domains comprising one or more modifications resulting in a modified binding to the neonatal Fc receptor (FcRn), and the like), one or more albumin, one or more unstructured biodegradable polypeptides (“XTEN”), or one or more polyethylene glycol (PEG).

In some embodiments, the compositions and methods described herein addresses the short half-life of IL-12 by providing IL-12 Fc fusion proteins with decreased binding affinity to IL-12Rβ2.

### III. Compositions

As will be appreciated by one skilled in the art, any of the aspects and embodiments of the compositions described herein can be used in any of the aspects and/or embodiments of the methods of making described below or in the methods of use also described below.

### A. Interleukin 12 (IL-12) Variants

In one aspect, the present disclosure provides a non-naturally occurring IL-12 variant, comprising: a) a variant IL-12p35 subunit, and b) an IL-12p40 subunit. Various configurations for the non-naturally occurring IL-12 variants, as well as the variant IL-12p35 subunit and the IL-12p40 subunit, are expanded upon below.

1. p35 Subunit:

In accordance with any of the aspects and embodiments described herein, the present disclosure provides a non-naturally occurring IL-12 variant, comprising: a) a variant IL-12p35 subunit wherein the variant IL-12p35 subunit comprises one or more amino acid substitutions selected from the group including: Y40A, T43A, D126A, P127A, R129A, K168A, and K170A; and b) an IL-12p40 subunit. In some embodiments, the one or more amino acid substitutions comprise Y40A. In some embodiments, the one or more amino acid substitutions comprise T43A. In some embodiments, the one or more amino acid substitutions comprise D126A. In some embodiments, the one or more amino acid substitutions comprise P127A. In some embodiments, the one or more amino acid substitutions comprise R129A. In some embodiments, the one or more amino acid substitutions comprise K168A. In some embodiments, the one or more amino acid substitutions comprise K170A.

In some embodiments, the variant IL-12p35 subunit comprises two or more amino acid substitutions selected from the group including: Y40A, T43A, D126A, P127A, R129A, K168A, and K170A. In some embodiments, the two or more amino acid substitutions comprise Y40A/T43A. In some embodiments, the two or more amino acid substitutions comprise Y40A/D126A. In some embodiments, the two or more amino acid substitutions comprise Y40A/P127A. In some embodiments, the two or more amino acid substitutions comprise Y40A/R129A. In some embodiments, the two or more amino acid substitutions comprise Y40A/K168A. In some embodiments, the two or more amino acid substitutions comprise T43A/D126A. In some embodiments, the two or more amino acid substitutions comprise T43A/P127A. In some embodiments, the two or more amino acid substitutions comprise T43A/R129A. In some embodiments, the two or more amino acid substitutions comprise T43A/K168A. In some embodiments, the two or more amino acid substitutions comprise D126A/P127A. In some embodiments, the two or more amino acid substitutions comprise D126A/R129A. In some embodiments, the two or more amino acid substitutions comprise D126A/K168A. In some embodiments, the two or more amino acid substitutions comprise P127A/R129A. In some embodiments, the two or more amino acid substitutions comprise P127A/K168A. In some embodiments, the two or more amino acid substitutions comprise R129A/K168A. In some embodiments, the two or more amino acid substitutions comprise Y40A/K170A. In some embodiments, the two or more amino acid substitutions comprise T43A/K170A. In some embodiments, the two or more amino acid substitutions comprise D126A/K170A. In some embodiments, the two or more amino acid substitutions comprise P127A/K170A. In some embodiments, the two or more amino acid substitutions comprise R129A/K170A. In some embodiments, the two or more amino acid substitutions comprise K168A/K170A.

In some embodiments, the variant IL-12p35 subunit comprises three or more amino acid substitutions selected from the group including: Y40A, T43A, D126A, P127A, R129A, K168A, and K170A. In some embodiments, the three or more amino acid substitutions comprise Y40A/T43A/D126A. In some embodiments, the three or more amino acid substitutions comprise Y40A/T43A/P127A. In some embodiments, the three or more amino acid substitutions comprise Y40A/T43A/R129A. In some embodiments, the three or more amino acid substitutions comprise Y40A/T43A/K168A. In some embodiments, the three or more amino acid substitutions comprise Y40A/D126A/P127A. In some embodiments, the three or more amino acid substitutions comprise Y40A/D126A/R129A. In some embodiments, the three or more amino acid substitutions comprise Y40A/D126A/K168A. In some embodiments, the three or more amino acid substitutions comprise Y40A/P127A/R129A. In some embodiments, the three or more amino acid substitutions comprise Y40A/P127A/K168A. In some embodiments, the three or more amino acid substitutions comprise Y40A/R129A/K168A. In some embodiments, the three or more amino acid substitutions comprise T43A/D126A/P127A. In some embodiments, the three or more amino acid substitutions comprise T43A/D126A/R129A. In some embodiments, the three or more amino acid substitutions comprise T43A/D126A/K168A. In some embodiments, the three or more amino acid substitutions comprise T43A/P127A/R129A. In some embodiments, the three or more amino acid substitutions comprise T43A/P127A/K168A.

In some embodiments, the three or more amino acid substitutions comprise T43A/R129A/K168A. In some embodiments, the three or more amino acid substitutions comprise D126A/P127A/R129A. In some embodiments, the three or more amino acid substitutions comprise D126A/P127A/K168A. In some embodiments, the three or more amino acid substitutions comprise D126A/R129A/K168A. In some embodiments, the three or more amino acid substitutions comprise P127A/R129A/K168A. In some embodiments, the three or more amino acid substitutions comprise Y40A/T43A/K170A. In some embodiments, the three or more amino acid substitutions comprise Y40A/D126A/K170A. In some embodiments, the three or more amino acid substitutions comprise Y40A/P127A/K170A. In some embodiments, the three or more amino acid substitutions comprise Y40A/R129A/K170A. In some embodiments, the three or more amino acid substitutions comprise Y40A/K168A/K170A. In some embodiments, the three or more amino acid substitutions comprise T43A/D126A/K170A. In some embodiments, the three or more amino acid substitutions comprise T43A/P127A/K170A.

In some embodiments, the three or more amino acid substitutions comprise T43A/R129A/K170A. In some embodiments, the three or more amino acid substitutions comprise T43A/K168A/K170A. In some embodiments, the three or more amino acid substitutions comprise D126A/P127A/K170A. In some embodiments, the three or more amino acid substitutions comprise D126A/R129A/K170A. In some embodiments, the three or more amino acid substitutions comprise D126A/K168A/K170A. In some embodiments, the three or more amino acid substitutions comprise P127A/R129A/K170A. In some embodiments, the three or more amino acid substitutions comprise P127A/K168A/K170A. In some embodiments, the three or more amino acid substitutions comprise R129A/K168A/K170A.

In some embodiments, the variant IL-12p35 subunit comprises four or more amino acid substitutions selected from the group including: Y40A, T43A, D126A, P127A, R129A, K168A, and K170A. In some embodiments, the four or more amino acid substitutions comprise Y40A/T43A/D126A/P127A. In some embodiments, the four or more amino acid substitutions comprise Y40A/T43A/D126A/R129A. In some embodiments, the four or more amino acid substitutions comprise Y40A/T43A/D126A/K168A. In some embodiments, the four or more amino acid substitutions comprise Y40A/T43A/P127A/R129A. In some embodiments, the four or more amino acid substitutions comprise Y40A/T43A/P127A/K168A. In some embodiments, the four or more amino acid substitutions comprise Y40A/T43A/R129A/K168A. In some embodiments, the four or more amino acid substitutions comprise Y40A/D126A/P127A/R129A.

In some embodiments, the four or more amino acid substitutions comprise Y40A/D126A/P127A/K168A. In some embodiments, the four or more amino acid substitutions comprise Y40A/D126A/R129A/K168A. In some embodiments, the four or more amino acid substitutions comprise Y40A/P127A/R129A/K168A. In some embodiments, the four or more amino acid substitutions comprise T43A/D126A/P127A/R129A. In some embodiments, the four or more amino acid substitutions comprise T43A/D126A/P127A/K168A. In some embodiments, the four or more amino acid substitutions comprise T43A/D126A/R129A/K168A. In some embodiments, the four or more amino acid substitutions comprise T43A/P127A/R129A/K168A.

In some embodiments, the four or more amino acid substitutions comprise D126A/P127A/R129A/K168A. In some embodiments, the four or more amino acid substitutions comprise Y40A/T43A/D126A/K170A. In some embodiments, the four or more amino acid substitutions comprise Y40A/T43A/P127A/K170A. In some embodiments, the four or more amino acid substitutions comprise Y40A/T43A/R129A/K170A. In some embodiments, the four or more amino acid substitutions comprise Y40A/T43A/K168A/K170A. In some embodiments, the four or more amino acid substitutions comprise Y40A/D126A/P127A/K170A. In some embodiments, the four or more amino acid substitutions comprise Y40A/D126A/R129A/K170A.

In some embodiments, the four or more amino acid substitutions comprise Y40A/D126A/K168A/K170A. In some embodiments, the four or more amino acid substitutions comprise Y40A/P127A/R129A/K170A. In some embodiments, the four or more amino acid substitutions comprise Y40A/P127A/K168A/K170A. In some embodiments, the four or more amino acid substitutions comprise Y40A/R129A/K168A/K170A. In some embodiments, the four or more amino acid substitutions comprise T43A/D126A/P127A/K170A. In some embodiments, the four or more amino acid substitutions comprise T43A/D126A/R129A/K170A. In some embodiments, the four or more amino acid substitutions comprise T43A/D126A/K168A/K170A.

In some embodiments, the four or more amino acid substitutions comprise T43A/P127A/R129A/K170A. In some embodiments, the four or more amino acid substitutions comprise T43A/P127A/K168A/K170A. In some embodiments, the four or more amino acid substitutions comprise T43A/R129A/K168A/K170A. In some embodiments, the four or more amino acid substitutions comprise D126A/P127A/R129A/K170A. In some embodiments, the four or more amino acid substitutions comprise D126A/P127A/K168A/K170A. In some embodiments, the four or more amino acid substitutions comprise D126A/R129A/K168A/K170A. In some embodiments, the four or more amino acid substitutions comprise P127A/R129A/K168A/K170A.

In some embodiments, the variant IL-12p35 subunit comprises five or more amino acid substitutions selected from the group including: Y40A, T43A, D126A, P127A, R129A, K168A, and K170A. In some embodiments, the five or more amino acid substitutions comprise Y40A/T43A/D126A/P127A/R129A. In some embodiments, the five or more amino acid substitutions comprise Y40A/T43A/D126A/P127A/K168A. In some embodiments, the five or more amino acid substitutions comprise Y40A/D126A/P127A/R129A/K168A. In some embodiments, the five or more amino acid substitutions comprise Y40A/T43A/P127A/R129A/K168A. In some embodiments, the five or more amino acid substitutions comprise Y40A/T43A/D126A/R129A/K168A. In some embodiments, the five or more amino acid substitutions comprise T43A/D126A/P127A/R129A/K168A. In some embodiments, the five or more amino acid substitutions comprise Y40A/T43A/D126A/P127A/K170A. In some embodiments, the five or more amino acid substitutions comprise Y40A/T43A/D126A/R129A/K170A. In some embodiments, the five or more amino acid substitutions comprise Y40A/T43A/D126A/K168A/K170A. In some embodiments, the five or more amino acid substitutions comprise Y40A/T43A/P127A/R129A/K170A. In some embodiments, the five or more amino acid substitutions comprise Y40A/T43A/P127A/K168A/K170A. In some embodiments, the five or more amino acid substitutions comprise Y40A/T43A/R129A/K168A/K170A. In some embodiments, the five or more amino acid substitutions comprise Y40A/D126A/P127A/R129A/K170A. In some embodiments, the five or more amino acid substitutions comprise Y40A/D126A/P127A/K168A/K170A. In some embodiments, the five or more amino acid substitutions comprise Y40A/D126A/R129A/K168A/K170A. In some embodiments, the five or more amino acid substitutions comprise Y40A/P127A/R129A/K168A/K170A. In some embodiments, the five or more amino acid substitutions comprise T43A/D126A/P127A/R129A/K170A. In some embodiments, the five or more amino acid substitutions comprise T43A/D126A/P127A/K168A/K170A. In some embodiments, the five or more amino acid substitutions comprise T43A/D126A/R129A/K168A/K170A. In some embodiments, the five or more amino acid substitutions comprise T43A/P127A/R129A/K168A/K170A. In some embodiments, the five or more amino acid substitutions comprise D126A/P127A/R129A/K168A/K170A.

In some embodiments, the variant IL-12p35 subunit comprises six or more amino acid substitutions selected from the group including: Y40A, T43A, D126A, P127A, R129A, K168A, and K170A. In some embodiments, the six or more amino acid substitutions comprise Y40A/T43A/D126A/P127A/R129A/K168A. In some embodiments, the six or more amino acid substitutions comprise Y40A/T43A/D126A/P127A/R129A/K170A. In some embodiments, the six or more amino acid substitutions comprise Y40A/T43A/D126A/P127A/K168A/K170A. In some embodiments, the six or more amino acid substitutions comprise Y40A/D126A/P127A/R129A/K168A/K170A. In some embodiments, the six or more amino acid substitutions comprise Y40A/T43A/P127A/R129A/K168A/K170A. In some embodiments, the six or more amino acid substitutions comprise Y40A/T43A/D126A/R129A/K168A/K170A. In some embodiments, the six or more amino acid substitutions comprise T43A/D126A/P127A/R129A/K168A/K170A.

In some embodiments, the variant IL-12p35 subunit comprises seven or more amino acid substitution selected from the group including: Y40A, T43A, D126A, P127A, R129A, K168A, and K170A.

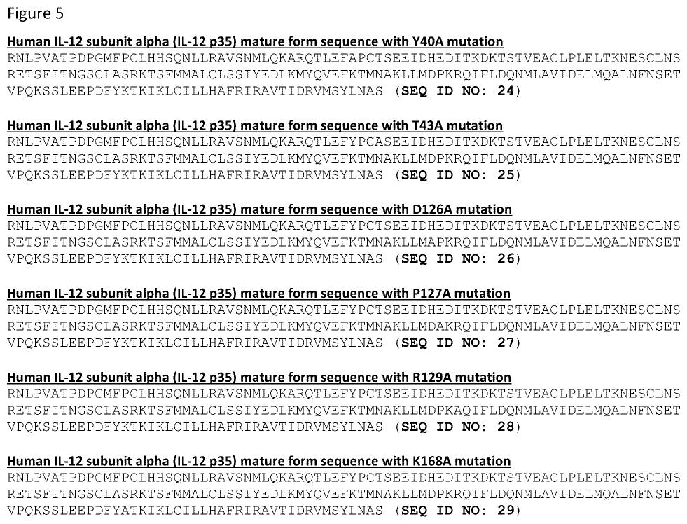

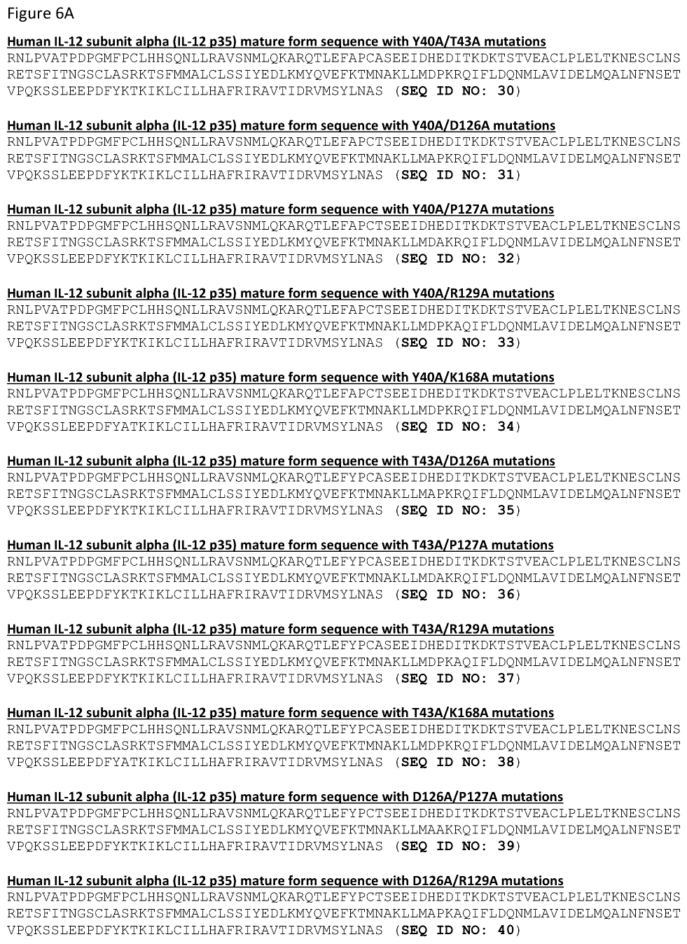

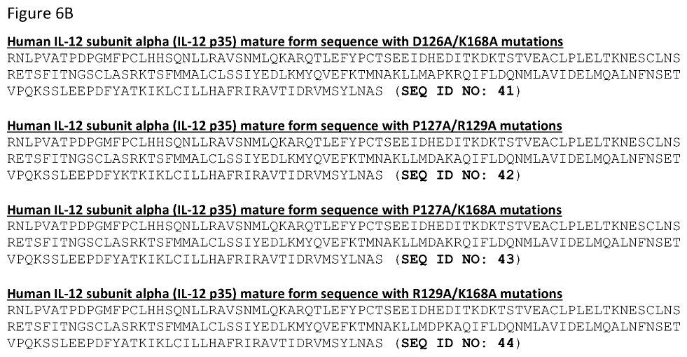

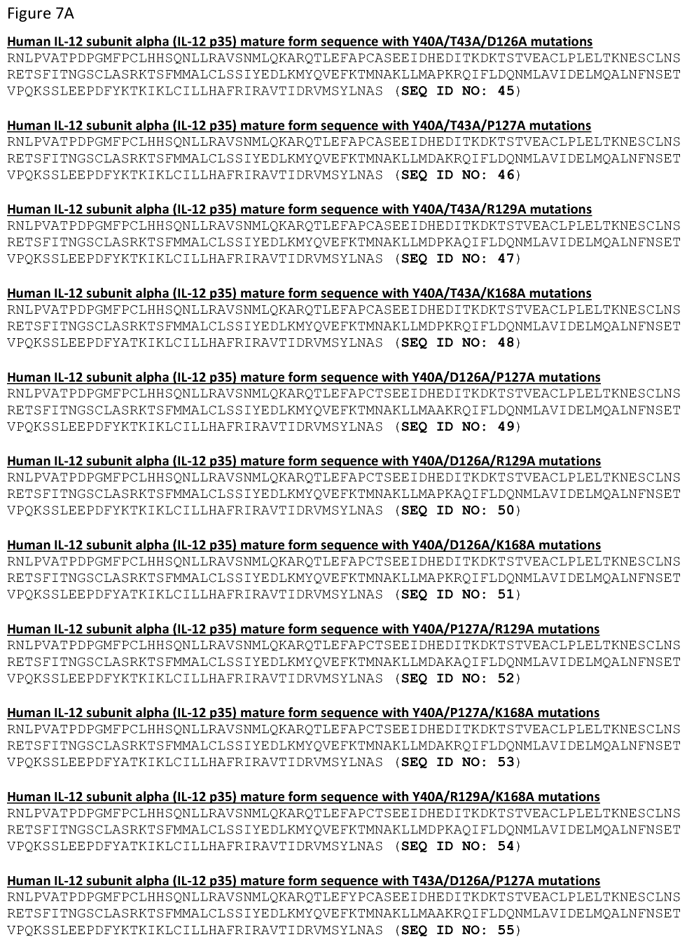

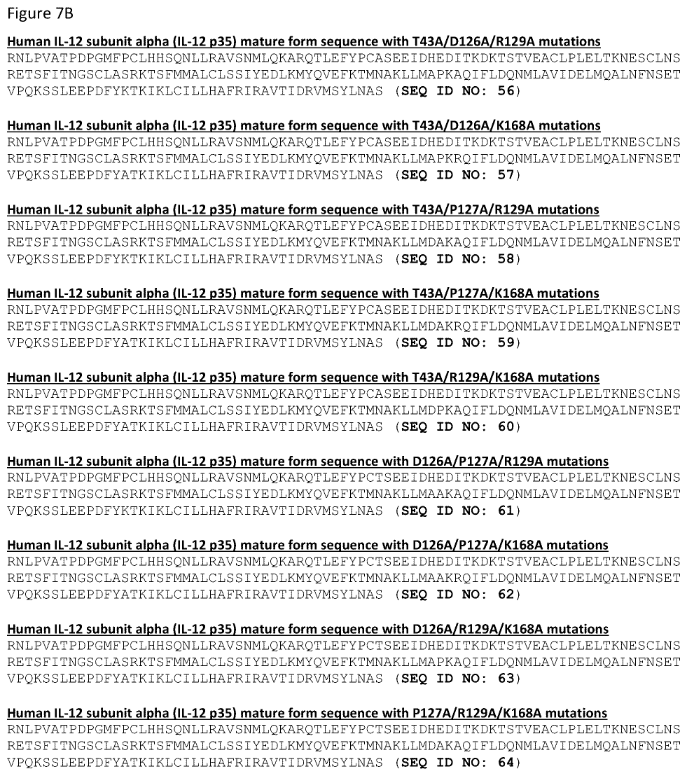

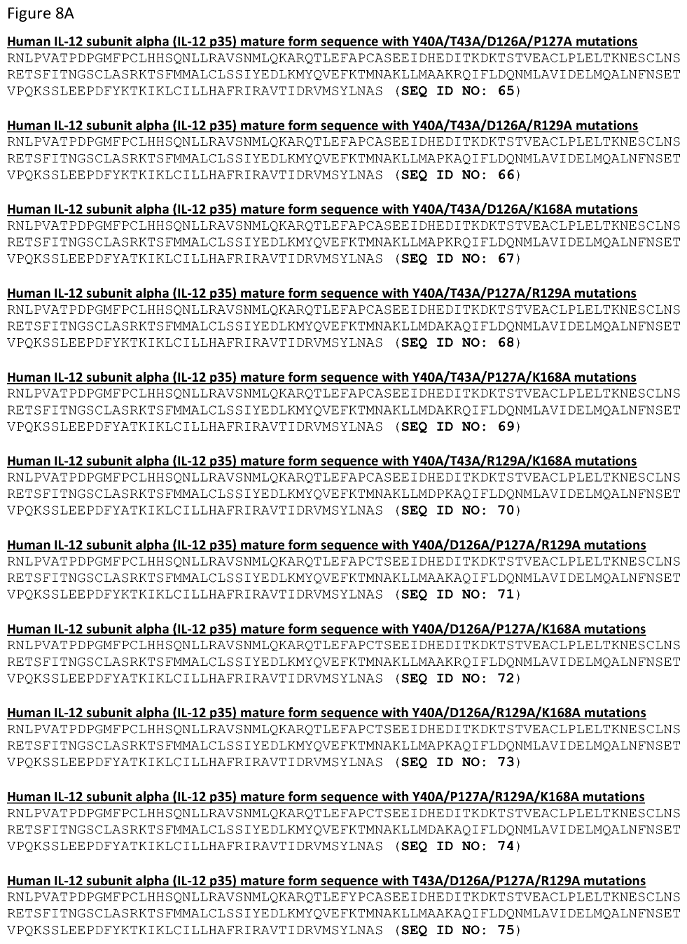

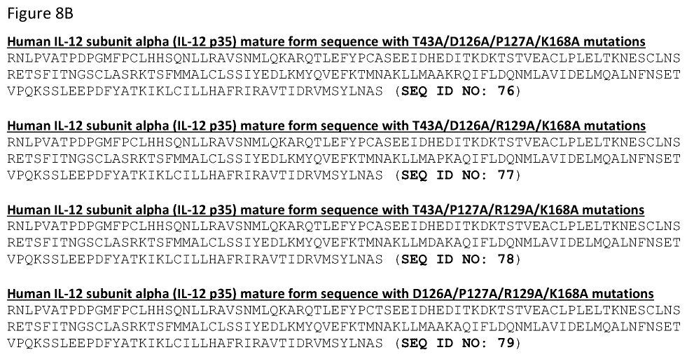

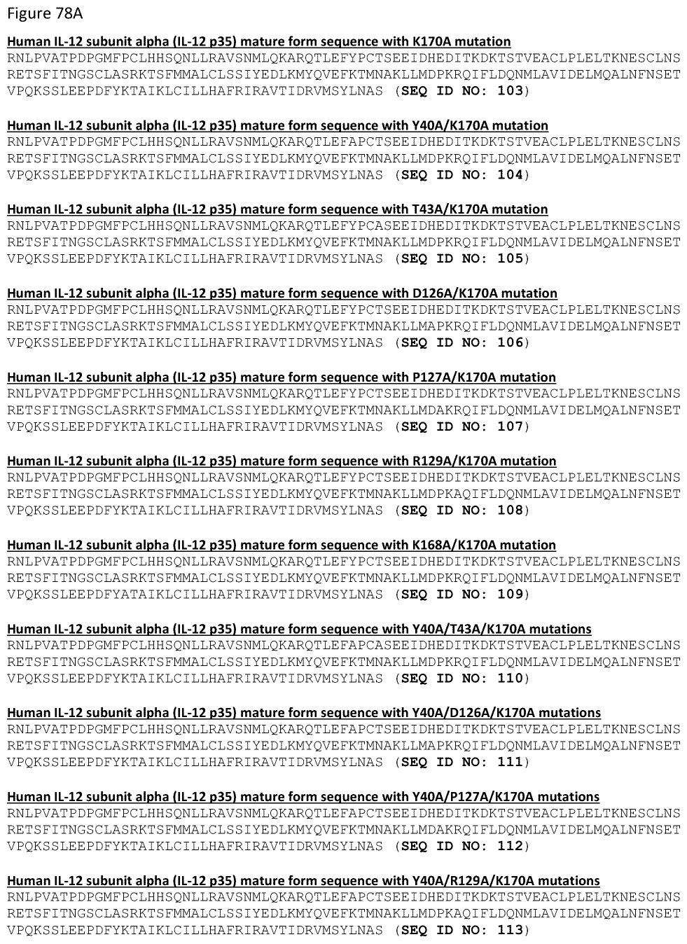

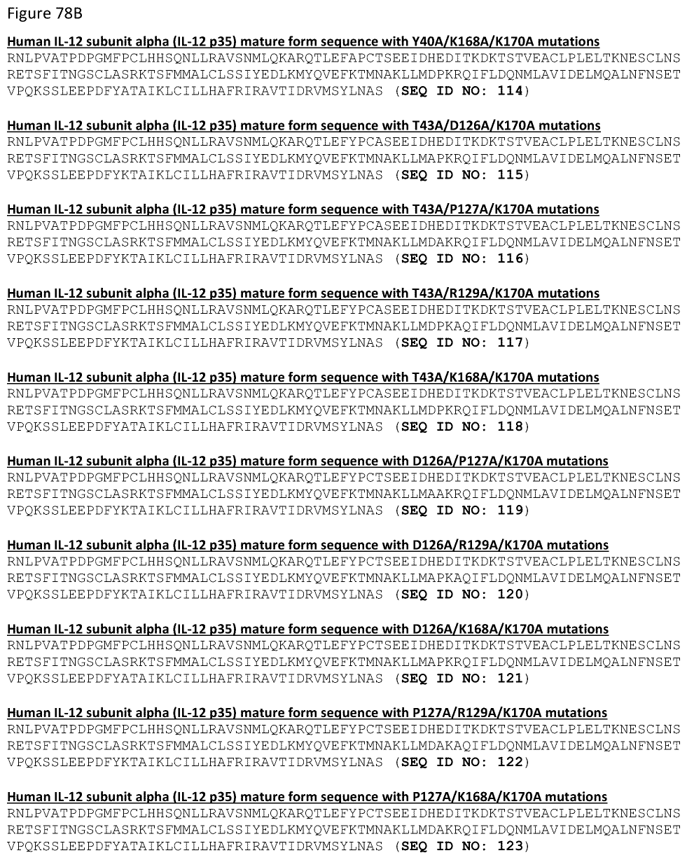

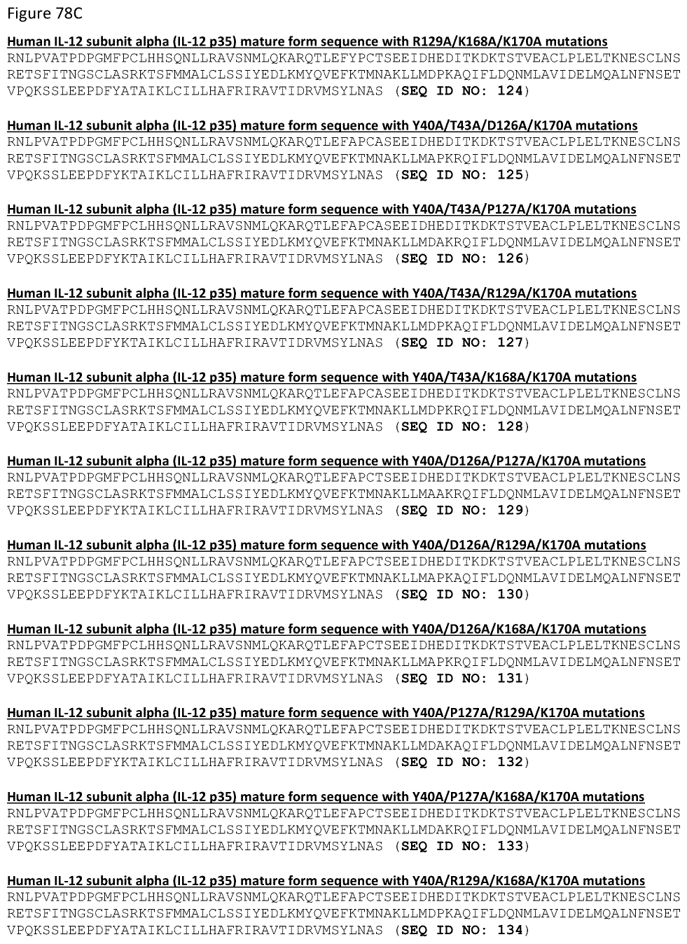

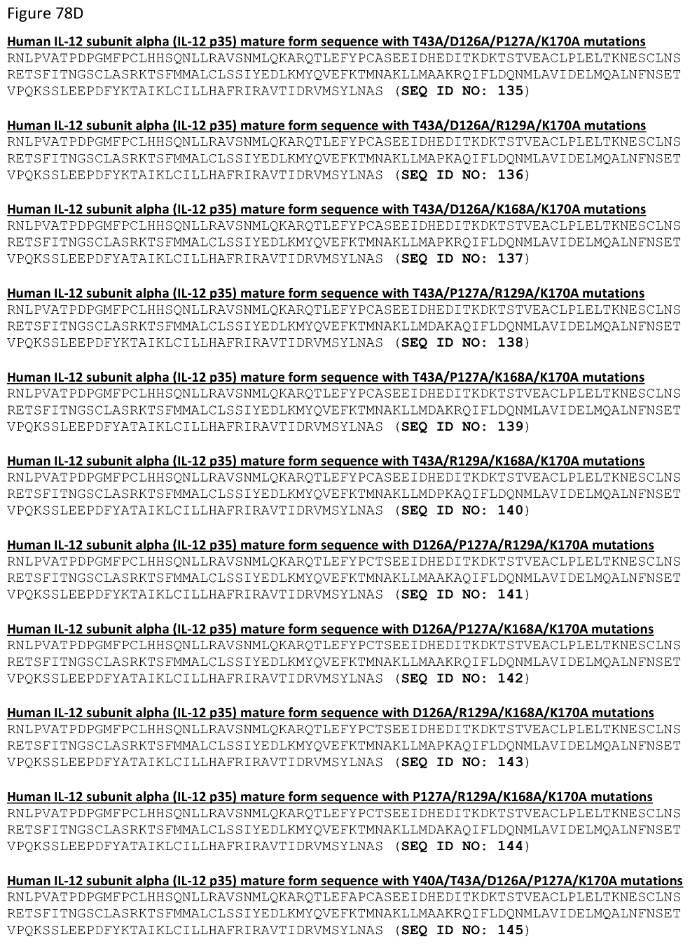

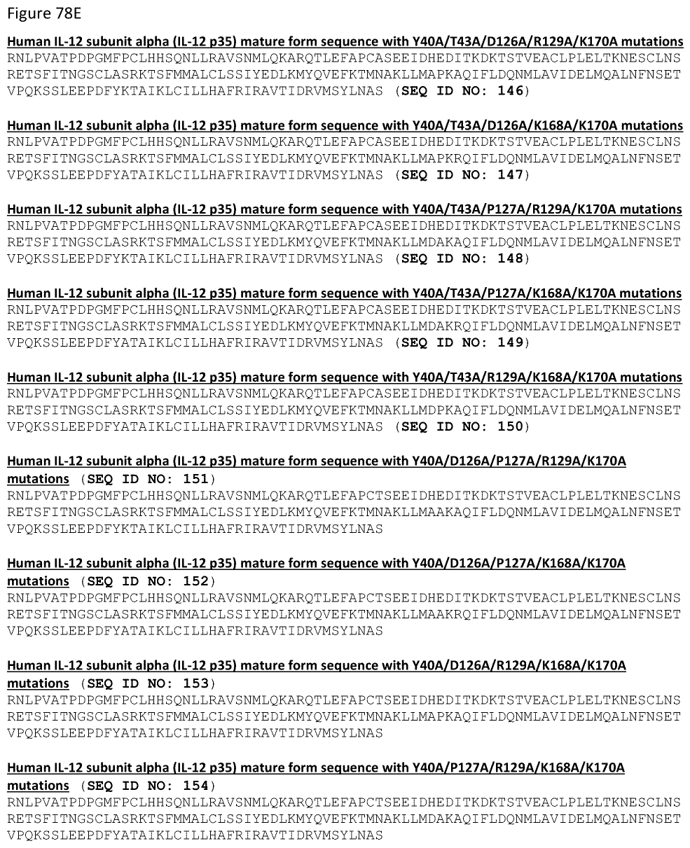

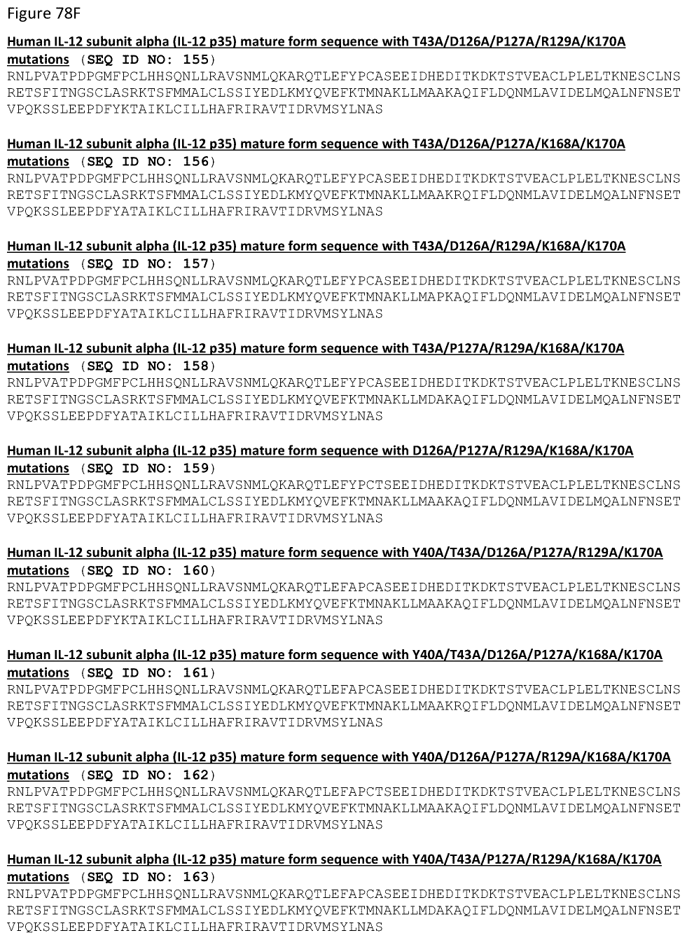

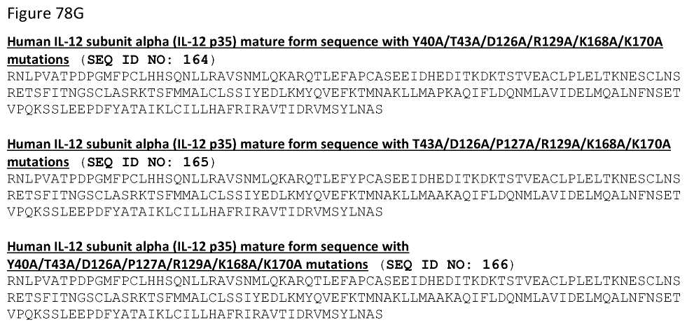

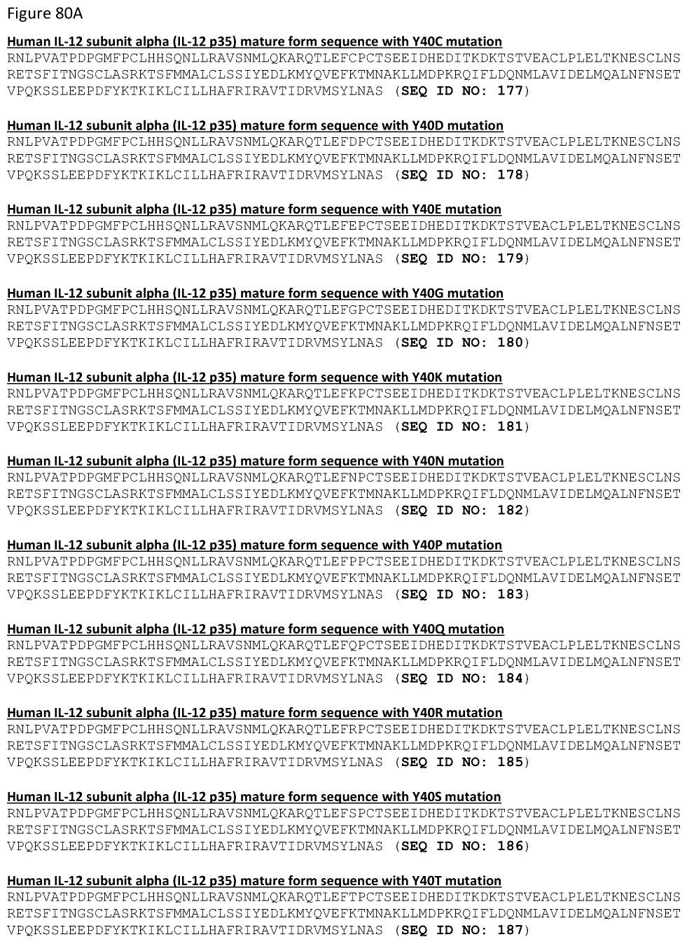

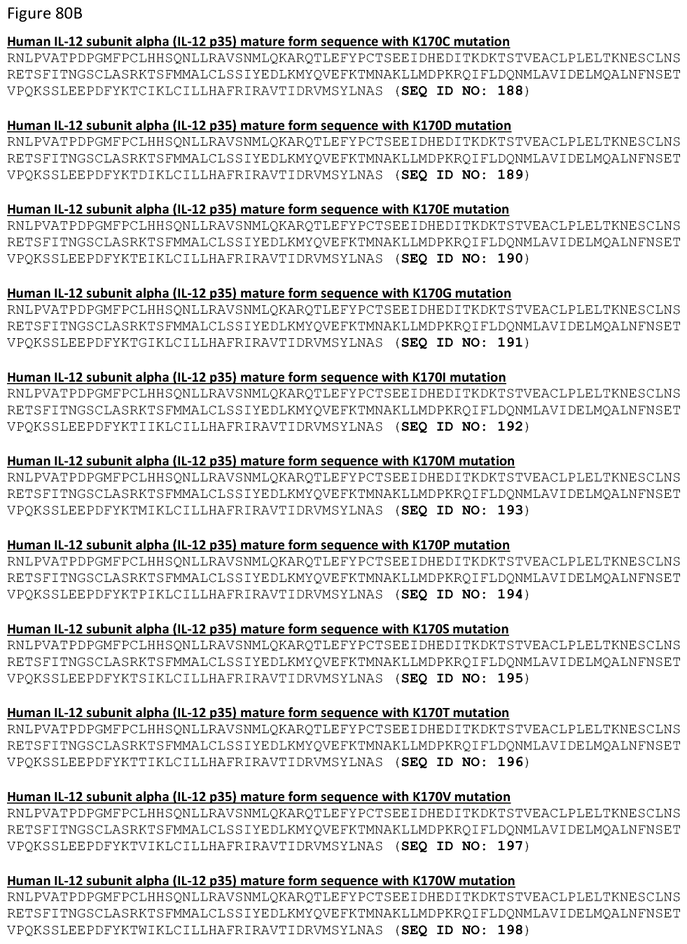

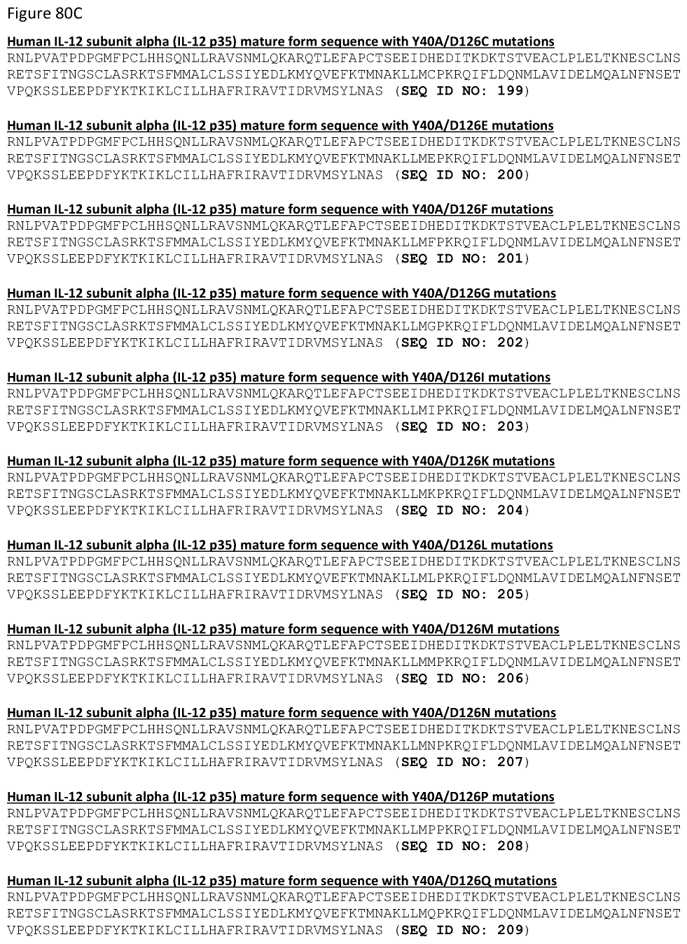

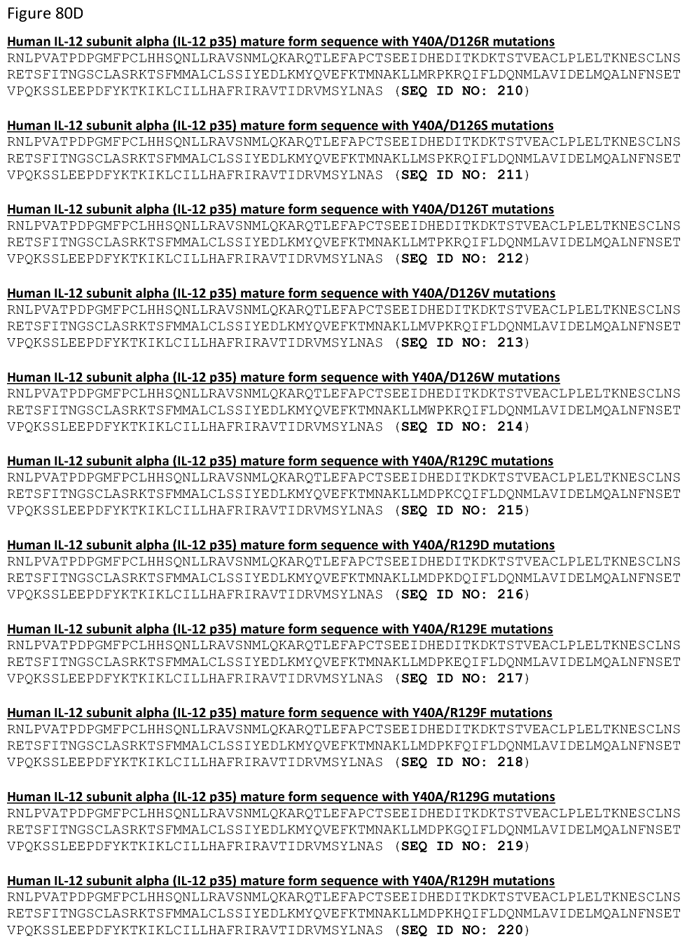

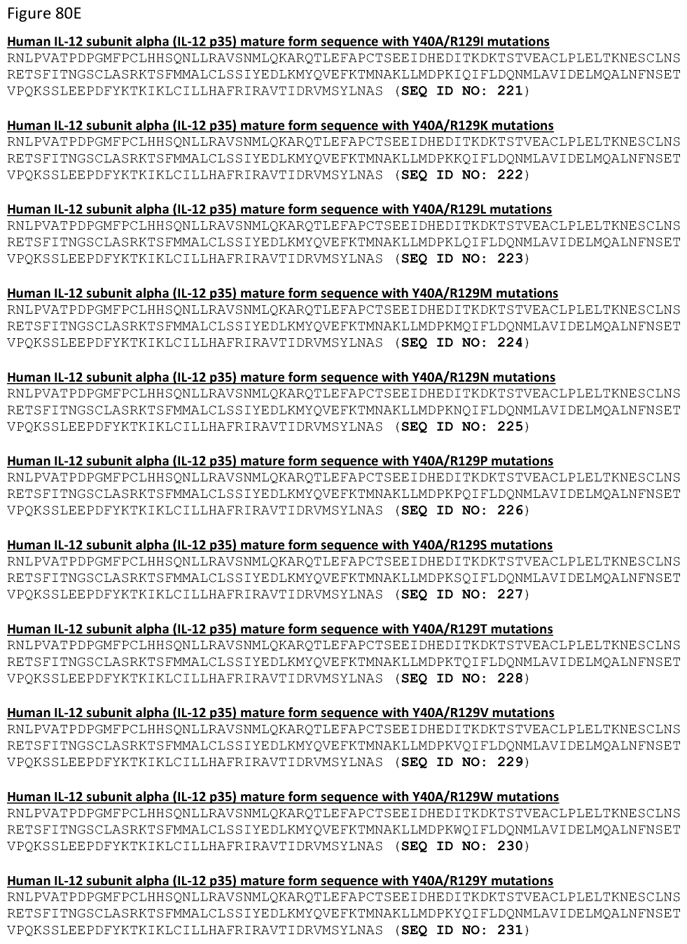

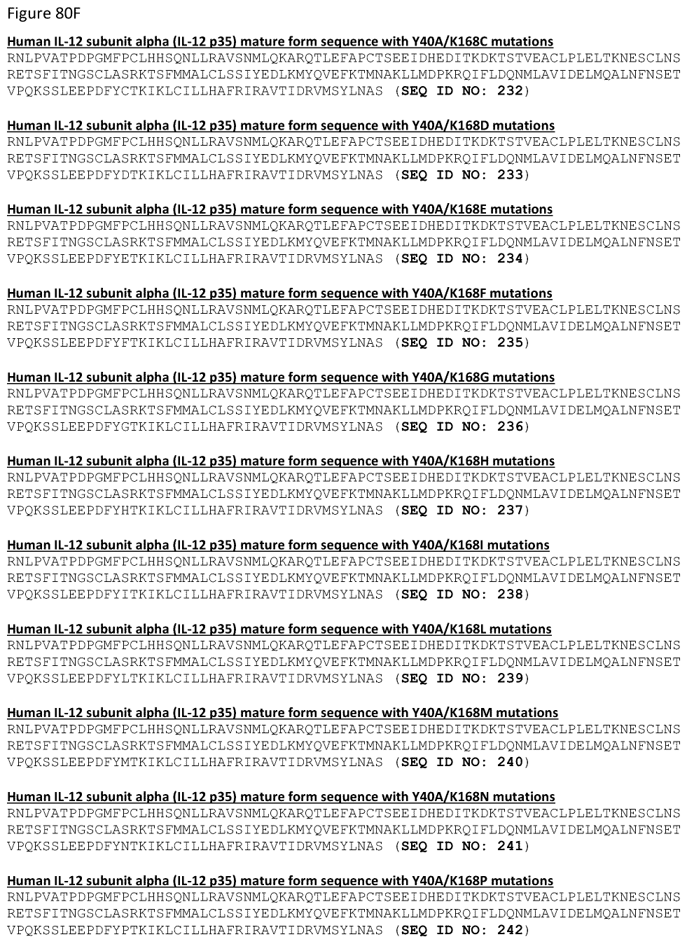

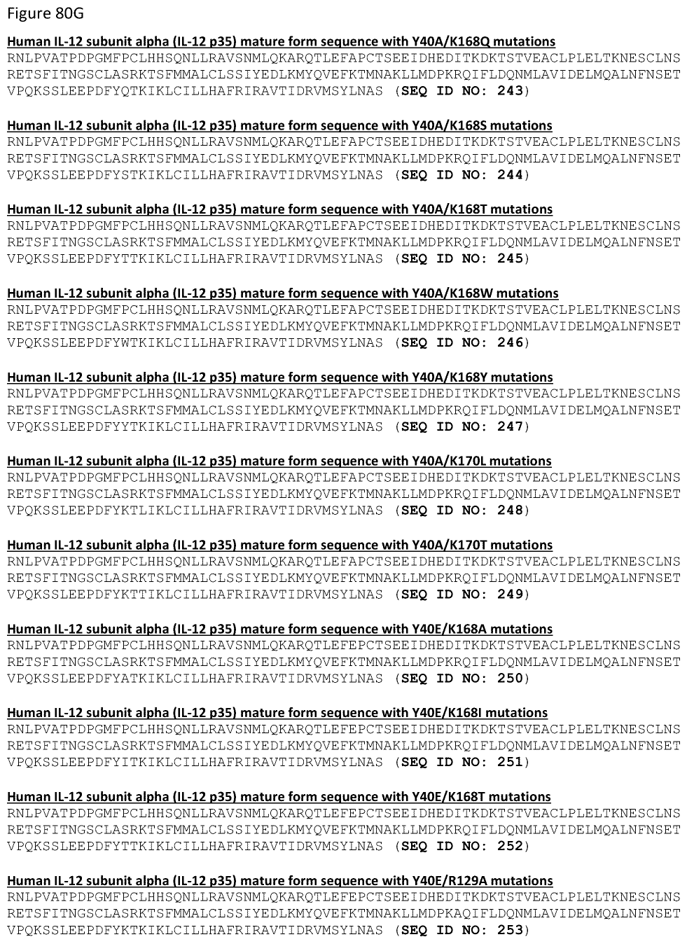

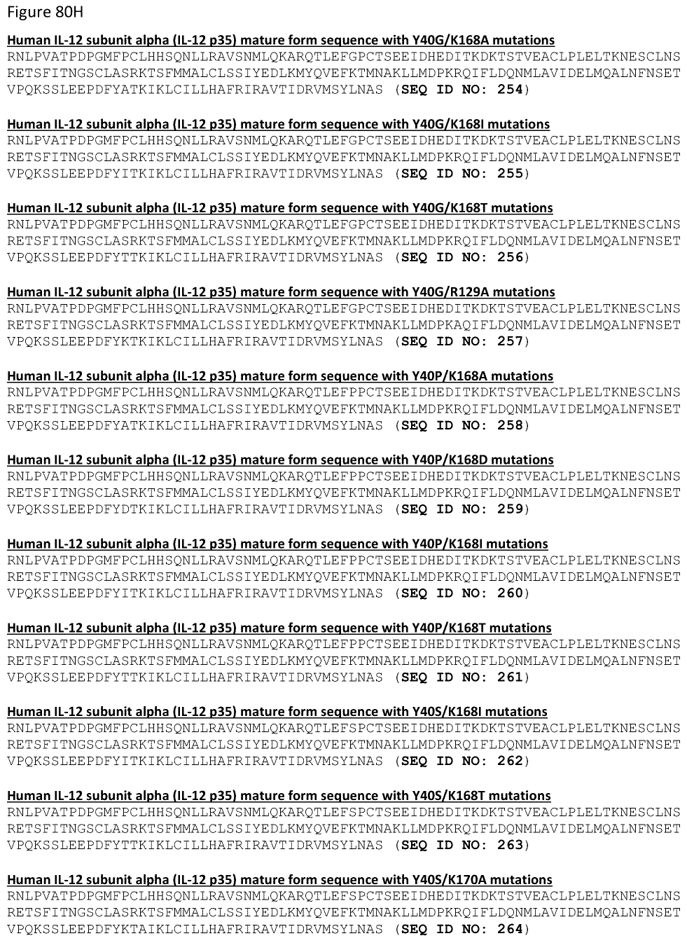

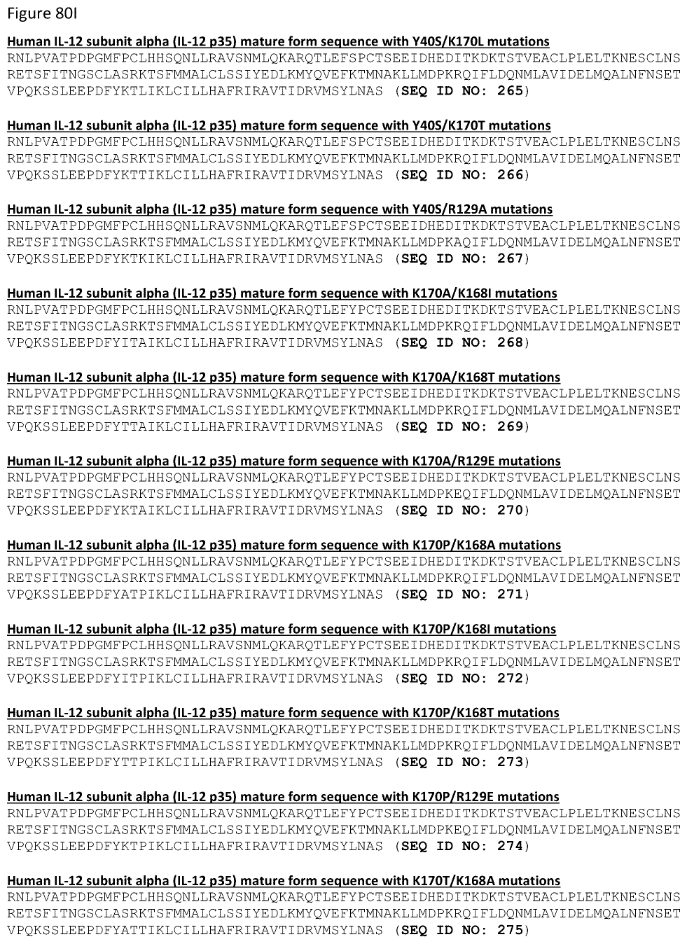

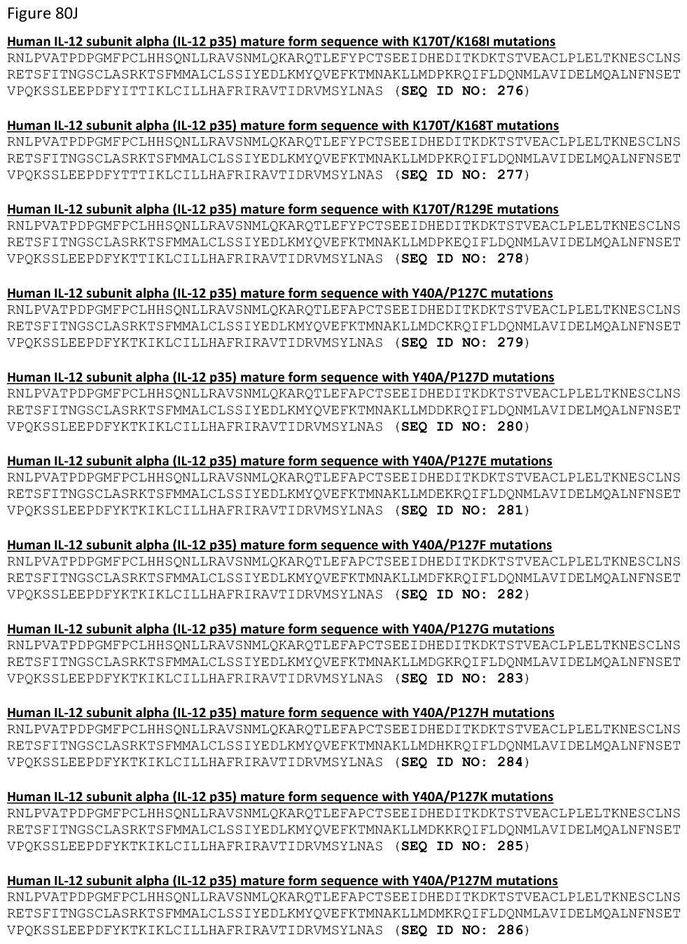

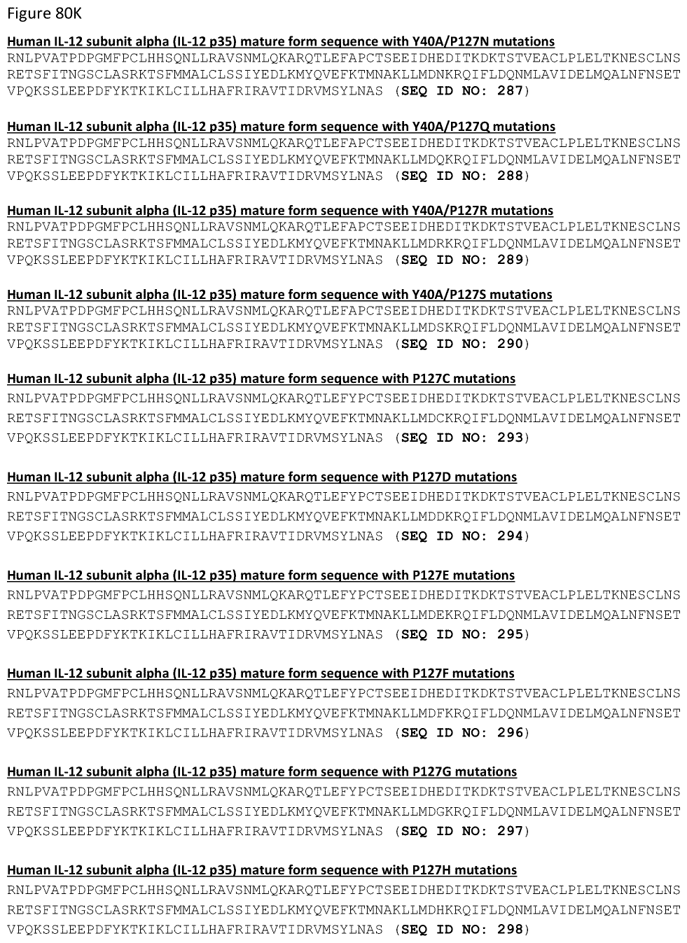

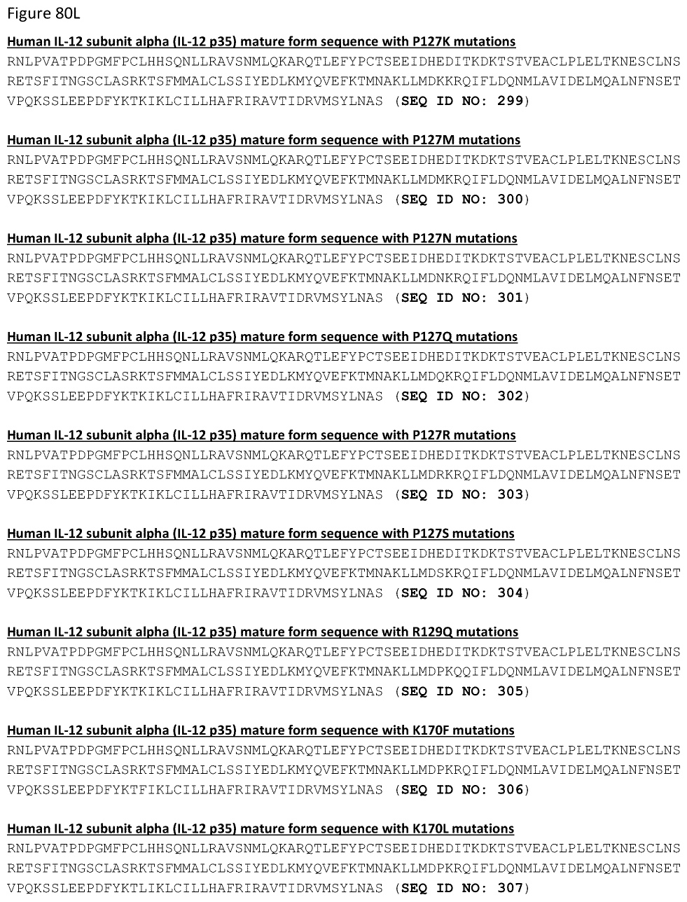

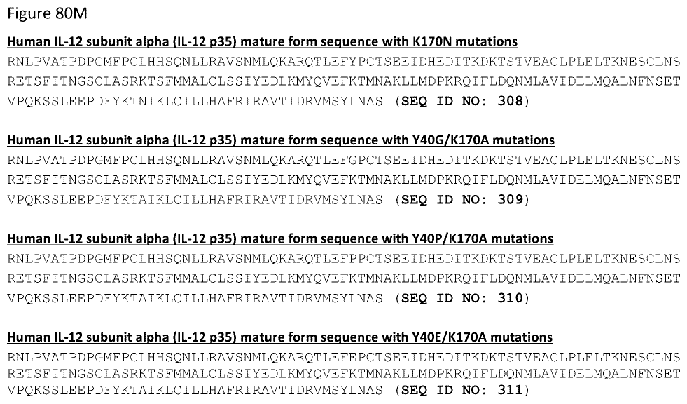

In some embodiments, the variant IL-12p35 subunit comprises an amino acid sequence selected from the group including: SEQ ID NO: 24 (Y40A), SEQ ID NO: 25 (T43A), SEQ ID NO: 26 (D126A), SEQ ID NO: 27 (P127A), SEQ ID NO: 28 (R129A), SEQ ID NO: 29 (K168A), SEQ ID NO: 30 (Y40A/T43A), SEQ ID NO: 31 (Y40A/D126A), SEQ ID NO: 32 (Y40A/P127A), SEQ ID NO: 33 (Y40A/R129A), SEQ ID NO: 34 (Y40A/K168A), SEQ ID NO: 35 (T43A/D126A), SEQ ID NO: 36 (T43A/P127A), SEQ ID NO: 37 (T43A/R129A), SEQ ID NO: 38 (T43A/K168A), SEQ ID NO: 39 (D126A/P127A), SEQ ID NO: 40 (D126A/R129A), SEQ ID NO: 41 (D126A/K168A), SEQ ID NO: 42 (P127A/R129A), SEQ ID NO: 43 (P127A/K168A), SEQ ID NO: 44 (R129A/K168A), SEQ ID NO: 45 (Y40A/T43A/D126A), SEQ ID NO: 46 (Y40A/T43A/P127A), SEQ ID NO: 47 (Y40A/T43A/R129A), SEQ ID NO: 48 (Y40A/T43A/K168A), SEQ ID NO: 49 (Y40A/D126A/P127A), SEQ ID NO: 50 (Y40A/D126A/R129A), SEQ ID NO: 51 (Y40A/D126A/K168A), SEQ ID NO: 52 (Y40A/P127A/R129A), SEQ ID NO: 53 (Y40A/P127A/K168A), SEQ ID NO: 54 (Y40A/R129A/K168A), SEQ ID NO: 55 (T43A/D126A/P127A), SEQ ID NO: 56 (T43A/D126A/R129A), SEQ ID NO: 57 (T43A/D126A/K168A), SEQ ID NO: 58 (T43A/P127A/R129A), SEQ ID NO: 59 (T43A/P127A/K168A), SEQ ID NO: 60 (T43A/R129A/K168A), SEQ ID NO: 61 (D126A/P127A/R129A), SEQ ID NO: 62 (D126A/P127A/K168A), SEQ ID NO: 63 (D126A/R129A/K168A), SEQ ID NO: 64 (P127A/R129A/K168A), SEQ ID NO: 65 (Y40A/T43A/D126A/P127A), SEQ ID NO: 66 (Y40A/T43A/D126A/R129A), SEQ ID NO: 67 (Y40A/T43A/D126A/K168A), SEQ ID NO: 68 (Y40A/T43A/P127A/R129A), SEQ ID NO: 69 (Y40A/T43A/P127A/K168A), SEQ ID NO: 70 (Y40A/T43A/R129A/K168A), SEQ ID NO: 71 (Y40A/D126A/P127A/R129A), SEQ ID NO: 72 (Y40A/D126A/P127A/K168A), SEQ ID NO: 73 (Y40A/D126A/R129A/K168A), SEQ ID NO: 74 (Y40A/P127A/R129A/K168A), SEQ ID NO: 75 (T43A/D126A/P127A/R129A), SEQ ID NO: 76 (T43A/D126A/P127A/K168A), SEQ ID NO: 77 (T43A/D126A/R129A/K168A), SEQ ID NO: 78 (T43A/P127A/R129A/K168A), SEQ ID NO: 79 (D126A/P127A/R129A/K168A), SEQ ID NO: 80 (Y40A/T43A/D126A/P127A/R129A), SEQ ID NO: 81 (Y40A/T43A/D126A/P127A/K168A), SEQ ID NO: 82 (Y40A/D126A/P127A/R129A/K168A), SEQ ID NO: 83 (Y40A/T43A/P127A/R129A/K168A), SEQ ID NO: 84 (Y40A/T43A/D126A/R129A/K168A), SEQ ID NO: 85 (T43A/D126A/P127A/R129A/K168A), SEQ ID NO: 103 (K170A), SEQ ID NO: 104 (Y40A/K170A), SEQ ID NO: 105 (T43A/K170A), SEQ ID NO: 106 (D126A/K170A), SEQ ID NO: 107 (P127A/K170A), SEQ ID NO: 108 (R129A/K170A), SEQ ID NO: 109 (K168A/K170A), SEQ ID NO: 110 (Y40A/T43A/K170A), SEQ ID NO: 111 (Y40A/D126A/K170A), SEQ ID NO: 112 (Y40A/P127A/K170A), SEQ ID NO: 113 (Y40A/R129A/K170A), SEQ ID NO: 114 (Y40A/K168A/K170A), SEQ ID NO: 115 (T43A/D126A/K170A), SEQ ID NO: 116 (T43A/P127A/K170A), SEQ ID NO: 117 (T43A/R129A/K170A), SEQ ID NO: 118 (T43A/K168A/K170A), SEQ ID NO: 119 (D126A/P127A/K170A), SEQ ID NO: 120 (D126A/R129A/K170A), SEQ ID NO: 121 (D126A/K168A/K170A), SEQ ID NO: 122 (P127A/R129A/K170A), SEQ ID NO: 123 (P127A/K168A/K170A), SEQ ID NO: 124 (R129A/K168A/K170A), SEQ ID NO: 125 (Y40A/T43A/D126A/K170A), SEQ ID NO: 126 (Y40A/T43A/P127A/K170A), SEQ ID NO: 127 (Y40A/T43A/R129A/K170A), SEQ ID NO: 128 (Y40A/T43A/K168A/K170A), SEQ ID NO: 129 (Y40A/D126A/P127A/K170A), SEQ ID NO: 130 (Y40A/D126A/R129A/K170A), SEQ ID NO: 131 (Y40A/D126A/K168A/K170A), SEQ ID NO: 132 (Y40A/P127A/R129A/K170A), SEQ ID NO: 133 (Y40A/P127A/K168A/K170A), SEQ ID NO: 134 (Y40A/R129A/K168A/K170A), SEQ ID NO: 135 (T43A/D126A/P127A/K170A), SEQ ID NO: 136 (T43A/D126A/R129A/K170A), SEQ ID NO: 137 (T43A/D126A/K168A/K170A), SEQ ID NO: 138 (T43A/P127A/R129A/K170A), SEQ ID NO: 139 (T43A/P127A/K168A/K170A), SEQ ID NO: 140 (T43A/R129A/K168A/K170A), SEQ ID NO: 141 (D126A/P127A/R129A/K170A), SEQ ID NO: 142 (D126A/P127A/K168A/K170A), SEQ ID NO: 143 (D126A/R129A/K168A/K170A), SEQ ID NO: 144 (P127A/R129A/K168A/K170A), SEQ ID NO: 145 (Y40A/T43A/D126A/P127A/K170A), SEQ ID NO: 146 (Y40A/T43A/D126A/R129A/K170A), SEQ ID NO: 147 (Y40A/T43A/D126A/K168A/K170A), SEQ ID NO: 148 (Y40A/T43A/P127A/R129A/K170A), SEQ ID NO: 149 (Y40A/T43A/P127A/K168A/K170A), SEQ ID NO: 150 (Y40A/T43A/R129A/K168A/K170A), SEQ ID NO: 151 (Y40A/D126A/P127A/R129A/K170A), SEQ ID NO: 152 (Y40A/D126A/P127A/K168A/K170A), SEQ ID NO: 153 (Y40A/D126A/R129A/K168A/K170A), SEQ ID NO: 154 (Y40A/P127A/R129A/K168A/K170A), SEQ ID NO: 155 (T43A/D126A/P127A/R129A/K170A), SEQ ID NO: 156 (T43A/D126A/P127A/K168A/K170A), SEQ ID NO: 157 (T43A/D126A/R129A/K168A/K170A), SEQ ID NO: 158 (T43A/P127A/R129A/K168A/K170A), SEQ ID NO: 159 (D126A/P127A/R129A/K168A/K170A), SEQ ID NO: 160 (Y40A/T43A/D126A/P127A/R129A/K170A), SEQ ID NO: 161 (Y40A/T43A/D126A/P127A/K168A/K170A), SEQ ID NO: 162 (Y40A/D126A/P127A/R129A/K168A/K170A), SEQ ID NO: 163 (Y40A/T43A/P127A/R129A/K168A/K170A), SEQ ID NO: 164 (Y40A/T43A/D126A/R129A/K168A/K170A), SEQ ID NO: 165 (T43A/D126A/P127A/R129A/K168A/K170A), SEQ ID NO: 166 (Y40A/T43A/D126A/P127A/R129A/K168A/K170A), and SEQ ID NO: 86 (Y40A/T43A/D126A/P127A/R129A/K168A) (as shown in FIGS. 5-9, 78, and 80).

In some embodiments, the variant IL-12p35 subunit comprises an amino acid sequence with at least 90%, 91%, 92%, 93%, 94%, 95%, 96%, 97%, 98%, 99% or higher sequence identity to an amino acid sequence selected from the group including: SEQ ID NO: 1 (wild-type precursor), SEQ ID NO: 2 (wild-type mature), SEQ ID NOs: 24-86, SEQ ID NOs: 103-166, and SEQ ID NO: 87 (C74S) (as shown in FIGS. 1, 5-9, 78, and 80).

In some embodiments, the variant IL-12p35 subunit comprises a substitution mutation at amino acid residue Y40. In some further embodiments, the substitution mutation at amino acid residue Y40 is selected from the group including: Y40C, Y40D, Y40E, Y40G, Y40K, Y40N, Y40P, Y40Q, Y40R, Y40S, and Y40T. In some embodiments, the variant IL-12p35 subunit comprises any one of SEQ ID NOs: 177-187.

In some embodiments, the variant IL-12p35 subunit comprises a substitution mutation at amino acid residue D126. In some further embodiments, the substitution mutation at amino acid residue D126 is selected from the group including: D126C, D126E, D126F, D126G, D1261, D126K, D126L, D126M, D126N, D126P, D126Q, D126R, D126S, D126T, D126V, and D126W.

In some embodiments, the variant IL-12p35 subunit comprises a first substitution mutation, comprising: Y40A, and further comprises a second substitution mutation selected from the group including: D126C, D126E, D126F, D126G, D1261, D126K, D126L, D126M, D126N, D126P, D126Q, D126R, D126S, D126T, D126V, and D126W. In some embodiments, the variant IL-12p35 subunit comprises any one of SEQ ID NOs: 199-214.

In some embodiments, the variant IL-12p35 subunit comprises a substitution mutation at amino acid residue P127. In some further embodiments, the substitution mutation at amino acid residue P127 is selected from the group including: P127C, P127D, P127E, P127F, P127G, P127H, P127K, P127M, P127N, P127Q, P127R, and P127S.

In some embodiments, the variant IL-12p35 subunit comprises a first substitution mutation, comprising: Y40A, and further comprises a second substitution mutation selected from the group including: P127C, P127D, P127E, P127F, P127G, P127H, P127K, P127M, P127N, P127Q, P127R, and P127S. In some embodiments, the variant IL-12p35 subunit comprises any one of SEQ ID NOs: 279-290.

In some embodiments, the variant IL-12p35 subunit comprises a substitution mutation at amino acid residue R129. In some further embodiments, the substitution mutation at amino acid residue R129 is selected from the group including: R129C, R129D, R129E, R129F, R129G, R129H, R129I, R129K, R129L, R129M, R129N, R129P, R129Q, R129S, R129T, R129V, R129W, and R129Y.

In some embodiments, the variant IL-12p35 subunit comprises a first substitution mutation, comprising: Y40A, and further comprises a second substitution mutation selected from the group including: R129C, R129D, R129E, R129F, R129G, R129H, R129I, R129K, R129L, R129M, R129N, R129P, R129Q, R129S, R129T, R129V, R129W, and R129Y. In some embodiments, the variant IL-12p35 subunit comprises any one of SEQ ID NOs: 215-231.

In some embodiments, the variant IL-12p35 subunit comprises a substitution mutation at amino acid residue K168. In some further embodiments, the substitution mutation at amino acid residue K168 is selected from the group including: K168C, K168D, K168E, K168F, K168G, K168H, K168I, K168L, K168M, K168N, K168P, K168Q, K168S, K168T, K168W, and K168Y.

In some embodiments, the variant IL-12p35 subunit comprises a first substitution mutation, comprising: Y40A, and further comprises a second substitution mutation selected from the group including: K168C, K168D, K168E, K168F, K168G, K168H, K168I, K168L, K168M, K168N, K168P, K168Q, K168S, K168T, K168W, and K168Y. In some embodiments, the variant IL-12p35 subunit comprises any one of SEQ ID NOs: 232-247.

In some embodiments, the variant IL-12p35 subunit comprises a substitution mutation at amino acid residue K170. In some further embodiments, the substitution mutation at amino acid residue K170 is selected from the group including: K170C, K170D, K170E, K170G, K170I, K170M, K170P, K170S, K170T, K170V, K170F, K170L, K170N, and K170W. In some embodiments, the variant IL-12p35 subunit comprises any one of SEQ ID NOs: 188-198 or 306-308.

In some embodiments, the variant IL-12p35 subunit comprises a first substitution mutation, comprising: Y40A, and further comprises a second substitution mutation selected from the group including: K170L and K170T. In some embodiments, the variant IL-12p35 subunit comprises any one of SEQ ID NOs: 248 or 249.

In some embodiments, the variant IL-12p35 subunit comprises: (i) a first substitution mutation selected from the group including: Y40A, Y40C, Y40D, Y40E, Y40G, Y40K, Y40N, Y40P, Y40Q, Y40R, Y40S, and Y40T, and (ii) a second substitution mutation selected from the group including: D126A, D126C, D126E, D126F, D126G, D1261, D126K, D126L, D126M, D126N, D126P, D126Q, D126R, D126S, D126T, D126V, D126W, R129A, R129C, R129D, R129E, R129F, R129G, R129H, R129I, R129K, R129L, R129M, R129N, R129P, R129Q, R129S, R129T, R129V, R129W, R129Y, K168A, K168C, K168D, K168E, K168F, K168G, K168H, K168I, K168L, K168M, K168N, K168P, K168Q, K168S, K168T, K168W, K168Y, K170A, K170C, K170D, K170E, K170G, K170I, K170M, K170P, K170S, K170T, K170V, K170F, K170L, K170N, and K170W.

In some embodiments, the variant IL-12p35 subunit comprises a first substitution mutation, comprising: Y40E, and further comprises a second substitution mutation selected from the group including: K170A, K168A, K168I, K168T, and R129A. In some embodiments, the variant IL-12p35 subunit comprises any one of SEQ ID NOs: 250-253 or 311.

In some embodiments, the variant IL-12p35 subunit comprises a first substitution mutation, comprising: Y40G, and further comprises a second substitution mutation selected from the group including: K170A, K168A, K168I, K168T, and R129A. In some embodiments, the variant IL-12p35 subunit comprises any one of SEQ ID NOs: 254-257 or 309.

In some embodiments, the variant IL-12p35 subunit comprises a first substitution mutation, comprising: Y40P, and further comprises a second substitution mutation selected from the group including: K170A, K168A, K168D, K168I, and K168T. In some embodiments, the variant IL-12p35 subunit comprises any one of SEQ ID NOs: 258-261 or 310.

In some embodiments, the variant IL-12p35 subunit comprises a first substitution mutation, comprising: Y40S, and further comprises a second substitution mutation selected from the group including: K168I, K168T, K170A, K170L, K170T, and R129A. In some embodiments, the variant IL-12p35 subunit comprises any one of SEQ ID NOs: 262-267.

In some embodiments, the variant IL-12p35 subunit comprises a first substitution mutation, comprising: K170A, and further comprises a second substitution mutation selected from the group including: K168I, K168T, and R129E. In some embodiments, the variant IL-12p35 subunit comprises any one of SEQ ID NOs: 268-270.

In some embodiments, the variant IL-12p35 subunit comprises a first substitution mutation, comprising: K170P, and further comprises a second substitution mutation selected from the group including: K168A, K168I, K168T, and R129E. In some embodiments, the variant IL-12p35 subunit comprises any one of SEQ ID NOs: 271-274.

In some embodiments, the variant IL-12p35 subunit comprises a first substitution mutation, comprising: K170T, and further comprises a second substitution mutation selected from the group including: K168A, K168I, K168T, and R129E. In some embodiments, the variant IL-12p35 subunit comprises any one of SEQ ID NOs: 275-278.

In some embodiments, the variant IL-12p35 subunit may further comprise a C74S substitution mutation.

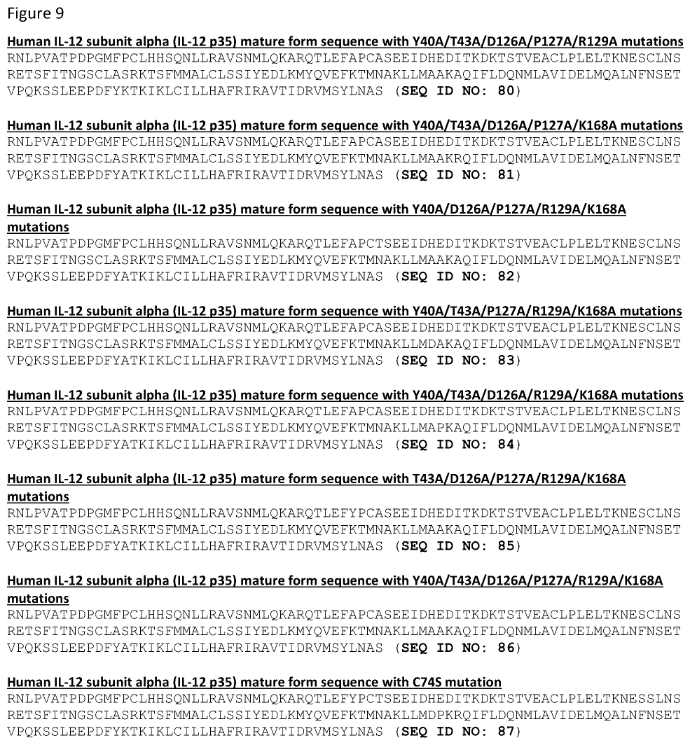

In addition to the novel, non-naturally occurring IL-12p35 variants described above, additional modifications may be included in the IL-12p35 variants disclosed herein. Non-limiting examples of residues that may be modified include: Q20, N21, Q35, E38, F39, P41, S44, E45, E46, E50, H49, K54, D55, T59, V60, E61, C63, L64, P65, E67, L68, T69, N71, S73, C74, L75, N76, E79, N85, L89, F96, M97, M98, A99, L124, M125, K128, Q130, Q135, N136, E143, Q146, N151, E153, K158, E162, E163, D165, F166, Y167, 1171, R181, 1182, R183, V185, T186, D188, R189, V190, M191, S192, Y193, N195, A196, and S197. In some embodiments, the variant IL-12p35 subunit described above, below, or in any one of FIGS. 1, 5-9, 78, and 80 comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more additional amino acid substitutions. In some embodiments, the variant IL-12p35 subunit described above, below, or in any one of FIGS. 1, 5-9, 78, and 80 comprises about 1 to about 5, about 6 to about 10, about 11 to about 15, about 16 to about 20, about 21 to about 25, about 26 to about 30, about 31 to about 35, about 36 to about 40, about 1 to about 10, about 11 to about 20, about 21 to about 30, about 31 to about 40, about 1 to about 20, or about 21 to about 40 additional amino acid substitutions. In some embodiments, the variant IL-12p35 subunit comprises SEQ ID NO: 87 (as shown in FIG. 9), and further comprises one, two, three, four, five, six, or all seven amino acid substitutions selected from the group including: Y40A, T43A, D126A, P127A, R129A, K168A, and K170A.

Mutations in the variant IL-12p35 subunit may result in changes to one or more of the following parameters: (i) binding affinity, (ii) potency, (iii) activity, (iv) manufacturability, or (v) stability; however, changes to one or more of the preceding parameters (such as, for example, binding affinity) may not necessarily be correlated with changes in one or more of the other preceding parameters (such as, for example, potency or activity). In some embodiments, the one or more amino acid substitutions in the variant IL-12p35 subunit result in an altered binding affinity to IL-12Rβ2, as compared to the binding affinity of a reference IL-12. In some embodiments, the one or more amino acid substitutions decreases the binding affinity of the variant IL-12p35 subunit to IL-12Rβ2 compared to a reference IL-12. In some embodiments, the reference IL-12 comprises one or more of the following: a wild-type IL-12, a human wild-type IL-12, a commercially available IL-12 molecule, or an IL-12 Fc fusion protein. In certain exemplary embodiments, the one or more amino acid substitutions in the variant IL-12p35 subunit do not affect binding affinity but may result in changes to one or more of the following parameters: (i) potency, (ii) activity, (iii) manufacturability, (iv) stability, or (v) any combination thereof.

A variety of assay formats may be used to select a non-naturally occurring IL-12 variant that binds to a ligand of interest (e.g., IL-12Rβ2 and/or IL-12Rβ1). Non-limiting examples include: solid-phase ELISA immunoassay, immunoprecipitation, a Biacore assay, a KinExA assay, fluorescence-activated cell sorting (FACS), an Octet assay, Western blot analysis, and the like. The binding activity of the non-naturally occurring IL-12 variants of the disclosure can be assayed by any suitable method known in the art, such as, for example, a surface plasmon resonance (SPR) assay, an enzyme-linked immunosorbent assay (ELISA), an ELISpot assay, Biacore assays, KinExA assays, and the like.

One of ordinary skill in the art will appreciate that binding affinity can also be used as a measure of the “strength” of a non-covalent interaction between two binding partners (e.g., a variant IL-12p35 subunit and IL-12Rβ2). Binding affinity between two molecules may be quantified by determination of the dissociation constant (K_{D}). In turn, K_{D} can be determined by measurement of the kinetics of complex formation and dissociation using suitable assays known in the art, such as, for example, an SPR assay. The rate constants corresponding to the association and the dissociation of a monovalent complex are referred to as the association rate constants kₐ (or kₒₙ) and dissociation rate constant kₐ (or k_{off}), respectively. K_{D} is related to kₐ and kₐ through the equation: K_{D}=_{d}/kₐ. The value of the dissociation constant can be determined directly by well-known methods.

As used herein, the term “potency” refers to the ability of a given protein, cytokine, fusion protein, antibody, and the like (such as, for example, a non-naturally occurring IL-12 variant) to elicit a response at a certain dose or concentration in a given biological system or experimental setting. The potency of the non-naturally occurring IL-12 variants of the disclosure can be assayed by any suitable method known in the art, such as, for example, an IL-12 HEK reporter assay (e.g., InvivoGen's IL-12 HEK reporter assay (Catalog No. hkb-il12)), a ligand-binding assay (e.g., ELISA or flow cytometry), and/or a functional assay. Generally, changes in potency may be demonstrated graphically as a leftward or rightward shift in a response curve compared to a control. A rightward shift of the response curve is generally indicative of a reduction in potency, whereas as a leftward shift of the response curve is generally indicative of an increase in potency. As used herein, the term “activity” refers to the specific response of a given protein, cytokine, fusion protein, antibody, and the like (such as, for example, a non-naturally occurring IL-12 variant) elicited in a particular biological system or experimental setting at a given dose or concentration. The activity of the non-naturally occurring IL-12 variants of the disclosure can be assayed by any suitable method known in the art, such as, for example, a ligand-binding assay and/or a functional assay. Generally, changes in activity may be demonstrated graphically as an upward or downward shift in a response curve compared to a control. An upward shift of the response curve is generally indicative of an increase in activity, whereas a downward shift of the response curve is generally indicative of a decrease in activity.

As used herein, the term “manufacturability” refers to any property that may impact the process of producing and/or storing a given protein, cytokine, fusion protein, antibody, and the like at a scale and quantity sufficient for administration to an individual. Examples of properties that impact manufacturability include, but are not limited to, the stability, purity, aggregation levels, and/or yield of expression of a given protein, cytokine, fusion protein, antibody, and the like (such as, for example, a non-naturally occurring IL-12 variant). As used herein, the term “stability” refers to the ability of a given protein, cytokine, fusion protein, antibody, and the like (such as, for example, a non-naturally occurring IL-12 variant) to retain the same properties and characteristics that it possessed at the time of its manufacture within specified limits and/or storage and/or use parameters. The stability of the non-naturally occurring IL-12 variants of the disclosure can be assayed by any suitable method known in the art, such as, for example ELISA, Western blot, Biacore assay, SDS-PAGE, size exclusion chromatography, dynamic light scattering, differential scanning calorimetry, and differential scanning fluorimetry. As used herein, the term “yield of expression” refers to the amount or quantity of a given protein, cytokine, fusion protein, antibody, and the like (such as, for example, a non-naturally occurring IL-12 variant) produced using a prokaryotic or eukaryotic host system. Recombinant expression of proteins, cytokines, fusion proteins, antibodies, and the like are well-known in relevant arts. Any suitable method for quantitating or determining a yield of expression may be employed, such as, for example, UV absorption measurements, colorimetric assays (e.g., Bradford assay, BCA assay, and Lowry assay), and fluorometric assays.

In some embodiments, the non-naturally occurring IL-12 variant and/or the variant IL-12p35 subunit has binding affinity for IL-12Rβ2 reduced by at least about 10%, 20%, 30%, 40%, 50%, 60%, 70%, 80%, 90%, 91%, 92%, 93%, 94%, 95%, 96%, 97%, 98%, 99%, 100% or more compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has binding affinity for IL-12Rβ2 below the lower level of detectability of an assay, and binding affinity of a reference IL-12 for IL-12Rβ2 is between or equivalent to the lower or upper level of detectability of an assay (i.e., the binding affinity of the reference IL-12 may be equal to the lower level of detectability, the upper level of detectability, or a value between the lower and upper levels of detectability; in other words, the binding affinity is “detectable”). In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has potency reduced by at least about 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold, 11.0-fold, 12.0-fold, 13.0-fold, 14.0-fold, 15.0-fold, 16.0-fold, 17.0-fold, 18.0-fold, 19.0-fold, 20.0-fold, 21.0-fold, 22.0-fold, 23.0-fold, 24.0-fold, 25.0-fold, 30.0-fold, 35.0-fold, 40.0-fold, 45.0-fold, 50.0-fold, 100.0-fold, 150.0-fold, 200.0-fold, 250.0-fold, 300.0-fold, 350.0-fold, 400.0-fold, 450.0-fold, 500.0-fold, 550.0-fold, 600.0-fold, 650.0-fold, 700.0-fold, 750.0-fold, 800.0-fold, 850.0-fold, 900.0-fold, 950.0-fold, 1000.0-fold, 2000.0-fold, 3000.0-fold, 4000.0-fold, 5000.0-fold, 6000.0-fold, 7000.0-fold, 8000.0-fold, 9000.0-fold, 10,000.0-fold or more compared to potency of a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has potency reduced by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12 as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has a reduced capability to stimulate STAT4 signaling compared to a reference IL-12, as determined by an assay. A reduction in the capability to stimulate STAT4 signaling may refer to a reduction in the maximal response observed and/or shifting in the EC₅₀ value. In some embodiments, the capability of the non-naturally occurring IL-12 variant to stimulate STAT4 signaling is reduced by at least about 10%, 20%, 30%, 40%, 50%, 60%, 70%, 80%, 90%, 91%, 92%, 93%, 94%, 95%, 96%, 97%, 98%, 99%, 100% or more compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the capability of the non-naturally occurring IL-12 variant and/or the variant IL-12p35 subunit to stimulate STAT4 signaling is reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the non-naturally occurring IL-12 variant and/or the variant IL-12p35 subunit has a reduced capability to stimulate IFNγ production compared to a reference IL-12, as determined by an assay. A reduction in the capability to stimulate IFNγ production may refer to a reduction in the maximal response observed and/or shifting in the EC₅₀ value. In some embodiments, the capability of the non-naturally occurring IL-12 variant and/or the variant IL-12p35 subunit to stimulate IFNγ production is reduced by at least about 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold, 11.0-fold, 12.0-fold, 13.0-fold, 14.0-fold, 15.0-fold, 16.0-fold, 17.0-fold, 18.0-fold, 19.0-fold, 20.0-fold, 21.0-fold, 22.0-fold, 23.0-fold, 24.0-fold, 25.0-fold, 30.0-fold, 35.0-fold, 40.0-fold, 45.0-fold, 50.0-fold, 100.0-fold, 150.0-fold, 200.0-fold, 250.0-fold, 300.0-fold, 350.0-fold, 400.0-fold, 450.0-fold, 500.0-fold, 550.0-fold, 600.0-fold, 650.0-fold, 700.0-fold, 750.0-fold, 800.0-fold, 850.0-fold, 900.0-fold, 950.0-fold, 1000.0-fold, 2000.0-fold, 3000.0-fold, 4000.0-fold, 5000.0-fold, 6000.0-fold, 7000.0-fold, 8000.0-fold, 9000.0-fold, 10,000.0-fold or more compared to a reference IL-12, as determined by an assay. In some embodiments, the assay comprises an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, or an IL-12 Fc fusion protein.

In some embodiments, the capability of the non-naturally occurring IL-12 variant and/or the variant IL-12p35 subunit to stimulate IFNγ production is reduced by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to a reference IL-12, as determined by an assay. In some embodiments, the assay comprises an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, or an IL-12 Fc fusion protein.

2. p40 Subunit:

In accordance with any of the aspects and embodiments described herein, the present disclosure provides a non-naturally occurring IL-12 variant, comprising: a) a variant IL-12p35 subunit wherein the variant IL-12p35 subunit comprises one or more amino acid substitutions selected from the group including: Y40A, T43A, D126A, P127A, R129A, K168A, and K170A; and b) an IL-12p40 subunit.

In some embodiments, the IL-12p40 subunit comprises a variant IL-12p40 subunit.

In some embodiments, the IL-12p40 subunit comprises a variant IL-12p40 subunit, and in further embodiments the variant IL-12p40 subunit comprises one or more amino acid substitutions selected from the group that includes: C177S, C252S, and C177S/C252S.

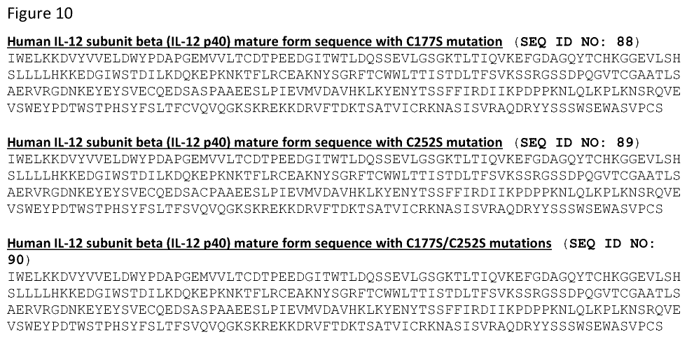

In some embodiments, the (variant) IL-12p40 subunit comprises an amino acid sequence selected from the group that includes: SEQ ID NO: 3 (wild-type precursor), SEQ ID NO: 4 (wild-type mature), SEQ ID NO: 88 (C177S), SEQ ID NO: 89 (C252S), and SEQ ID NO: 90 (C177S/C252S) (as shown in FIGS. 1 and 10).

In some embodiments, the (variant) IL-12p40 subunit comprises an amino acid sequence with at least 90%, 91%, 92%, 93%, 94%, 95%, 96%, 97%, 98%, 99% or higher sequence identity to an amino acid sequence selected from the group that includes: SEQ ID NO: 3, SEQ ID NO: 4, SEQ ID NO: 88, SEQ ID NO: 89, and SEQ ID NO: 90 (as shown in FIGS. 1 and 10).

In addition to the novel, non-naturally occurring IL-12p35 variants described above, various modifications to the IL-12p40 subunit are known in the art. Non-limiting examples of residues that may be modified include: E3, D7, E12, D14, W15, P17, D18, A19, P20, G21, E22, M23, D29, E32, E33, D34, L40, D41, Q42, S43, E45, L47, S49, T54, Q56, 155, Q56, K58, E59, F60, G61, D62, Y66, E73, H77, K84, E86, D87, G88, 189, W90, D93, K99, E100, K102, N103, K104, T105, F106, R108, E110, N113, Y114, D129, D142, Q144, E156, R159, D161, N162, K163, E164, Y165, E166, S168, D170, Q172, D174, A176, C177, P178, A179, A180, E181, S183, P185, E187, M189, H194, K195, L196, K197, N200, S204, F206, R208, D209, D214, N218, Q220, N226, Q229, E231, E235, T242, P243, S245, Y246, F247, S248, C252, Q256, K258, S259, K260, R261, E262, K264, D265, V267, D270, N281, S283, S285, R287, Q289, D290, R291, Y292, Y293, and E299. In some embodiments, the (variant) IL-12p40 subunit described above, below, or in any one of FIGS. 1 and 10 comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more additional amino acid substitutions. In some embodiments, the (variant) IL-12p40 subunit described above, below, or in any one of FIGS. 1 and 10 comprises about 1 to about 5, about 6 to about 10, about 11 to about 15, about 16 to about 20, about 21 to about 25, about 26 to about 30, about 31 to about 35, about 36 to about 40, about 41 to about 45, about 46 to about 50, about 51 to about 55, about 56 to about 60, about 1 to about 10, about 11 to about 20, about 21 to about 30, about 31 to about 40, about 41 to about 50, about 51 to about 60, about 1 to about 20, or about 21 to about 40, or about 41 to about 60 additional amino acid substitutions.

3. Single-Chain IL-12 Complex and Domain Linkers:

In accordance with any of the aspects and embodiments described herein, the present disclosure provides a non-naturally occurring IL-12 variant, comprising: a) a variant IL-12p35 subunit; and b) an IL-12p40 subunit.

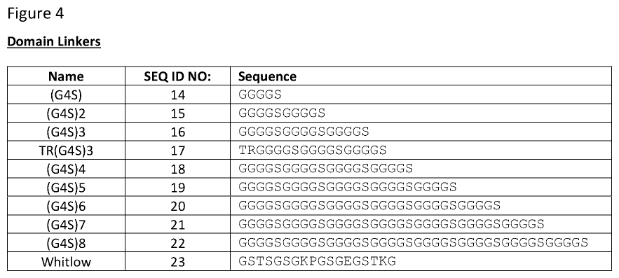

In some embodiments, the non-naturally occurring IL-12 variant comprises a single chain IL-12 complex (sc-IL-12), wherein the variant IL-12p35 subunit and the (variant) IL-12p40 subunit are linked. In further embodiments, the subunits are linked together using a domain linker (also referred to herein as a “linker” or a “linker domain”). In yet further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments, the non-naturally occurring IL-12 variant comprises a single chain IL-12 complex (sc-IL-12), wherein the variant IL-12p35 subunit and the (variant) IL-12p40 subunit are linked. In further embodiments, the C-terminus of the IL-12p35 subunit is linked to the N-terminus of the (variant) IL-12p40 subunit (sc-IL-12(p35/p40)). In yet further embodiments, the sc-IL-12(p35/p40) further comprises a linker domain, wherein the C-terminus of the IL-12p35 subunit is linked to the N-terminus of the linker domain and the C-terminus of the linker domain is linked to the N-terminus of the (variant) IL-12p40 subunit. In even further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments, the non-naturally occurring IL-12 variant comprises a single chain IL-12 complex (sc-IL-12), wherein the variant IL-12p35 subunit and the (variant) IL-12p40 subunit are linked. In some embodiments, the C-terminus of the (variant) IL-12p40 subunit is connected to the N-terminus of the IL-12p35 subunit (sc-IL-12(p40/p35)). In yet further embodiments, the sc-IL-12(p40/p35) further comprises a linker domain, wherein the C-terminus of the (variant) IL-12p40 subunit is linked to the N-terminus of the linker domain and the C-terminus of the linker domain is linked to the N-terminus of the IL-12p35 subunit. In even further embodiments, the linker domain comprises an amino acid selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments, the subunits are not attached via a linker domain.

Any of the sc-IL-12 complexes described herein may be utilized to generate a heterodimeric IL-12 Fc fusion protein and/or a homodimeric IL-12 Fc fusion protein, as discussed in further detail below.

4. Compositions with Improved Half-Life:

In accordance with any of the aspects and embodiments described herein, the present disclosure provides a non-naturally occurring IL-12 variant, comprising: a) a variant IL-12p35 subunit; and b) an IL-12p40 subunit.

In some embodiments, a non-naturally occurring IL-12 variant is provided, wherein the one or more amino acid substitutions of the variant IL-12p35 subunit improves half-life, as compared to half-life of a reference IL-12. In some embodiments, the half-life of the non-naturally occurring IL-12 variant is increased 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold or more, as compared to the half-life of a reference IL-12. In some embodiments, the reference IL-12 comprises one or more of the following: a wild-type IL-12, a human wild-type IL-12, a commercially available IL-12 molecule, an IL-12 Fc fusion protein, or any combination thereof.

Various methods for determining the half-life of an agent, such as, for example, IL-12, are known in the art. Those skilled in the relevant art will readily be able to determine and employ any number of such methods for determining changes in half-life. In some embodiments, at least one sample selected from the group comprising: (i) one or more blood samples, (ii) one or more plasma samples, (iii) one or more serum samples, (iv) one or more tissue samples, and (v) any combination thereof, are utilized to measure half-life. To measure the half-life, one, two, three, four, five, six, seven, eight, nine, 10 or more samples (as described above) may be utilized to measure half-life.

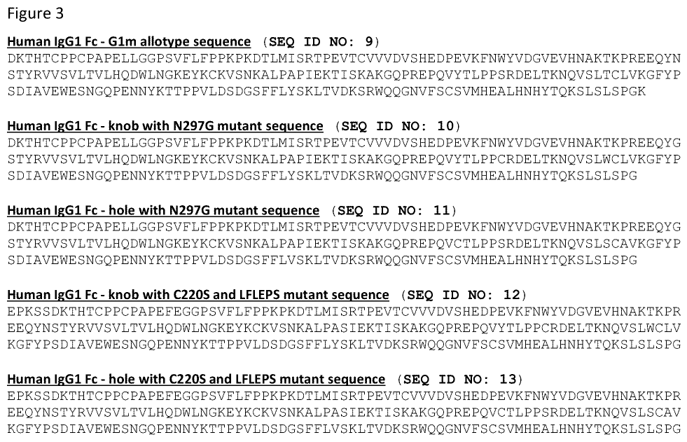

In some embodiments, the non-naturally occurring IL-12 variant further comprises one or more of the following fused to the variant IL-12p35 subunit and/or the (variant) IL-12p40 subunit: (i) a Fc domain, wherein the Fc domain comprises one or more amino acid sequences selected from the group that includes: SEQ ID NO: 9, SEQ ID NO: 10, SEQ ID NO: 11, SEQ ID NO: 12, and SEQ ID NO: 13 (as shown in FIG. 3), (ii) an albumin, (iii) one or more unstructured biodegradable polypeptides (“XTEN”), or (iv) a polyethylene glycol (PEG).

5. Nucleic Acids and Vectors:

In accordance with any of the aspects and embodiments described herein, the present disclosure provides nucleic acid compositions encoding: (i) non-naturally occurring IL-12 variants, (ii) variant IL-12p35 subunits, (iii) (variant) IL-12p40 subunits, (iv) sc-IL-12 complexes, and (v) domain linkers, as described above.

In some embodiments, one or more of the nucleic acids encoding the components of the present invention are incorporated into an expression cassette or an expression vector. It will be understood by one skilled in the art that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure includes a coding sequence for the polypeptide as disclosed herein, which is operably linked to expression control elements, such as a promoter, and optionally, any or a combination of other nucleic acid sequences that affect the transcription or translation of the coding sequence (such as, for example, an origin of replication, selectable markers, ribosomal binding sites, inducers, and the like).

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation (e.g., the introduction of heterologous DNA into a host cell). As such, in some embodiments, the vector can be a replicon, such as, for example, a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitates transfer of the nucleic acid molecule or integration into the genome of a cell or to a virus particle that mediates nucleic acid transfer, and may further include oncolytic viruses (both those naturally occurring and those that are recombinantly produced or modified in a laboratory or clinical setting). Viral particles typically include various viral components, and sometimes also host cell components, in addition to the nucleic acid of interest. The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a baculoviral vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including long terminal repeats (LTRs) that are primarily derived from a lentivirus, which is a genus of retrovirus.

Accordingly, also provided herein are vectors, plasmids, or viruses containing one or more of the nucleic acids encoding any of the non-naturally occurring IL-12 variants, variant IL-12p35 subunits, (variant) IL-12p40 subunits, sc-IL-12 complexes, and/or domain linkers disclosed herein. The nucleic acids can be contained within a vector that is capable of directing their expression in, for example, a cell that has been transformed/transduced with the vector. Suitable vectors for use in prokaryotic and eukaryotic cells are known in the art and are commercially available, or readily prepared by a skilled artisan.

As will be appreciated by those skilled in the art, the nucleic acid compositions will depend on the configuration of the non-naturally occurring IL-12 variant. Thus, for example, when the configuration requires three amino acid sequences (e.g., a non-naturally occurring IL-12 variant wherein the variant IL-12p35 and the (variant) IL-12p40 subunits are linked using a domain linker), three nucleic acid sequences can be incorporated into one or more expression vectors for expression. Similarly for other configurations, when only two nucleic acids are needed, they can be incorporated into one or two expression vectors.

DNA vectors can be introduced into host cells, such as, for example, eukaryotic cells or prokaryotic cells, via conventional transformation or transfection techniques, including, but not limited to, one or more of the following: transfection, calcium phosphate transfection, DEAE-dextran-mediated transfection, microinjection, cationic lipid-mediated transfection, electroporation, transduction, scrape loading, ballistic introduction, nucleoporation, hydrodynamic shock, infection, and the like.

Viral vectors that can be used in the disclosure include, but are not limited to, baculoviral vectors, retrovirus vectors, adenovirus vectors, adeno-associated virus vectors, lentivirus vectors, herpes virus, simian virus 40 (SV40), bovine papilloma virus vectors, and the like.

6. Recombinant Cells and Cell Cultures:

In another aspect, provided herein are cell cultures including at least one recombinant cell (also referred to herein as a “host cell”) as disclosed herein, and a culture medium. Generally, the culture medium can be any suitable culture medium for culturing the cells described herein. Techniques for transforming a wide variety of the above-mentioned cells and species are known in the art. Accordingly, cell cultures including at least one recombinant cell as disclosed herein are also within the scope of this application. Methods and systems suitable for generating and maintaining cell cultures are known in the art.

A host cell can be used for preparative purposes to propagate one or more nucleic acids encoding (i) non-naturally occurring IL-12 variants, (ii) variant IL-12p35 subunits, (iii) (variant) IL-12p40 subunits, (iv) sc-IL-12 complexes, and (v) domain linkers, as well as combinations and/or components thereof. A host cell can include a prokaryotic or eukaryotic cell in which production of the non-naturally occurring IL-12 variant is specifically intended. Non-limiting examples of host cells include bacterial cells (such as, for example, cells of Gram-positive bacteria (e.g., species of the genus Bacillus, Streptomyces, and Staphylococcus) or cells of Gram-negative bacteria (e.g., cells of the genus Escherichia and Pseudomonas)), fungal cells or yeast cells (e.g., Saccharomyces, Pichia pastoris, and Hansenula polymorpha), insect cells (e.g., cells of Drosophila and Sf9 cells), plant cells (e.g., cells from crop plants, medicinal or ornamental plants or bulbs), mammalian cells (e.g., epithelial cell lines, osteosarcoma cell lines, neuroblastoma cell lines, epithelial carcinomas, glial cells, liver cell lines, Chinese hamster ovary (CHO) cells, COS cells, BHK cells, HeLa cells, D3 cells of the line of murine embryonic stem cells (mESCs), human embryonic stem cells (e.g., HS293 cells and BG01V cells), NIH 3T3 cells, human embryonic kidney (HEK) 293T cells, human mesenchymal stem cells (hMSCSs), and the like), and the like.

### B. Heterodimeric RL 2 Fc Fusion Proteins

In one aspect, the present disclosure provides a heterodimeric Fc fusion protein comprising: a) a first fusion construct, comprising: a variant IL-12p35 subunit domain and a first Fc domain, wherein the C-terminus of the variant IL-12p35 subunit domain is covalently attached to the N-terminus of the first Fc domain, and b) a second fusion construct, comprising: an IL-12p40 subunit domain and a second Fc domain, wherein the C-terminus of the IL-12p40 subunit domain is covalently attached to the N-terminus of the second Fc domain.

In another aspect, the present disclosure provides a heterodimeric Fc fusion protein comprising: a) a first fusion construct, comprising: a variant IL-12p35 subunit domain and a first Fc domain, wherein the N-terminus of the variant IL-12p35 subunit domain is covalently attached to the C-terminus of the first Fc domain, and b) a second fusion construct, comprising: an IL-12p40 subunit domain and a second Fc domain, wherein the N-terminus of the IL-12p40 subunit domain is covalently attached to the C-terminus of the second Fc domain.

Various configurations for the heterodimeric Fc fusion proteins, as well as the variant IL-12p35 subunit domain, the IL-12p40 subunit domain, and the Fc domains, are expanded upon below.

1. p35 Subunit Domain:

In accordance with any of the aspects and embodiments described herein, the present disclosure provides a heterodimeric Fc fusion protein comprising: a) a first fusion construct, comprising: a variant IL-12p35 subunit domain and a first Fc domain, wherein the variant IL-12 p35 subunit domain is covalently attached to the N-terminus or the C-terminus of the first Fe domain, and b) a second fusion construct, comprising: an IL-12p40 subunit domain and a second Fc domain, wherein the IL-12p40 subunit domain is covalently attached to the N-terminus or the C-terminus of the second Fc domain.

In some embodiments, the variant IL-12p35 subunit domain comprises one or more amino acid substitutions selected from the group including: Y40A, T43A, D126A, P127A, R129A, K168A, and K170A. In some embodiments, the one or more amino acid substitutions comprise Y40A. In some embodiments, the one or more amino acid substitutions comprise T43A. In some embodiments, the one or more amino acid substitutions comprise D126A. In some embodiments, the one or more amino acid substitutions comprise P127A. In some embodiments, the one or more amino acid substitutions comprise R129A. In some embodiments, the one or more amino acid substitutions comprise K168A. In some embodiments, the one or more amino acid substitutions comprise K170A.

In some embodiments, the variant IL-12p35 subunit domain comprises two or more amino acid substitutions selected from the group including: Y40A, T43A, D126A, P127A, R129A, K168A, and K170A. In some embodiments, the two or more amino acid substitutions comprise Y40A/T43A. In some embodiments, the two or more amino acid substitutions comprise Y40A/D126A. In some embodiments, the two or more amino acid substitutions comprise Y40A/P127A. In some embodiments, the two or more amino acid substitutions comprise Y40A/R129A. In some embodiments, the two or more amino acid substitutions comprise Y40A/K168A. In some embodiments, the two or more amino acid substitutions comprise T43A/D126A. In some embodiments, the two or more amino acid substitutions comprise T43A/P127A. In some embodiments, the two or more amino acid substitutions comprise T43A/R129A. In some embodiments, the two or more amino acid substitutions comprise T43A/K168A. In some embodiments, the two or more amino acid substitutions comprise D126A/P127A. In some embodiments, the two or more amino acid substitutions comprise D126A/R129A. In some embodiments, the two or more amino acid substitutions comprise D126A/K168A. In some embodiments, the two or more amino acid substitutions comprise P127A/R129A. In some embodiments, the two or more amino acid substitutions comprise P127A/K168A. In some embodiments, the two or more amino acid substitutions comprise R129A/K168A. In some embodiments, the two or more amino acid substitutions comprise Y40A/K170A. In some embodiments, the two or more amino acid substitutions comprise T43A/K170A. In some embodiments, the two or more amino acid substitutions comprise D126A/K170A. In some embodiments, the two or more amino acid substitutions comprise P127A/K170A. In some embodiments, the two or more amino acid substitutions comprise R129A/K170A. In some embodiments, the two or more amino acid substitutions comprise K168A/K170A.

In some embodiments, the variant IL-12p35 subunit domain comprises three or more amino acid substitutions selected from the group including: Y40A, T43A, D126A, P127A, R129A, K168A, and K170A. In some embodiments, the three or more amino acid substitutions comprise Y40A/T43A/D126A. In some embodiments, the three or more amino acid substitutions comprise Y40A/T43A/P127A. In some embodiments, the three or more amino acid substitutions comprise Y40A/T43A/R129A. In some embodiments, the three or more amino acid substitutions comprise Y40A/T43A/K168A. In some embodiments, the three or more amino acid substitutions comprise Y40A/D126A/P127A. In some embodiments, the three or more amino acid substitutions comprise Y40A/D126A/R129A. In some embodiments, the three or more amino acid substitutions comprise Y40A/D126A/K168A. In some embodiments, the three or more amino acid substitutions comprise Y40A/P127A/R129A. In some embodiments, the three or more amino acid substitutions comprise Y40A/P127A/K168A. In some embodiments, the three or more amino acid substitutions comprise Y40A/R129A/K168A. In some embodiments, the three or more amino acid substitutions comprise T43A/D126A/P127A. In some embodiments, the three or more amino acid substitutions comprise T43A/D126A/R129A. In some embodiments, the three or more amino acid substitutions comprise T43A/D126A/K168A. In some embodiments, the three or more amino acid substitutions comprise T43A/P127A/R129A.

In some embodiments, the three or more amino acid substitutions comprise T43A/P127A/K168A. In some embodiments, the three or more amino acid substitutions comprise T43A/R129A/K168A. In some embodiments, the three or more amino acid substitutions comprise D126A/P127A/R129A. In some embodiments, the three or more amino acid substitutions comprise D126A/P127A/K168A. In some embodiments, the three or more amino acid substitutions comprise D126A/R129A/K168A. In some embodiments, the three or more amino acid substitutions comprise P127A/R129A/K168A. In some embodiments, the three or more amino acid substitutions comprise Y40A/T43A/K170A. In some embodiments, the three or more amino acid substitutions comprise Y40A/D126A/K170A. In some embodiments, the three or more amino acid substitutions comprise Y40A/P127A/K170A. In some embodiments, the three or more amino acid substitutions comprise Y40A/R129A/K170A. In some embodiments, the three or more amino acid substitutions comprise Y40A/K168A/K170A. In some embodiments, the three or more amino acid substitutions comprise T43A/D126A/K170A.

In some embodiments, the three or more amino acid substitutions comprise T43A/P127A/K170A. In some embodiments, the three or more amino acid substitutions comprise T43A/R129A/K170A. In some embodiments, the three or more amino acid substitutions comprise T43A/K168A/K170A. In some embodiments, the three or more amino acid substitutions comprise D126A/P127A/K170A. In some embodiments, the three or more amino acid substitutions comprise D126A/R129A/K170A. In some embodiments, the three or more amino acid substitutions comprise D126A/K168A/K170A. In some embodiments, the three or more amino acid substitutions comprise P127A/R129A/K170A. In some embodiments, the three or more amino acid substitutions comprise P127A/K168A/K170A. In some embodiments, the three or more amino acid substitutions comprise R129A/K168A/K170A.

In some embodiments, the variant IL-12p35 subunit domain comprises four or more amino acid substitutions selected from the group including: Y40A, T43A, D126A, P127A, R129A, K168A, and K170A. In some embodiments, the four or more amino acid substitutions comprise Y40A/T43A/D126A/P127A. In some embodiments, the four or more amino acid substitutions comprise Y40A/T43A/D126A/R129A. In some embodiments, the four or more amino acid substitutions comprise Y40A/T43A/D126A/K168A. In some embodiments, the four or more amino acid substitutions comprise Y40A/T43A/P127A/R129A. In some embodiments, the four or more amino acid substitutions comprise Y40A/T43A/P127A/K168A. In some embodiments, the four or more amino acid substitutions comprise Y40A/T43A/R129A/K168A.

In some embodiments, the four or more amino acid substitutions comprise Y40A/D126A/P127A/R129A. In some embodiments, the four or more amino acid substitutions comprise Y40A/D126A/P127A/K168A. In some embodiments, the four or more amino acid substitutions comprise Y40A/D126A/R129A/K168A. In some embodiments, the four or more amino acid substitutions comprise Y40A/P127A/R129A/K168A. In some embodiments, the four or more amino acid substitutions comprise T43A/D126A/P127A/R129A. In some embodiments, the four or more amino acid substitutions comprise T43A/D126A/P127A/K168A. In some embodiments, the four or more amino acid substitutions comprise T43A/D126A/R129A/K168A.

In some embodiments, the four or more amino acid substitutions comprise T43A/P127A/R129A/K168A. In some embodiments, the four or more amino acid substitutions comprise D126A/P127A/R129A/K168A. In some embodiments, the four or more amino acid substitutions comprise Y40A/T43A/D126A/K170A. In some embodiments, the four or more amino acid substitutions comprise Y40A/T43A/P127A/K170A. In some embodiments, the four or more amino acid substitutions comprise Y40A/T43A/R129A/K170A. In some embodiments, the four or more amino acid substitutions comprise Y40A/T43A/K168A/K170A. In some embodiments, the four or more amino acid substitutions comprise Y40A/D126A/P127A/K170A.

In some embodiments, the four or more amino acid substitutions comprise Y40A/D126A/R129A/K170A. In some embodiments, the four or more amino acid substitutions comprise Y40A/D126A/K168A/K170A. In some embodiments, the four or more amino acid substitutions comprise Y40A/P127A/R129A/K170A. In some embodiments, the four or more amino acid substitutions comprise Y40A/P127A/K168A/K170A. In some embodiments, the four or more amino acid substitutions comprise Y40A/R129A/K168A/K170A. In some embodiments, the four or more amino acid substitutions comprise T43A/D126A/P127A/K170A. In some embodiments, the four or more amino acid substitutions comprise T43A/D126A/R129A/K170A.

In some embodiments, the four or more amino acid substitutions comprise T43A/D126A/K168A/K170A. In some embodiments, the four or more amino acid substitutions comprise T43A/P127A/R129A/K170A. In some embodiments, the four or more amino acid substitutions comprise T43A/P127A/K168A/K170A. In some embodiments, the four or more amino acid substitutions comprise T43A/R129A/K168A/K170A. In some embodiments, the four or more amino acid substitutions comprise D126A/P127A/R129A/K170A. In some embodiments, the four or more amino acid substitutions comprise D126A/P127A/K168A/K170A. In some embodiments, the four or more amino acid substitutions comprise D126A/R129A/K168A/K170A. In some embodiments, the four or more amino acid substitutions comprise P127A/R129A/K168A/K170A.

In some embodiments, the variant IL-12p35 subunit domain comprises five or more amino acid substitutions selected from the group including: Y40A, T43A, D126A, P127A, R129A, K168A, and K170A. In some embodiments, the five or more amino acid substitutions comprise Y40A/T43A/D126A/P127A/R129A. In some embodiments, the five or more amino acid substitutions comprise Y40A/T43A/D126A/P127A/K168A. In some embodiments, the five or more amino acid substitutions comprise Y40A/D126A/P127A/R129A/K168A. In some embodiments, the five or more amino acid substitutions comprise Y40A/T43A/P127A/R129A/K168A. In some embodiments, the five or more amino acid substitutions comprise Y40A/T43A/D126A/R129A/K168A. In some embodiments, the five or more amino acid substitutions comprise T43A/D126A/P127A/R129A/K168A. In some embodiments, the five or more amino acid substitutions comprise Y40A/T43A/D126A/P127A/K170A. In some embodiments, the five or more amino acid substitutions comprise Y40A/T43A/D126A/R129A/K170A. In some embodiments, the five or more amino acid substitutions comprise Y40A/T43A/D126A/K168A/K170A. In some embodiments, the five or more amino acid substitutions comprise Y40A/T43A/P127A/R129A/K170A. In some embodiments, the five or more amino acid substitutions comprise Y40A/T43A/P127A/K168A/K170A. In some embodiments, the five or more amino acid substitutions comprise Y40A/T43A/R129A/K168A/K170A. In some embodiments, the five or more amino acid substitutions comprise Y40A/D126A/P127A/R129A/K170A. In some embodiments, the five or more amino acid substitutions comprise Y40A/D126A/P127A/K168A/K170A. In some embodiments, the five or more amino acid substitutions comprise Y40A/D126A/R129A/K168A/K170A. In some embodiments, the five or more amino acid substitutions comprise Y40A/P127A/R129A/K168A/K170A. In some embodiments, the five or more amino acid substitutions comprise T43A/D126A/P127A/R129A/K170A. In some embodiments, the five or more amino acid substitutions comprise T43A/D126A/P127A/K168A/K170A. In some embodiments, the five or more amino acid substitutions comprise T43A/D126A/R129A/K168A/K170A. In some embodiments, the five or more amino acid substitutions comprise T43A/P127A/R129A/K168A/K170A. In some embodiments, the five or more amino acid substitutions comprise D126A/P127A/R129A/K168A/K170A.

In some embodiments, the variant IL-12p35 subunit domain comprises six or more amino acid substitutions selected from the group including: Y40A, T43A, D126A, P127A, R129A, K168A, and K170A. In some embodiments, the six or more amino acid substitutions comprise Y40A/T43A/D126A/P127A/R129A/K168A. In some embodiments, the six or more amino acid substitutions comprise Y40A/T43A/D126A/P127A/R129A/K170A. In some embodiments, the six or more amino acid substitutions comprise Y40A/T43A/D126A/P127A/K168A/K170A. In some embodiments, the six or more amino acid substitutions comprise Y40A/D126A/P127A/R129A/K168A/K170A. In some embodiments, the six or more amino acid substitutions comprise Y40A/T43A/P127A/R129A/K168A/K170A. In some embodiments, the six or more amino acid substitutions comprise Y40A/T43A/D126A/R129A/K168A/K170A. In some embodiments, the six or more amino acid substitutions comprise T43A/D126A/P127A/R129A/K168A/K170A.

In some embodiments, the variant IL-12p35 subunit domain comprises seven or more amino acid substitutions selected from the group including: Y40A, T43A, D126A, P127A, R129A, K168A, and K170A.

In some embodiments, the variant IL-12p35 subunit domain comprises an amino acid sequence selected from the group including: SEQ ID NOs: 24-86 and SEQ ID NOs: 103-166 (as shown in FIGS. 5-9, 78, and 80).

In some embodiments, the variant IL-12p35 subunit domain comprises an amino acid sequence with at least 90%, 91%, 92%, 93%, 94%, 95%, 96%, 97%, 98%, 99% or higher sequence identity to an amino acid sequence selected from the group including: SEQ ID NO: 1, SEQ ID NO: 2, SEQ ID NOs: 24-87, and SEQ ID NOs: 103-166 (as shown in FIGS. 1, 5-9, 78, and 80).

In some embodiments, the variant IL-12p35 subunit domain comprises a substitution mutation at amino acid residue Y40. In some further embodiments, the substitution mutation at amino acid residue Y40 is selected from the group including: Y40C, Y40D, Y40E, Y40G, Y40K, Y40N, Y40P, Y40Q, Y40R, Y40S, and Y40T. In some embodiments, the variant IL-12p35 subunit domain comprises any one of SEQ ID NOs: 177-187.

In some embodiments, the variant IL-12p35 subunit domain comprises a substitution mutation at amino acid residue D126. In some further embodiments, the substitution mutation at amino acid residue D126 is selected from the group including: D126C, D126E, D126F, D126G, D1261, D126K, D126L, D126M, D126N, D126P, D126Q, D126R, D126S, D126T, D126V, and D126W.

In some embodiments, the variant IL-12p35 subunit domain comprises a first substitution mutation, comprising: Y40A, and further comprises a second substitution mutation selected from the group including: D126C, D126E, D126F, D126G, D1261, D126K, D126L, D126M, D126N, D126P, D126Q, D126R, D126S, D126T, D126V, and D126W. In some embodiments, the variant IL-12p35 subunit domain comprises any one of SEQ ID NOs: 199-214.

In some embodiments, the variant IL-12p35 subunit domain comprises a substitution mutation at amino acid residue P127. In some further embodiments, the substitution mutation at amino acid residue P127 is selected from the group including: P127C, P127D, P127E, P127F, P127G, P127H, P127K, P127M, P127N, P127Q, P127R, and P127S.

In some embodiments, the variant IL-12p35 subunit domain comprises a first substitution mutation, comprising: Y40A, and further comprises a second substitution mutation selected from the group including: P127C, P127D, P127E, P127F, P127G, P127H, P127K, P127M, P127N, P127Q, P127R, and P127S. In some embodiments, the variant IL-12p35 subunit domain comprises any one of SEQ ID NOs: 279-290.

In some embodiments, the variant IL-12p35 subunit domain comprises a substitution mutation at amino acid residue R129. In some further embodiments, the substitution mutation at amino acid residue R129 is selected from the group including: R129C, R129D, R129E, R129F, R129G, R129H, R129I, R129K, R129L, R129M, R129N, R129P, R129Q, R129S, R129T, R129V, R129W, and R129Y.

In some embodiments, the variant IL-12p35 subunit domain comprises a first substitution mutation, comprising: Y40A, and further comprises a second substitution mutation selected from the group including: R129C, R129D, R129E, R129F, R129G, R129H, R129I, R129K, R129L, R129M, R129N, R129P, R129Q, R129S, R129T, R129V, R129W, and R129Y.

In some embodiments, the variant IL-12p35 subunit domain comprises any one of SEQ ID NOs: 215-231.

In some embodiments, the variant IL-12p35 subunit domain comprises a substitution mutation at amino acid residue K168. In some further embodiments, the substitution mutation at amino acid residue K168 is selected from the group including: K168C, K168D, K168E, K168F, K168G, K168H, K168I, K168L, K168M, K168N, K168P, K168Q, K168S, K168T, K168W, and K168Y.

In some embodiments, the variant IL-12p35 subunit domain comprises a first substitution mutation, comprising: Y40A, and further comprises a second substitution mutation selected from the group including: K168C, K168D, K168E, K168F, K168G, K168H, K168I, K168L, K168M, K168N, K168P, K168Q, K168S, K168T, K168W, and K168Y. In some embodiments, the variant IL-12p35 subunit domain comprises any one of SEQ ID NOs: 232-247.

In some embodiments, the variant IL-12p35 subunit domain comprises a substitution mutation at amino acid residue K170. In some further embodiments, the substitution mutation at amino acid residue K170 is selected from the group including: K170C, K170D, K170E, K170G, K170I, K170M, K170P, K170S, K170T, K170V, K170F, K170L, K170N, and K170W. In some embodiments, the variant IL-12p35 subunit domain comprises any one of SEQ ID NOs: 188-198 or 306-308.

In some embodiments, the variant IL-12p35 subunit domain comprises a first substitution mutation, comprising: Y40A, and further comprises a second substitution mutation selected from the group including: K170L and K170T. In some embodiments, the variant IL-12p35 subunit domain comprises any one of SEQ ID NOs: 248 or 249.

In some embodiments, the variant IL-12p35 subunit domain comprises: (i) a first substitution mutation selected from the group including: Y40A, Y40C, Y40D, Y40E, Y40G, Y40K, Y40N, Y40P, Y40Q, Y40R, Y40S, and Y40T, and (ii) a second substitution mutation selected from the group including: D126A, D126C, D126E, D126F, D126G, D1261, D126K, D126L, D126M, D126N, D126P, D126Q, D126R, D126S, D126T, D126V, D126W, R129A, R129C, R129D, R129E, R129F, R129G, R129H, R129I, R129K, R129L, R129M, R129N, R129P, R129Q, R129S, R129T, R129V, R129W, R129Y, K168A, K168C, K168D, K168E, K168F, K168G, K168H, K168I, K168L, K168M, K168N, K168P, K168Q, K168S, K168T, K168W, K168Y, K170A, K170C, K170D, K170E, K170G, K170I, K170M, K170P, K170S, K170T, K170V, K170F, K170L, K170N, and K170W.

In some embodiments, the variant IL-12p35 subunit domain comprises a first substitution mutation, comprising: Y40E, and further comprises a second substitution mutation selected from the group including: K170A, K168A, K168I, K168T, and R129A. In some embodiments, the variant IL-12p35 subunit domain comprises any one of SEQ ID NOs: 250-253 or 311.

In some embodiments, the variant IL-12p35 subunit domain comprises a first substitution mutation, comprising: Y40G, and further comprises a second substitution mutation selected from the group including: K170A, K168A, K168I, K168T, and R129A. In some embodiments, the variant IL-12p35 subunit domain comprises any one of SEQ ID NOs: 254-257 or 309.

In some embodiments, the variant IL-12p35 subunit domain comprises a first substitution mutation, comprising: Y40P, and further comprises a second substitution mutation selected from the group including: K170A, K168A, K168D, K168I, and K168T. In some embodiments, the variant IL-12p35 subunit domain comprises any one of SEQ ID NOs: 258-261 or 310.

In some embodiments, the variant IL-12p35 subunit domain comprises a first substitution mutation, comprising: Y40S, and further comprises a second substitution mutation selected from the group including: K168I, K168T, K170A, K170L, K170T, and R129A. In some embodiments, the variant IL-12p35 subunit domain comprises any one of SEQ ID NOs: 262-267.

In some embodiments, the variant IL-12p35 subunit domain comprises a first substitution mutation, comprising: K170A, and further comprises a second substitution mutation selected from the group including: K168I, K168T, and R129E. In some embodiments, the variant IL-12p35 subunit domain comprises any one of SEQ ID NOs: 268-270.

In some embodiments, the variant IL-12p35 subunit domain comprises a first substitution mutation, comprising: K170P, and further comprises a second substitution mutation selected from the group including: K168A, K168I, K168T, and R129E. In some embodiments, the variant IL-12p35 subunit domain comprises any one of SEQ ID NOs: 271-274.

In some embodiments, the variant IL-12p35 subunit domain comprises a first substitution mutation, comprising: K170T, and further comprises a second substitution mutation selected from the group including: K168A, K168I, K168T, and R129E. In some embodiments, the variant IL-12p35 subunit domain comprises any one of SEQ ID NOs: 275-278.

In some embodiments, the variant IL-12p35 subunit domain may further comprise a C74S substitution mutation.

In addition to the novel, heterodimeric Fc fusion proteins described above, additional modifications to various residues are known in relevant arts. Non-limiting examples of residues that may be modified include: Q20, N21, Q35, E38, F39, P41, S44, E45, E46, E50, H49, K54, D55, T59, V60, E61, C63, L64, P65, E67, L68, T69, N71, S73, C74, L75, N76, E79, N85, L89, F96, M97, M98, A99, L124, M125, K128, Q130, Q135, N136, E143, Q146, N151, E153, K158, E162, E163, D165, F166, Y167, 1171, R181, 1182, R183, V185, T186, D188, R189, V190, M191, S192, Y193, N195, A196, and S197. In some embodiments, the variant IL-12p35 subunit domain described above, below, or in any one of FIGS. 1, 5-9, 78, and 80 comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more additional amino acid substitutions. In some embodiments, the variant IL-12p35 subunit domain described above, below, or in any one of FIGS. 1, 5-9, 78, and 80 comprises about 1 to about 5, about 6 to about 10, about 11 to about 15, about 16 to about 20, about 21 to about 25, about 26 to about 30, about 31 to about 35, about 36 to about 40, about 1 to about 10, about 11 to about 20, about 21 to about 30, about 31 to about 40, about 1 to about 20, or about 21 to about 40 additional amino acid substitutions. In some embodiments, the variant IL-12p35 subunit domain comprises SEQ ID NO: 87 (as shown in FIG. 9), and further comprises one, two, three, four, five, six, or all seven amino acid substitutions selected from the group including: Y40A, T43A, D126A, P127A, R129A, K168A, and K170A.

Mutations in the variant IL-12p35 subunit domain may result in changes to one or more of the following parameters: (i) binding affinity, (ii) potency, (iii) activity, (iv) manufacturability, or (v) stability; however, changes to one or more of the preceding parameters (such as, for example, binding affinity) may not necessarily be correlated with changes in one or more of the other preceding parameters (such as, for example, potency or activity). In some embodiments, the one or more amino acid substitutions in the variant IL-12p35 subunit domain result in an altered binding affinity to IL-12Rβ2, as compared to the binding affinity of a reference IL-12. In some embodiments, the one or more amino acid substitutions decreases the binding affinity of the variant IL-12p35 subunit domain to IL-12Rβ2 compared to a reference IL-12. In some embodiments, the reference IL-12 comprises one or more of the following: a wild-type IL-12, a human wild-type IL-12, a commercially available IL-12 molecule, an IL-12 Fc fusion protein, or any combination thereof. In other embodiments, the one or more amino acid substitutions in the variant IL-12p35 subunit do not affect binding affinity but may result in changes to one or more of the following parameters: (i) potency, (ii) activity, (iii) manufacturability, (iv) stability, or (v) any combination thereof.

A variety of assay formats may be used to select a heterodimeric Fc fusion protein that binds to a ligand of interest (e.g., IL-12Rβ2 and/or IL-12Rβ1). Non-limiting examples include: solid-phase ELISA immunoassay, immunoprecipitation, a Biacore assay, a KinExA assay, fluorescence-activated cell sorting (FACS), an Octet assay, Western blot analysis, and the like.

The binding activity of the heterodimeric Fc fusion proteins of the disclosure can be assayed by any suitable method known in the art, such as, for example, a surface plasmon resonance (SPR) assay, an enzyme-linked immunosorbent assay (ELISA), an ELISpot assay, Biacore assays, KinExA assays, and the like.

One of ordinary skill in the art will appreciate that binding affinity can also be used as a measure of the “strength” of a non-covalent interaction between two binding partners (e.g., a variant IL-12p35 subunit domain and IL-12Rβ2). Binding affinity between two molecules may be quantified by determination of the dissociation constant (K_{D}). In turn, K_{D} can be determined by measurement of the kinetics of complex formation and dissociation using suitable assays known in the art, such as, for example, an SPR assay. The rate constants corresponding to the association and the dissociation of a monovalent complex are referred to as the association rate constants kₐ (or kₒₙ) and dissociation rate constant kₐ (or k_{off}), respectively. K_{D} is related to kₐ and k_{d} through the equation: K_{D}=k_{d}/kₐ. The value of the dissociation constant can be determined directly by well-known methods.

As used herein, the term “potency” refers to the ability of a given protein, cytokine, fusion protein, antibody, and the like (such as, for example, a heterodimeric Fc fusion protein) to elicit a response at a certain dose or concentration in a given biological system or experimental setting. The potency of the heterodimeric Fc fusion proteins of the disclosure can be assayed by any suitable method known in the art, such as, for example, an IL-12 HEK reporter assay (e.g., InvivoGen's IL-12 HEK reporter assay (Catalog No. hkb-il12)), a ligand-binding assay (e.g., ELISA or flow cytometry), and/or a functional assay. Generally, changes in potency may be demonstrated graphically as a leftward or rightward shift in a response curve compared to a control. A rightward shift of the response curve is generally indicative of a reduction in potency, whereas as a leftward shift of the response curve is generally indicative of an increase in potency. As used herein, the term “activity” refers to the specific response of a given protein, cytokine, fusion protein, antibody, and the like (such as, for example, a heterodimeric Fc fusion protein) elicited in a particular biological system or experimental setting at a given dose or concentration. The activity of the heterodimeric Fc fusion proteins of the disclosure can be assayed by any suitable method known in the art, such as, for example, a ligand-binding assay and/or a functional assay. Generally, changes in activity may be demonstrated graphically as an upward or downward shift in a response curve compared to a control. An upward shift of the response curve is generally indicative of an increase in activity, whereas a downward shift of the response curve is generally indicative of a decrease in activity.

As used herein, the term “manufacturability” refers to any property that may impact the process of producing and/or storing a given protein, cytokine, fusion protein, antibody, and the like at a scale and quantity sufficient for administration to an individual. Examples of properties that impact manufacturability include, but are not limited to, the stability, purity, aggregation levels, and/or yield of expression of a given protein, cytokine, fusion protein, antibody, and the like (such as, for example, a heterodimeric Fc fusion protein). As used herein, the term “stability” refers to the ability of a given protein, cytokine, fusion protein, antibody, and the like (such as, for example, a heterodimeric Fc fusion protein) to retain the same properties and characteristics that it possessed at the time of its manufacture within specified limits and/or storage and/or use parameters. The stability of the heterodimeric Fc fusion proteins of the disclosure can be assayed by any suitable method known in the art, such as, for example ELISA, Western blot, Biacore assay, SDS-PAGE, size exclusion chromatography, dynamic light scattering, differential scanning calorimetry, and differential scanning fluorimetry. As used herein, the term “yield of expression” refers to the amount or quantity of a given protein, cytokine, fusion protein, antibody, and the like (such as, for example, a heterodimeric Fc fusion protein) produced using a prokaryotic or eukaryotic host system. Recombinant expression of proteins, cytokines, fusion proteins, antibodies, and the like are well-known in relevant arts. Any suitable method for quantitating or determining a yield of expression may be employed, such as, for example, UV absorption measurements, colorimetric assays (e.g., Bradford assay, BCA assay, and Lowry assay), and fluorometric assays.

In some embodiments, the heterodimeric Fc fusion protein and/or the variant IL-12p35 subunit domain has binding affinity for IL-12Rβ2 reduced by at least about 10%, 20%, 30%, 40%, 50%, 60%, 70%, 80%, 90%, 91%, 92%, 93%, 94%, 95%, 96%, 97%, 98%, 99%, 100% or more compared to binding affinity of a reference IL-12, as determined by an SPR assay. In some embodiments, the heterodimeric Fc fusion protein and/or the variant IL-12p35 subunit domain has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has binding affinity for IL-12Rβ2 reduced by at least about 10%, 20%, 30%, 40%, 50%, 60%, 70%, 80%, 90%, 91%, 92%, 93%, 94%, 95%, 96%, 97%, 98%, 99%, 100% or more compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has binding affinity for IL-12Rβ2 reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has binding affinity for IL-12Rβ2 below the lower level of detectability of an assay, and binding affinity of a reference IL-12 for IL-12Rβ2 is between or equivalent to the lower or upper level of detectability of an assay (i.e., the binding affinity of the reference IL-12 may be equal to the lower level of detectability, the upper level of detectability, or a value between the lower and upper levels of detectability; in other words, the binding affinity is “detectable”). In some embodiments, the assay comprises an SPR assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has potency reduced by at least about 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold, 11.0-fold, 12.0-fold, 13.0-fold, 14.0-fold, 15.0-fold, 16.0-fold, 17.0-fold, 18.0-fold, 19.0-fold, 20.0-fold, 21.0-fold, 22.0-fold, 23.0-fold, 24.0-fold, 25.0-fold, 30.0-fold, 35.0-fold, 40.0-fold, 45.0-fold, 50.0-fold, 100.0-fold, 150.0-fold, 200.0-fold, 250.0-fold, 300.0-fold, 350.0-fold, 400.0-fold, 450.0-fold, 500.0-fold, 550.0-fold, 600.0-fold, 650.0-fold, 700.0-fold, 750.0-fold, 800.0-fold, 850.0-fold, 900.0-fold, 950.0-fold, 1000.0-fold, 2000.0-fold, 3000.0-fold, 4000.0-fold, 5000.0-fold, 6000.0-fold, 7000.0-fold, 8000.0-fold, 9000.0-fold, 10,000.0-fold or more compared to potency of a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has potency reduced by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to potency of a reference IL-12 as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has a reduced capability to stimulate STAT4 signaling compared to a reference IL-12, as determined by an assay. A reduction in the capability to stimulate STAT4 signaling may refer to a reduction in the maximal response observed and/or shifting in the EC₅₀ value. In some embodiments, the capability of the heterodimeric Fc fusion protein to stimulate STAT4 signaling is reduced by at least about 10%, 20%, 30%, 40%, 50%, 60%, 70%, 80%, 90%, 91%, 92%, 93%, 94%, 95%, 96%, 97%, 98%, 99%, 100% or more compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the capability of the heterodimeric Fc fusion protein and/or the variant IL-12p35 subunit domain to stimulate STAT4 signaling is reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, or an IL-12 Fc fusion protein.

In some embodiments, the heterodimeric Fc fusion protein and/or the variant IL-12p35 subunit domain has a reduced capability to stimulate IFNγ production compared to a reference IL-12, as determined by an assay. A reduction in the capability to stimulate IFNγ production may refer to a reduction in the maximal response observed and/or shifting in the EC₅₀ value. In some embodiments, the capability of the heterodimeric Fc fusion protein and/or the variant IL-12p35 subunit domain to stimulate IFNγ production is reduced by at least about 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold, 11.0-fold, 12.0-fold, 13.0-fold, 14.0-fold, 15.0-fold, 16.0-fold, 17.0-fold, 18.0-fold, 19.0-fold, 20.0-fold, 21.0-fold, 22.0-fold, 23.0-fold, 24.0-fold, 25.0-fold, 30.0-fold, 35.0-fold, 40.0-fold, 45.0-fold, 50.0-fold, 100.0-fold, 150.0-fold, 200.0-fold, 250.0-fold, 300.0-fold, 350.0-fold, 400.0-fold, 450.0-fold, 500.0-fold, 550.0-fold, 600.0-fold, 650.0-fold, 700.0-fold, 750.0-fold, 800.0-fold, 850.0-fold, 900.0-fold, 950.0-fold, 1000.0-fold, 2000.0-fold, 3000.0-fold, 4000.0-fold, 5000.0-fold, 6000.0-fold, 7000.0-fold, 8000.0-fold, 9000.0-fold, 10,000.0-fold or more compared to a reference IL-12, as determined by an assay. In some embodiments, the assay comprises an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, or an IL-12 Fc fusion protein.

In some embodiments, the capability of the heterodimeric Fc fusion protein and/or the variant IL-12p35 subunit domain to stimulate IFNγ production is reduced by at least about 0.5-fold to about 50.0-fold, about 0.5-fold to about 5.0-fold, about 5.0 to about 10.0-fold, about 10.0-fold to about 15.0-fold, about 15.0-fold to about 20.0-fold, about 20.0-fold to about 25.0-fold, about 25.0-fold to about 30.0-fold, about 30.0-fold to about 35.0-fold, about 35.0-fold to about 40.0-fold, about 40.0-fold to about 45.0-fold, about 45.0-fold to about 50.0-fold, about 50.0-fold to about 100.0-fold, about 100.0-fold to about 200.0-fold, about 200.0-fold to about 300.0-fold, about 300.0-fold to about 400.0-fold, about 400.0-fold to about 500.0-fold, about 500.0-fold to about 600.0-fold, about 600.0-fold to about 700.0-fold, about 700.0-fold to about 800.0-fold, about 800.0-fold to about 900.0-fold, about 900.0-fold to about 1000.0-fold, about 1000.0-fold to about 2000.0-fold, about 2000.0-fold to about 3000.0-fold, about 3000.0-fold to about 4000.0-fold, about 4000.0-fold to about 5000.0-fold, about 5000.0-fold to about 6000.0-fold, about 6000.0-fold to about 7000.0-fold, about 7000.0-fold to about 8000.0-fold, about 8000.0-fold to about 9000.0-fold, about 9000.0-fold to about 10,000.0-fold, about 10,000.0-fold to about 50,000.0-fold, about 50,000.0-fold to about 100,000.0-fold, about 100,000.0-fold to about 200,000.0-fold, about 200,000.0-fold to about 300,000.0-fold, about 300,000.0-fold or higher compared to a reference IL-12, as determined by an assay. In some embodiments, the assay comprises an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, or an IL-12 Fc fusion protein.

In some embodiments, heterodimeric Fc fusion proteins and/or variant IL-12p35 subunit domains are provided, wherein one or more amino acid substitutions of the variant IL-12p35 subunit domain improves half-life, as compared to half-life of a reference IL-12. In some embodiments, the fusion of the variant IL-12p35 subunit domain and/or the (variant) IL-12p40 subunit domain improves half-life, as compared to half-life of a reference. In some embodiments, the half-life of the heterodimeric Fc fusion protein and/or variant IL-12p35 subunit domain is decreased or increased 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 6.0-fold, 7.0-fold, 8.0-fold, 9.0-fold, 10.0-fold or more, as compared to the half-life of a reference IL-12. In some embodiments, the reference IL-12 comprises one or more of the following: a wild-type IL-12, a human wild-type IL-12, a commercially available IL-12 molecule, an IL-12 Fc fusion protein, or any combination thereof.

Various methods for determining the half-life of an agent, such as, for example, IL-12, are known in the art. Those skilled in the relevant art will readily be able to determine and employ any number of such methods for determining changes in half-life. In some embodiments, at least one sample selected from the group comprising: (i) one or more blood samples, (ii) one or more plasma samples, (iii) one or more serum samples, (iv) one or more tissue samples, and (v) any combination thereof, are utilized to measure half-life. To measure the half-life, one, two, three, four, five, six, seven, eight, nine, 10 or more samples (as described above) may be utilized to measure half-life.

2. p40 Subunit Domain:

In accordance with any of the aspects and embodiments described herein, the present disclosure provides a heterodimeric Fc fusion protein comprising: a) a first fusion construct, comprising: a variant IL-12p35 subunit domain and a first Fc domain, wherein the variant IL-12p35 subunit domain is covalently attached to the N-terminus or the C-terminus of the first Fc domain, and b) a second fusion construct, comprising: an IL-12p40 subunit domain and a second Fc domain, wherein the IL-12p40 subunit domain is covalently attached to the N-terminus or the C-terminus of the second Fc domain.

In some embodiments, the IL-12p40 subunit domain comprises a variant IL-12p40 subunit domain.

In some embodiments, the IL-12p40 subunit domain comprises a variant IL-12p40 subunit domain, and in further embodiments the variant IL-12p40 subunit domain comprises one or more amino acid substitutions selected from the group that includes: C177S, C252S, and C177S/C252S.

In some embodiments, the (variant) IL-12p40 subunit domain comprises an amino acid sequence selected from the group that includes: SEQ ID NO: 3, SEQ ID NO: 4, SEQ ID NO: 88, SEQ ID NO: 89, and SEQ ID NO: 90 (as shown in FIGS. 1 and 10).

In some embodiments, the (variant) IL-12p40 subunit domain comprises an amino acid sequence with at least 90%, 91%, 92%, 93%, 94%, 95%, 96%, 97%, 98%, 99% or higher sequence identity to an amino acid sequence selected from the group that includes: SEQ ID NO: 3, SEQ ID NO: 4, SEQ ID NO: 88, SEQ ID NO: 89, and SEQ ID NO: 90 (as shown in FIGS. 1 and 10).

In addition to the novel, heterodimeric Fc fusion proteins described above, various modifications to the IL-12p40 subunit domain are known in the art and may further be included in heterodimeric Fc fusion proteins described herein. Non-limiting examples of residues that may be modified include: E3, D7, E12, D14, W15, P17, D18, A19, P20, G21, E22, M23, D29, E32, E33, D34, L40, D41, Q42, S43, E45, L47, S49, T54, Q56, 155, Q56, K58, E59, F60, G61, D62, Y66, E73, H77, K84, E86, D87, G88, 189, W90, D93, K99, E100, K102, N103, K104, T105, F106, R108, E110, N113, Y114, D129, D142, Q144, E156, R159, D161, N162, K163, E164, Y165, E166, S168, D170, Q172, D174, A176, C177, P178, A179, A180, E181, S183, P185, E187, M189, H194, K195, L196, K197, N200, S204, F206, R208, D209, D214, N218, Q220, N226, Q229, E231, E235, T242, P243, S245, Y246, F247, S248, C252, Q256, K258, S259, K260, R261, E262, K264, D265, V267, D270, N281, S283, S285, R287, Q289, D290, R291, Y292, Y293, and E299. In some embodiments, the (variant) IL-12p40 subunit domain described above, below, or in any one of FIGS. 1 and 10 comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more additional amino acid substitutions. In some embodiments, the (variant) IL-12p40 subunit domain described above, below, or in any one of FIGS. 1 and 10 comprises about 1 to about 5, about 6 to about 10, about 11 to about 15, about 16 to about 20, about 21 to about 25, about 26 to about 30, about 31 to about 35, about 36 to about 40, about 41 to about 45, about 46 to about 50, about 51 to about 55, about 56 to about 60, about 1 to about 10, about 11 to about 20, about 21 to about 30, about 31 to about 40, about 41 to about 50, about 51 to about 60, about 1 to about 20, or about 21 to about 40, or about 41 to about 60 additional amino acid substitutions.

3. Fc Domains:

In accordance with any of the aspects and the embodiments described herein, the present disclosure provides a heterodimeric Fc fusion protein comprising: a) a first fusion construct, comprising: a variant IL-12p35 subunit domain and a first Fc domain, wherein the variant IL-12p35 subunit domain is covalently attached to the N-terminus or the C-terminus of the first Fc domain, and b) a second fusion construct, comprising: a(n) (variant) IL-12p40 subunit domain and a second Fc domain, wherein the (variant) IL-12p40 subunit domain is covalently attached to the N-terminus or the C-terminus of the second Fc domain.

In some embodiments, the first Fc domain and/or the second Fc domain comprises the CH2-CH3 domains of human immunoglobulin G1 (IgG1), and optionally all or part of the hinge of human IgG1, as well as fragments thereof. In EU numbering, the CH2-CH3 domain for human IgG1 comprises amino acids 231 to 447, and the hinge comprises amino acids 216 to 230. A “Fc fragment” may contain one, two, three, four, five, six, seven, eight, nine, 10, 11, 12, 13, 14, 15, 16, 17, 18, 19, 20 or fewer amino acids from either or both of the N- and C-termini, but still retains the ability to form a dimer with another Fc domain or Fc fragment as can be detected using standard methods (such as, for example, non-denaturing chromatography, size exclusion chromatograph, and the like).

In some embodiments, the first Fc domain and/or the second Fc domain comprises an amino acid sequence selected from the group that includes: SEQ ID NO: 9, SEQ ID NO: 10, SEQ ID NO: 11, SEQ ID NO: 12, and SEQ ID NO: 13 (as shown in FIG. 3). In some embodiments, the first Fc domain comprises SEQ ID NO: 9, and the second Fc domain comprises SEQ ID NO: 9. In some embodiments, the first Fc domain comprises SEQ ID NO: 10, and the second Fc domain comprises SEQ ID NO: 11. In some embodiments, the first Fc domain comprises SEQ ID NO 11, and the second Fc domain comprises SEQ ID NO: 10. In some embodiments, the first Fc domain comprises SEQ ID NO: 12, and the second Fc domain comprises SEQ ID NO: 13. In some embodiments, the first Fc domain comprises SEQ ID NO: 13, and the second Fc domain comprises SEQ ID NO: 12.

In some embodiments, the first Fc domain and/or the second Fc domain comprises an amino acid sequence with at least 90%, 91%, 92%, 93%, 94%, 95%, 96%, 97%, 98%, 99% or higher identity to an amino acid sequence selected from the group that includes: SEQ ID NO: 9, SEQ ID NO: 10, SEQ ID NO: 11, SEQ ID NO: 12, and SEQ ID NO: 13 (as shown in FIG. 3). In some embodiments, the first Fc domain comprises an amino acid sequence with at least 90%, 91%, 92%, 93%, 94%, 95%, 96%, 97%, 98%, 99% or higher identity to SEQ ID NO: 9, and the second Fc domain comprises an amino acid sequence with at least 90%, 91%, 92%, 93%, 94%, 95%, 96%, 97%, 98%, 99% or higher identity to SEQ ID NO: 9. In some embodiments, the first Fc domain comprises an amino acid sequence with at least 90%, 91%, 92%, 93%, 94%, 95%, 96%, 97%, 98%, 99% or higher identity to SEQ ID NO: 10, and the second Fc domain comprises an amino acid sequence with at least 90%, 91%, 92%, 93%, 94%, 95%, 96%, 97%, 98%, 99% or higher identity to SEQ ID NO: 11. In some embodiments, the first Fc domain comprises an amino acid sequence with at least 90%, 91%, 92%, 93%, 94%, 95%, 96%, 97%, 98%, 99% or higher identity to SEQ ID NO: 11, and the second Fc domain comprises an amino acid sequence with at least 90%, 91%, 92%, 93%, 94%, 95%, 96%, 97%, 98%, 99% or higher identity to SEQ ID NO: 10. In some embodiments, the first Fc domain comprises an amino acid sequence with at least 90%, 91%, 92%, 93%, 94%, 95%, 96%, 97%, 98%, 99% or higher identity to SEQ ID NO: 12, and the second Fc domain comprises an amino acid sequence with at least 90%, 91%, 92%, 93%, 94%, 95%, 96%, 97%, 98%, 99% or higher identity to SEQ ID NO: 13. In some embodiments, the first Fc domain comprises an amino acid sequence with at least 90%, 91%, 92%, 93%, 94%, 95%, 96%, 97%, 98%, 99% or higher identity to SEQ ID NO: 13, and the second Fc domain comprises an amino acid sequence with at least 90%, 91%, 92%, 93%, 94%, 95%, 96%, 97%, 98%, 99% or higher identity to SEQ ID NO: 12.

In some embodiments, the first Fc domain and/or the second Fc domain comprises one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions.

In some embodiments, the first Fc domain and the second Fc domain comprise modifications that promote heterodimerization of the first and second Fc domains. In further embodiments, the first Fc domain comprises an amino acid sequence selected from the group that includes: SEQ ID NO: 10 and SEQ ID NO: 12, and the second Fc domain comprises an amino acid sequence selected from the group that includes: SEQ ID NO: 11 and SEQ ID NO: 13.

In other embodiments, the first Fc domain and the second Fc domain do not comprise modifications that promote heterodimerization of the first and second Fc domains.

In some embodiments, the first Fc domain and/or the second Fc domain comprises one or more modifications that alter Fc binding. In some embodiments, the first Fc domain and/or the second Fc domain comprises one or more modifications that alter Fc half-life. In some embodiments, the first Fc domain and/or the second Fc domain comprises one or more modifications that alter the binding of the first Fc domain and/or the second Fc domain to the neonatal Fc receptor (FcRn).

Described above are various human IgG1 Fc domains, at least some of which comprise one or more modifications. For clarity, while the modifications are discussed in the context of human IgG1 Fc domains for the sake of brevity, it is expressly contemplated that mutations to other immunoglobulins, such as, for example, IgG2, IgG3, IgG4, IgA, IgM, and IgE, can be made at residue positions corresponding to the mutations in human IgG1 that are described herein. Those skilled in the relevant art will readily be able to determine residue locations in other immunoglobulins that correspond with the modifications to human IgG1 described herein.

4. Domain Linkers:

In accordance with any of the aspects and the embodiments described herein, the present disclosure provides a heterodimeric Fc fusion protein comprising: a) a first fusion construct, comprising: a variant IL-12p35 subunit domain and a first Fc domain, wherein the C-terminus of the variant IL-12p35 subunit domain is covalently attached to the N-terminus of the first Fc domain, and b) a second fusion construct, comprising: a(n) (variant) IL-12p40 subunit domain and a second Fc domain, wherein the C-terminus of the (variant) IL-12p40 subunit domain is covalently attached to the N-terminus of the second Fc domain.

In some embodiments wherein the C-terminus of the variant IL-12p35 subunit domain is covalently attached to the N-terminus of the first Fc domain, the variant IL-12p35 subunit domain and the first Fc domain are linked together using a linker domain, wherein the C-terminus of the variant IL-12p35 subunit domain is linked to the N-terminus of the linker domain and the C-terminus of the linker domain is linked to the N-terminus of the first Fc domain. In further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments wherein the C-terminus of the (variant) IL-12p40 subunit domain is covalently attached to the N-terminus of the second Fc domain, the (variant) IL-12p40 subunit domain and the second Fc domain are linked together using a linker domain, wherein the C-terminus of the (variant) IL-12p40 subunit domain is linked to the N-terminus of the linker domain and the C-terminus of the linker domain is linked to the N-terminus of the second Fc domain. In further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments, the variant IL-12p35 subunit domain is not attached to the first Fc domain via a linker domain.

In some embodiments, the (variant) IL-12p40 subunit domain is not attached to the second Fc domain via a linker domain.

In accordance with any of the aspects and the embodiments described herein, the present disclosure provides a heterodimeric Fc fusion protein comprising: a) a first fusion construct, comprising: a variant IL-12p35 subunit domain and a first Fc domain, wherein the N-terminus of the variant IL-12p35 subunit domain is covalently attached to the C-terminus of the first Fc domain, and b) a second fusion construct, comprising: a(n) (variant) IL-12p40 subunit domain and a second Fc domain, wherein the N-terminus of the (variant) IL-12p40 subunit domain is covalently attached to the C-terminus of the second Fc domain.

In some embodiments wherein the N-terminus of the variant IL-12p35 subunit domain is covalently attached to the C-terminus of the first Fc domain, the variant IL-12p35 subunit domain and the first Fc domain are linked together using a linker domain, wherein the C-terminus of the first Fc domain is linked to the N-terminus of the linker domain and the C-terminus of the linker domain is linked to the N-terminus of the variant IL-12p35 subunit domain. In further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments wherein the N-terminus of the (variant) IL-12p40 subunit domain is covalently attached to the C-terminus of the second Fc domain, the (variant) IL-12p40 subunit domain and the second Fc domain are linked together using a linker domain, wherein the C-terminus of the second Fc domain is linked to the N-terminus of the linker domain and the C-terminus of the linker domain is linked to the N-terminus of the (variant) IL-12p40 subunit domain. In further embodiments, the linker domain comprises an amino acid sequence selected from the group that includes: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23 (as shown in FIG. 4).

In some embodiments, the variant IL-12p35 subunit domain is not attached to the first Fc domain via a linker domain.

In some embodiments, the (variant) IL-12p40 subunit domain is not attached to the second Fc domain via a linker domain.

5. Nucleic Acids and Vectors:

In accordance with any of the aspects and the embodiments described herein, the present disclosure provides nucleic acid compositions encoding: (i) heterodimeric Fc fusion proteins, (ii) fusion constructs, (iii) variant IL-12p35 subunit domains, (iv) (variant) IL-12p40 subunit domains, (v) Fc domains, (vi) domain linkers, and (vii) one or more additional proteins, such as, for example, albumin, as described above.

In some embodiments, one or more of the nucleic acids encoding the components of the present invention are incorporated into an expression cassette or an expression vector. It will be understood by one skilled in the art that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure includes a coding sequence for the polypeptide as disclosed herein, which is operably linked to expression control elements, such as a promoter, and optionally, any or a combination of other nucleic acid sequences that affect the transcription or translation of the coding sequence (such as, for example, an origin of replication, selectable markers, ribosomal binding sites, inducers, and the like).

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation (e.g., the introduction of heterologous DNA into a host cell). As such, in some embodiments, the vector can be a replicon, such as, for example, a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitates transfer of the nucleic acid molecule or integration into the genome of a cell or to a virus particle that mediates nucleic acid transfer. Viral particles typically include various viral components, and sometimes also host cell components, in addition to the nucleic acid of interest. The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a baculoviral vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acids encoding any of the heterodimeric Fc fusion proteins, fusion constructs, variant IL-12p35 subunit domains, (variant) IL-12p40 subunit domains, Fc domains, domain linkers, and/or one or more additional proteins, such as, for example, albumin, disclosed herein. The nucleic acids can be contained within a vector that is capable of directing their expression in, for example, a cell line that has been transformed/transduced with the vector. Suitable vectors for use in prokaryotic and eukaryotic cells are known in the art and are commercially available, or readily prepared by a skilled artisan.

As will be appreciated by those skilled in the art, the nucleic acid compositions will depend on the configuration of the heterodimeric Fc fusion protein. Thus, for example, when the configuration requires three amino acid sequences, three nucleic acid sequences can be incorporated into one or more expression vectors for expression. Similarly for other configurations, when only two nucleic acids are needed, they can be incorporated into one or two expression vectors.

DNA vectors can be introduced into host cells, such as, for example, eukaryotic cells or prokaryotic cells, via conventional transformation or transfection techniques, including, but not limited to, one or more of the following: transfection, calcium phosphate transfection, DEAE-dextran-mediated transfection, microinjection, cationic lipid-mediated transfection, electroporation, transduction, scrape loading, ballistic introduction, nucleoporation, hydrodynamic shock, infection, and the like.

Viral vectors that can be used in the disclosure include, but are not limited to, baculoviral vectors, retrovirus vectors, adenovirus vectors, adeno-associated virus vectors, lentivirus vectors, herpes virus, simian virus 40 (SV40), bovine papilloma virus vectors, and the like.

6. Recombinant Cells and Cell Cultures:

In another aspect, provided herein are cell cultures including at least one recombinant cell (also referred to herein as a “host cell”) as disclosed herein, and a culture medium. Generally, the culture medium can be any suitable culture medium for culturing the cells described herein. Techniques for transforming a wide variety of the above-mentioned cells and species are known in the art. Accordingly, cell cultures including at least one recombinant cell as disclosed herein are also within the scope of this application. Methods and systems suitable for generating and maintaining cell cultures are known in the art.

A host cell can be used for preparative purposes to propagate one or more nucleic acids encoding (i) heterodimeric Fc fusion proteins, (ii) fusion constructs, (iii) variant IL-12p35 subunit domains, (iv) (variant) IL-12p40 subunit domains, (v) Fc domains, (vi) domain linkers, and (vii) one or more additional proteins, such as, for example, albumin, as well as combinations and/or components thereof. A host cell can include a prokaryotic cell or eukaryotic cell in which production of the heterodimeric Fc fusion proteins and/or components thereof is specifically intended. Non-limiting examples of host cells include bacterial cells (such as, for example, cells of Gram-positive bacteria (e.g., species of the genus Bacillus, Streptomyces, and Staphylococcus) or cells of Gram-negative bacteria (e.g., cells of the genus Escherichia and Pseudomonas)), fungal cells or yeast cells (e.g., Saccharomyces, Pichia pastoris, and Hansenula polymorpha), insect cells (e.g., cells of Drosophila and Sf9 cells), plant cells (e.g., cells from crop plants, medicinal or ornamental plants or bulbs), mammalian cells (e.g., epithelial cell lines, osteosarcoma cell lines, neuroblastoma cell lines, epithelial carcinomas, glial cells, liver cell lines, Chinese hamster ovary (CHO) cells, COS cells, BHK cells, HeLa cells, D3 cells of the line of murine embryonic stem cells (mESCs), human embryonic stem cells (e.g., HS293 cells and BG01V cells), NIH 3T3 cells, human embryonic kidney (HEK) 293T cells, human mesenchymal stem cells (hMSCSs), and the like), and the like.

### C. Homodimeric IL-12 Fc Fusion Proteins

In one aspect, the present disclosure provides a homodimeric IL-12 Fc fusion protein comprising two instances of a fusion construct, wherein individual instances of the fusion construct comprise a Fc domain and a sc-IL-12 complex, wherein the C-terminus of the Fc domain is covalently attached to the N-terminus of the sc-IL-12 complex, optionally through a domain linker. The sc-IL-12 complex can comprise any suitable sc-IL-12 complex disclosed herein, and the Fc domain can comprise any suitable Fc domain described herein. In such embodiments wherein the Fc domain is linked to the sc-IL-12 complex using a domain linker, any suitable domain linker disclosed herein may be used.

In another aspect, the present disclosure provides a homodimeric IL-12 Fc fusion protein comprising two instances of a fusion construct, wherein the individual instances of the fusion construct comprise a Fc domain and a sc-IL-12 complex, wherein the C-terminus of the sc-IL-12 complex is covalently attached to the N-terminus of the Fc domain, optionally through a domain linker or through (all or part of) the hinge region of the Fc domain. In such embodiments wherein the Fc domain is linked to the sc-IL-12 complex using a domain linker, any suitable domain linker disclosed herein may be used.

### D. Useful Embodiments of the Invention

As will be appreciated by those in the art, and discussed more fully above, the non-naturally occurring IL-12 variants, the homodimeric Fc fusion proteins, and the heterodimeric Fc fusion proteins of the invention can take on a wide variety of configurations. In some embodiments, the non-naturally occurring IL-12 variants bind to and/or have a binding affinity for IL-12Rβ2 and IL-12Rβ1. In some embodiments, the heterodimeric Fc fusion proteins bind to and/or have a binding affinity for IL-12Rβ2 and IL-12Rβ1.

As described above, provided herein are non-naturally occurring IL-12 variants, comprising: a) a variant IL-12p35 subunit, wherein the IL-12p35 subunit comprises one or more amino acid substitutions selected from the group including: Y40A, T43A, D126A, P127A, R129A, K168A, and K170A, and b) an IL-12p40 subunit. In an exemplary embodiment, the variant IL-12p35 subunit comprises SEQ ID NO: 24. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO: 24. In an exemplary embodiment, the variant IL-12p35 subunit comprises SEQ ID NO: 30. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO: 30. In an exemplary embodiment, the variant IL-12p35 subunit comprises SEQ ID NO: 31. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO:31. In an exemplary embodiment, the variant IL-12p35 subunit comprises SEQ ID NO: 32. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO:32. In an exemplary embodiment, the variant IL-12p35 subunit comprises SEQ ID NO: 49. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO: 49. In an exemplary embodiment, the variant IL-12p35 subunit comprises SEQ ID NO: 65. In an exemplary embodiment, the variant IL-12p35 subunit comprises an amino acid sequence with at least 90%, 91%, 92%, 93%, 94%, 95%, 96%, 97%, 98%, 99% or higher to SEQ ID NO: 65.

As described above, provided herein are homodimeric Fc fusion proteins comprising: two instances of a fusion construct, wherein individual instances of the fusion construct comprise: a Fc domain and a sc-IL-12 complex, wherein the C-terminus of the Fc domain is covalently attached to the N-terminus of the sc-IL-12 complex, optionally through a domain linker. The sc-IL-12 complex can comprise any suitable sc-IL-12 complex disclosed herein, and the Fc domain can comprise any suitable Fc domain described herein. In such embodiments wherein the Fc domain is linked to the sc-IL-12 complex using a domain linker, any suitable domain linker disclosed herein may be used.

As described above, provided herein are homodimeric Fc fusion proteins comprising: two instances of a fusion construct, wherein individual instances of the fusion construct comprise: a Fc domain and a sc-IL-12 complex, wherein the C-terminus of the sc-IL-12 complex is covalently attached to the N-terminus of the Fc domain, optionally through a domain linker or through (all or part of) the hinge region of the Fc domain. In such embodiments wherein the Fc domain is linked to the sc-IL-12 complex using a domain linker, any suitable domain linker disclosed herein may be used.

As described above, provided herein are heterodimeric Fc fusion proteins comprising: a) a first fusion construct, comprising: a variant IL-12p35 subunit domain and a first Fc domain, wherein the C-terminus of the variant IL-12p35 subunit domain is covalently attached to the N-terminus of the first Fc domain or wherein the C-terminus of the first Fc domain is covalently attached to the N-terminus of the variant IL-12p35 subunit domain, and b) a second fusion construct, comprising: an IL-12p40 subunit domain and a second Fc domain, wherein the C-terminus of the IL-12p40 subunit domain is covalently attached to the N-terminus of the second Fc domain or the C-terminus of the second Fc domain is covalently attached to the N-terminus of the IL-12p40 subunit domain, and optionally wherein the first Fc domain and the second Fc domain comprise modifications that (i) promote heterodimerization of the first and second Fc domains and/or (ii) silence or inhibit effector function. In an exemplary embodiment, the variant IL-12p35 subunit domain comprises SEQ ID NO: 24. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO: 24. In an exemplary embodiment, the variant IL-12p35 subunit domain comprises SEQ ID NO: 30. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO: 30. In an exemplary embodiment, the variant IL-12p35 subunit domain comprises SEQ ID NO: 31. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO:31. In an exemplary embodiment, the variant IL-12p35 subunit domain comprises SEQ ID NO: 32. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO:32. In an exemplary embodiment, the variant IL-12p35 subunit domain comprises SEQ ID NO: 49. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO: 49. In an exemplary embodiment, the variant IL-12p35 subunit domain comprises SEQ ID NO: 65. In an exemplary embodiment, the variant IL-12p35 subunit domain comprises an amino acid sequence with at least 90%, 91%, 92%, 93%, 94%, 95%, 96%, 97%, 98%, 99% or higher to SEQ ID NO: 65.

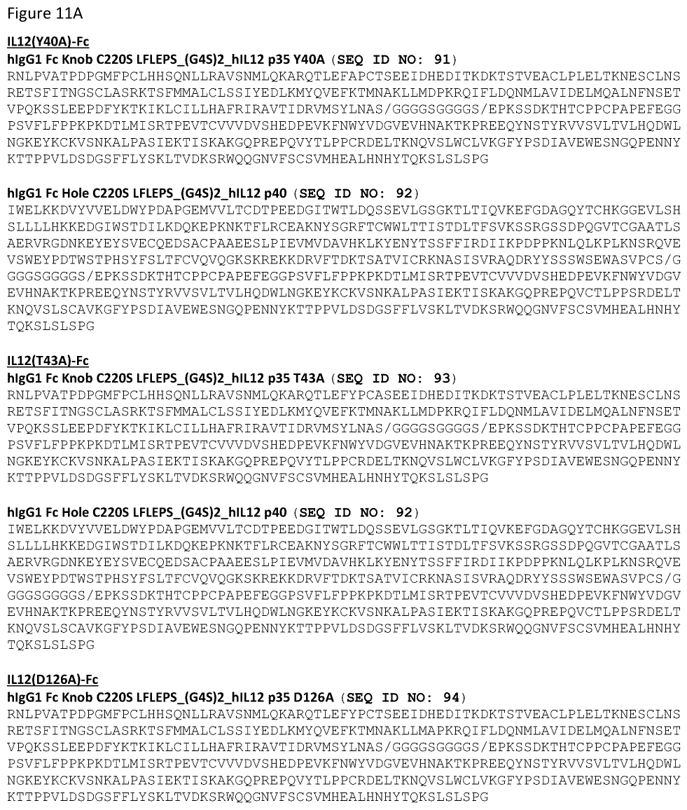

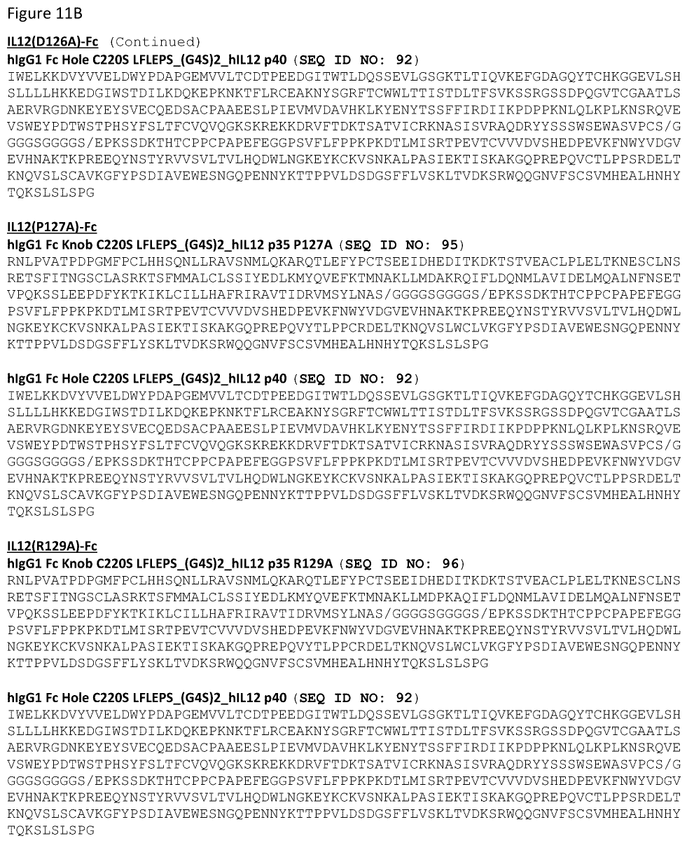

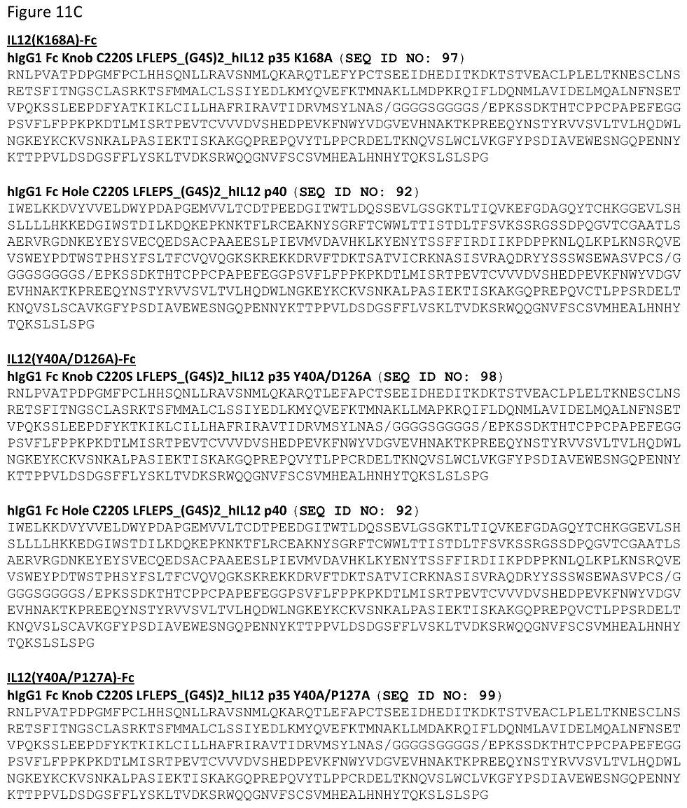

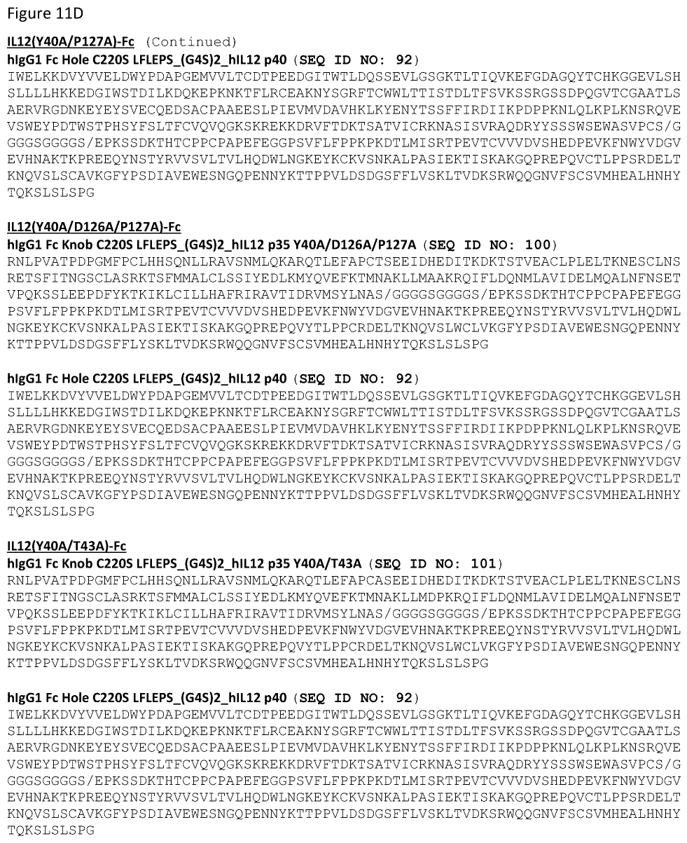

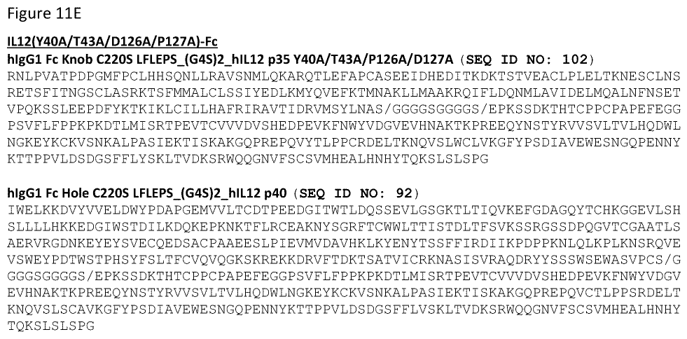

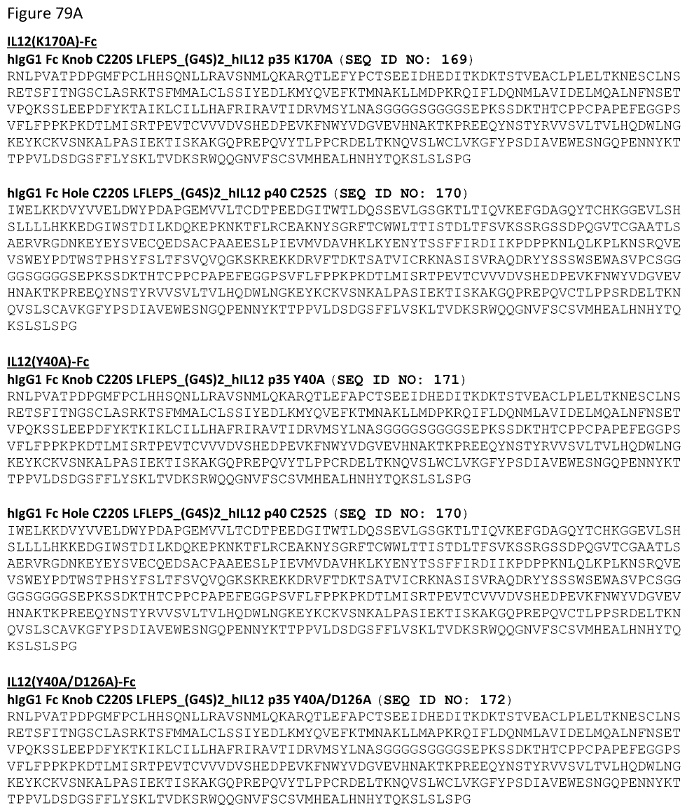

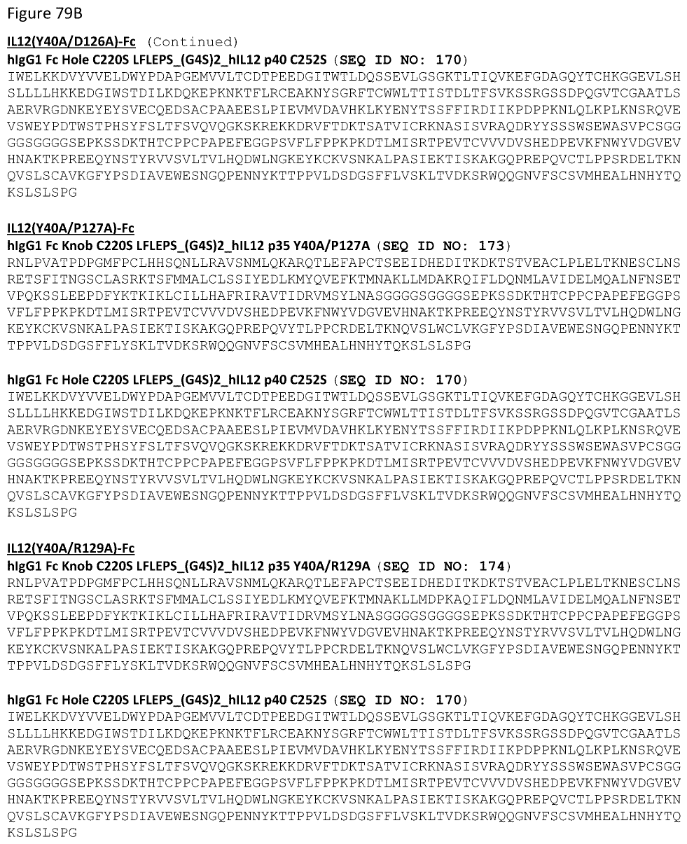

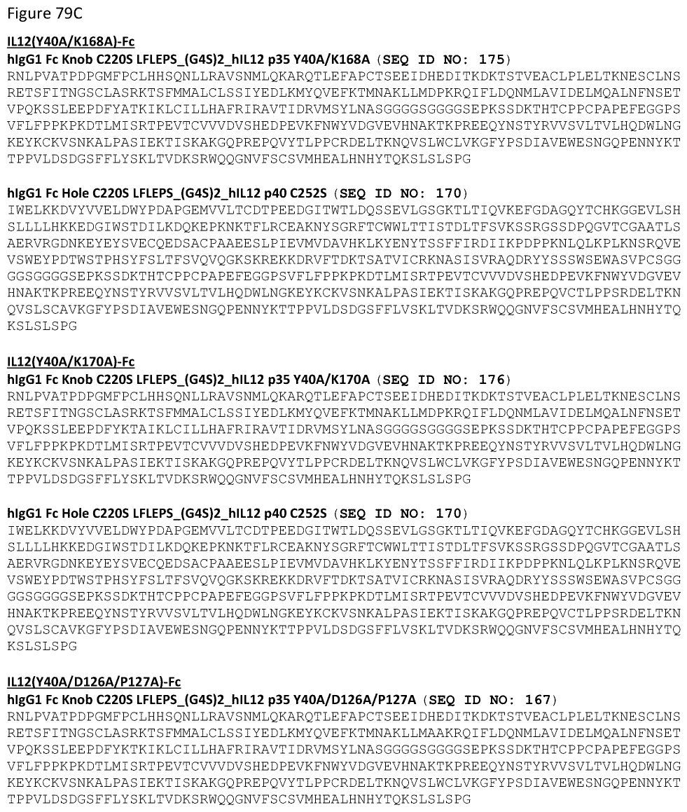

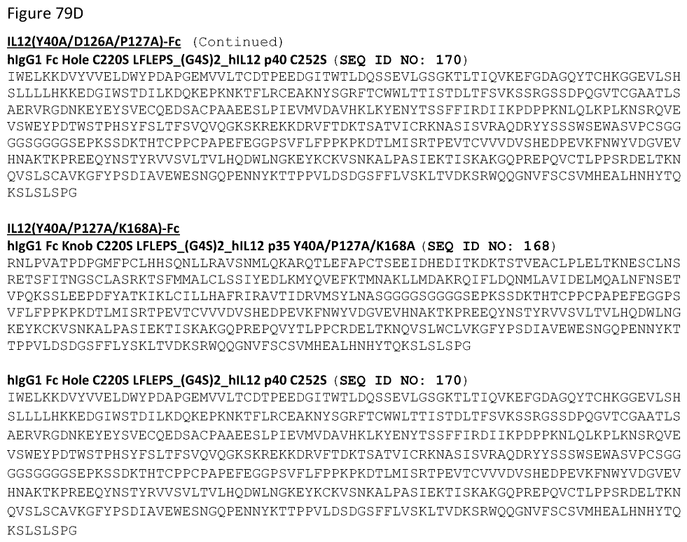

Further, provided herein are heterodimeric Fc fusion proteins comprising: a) a first fusion construct, and b) a second fusion construct. In an exemplary embodiment, the first fusion construct comprises SEQ ID NO: 91, and the second fusion construct comprises SEQ ID NO: 92 (as shown in FIG. 11). In an exemplary embodiment, the first fusion construct comprises SEQ ID NO: 93, and the second fusion construct comprises SEQ ID NO: 92 (as shown in FIG. 11). In an exemplary embodiment, the first fusion construct comprises SEQ ID NO: 94, and the second fusion construct comprises SEQ ID NO: 92 (as shown in FIG. 11). In an exemplary embodiment, the first fusion construct comprises SEQ ID NO: 95, and the second fusion construct comprises SEQ ID NO: 92 (as shown in FIG. 11). In an exemplary embodiment, the first fusion construct comprises SEQ ID NO: 96, and the second fusion construct comprises SEQ ID NO: 92 (as shown in FIG. 11). In an exemplary embodiment, the first fusion construct comprises SEQ ID NO: 97, and the second fusion construct comprises SEQ ID NO: 92 (as shown in FIG. 11). In an exemplary embodiment, the first fusion construct comprises SEQ ID NO: 98, and the second fusion construct comprises SEQ ID NO: 92 (as shown in FIG. 11). In an exemplary embodiment, the first fusion construct comprises SEQ ID NO: 99, and the second fusion construct comprises SEQ ID NO: 92 (as shown in FIG. 11). In an exemplary embodiment, the first fusion construct comprises SEQ ID NO: 100, and the second fusion construct comprises SEQ ID NO: 92 (as shown in FIG. 11). In an exemplary embodiment, the first fusion construct comprises SEQ ID NO: 101, and the second fusion construct comprises SEQ ID NO: 92 (as shown in FIG. 11). In an exemplary embodiment, the first fusion construct comprises SEQ ID NO: 102, and the second fusion construct comprises SEQ ID NO: 92 (as shown in FIG. 11). In an exemplary embodiment, the first fusion construct comprises SEQ ID NO: 92, and the second fusion construct comprises SEQ ID NO: 91. In an exemplary embodiment, the first fusion construct comprises SEQ ID NO: 92, and the second fusion construct comprises SEQ ID NO: 93. In an exemplary embodiment, the first fusion construct comprises SEQ ID NO: 92, and the second fusion construct comprises SEQ ID NO: 94. In an exemplary embodiment, the first fusion construct comprises SEQ ID NO: 92, and the second fusion construct comprises SEQ ID NO: 95. In an exemplary embodiment, the first fusion construct comprises SEQ ID NO: 92, and the second fusion construct comprises SEQ ID NO: 96. In an exemplary embodiment, the first fusion construct comprises SEQ ID NO: 92, and the second fusion construct comprises SEQ ID NO: 97. In an exemplary embodiment, the first fusion construct comprises SEQ ID NO: 92, and the second fusion construct comprises SEQ ID NO: 98. In an exemplary embodiment, the first fusion construct comprises SEQ ID NO: 92, and the second fusion construct comprises SEQ ID NO: 99. In an exemplary embodiment, the first fusion construct comprises SEQ ID NO: 92, and the second fusion construct comprises SEQ ID NO: 100. In an exemplary embodiment, the first fusion construct comprises SEQ ID NO: 92, and the second fusion construct comprises SEQ ID NO: 101. In an exemplary embodiment, the first fusion construct comprises SEQ ID NO: 92, and the second fusion construct comprises SEQ ID NO: 102. In an exemplary embodiment, the first fusion construct comprises SEQ ID NO: 169 (as shown in FIG. 79), and the second fusion construct comprises SEQ ID NO: 170 (as shown in FIG. 79). In an exemplary embodiment, the first fusion construct comprises SEQ ID NO: 171 (as shown in FIG. 79), and the second fusion construct comprises SEQ ID NO: 170. In an exemplary embodiment, the first fusion construct comprises SEQ ID NO: 172 (as shown in FIG. 79), and the second fusion construct comprises SEQ ID NO: 170. In an exemplary embodiment, the first fusion construct comprises SEQ ID NO: 173 (as shown in FIG. 79), and the second fusion construct comprises SEQ ID NO: 170. In an exemplary embodiment, the first fusion construct comprises SEQ ID NO: 174 (as shown in FIG. 79), and the second fusion construct comprises SEQ ID NO: 170. In an exemplary embodiment, the first fusion construct comprises SEQ ID NO: 175 (as shown in FIG. 79), and the second fusion construct comprises SEQ ID NO: 170. In an exemplary embodiment, the first fusion construct comprises SEQ ID NO: 176 (as shown in FIG. 79), and the second fusion construct comprises SEQ ID NO: 170. In an exemplary embodiment, the first fusion construct comprises SEQ ID NO: 167 (as shown in FIG. 79), and the second fusion construct comprises SEQ ID NO: 170. In an exemplary embodiment, the first fusion construct comprises SEQ ID NO: 168 (as shown in FIG. 79), and the second fusion construct comprises SEQ ID NO: 170. In an exemplary embodiment, the first fusion construct comprises SEQ ID NO: 170, and the second fusion construct comprises SEQ ID NO: 169. In an exemplary embodiment, the first fusion construct comprises SEQ ID NO: 170, and the second fusion construct comprises SEQ ID NO: 171. In an exemplary embodiment, the first fusion construct comprises SEQ ID NO: 170, and the second fusion construct comprises SEQ ID NO: 172. In an exemplary embodiment, the first fusion construct comprises SEQ ID NO: 170, and the second fusion construct comprises SEQ ID NO: 173. In an exemplary embodiment, the first fusion construct comprises SEQ ID NO: 170, and the second fusion construct comprises SEQ ID NO: 174. In an exemplary embodiment, the first fusion construct comprises SEQ ID NO: 170, and the second fusion construct comprises SEQ ID NO: 175. In an exemplary embodiment, the first fusion construct comprises SEQ ID NO: 170, and the second fusion construct comprises SEQ ID NO: 176. In an exemplary embodiment, the first fusion construct comprises SEQ ID NO: 170, and the second fusion construct comprises SEQ ID NO: 167. In an exemplary embodiment, the first fusion construct comprises SEQ ID NO: 170, and the second fusion construct comprises SEQ ID NO: 168.

In an exemplary embodiment, a heterodimeric Fc fusion protein is provided, wherein the heterodimeric Fc fusion protein comprises: a) a first fusion construct, comprising: (i) a variant IL-12p35 subunit domain, wherein the variant IL-12p35 subunit domain is selected from a group including SEQ ID NOs: 24, 30-34, 49, 52, 53, 65, 103, 104, 112, 177-247, (ii) a first Fc domain, wherein the first Fc domain is selected from a group including SEQ ID NOs: 12 and 13, and (iii) a linker, wherein the linker comprises SEQ ID NO: 15, the C-terminus of the variant IL-12p35 subunit domain is covalently attached to the N-terminus of the linker, and the C-terminus of the linker is covalently attached to the N-terminus of the first Fc domain; and b) a second fusion construct, comprising: (i) a (variant) IL-12p40 subunit domain, wherein the (variant) IL-12p40 subunit domain is selected from a group including SEQ ID NOs: 4, 89, and 90, (ii) a second Fc domain, wherein the second Fc domain is selected from a group including SEQ ID NOs: 13 and 12, and (iii) a linker, wherein the linker comprises SEQ ID NO: 15, the C-terminus of the (variant) IL-12p40 subunit domain is covalently attached to the N-terminus of the linker, and the C-terminus of the linker is covalently attached to the N-terminus of the second Fc domain. In some further exemplary embodiments, the variant IL-12p35 subunit domain may further comprise a C74S substitution mutation, and the (variant) IL-12p40 subunit domain may further comprise a C177S substitution mutation, such that the inter-chain disulfide bond between the variant IL-12p35 subunit domain and the (variant) IL-12p40 subunit domain is removed.

### IV. Methods of Making

As will be appreciated by one skilled in the art, any aspects and embodiments of the methods of making non-naturally occurring IL-12 variants, homodimeric Fc fusion proteins, and heterodimeric Fc fusion proteins described herein can utilize any of the aspects and/or embodiments of the compositions described herein and can further be used in any of the methods of use also described herein.

### A. Interleukin 12 (IL-12) Variants

In another aspect, the present disclosure provides a method of producing non-naturally occurring IL-12 variants of the disclosure, the method comprising: culturing a host cell with one or more nucleic acids or vectors under conditions whereby the non-naturally occurring IL-12 variant (and/or fragments or components thereof) is produced, wherein the nucleic acid or vector comprises one or more nucleic acids encoding one or more of the amino acid sequences described above in the compositions section.

Nucleic acids encoding a desired protein, such as, for example, a non-naturally occurring IL-12 variant (and/or fragments or components thereof), can be introduced into a host cell by standard techniques for transforming or transfecting cells. Alternatively, or in addition to, nucleic acids encoding a desired protein, such as, for example, a non-naturally occurring IL-12 variant (and/or fragments or components thereof), can be incorporated into a vector or “expression cassette,” as described in further detail above. Vectors (or expression cassettes) can be introduced into a host cell via conventional transformation or transfection techniques. Suitable methods for transforming or transfecting host cells using a nucleic acid or vector include, but are not limited to, calcium phosphate transfection, DEAE-dextran-mediated transfection, transfection, microinjection, cationic lipid-mediated transfection, lipofection, electroporation, transduction, scrape loading, ballistic introduction, nucleoporation, hydrodynamic shock, infection, and the like.

In some embodiments, the method further comprises isolating and/or purifying the non-naturally occurring IL-12 variant. In the process, a host cell (as described above), into which has been introduced a nucleic acid encoding the protein of interest operatively linked to a regulatory sequence, is grown at production scale in a culture medium to stimulate transcription of the nucleotide sequence(s) encoding the protein of interest. Subsequently, the protein is isolated from harvested host cells or from the culture medium. Standard protein purification techniques can be used to isolate the protein of interest from the medium or from the harvested cells. In particular, the purification techniques can be used to express and purify a desired protein, such as, for example, a non-naturally occurring IL-12 variant, on a large-scale (i.e., in at least milligram (1.00 mg) quantities) or on a small-scale (i.e., in at least nanogram (1.00 ng) quantities up to milligram (<1.00 mg) quantities) from a variety of implementations including, but not limited to, roller bottles, spinner flasks, tissue culture plates, one or more bioreactors, one or more fermenters, and/or any combination thereof.

An expressed protein, such as, for example, a non-naturally occurring IL-12 variant, can be isolated and purified by known methods. Generally, the culture medium is centrifuged and/or filtered, and then the supernatant is purified by affinity or immunoaffinity chromatography. The proteins of the present invention can be separated and/or purified by appropriate combination(s) of known techniques. These methods include, but are not limited to, methods utilizing solubility (e.g., salt precipitation or solvent precipitation), methods utilizing a difference in molecular weight (e.g., dialysis, ultra-filtration, gel-filtration, SDS-polyacrylamide gel electrophoresis (SDS-PAGE)), methods utilizing a difference in electrical charge (e.g., ion-exchange column chromatography), methods utilizing specific affinity (e.g., affinity chromatography), methods utilizing a difference in hydrophobicity (e.g., reverse-phase high-performance liquid chromatography), methods utilizing a difference in isoelectric point (e.g., isoelectric focusing electrophoresis or metal affinity columns, such as, for example, Ni-NTA), and the like.

In some embodiments, the methods for producing the non-naturally occurring IL-12 variant includes structural modifications to the polypeptide being produced to increase half-life. In some embodiments, the non-naturally occurring IL-12 variant further comprises one or more of the following fused to the variant IL-12p35 subunit and/or the (variant) IL-12p40 subunit: (i) a Fc domain, wherein the Fc domain comprises one or more amino acid sequences selected from the group that includes: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

### B. Heterodimeric IL-12 Fc Fusion Proteins

In another aspect, the present disclosure provides a method of producing heterodimeric IL-12 Fc fusion proteins of the disclosure, the method comprising: culturing a host cell with one or more nucleic acids or vectors under conditions whereby the heterodimeric IL-12 Fc fusion protein (and/or fragments or components thereof) is produced, wherein the one or more nucleic acids or vector comprise one or more nucleic acids encoding one or more of the amino acid sequences described above in the compositions section, wherein the heterodimeric IL-12 Fc fusion protein has an increased half-life compared to half-life of a reference IL-12. In some embodiments, the reference IL-12 comprises one or more of: a wild-type IL-12, a human wild-type IL-12, a commercially available IL-12 molecule, an IL-12 Fc fusion protein, or any combination thereof.

Nucleic acids encoding a desired fusion protein, such as, for example, a heterodimeric IL-12 Fc fusion protein (and/or fragments or components thereof), can be introduced into a host cell by standard techniques for transforming or transfecting cells. Alternatively, or in addition to, nucleic acids encoding a desired protein, such as, for example, a heterodimeric IL-12 Fc fusion protein (and/or fragments or components thereof), can be incorporated into a vector or “expression cassette,” as described in further detail above. Vectors (or expression cassettes) can be introduced into a host cell via conventional transformation or transfection techniques. Suitable methods for transforming or transfecting host cells using a nucleic acid or vector include, but are not limited to, calcium phosphate transfection, DEAE-dextran-mediated transfection, transfection, microinjection, cationic lipid-mediated transfection, lipofection, electroporation, transduction, scrape loading, ballistic introduction, nucleoporation, hydrodynamic shock, infection, and the like.

In some embodiments, the method further comprises isolating and/or purifying the heterodimeric IL-12 Fc fusion protein. In the process, a host cell (as described above), into which has been introduced a nucleic acid encoding the fusion protein of interest operatively linked to a regulatory sequence, is grown at production scale in a culture medium to stimulate transcription of the nucleotide sequence(s) encoding the fusion protein of interest. Subsequently, the fusion protein is isolated from harvested host cells or from the culture medium. Standard protein purification techniques can be used to isolate the fusion protein of interest from the medium or from the harvested cells. In particular, the purification techniques can be used to express and purify a desired fusion protein, such as, for example, a heterodimeric Fc fusion protein, on a large-scale (i.e., in at least milligram (1.00 mg) quantities) or on a small-scale (i.e., in at least nanogram (1.00 ng) quantities up to milligram (<1.00 mg) quantities) from a variety of implementations including, but not limited to, roller bottles, spinner flasks, tissue culture plates, one or more bioreactors, one or more fermenters, and/or any combination thereof.

An expressed fusion protein, such as, for example, a heterodimeric IL-12 Fc fusion protein, can be isolated and purified by known methods. Generally, the culture medium is centrifuged and/or filtered, and then the supernatant is purified by affinity or immunoaffinity chromatography. The fusion proteins of the present invention can be separated and/or purified by appropriate combination(s) of known techniques. These methods include, but are not limited to, methods utilizing solubility (e.g., salt precipitation or solvent precipitation), methods utilizing a difference in molecular weight (e.g., dialysis, ultra-filtration, gel-filtration, SDS-polyacrylamide gel electrophoresis (SDS-PAGE)), methods utilizing a difference in electrical charge (e.g., ion-exchange column chromatography), methods utilizing specific affinity (e.g., affinity chromatography), methods utilizing a difference in hydrophobicity (e.g., reverse-phase high-performance liquid chromatography), methods utilizing a difference in isoelectric point (e.g., isoelectric focusing electrophoresis or metal affinity columns, such as, for example, Ni-NTA), and the like.

### C. Homodimeric IL-12 Fc Fusion Proteins

In another aspect, the present disclosure provides a method of producing homodimeric IL-12 Fc fusion proteins of the disclosure, the method comprising: culturing a host cell with one or more nucleic acids or vectors under conditions whereby the homodimeric IL-12 Fc fusion protein (and/or fragments or components thereof) is produced, wherein the one or more nucleic acids or vectors comprise one or more nucleic acids encoding one or more of the amino acid sequences described above in the compositions section. In some embodiments, the generated homodimeric IL-12 Fc fusion protein has an increased half-life compared to half-life of a reference IL-12. In some embodiments, the reference IL-12 comprises one or more of: a wild-type IL-12, a human wild-type IL-12, a commercially available IL-12 molecule, an IL-12 Fc fusion protein, or any combination thereof.

Nucleic acids encoding a desired fusion protein, such as, for example, a homodimeric IL-12 Fc fusion protein (and/or fragments or components thereof), can be introduced into a host cell by standard techniques for transforming or transfecting cells. Alternatively, or in addition to, nucleic acids encoding a desired protein, such as, for example, a homodimeric IL-12 Fc fusion protein (and/or fragments or components thereof), can be incorporated into a vector or “expression cassette,” as described in further detail above. Vectors (or expression cassettes) can be introduced into a host cell via conventional transformation or transfection techniques. Suitable methods for transforming or transfecting host cells using a nucleic acid or vector include, but are not limited to, calcium phosphate transfection, DEAE-dextran-mediated transfection, transfection, microinjection, cationic lipid-mediated transfection, lipofection, electroporation, transduction, scrape loading, ballistic introduction, nucleoporation, hydrodynamic shock, infection, and the like.

In some embodiments, the method further comprises isolating and/or purifying the homodimeric IL-12 Fc fusion protein. In the process, a host cell (as described above), into which has been introduced a nucleic acid encoding the fusion protein of interest operatively linked to a regulatory sequence, is grown at production scale in a culture medium to stimulate transcription of the nucleotide sequence(s) encoding the fusion protein of interest. Subsequently, the fusion protein is isolated from harvested host cells or from the culture medium. Standard protein purification techniques can be used to isolate the fusion protein of interest from the medium or from the harvested cells. In particular, the purification techniques can be used to express and purify a desired fusion protein, such as, for example, a homodimeric IL-12 Fc fusion protein, on a large-scale (i.e., in at least milligram (1.00 mg) quantities) or on a small-scale (i.e., in at least nanogram (1.00 ng) quantities up to milligram (<1.00 mg) quantities) from a variety of implementations including, but not limited to, roller bottles, spinner flasks, tissue culture plates, one or more bioreactors, one or more fermenters, and/or any combination thereof.

An expressed fusion protein, such as, for example, a homodimeric IL-12 Fc fusion protein, can be isolated and purified by known methods. Generally, the culture medium is centrifuged and/or filtered, and then the supernatant is purified by affinity or immunoaffinity chromatography. The fusion proteins of the present invention can be separated and/or purified by appropriate combination(s) of known techniques. These methods include, but are not limited to, methods utilizing solubility (e.g., salt precipitation or solvent precipitation), methods utilizing a difference in molecular weight (e.g., dialysis, ultra-filtration, gel-filtration, SDS-polyacrylamide gel electrophoresis (SDS-PAGE)), methods utilizing a difference in electrical charge (e.g., ion-exchange column chromatography), methods utilizing specific affinity (e.g., affinity chromatography), methods utilizing a difference in hydrophobicity (e.g., reverse-phase high-performance liquid chromatography), methods utilizing a difference in isoelectric point (e.g., isoelectric focusing electrophoresis or metal affinity columns, such as, for example, Ni-NTA), and the like.

### V. Methods of Use

As will be appreciated by one skilled in the art, any aspects and embodiments of the methods of use described herein can utilize any of the aspects and/or embodiments of the compositions described above and can further utilize any of the methods of making the non-naturally occurring IL-12 variants, homodimeric Fc fusion proteins, and heterodimeric Fc fusion proteins also described above.

### A. Interleukin 12 (IL-12) Variants

In another aspect, the present disclosure provides methods of using one or more of the non-naturally occurring IL-12 variants of the disclosure.

In some embodiments, a therapeutically effective amount of a non-naturally occurring IL-12 variant, as described above in the compositions and/or methods of making sections, is administered to a subject in need thereof. As used herein, the term “therapeutically effective amount” generally refers to an amount sufficient for a composition to accomplish a stated purpose relative to the absence of the composition (e.g., achieve the effect for which it is administered, treat a disease/disorder/condition, reduce a signaling pathway, or reduce one or more symptoms of a disease/disorder/condition). An example of a “therapeutically effective amount” is an amount sufficient to contribute to the treatment, prevention, or reduction of one or more symptoms of a disease/disorder/condition. A “reduction” of a symptom means decreasing the severity and/or frequency of the symptom, and/or an elimination of the symptom. The exact amount of a composition including a “therapeutically effective amount” will depend on the purpose of the treatment, and will be ascertainable by one skilled in the relevant art.

In some embodiments, a therapeutically effective amount of a non-naturally occurring IL-12 variant, as described above in the compositions and/or methods of making sections, is administered to a subject in need thereof. In further embodiments, the non-naturally occurring IL-12 variant is administered to the subject in need thereof to treat a disease, a disorder, or a condition. In yet further embodiments, the disease, disorder, or condition comprises cancer.

In some embodiments, a therapeutically effective amount of a non-naturally occurring IL-12 variant, as described above in the compositions and/or methods of making sections, is administered to a subject in need thereof. In further embodiments, the non-naturally occurring IL-12 variant is administered to the subject in need thereof to treat a disease, a disorder, or a condition. In yet further embodiments, the disease, disorder, or condition comprises cancer. In even further embodiments, the cancer comprises a benign tumor, a metastatic tumor, or a mixed-type tumor.

In some embodiments, a therapeutically effective amount of a non-naturally occurring IL-12 variant, as described above in the compositions and/or methods of making sections, is administered to a subject in need thereof. In further embodiments, the non-naturally occurring IL-12 variant is administered to the subject in need thereof to treat a disease, a disorder, or a condition. In yet further embodiments, the disease, disorder, or condition comprises cancer. In even further embodiments, the cancer comprises recurrent cancer and/or refractory cancer. In some embodiments, the refractory cancer is a primary refractory cancer (i.e., a cancer that is resistant or immune to a given therapy at baseline). In other embodiments, the refractory cancer is a secondary refractory cancer (i.e., a cancer that was previously susceptible or partially susceptible to a given therapy at a first time-point but has subsequently become resistant or immune to a given therapy at a second time point, such as, for example, at or after recurrence).

In some embodiments, the non-naturally occurring IL-12 variant is administered as a first-line, second-line, third-line, fourth-line, fifth-line, or higher therapy.

In some embodiments, the non-naturally occurring IL-12 variant is administered alone. In other embodiments, the non-naturally occurring IL-12 variant is administered as a part of a combination therapy, including combinations with treatments known in the art and disclosed herein for the treatment of cancer, such as, in some non-limiting examples, surgery, chemotherapy, radiotherapy, phototherapy, targeted therapies and immunotherapies.

### B. Heterodimeric IL-12 Fc Fusion Proteins

In another aspect, the present disclosure provides methods of using one or more of the heterodimeric Fc fusion proteins of the disclosure.

In some embodiments, a therapeutically effective amount of a heterodimeric Fc fusion protein, as described above in the compositions and/or methods of making sections, is administered to a subject in need thereof. As used herein, the term “therapeutically effective amount” generally refers to an amount sufficient for a composition to accomplish a stated purpose relative to the absence of the composition (e.g., achieve the effect for which it is administered, treat a disease/disorder/condition, reduce a signaling pathway, or reduce one or more symptoms of a disease/disorder/condition). An example of a “therapeutically effective amount” is an amount sufficient to contribute to the treatment, prevention, or reduction of one or more symptoms of a disease/disorder/condition. A “reduction” of a symptom means decreasing the severity and/or frequency of the symptom, and/or an elimination of the symptom. The exact amount of a composition including a “therapeutically effective amount” will depend on the purpose of the treatment, and will be ascertainable by one skilled in the relevant art.

In some embodiments, a therapeutically effective amount of a heterodimeric Fc fusion protein, as described above in the compositions and/or methods of making sections, is administered to a subject in need thereof. In further embodiments, the heterodimeric Fc fusion protein is administered to the subject in need thereof to treat a disease, a disorder, or a condition. In yet further embodiments, the disease, disorder, or condition comprises cancer.

In some embodiments, a therapeutically effective amount of a heterodimeric Fc fusion protein, as described above in the compositions and/or methods of making sections, is administered to a subject in need thereof. In further embodiments, the heterodimeric Fc fusion protein is administered to the subject in need thereof to treat a disease, a disorder, or a condition. In yet further embodiments, the disease, disorder, or condition comprises cancer. In even further embodiments, the cancer comprises a benign tumor, a metastatic tumor, or a mixed-type tumor.

In some embodiments, a therapeutically effective amount of a heterodimeric Fc fusion protein, as described above in the compositions and/or methods of making sections, is administered to a subject in need thereof. In further embodiments, the heterodimeric Fc fusion protein is administered to the subject in need thereof to treat a disease, a disorder, or a condition. In yet further embodiments, the disease, disorder, or condition comprises cancer. In even further embodiments, the cancer comprises recurrent cancer and/or refractory cancer. In some embodiments, the refractory cancer is a primary refractory cancer (i.e., a cancer that is resistant or immune to a given therapy at baseline). In other embodiments, the refractory cancer is a secondary refractory cancer (i.e., a cancer that was previously susceptible or partially susceptible to a given therapy at a first time-point but has subsequently become resistant or immune to a given therapy at a second time point, such as, for example, at or after recurrence).

In some embodiments, the heterodimeric Fc fusion protein is administered as a first-line, second-line, third-line, fourth-line, fifth-line, or higher therapy.

In some embodiments, the heterodimeric Fc fusion protein is administered alone. In other embodiments, the heterodimeric Fc fusion protein is administered as a part of a combination therapy.

### C. Homodimeric IL-12 Fc Fusion Proteins

In another aspect, the present disclosure provides methods of using one or more of the homodimeric IL-12 Fc fusion proteins of the disclosure.

In some embodiments, a therapeutically effective amount of a homodimeric IL-12 Fc fusion protein, as described above in the compositions and/or methods of making sections, is administered to a subject in need thereof. As used herein, the term “therapeutically effective amount” generally refers to an amount sufficient for a composition to accomplish a stated purpose relative to the absence of the composition (e.g., achieve the effect for which it is administered, treat a disease/disorder/condition, reduce a signaling pathway, or reduce one or more symptoms of a disease/disorder/condition). An example of a “therapeutically effective amount” is an amount sufficient to contribute to the treatment, prevention, or reduction of one or more symptoms of a disease/disorder/condition. A “reduction” of a symptom means decreasing the severity and/or frequency of the symptom, and/or an elimination of the symptom. The exact amount of a composition including a “therapeutically effective amount” will depend on the purpose of the treatment, and will be ascertainable by one skilled in the relevant art.

In some embodiments, a therapeutically effective amount of a homodimeric IL-12 Fc fusion protein, as described above in the compositions and/or methods of making sections, is administered to a subject in need thereof. In further embodiments, the homodimeric IL-12 Fc fusion protein is administered to the subject in need thereof to treat a disease, a disorder, or a condition. In yet further embodiments, the disease, disorder, or condition comprises cancer.

In some embodiments, a therapeutically effective amount of a homodimeric IL-12 Fc fusion protein, as described above in the compositions and/or methods of making sections, is administered to a subject in need thereof. In further embodiments, the homodimeric IL-12 Fc fusion protein is administered to the subject in need thereof to treat a disease, a disorder, or a condition. In yet further embodiments, the disease, disorder, or condition comprises cancer. In even further embodiments, the cancer comprises a benign tumor, a metastatic tumor, or a mixed-type tumor.

In some embodiments, a therapeutically effective amount of a homodimeric IL-12 Fc fusion protein, as described above in the compositions and/or methods of making sections, is administered to a subject in need thereof. In further embodiments, the homodimeric IL-12 Fc fusion protein is administered to the subject in need thereof to treat a disease, a disorder, or a condition. In yet further embodiments, the disease, disorder, or condition comprises cancer. In even further embodiments, the cancer comprises recurrent cancer and/or refractory cancer. In some embodiments, the refractory cancer is a primary refractory cancer (i.e., a cancer that is resistant or immune to a given therapy at baseline). In other embodiments, the refractory cancer is a secondary refractory cancer (i.e., a cancer that was previously susceptible or partially susceptible to a given therapy at a first time-point but has subsequently become resistant or immune to a given therapy at a second time point, such as, for example, at or after recurrence).

In some embodiments, the homodimeric IL-12 Fc fusion protein is administered as a first-line, second-line, third-line, fourth-line, fifth-line, or higher therapy.

In some embodiments, the homodimeric IL-12 Fc fusion protein is administered alone. In other embodiments, the homodimeric IL-12 Fc fusion protein is administered as a part of a combination therapy.

### Examples

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### A. Example 1—Expression and Purification of IL-12 in a Heterodimeric Fc Format

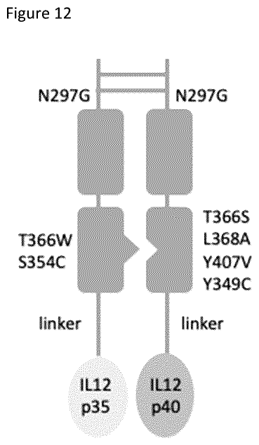

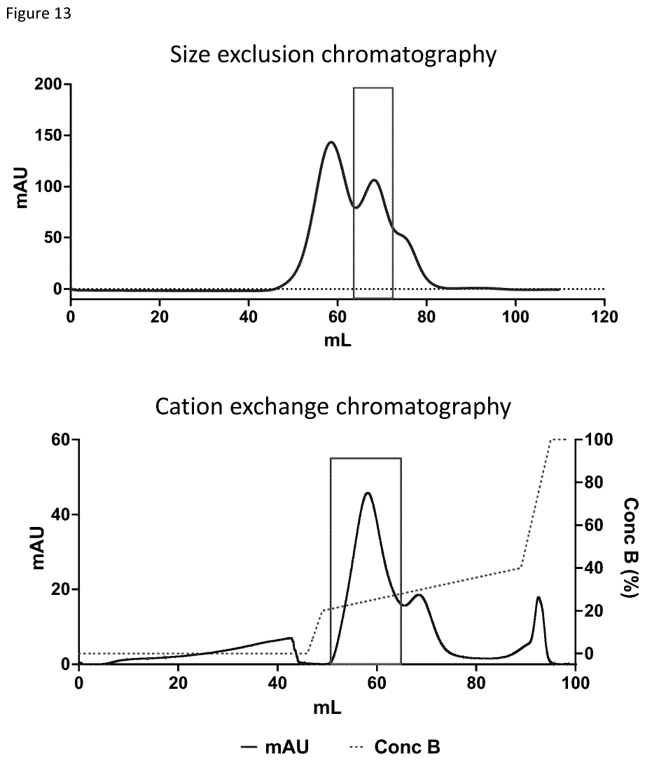

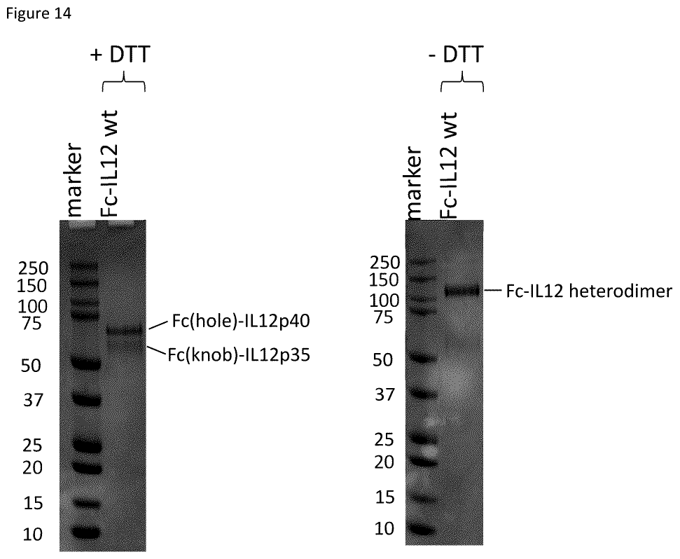

For expression in mammalian cells, IL-12 was recombinantly fused to the carboxy-terminus of human IgG1-Fc via a flexible linker (GGGGSGGGGSGGGGSTR). Fusion of IL-12 to an Fc-domain can be advantageous for several reasons, including, but not limited to: i) increased stability, ii) ease of purification via protein A chromatography, iii) increased serum half-life due to recycling via the neonatal Fc receptor, and iv) the capacity of Fc to be expressed as a heterodimer thereby allowing presentation of IL-12 in a heterodimeric form that mimics its natural physiological state. In the initial experiments, knob-in-hole mutations and an artificial disulfide to promote Fc-heterodimer formation and included an N297G substitution within each Fc chain to prevent N-linked glycosylation and thereby reduce Fc effector function were utilized. The configuration of the heterodimeric IL-12 Fc fusion protein used is shown in FIG. 12. The heterodimeric IL-12 Fc fusion protein was expressed by lentiviral transduction of the two chains (encoding “IgG1Fc(knob)-IL12p35” and “IgG1Fc(hole)-IL12p40”) into CHO cells, and was purified using a 3-step procedure, comprising: protein A, size-exclusion chromatography, and cation exchange chromatography. Representative chromatographs for exemplary wild-type IL-12 heterodimeric Fc fusion protein are shown in FIG. 13. Fractions corresponding to the heterodimeric IL-12 Fc fusion protein that were pooled for further purification/analysis are indicated by rectangles. Both “Fc(knob)-IL12p35” and “Fc(hole)-IL12p40” chains co-eluted throughout the purification process and were evident as a single species under non-reducing SDS-PAGE conditions in the final purified samples, indicating proper formation of a disulfide-linked heterodimer (FIG. 14).

### B. Example 2—Identification and Production of Reduced Potency IL-12 Fc Single Mutants

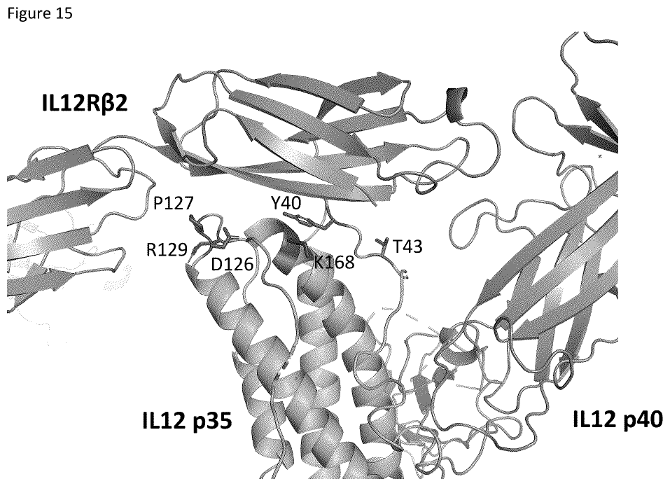

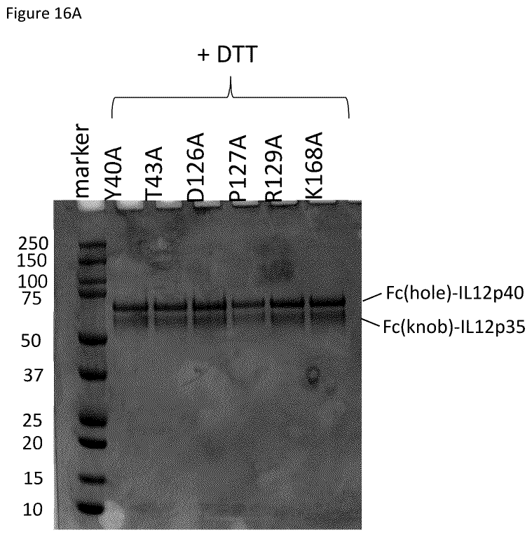

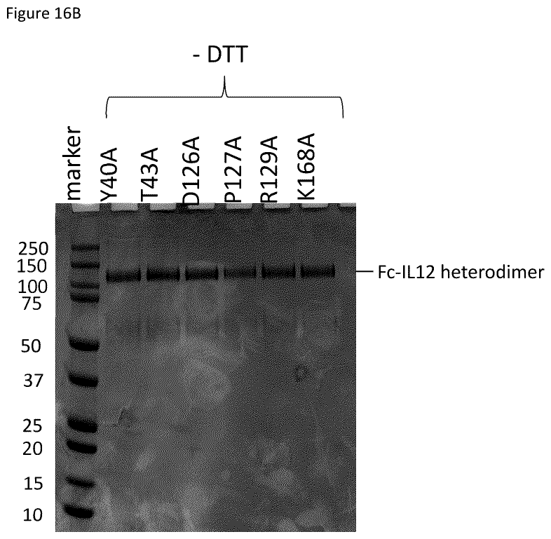

To enable rational selection of non-naturally occurring IL-12p35 mutants, a model of the IL-12/IL-12R complex was generated by fitting the crystal structure of IL-12 (PDB ID 3HMX) and an AlphaFold model of IL-12Rβ2 into the low resolution cryo-EM map of the IL-12/IL-12R quaternary complex (EMD-21645) (FIG. 15). Six surface-exposed IL-12p35 residues that were likely to influence binding to IL-12Rβ2 based on their spatial proximity were selected. These included residues derived from the A-B (Y40 and T43) and B-C (D126, P127 and R128) loops, as well as K168 within the D-helix. Alanine mutants at each of these positions (Y40A, T43A, D126A, P127A, R128A, K168A) were individually introduced into the IL-12 Fc construct and expressed these in CHO cells, as described above in Example 1. The final purity of each of the individual non-naturally occurring IL-12 mutants (comprising one of six aforementioned substitution mutations in the p35 subunit domain (also referred to as “IL-12 Fc single mutants” or “single mutants”) is shown in FIG. 16.

### C. Example 3—Binding Affinity of IL-12 F Single Mutants for IL-12R12

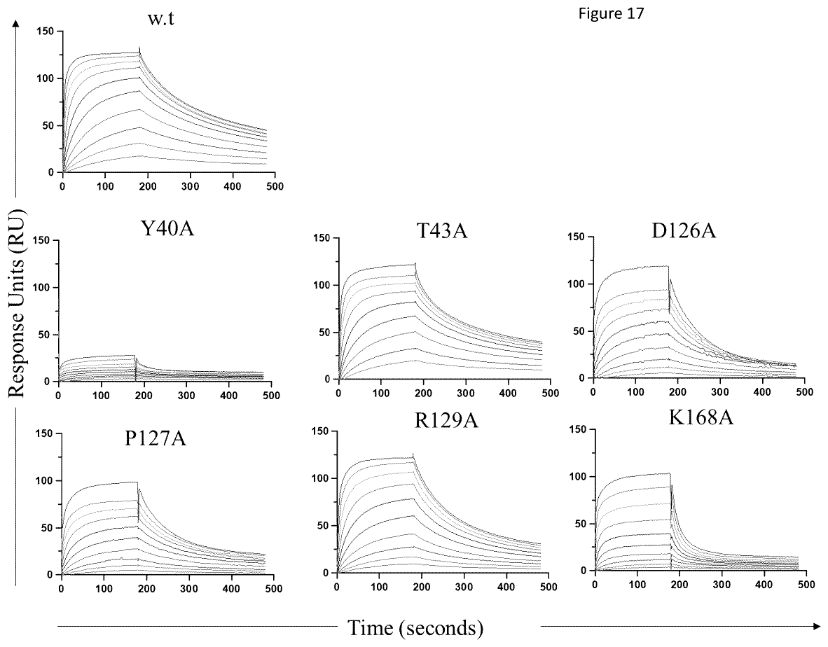

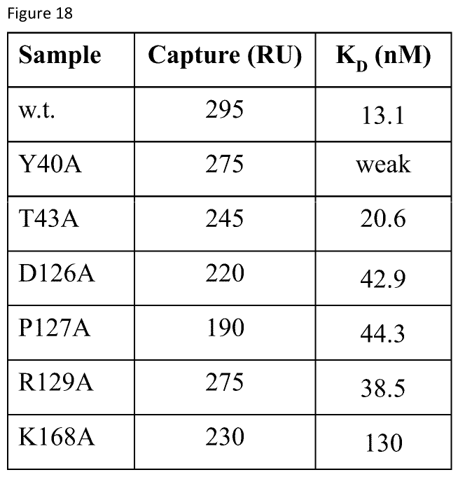

The affinity of the various IL-12 Fc single mutants for IL-12Rβ2 was assessed using a surface plasmon resonance (SPR) assay. SPR measurements were recorded on a Biacore T100 at 20° C. in a buffer comprising 20 mM sodium phosphate pH 7.4, 150 mM NaCl and 0.05% Tween20. Approximately 200-300 response units (RU) of IL-12 Fc w.t. or mutants (IL-12 Fc single mutants) thereof were immobilized on to protein A series S chips and binding of a truncated form of the IL-12Rβ2 extracellular region (domains 1-3) was measured at a flow rate of 30 μl/min. Following a 300 second dissociation phase, the remaining bound IL-12Rβ2 was washed off the chip using 50 mM Glycine pH 2.5, followed by 50 mM NaOH (30 second injections for each). Kinetic parameters were calculated using BIAevaluation software using a 1:1 Langmuir model, following subtraction of binding to a reference flow cell containing an isotype control antibody. Raw sensorgrams and a summary of SPR measurements are shown in FIGS. 17 and 18, respectively. While IL-12Rβ2 bound to IL-12 Fc w.t. with a relatively high affinity (K_{D}=13.1 nM), binding was diminished in several of the IL-12 Fc single mutants. In particular, the Y40A variant exhibited markedly reduced binding, such that a reliable affinity could not be determined (undetectable or “weak” binding), whereas a more modest reduction in affinity was apparent for the IL-12 Fc single mutants comprising K168A, D126A, or P127A substitution mutations.

### D. Example 4—Potency of IL-12 Fc Single Mutants Using an IL-12 HEK Reporter

As described above, binding of IL-12 to IL-12 receptor triggers an intracellular signaling cascade that results in, inter alia, TyK2 (tyrosine kinase 2), JAK2 (Janus kinase 2) and STAT4 (signal transducer and activator of transcription 4) activation, which leads to the production and secretion of IFNγ. Thus, to measure the potency of the IL-12 Fc single mutants, a commercially available reporter system (HEK-Blue IL-12 cells, InvivoGen) was utilized, whereby IL-12-induced triggering of the STAT4 pathway leads to the production of secreted embryonic alkaline phosphatase (SEAP), which can readily be detected using QUANTI-Blue reagent.

To determine the potency of the IL-12 Fc single mutants, varying concentrations of individual IL-12 Fc mutants, IL-12 Fc w.t., and a commercially available IL-12 not fused to a Fc (Miltenyi) were incubated with 12,500 HEK-Blue IL-12 cells overnight at 37° C. in a 384-well plate resuspended with DMEM containing 4.5 g/L glucose, 2 mM L-glutamine, 10% heat inactivated FBS, and 100 U/ml penicillin/streptomycin. The following day, SEAP was quantified using QUANTI-Blue detection reagent by measuring optical density (OD) at 620 nm using a PHERAstar plate reader and results were plotted in Prism software.

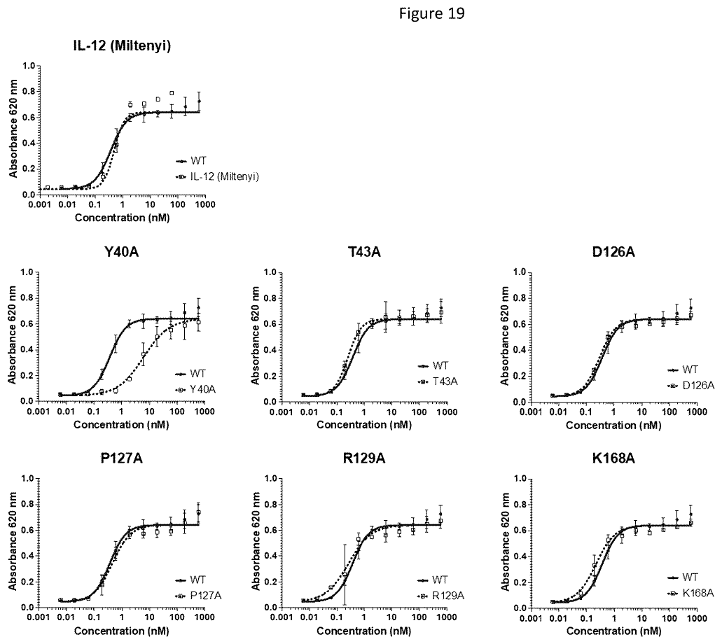

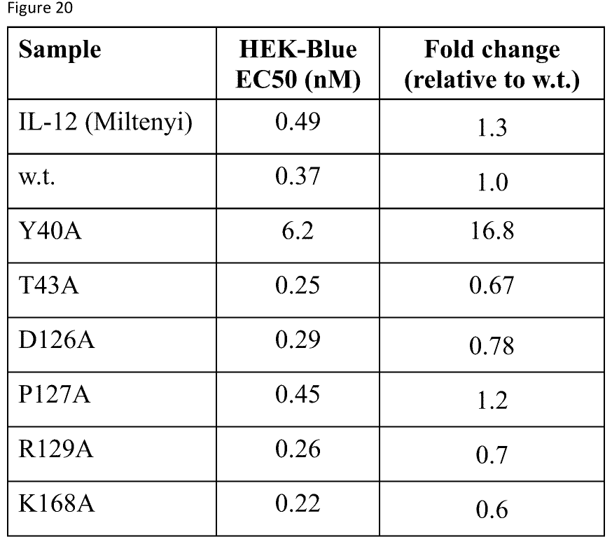

The results of the HEK-Blue IL-12 assay are shown in FIG. 19 and summarized in FIG. 20. Wild-type IL-12 Fc exhibited a similar potency to the commercially sourced IL-12 (Miltenyi), indicating that the monovalent Fc fusion format used does not alter IL-12 activity. Some of the assessed mutations resulted in no significant change or a modest decrease in IL-12Rβ2 affinity. Unexpectedly, the IL-12 Fc single mutant containing a Y40A substitution mutation in the p35 subunit domain exhibited a marked reduction in potency and activity (approximately a 17-fold reduction in the HEK-Blue EC₅₀ value).

### E. Example 5—Capacity of IL-12 Fc Single Mutants to Trigger IFNγ Release in Primary T Cells

The anti-tumor activity of IL-12 is at least in part due to its capacity to induce the secretion of IFNγ by immune cells, which is cytostatic/cytotoxic, anti-angiogenic and can upregulate MHC; I and II expression on tumor cells to enable immune recognition. Accordingly, the capacity of the IL--12 Fc single mutants to induce IFNγ secretion by primary T cells was further investigated. Peripheral blood mononuclear cells (PBMCs) from healthy donors were expanded/activated for 2 days in RPMI 1640 media containing 10% FCS, 1×GlutaMAX, 1% pen/strep, 0.1% β-mercaptoethanol, 25 mM HEPES, 1% non-essential amino acids, 1% sodium pyruvate, 600 U/ml IL-2, and 32 ng/ml OKT3 prior to being maintained for 3 days in a similar medium characterized above except that it lacked OKT3 and contained 50 U/ml IL-2. For the assay, 100,000 cells were dispensed into 96-well plates and incubated with the indicated concentration of IL-12 Fc single mutants, IL-12 Fc w.t., or the commercially available IL-12 (Miltenyi) for 45 hours. Supernatants were collected and IFNγ levels were quantified using a Bio-Plex PIX Multiplex Reader.

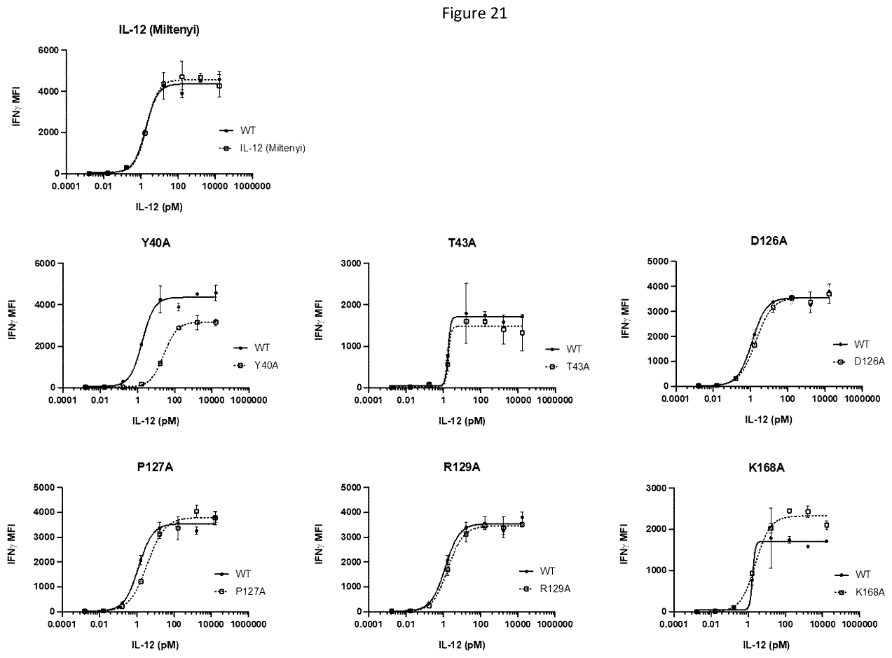

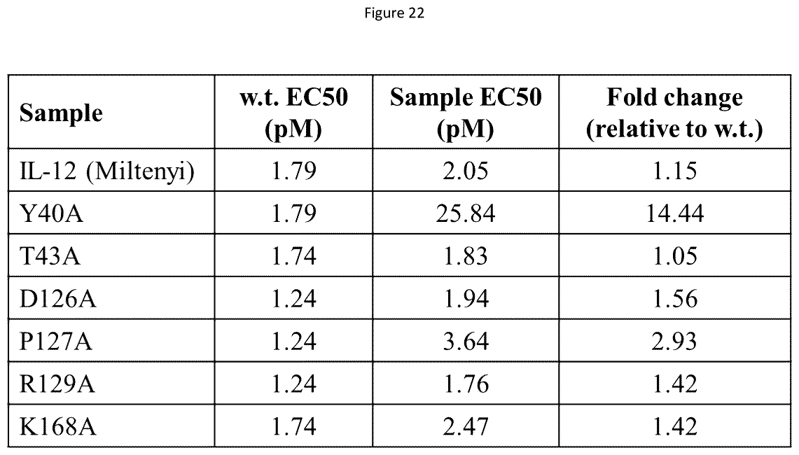

The results of the IFNγ assay are shown in FIG. 21 and summarized in FIG. 22. IL-12 Fc w.t. induced IFNγ to a similar extent as the commercially available IL-12. The IL-12 Fc single mutant containing the Y40A substitution mutation in the p35 subunit domain exhibited a decrease in EC₅₀ by approximately 14-fold, relative to IL-12 Fc w.t. The IL-12 Fc single mutant containing the P127A substitution mutation in the p35 subunit domain exhibited a decrease in EC₅₀ by approximately 3-fold, relative to IL-12 Fc w.t. The remainder of the IL-12 Fc single mutants exhibited a slight reduction in capacity to stimulate the secretion of IFNγ.

### F. Example 6—IL-12-Fc Combination Mutants

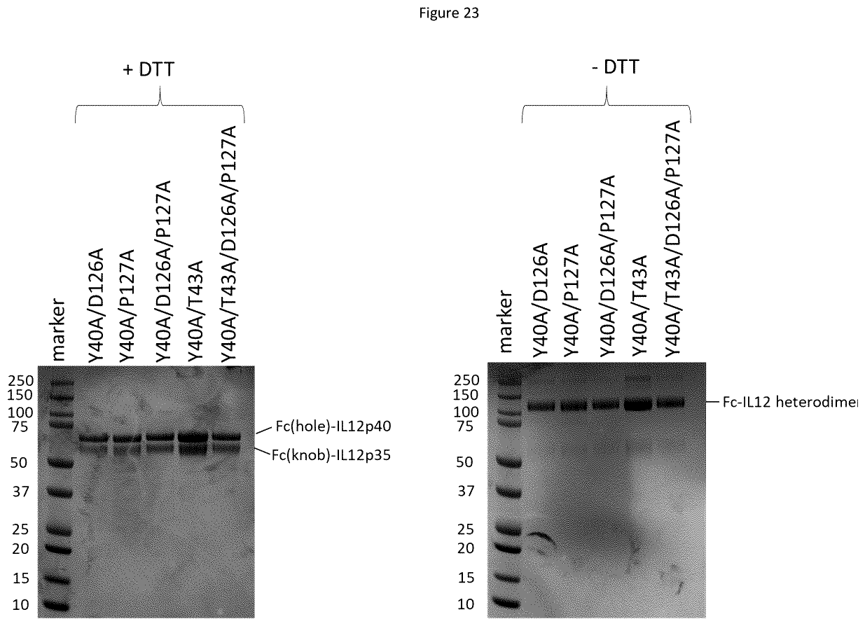

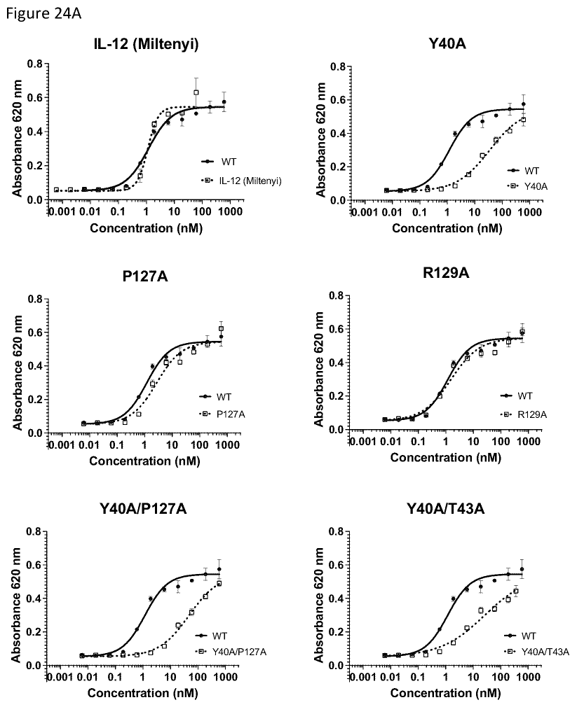

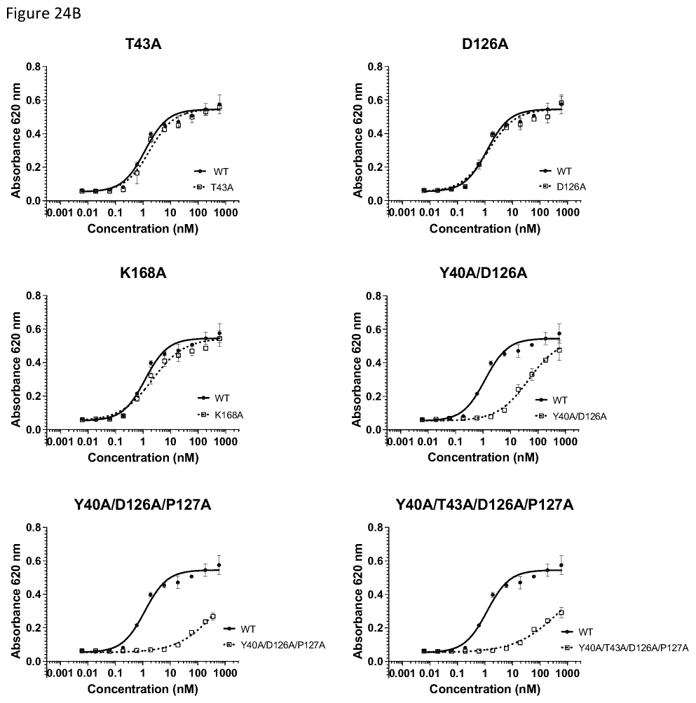

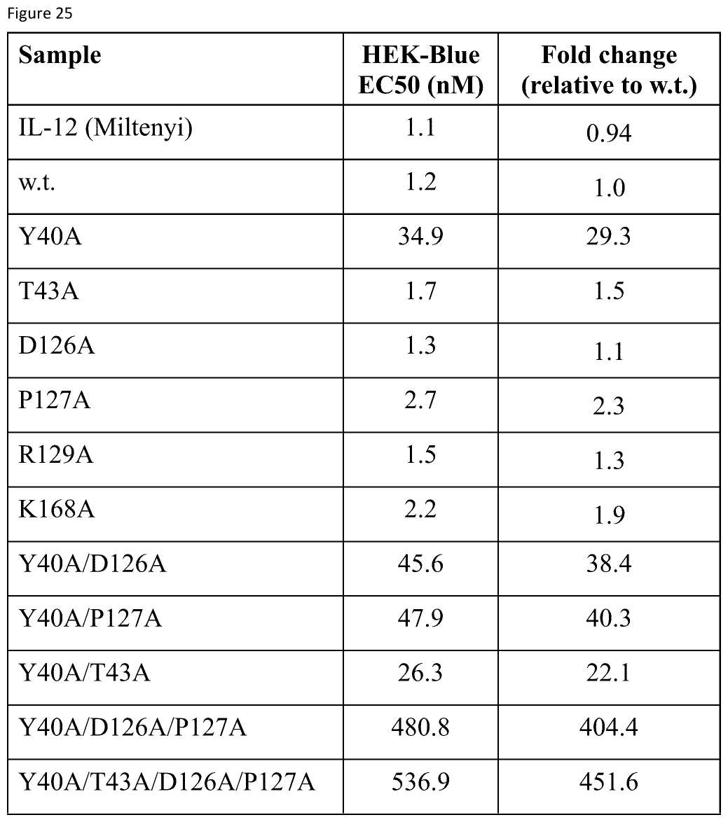

To identify additional mutants that confer further potency reductions in IL-12, the effects of combining mutations at more than one position within the IL-12p35 subunit domain were explored. Variant IL-12 mutants were expressed in monovalent, heterodimeric IL-12 Fc format, as described above in Example 1 (FIG. 23), wherein the p35 subunit domain comprised two (“IL-12 Fc double mutants” or “double mutants”), three (“IL-12 Fc triple mutants” or “triple mutants”), or four (“IL-12 Fc quadruple mutants” or “quadruple mutants”) substitution mutations, collectively referred to as “IL-12 Fc combination mutants.” Since Y40A was associated with the greatest loss in IL-12 activity, this mutant position was combined with either T43A, D126A, P127A, or combinations thereof. The potency of the IL-12 Fc combination mutants was then assessed in the HEK-Blue IL-12 reporter assay, as described above in Example 4, alongside the individual IL-12 Fc single mutants, the IL-12 Fc w.t., and the commercially available IL-12 not linked to a Fc domain (Miltenyi), to provide a direct comparison in the same experiment. The results of the HEK-Blue IL-12 reporter assay is shown in FIG. 24 and summarized in FIG. 25. Consistent with the previous HEK-Blue IL-12 assay, Y40A demonstrated the greatest reduction in potency of the single mutants (a 29-fold reduction, approximately), while P127A was associated with a moderate loss in potency (a 2-fold reduction, approximately). Unexpectedly, some of the double mutants exhibited a synergistic effect. The double mutants comprising Y40A/D126A or Y40A/P127A further reduced IL-12 potency below that of the individual Y40A mutation (a 38-fold and 40-fold reduction (approximately), respectively). This synergistic effect was not apparent for all mutant combinations, as evidenced by Y40A/T43A which exhibited a similar EC₅₀ value to that of Y40A alone. Remarkably, the Y40A/D126A/P127A triple mutant demonstrated a further reduction in potency (a 404-fold reduction, approximately) compared to Y40A/D126A or Y40A/P127A. Further, the Y40A/T43A/D126A/P127A quadruple mutant exhibited a 451-fold reduction (approximately) in potency.

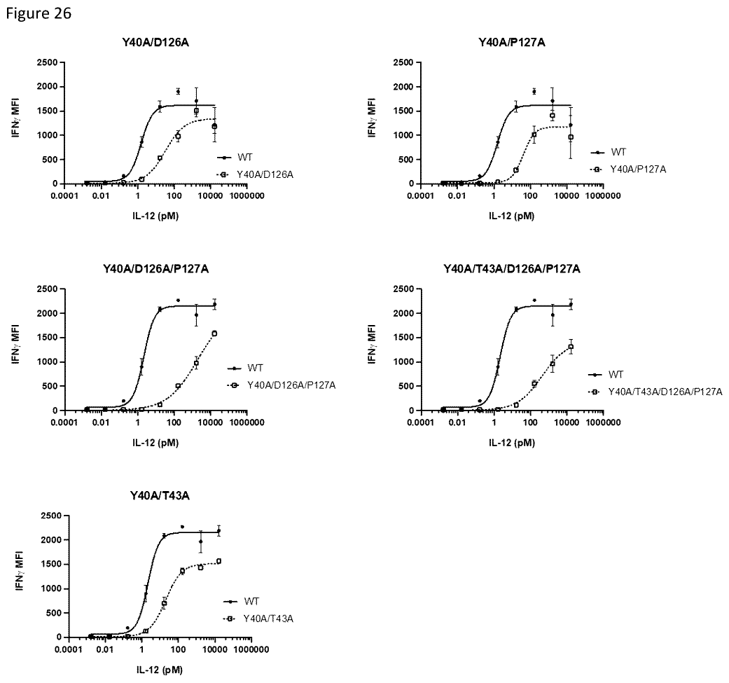

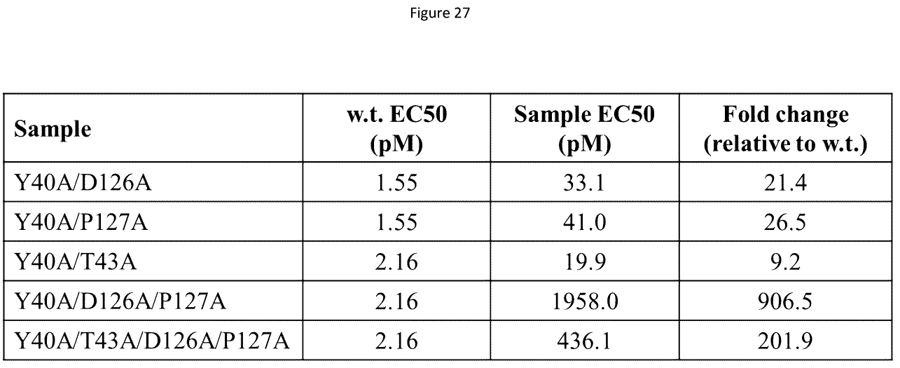

The activity of the double, triple, and quadruple IL-12 Fc mutants was also investigated in a primary T cell IFNγ release assay and compared to the activity of IL-12 Fc w.t., as described above in Example 5. The results are shown in FIG. 26 and summarized in FIG. 27. Overall, the capacity of mutants to stimulate IFNγ release correlated with their potency in the HEK-Blue IL-12 reporter assay, whereby Y40A/D126A (a 21-fold reduction, approximately) and Y40A/P127A (a 27-fold reduction, approximately) resulted in a modest decrease in potency compared to the Y40A single mutant (a 14-fold reduction (approximately), shown in FIG. 22), whereas a dramatic reduction in potency was observed for the Y40A/D126A/P127A triple mutant (a 907-fold reduction, approximately).

### G. Example 7—Additional IL-12-Fc Alanine Mutants

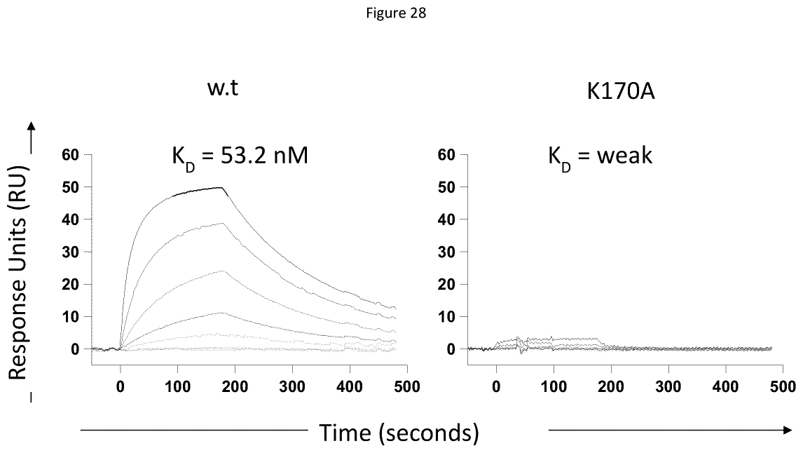

Based on a structural model of the IL-12/IL-12Rβ2 complex, an additional IL-12p35 residue, K170, that may play a role in IL-12Rβ2 binding was identified. Accordingly, an IL-12p35 K170A mutation was introduced into the heterodimeric IL-12 Fc fusion protein described in Example 1 and its binding to IL-12Rβ2 assessed using an SPR assay, as described in Example 3. Remarkably, introduction of the K170A mutation into the IL-12p35 subunit domain dramatically reduced IL-12Rβ2 binding, such that an affinity could not be accurately determined (i.e., the K170A mutant had “weak” binding affinity, as described above; see, e.g., FIG. 28).

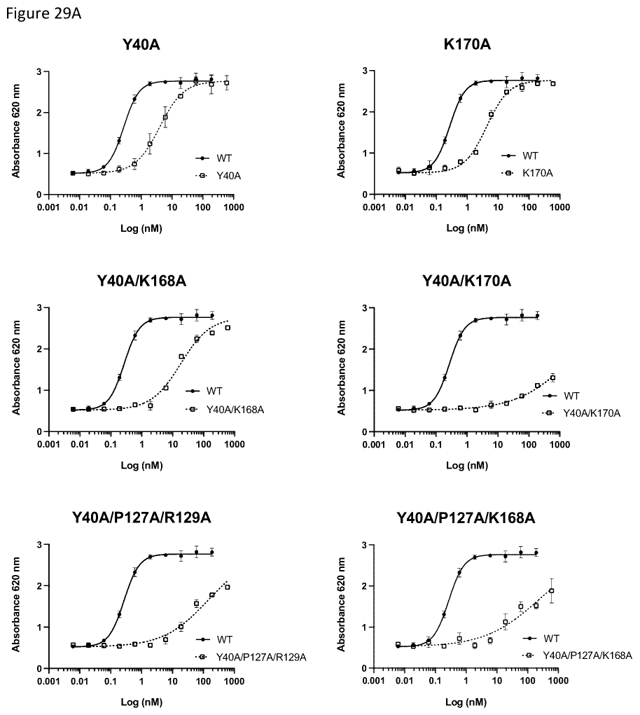

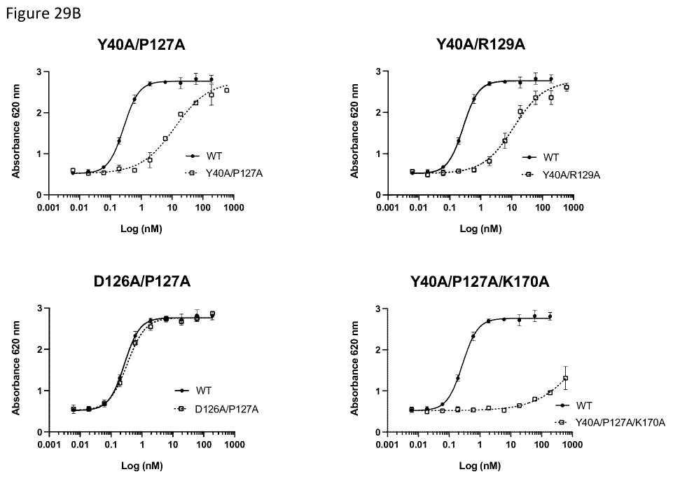

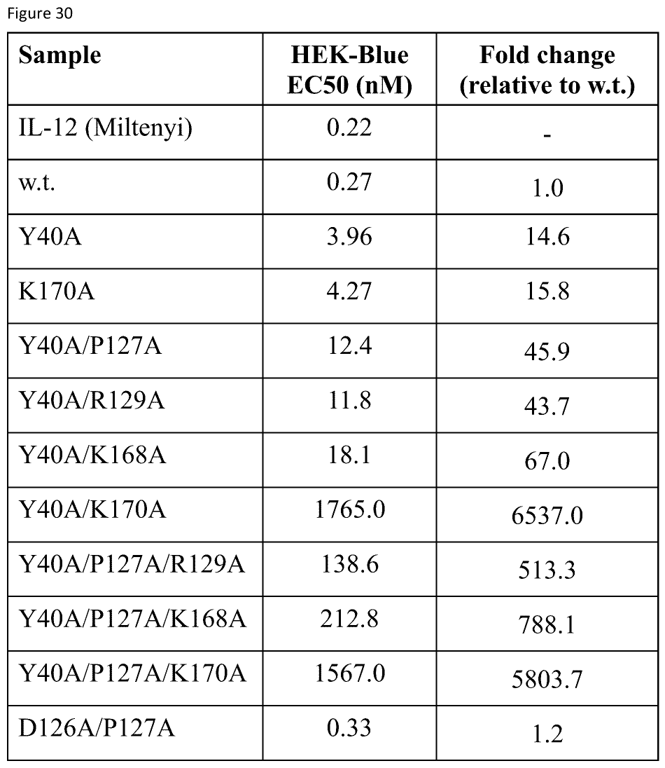

In addition to the K170A single mutant, additional double mutant and triple mutant combinations were generated, wherein a subset of the double and triple mutants included a K170A substitution mutation. The potency of these additional IL-12 Fc mutants was initially assessed in the HEK-Blue IL-12 reporter assay, as described in Example 4. As a frame of reference, the Y40A single mutant and the Y40A/P127A double mutant was also included to allow for a direct comparison within the same experiment. The results of the HEK-Blue IL-12 assay are shown in FIG. 29 and summarized in FIG. 30. Consistent with the loss in affinity for IL-12Rβ2 observed by SPR, the K170A single mutant resulted in a marked reduction in potency (approximately 16-fold reduction in the HEK-Blue EC₅₀ value). Surprisingly, each of R129A, K168A, and K170A conferred a further reduction in potency when combined with Y40A (in a double mutant), or when combined with Y40A/P127A (in a triple mutant). The magnitude of the potency loss was largest for K170A (approximately 6537-fold reduction in the HEK-Blue EC₅₀ value for Y40A/K170A compared to wild-type), intermediate for K168A (approximately 67-fold reduction in the HEK-Blue EC₅₀ value for Y40A/K168A compared to wild-type), and smallest for R129A (approximately 44-fold reduction in the HEK-Blue EC₅₀ value for Y40A/R129A compared to wild-type). Notably, the D126A/P127A double mutant did not impact IL-12-Fc potency, highlighting the unpredictability involved in selecting mutants whose effects will synergize to produce a reduction in IL-12 potency.

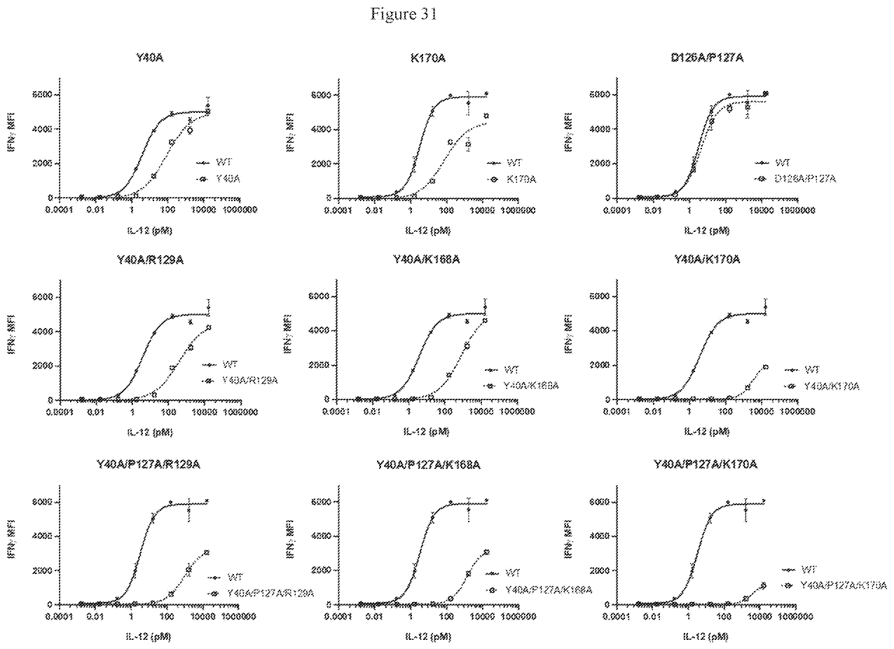

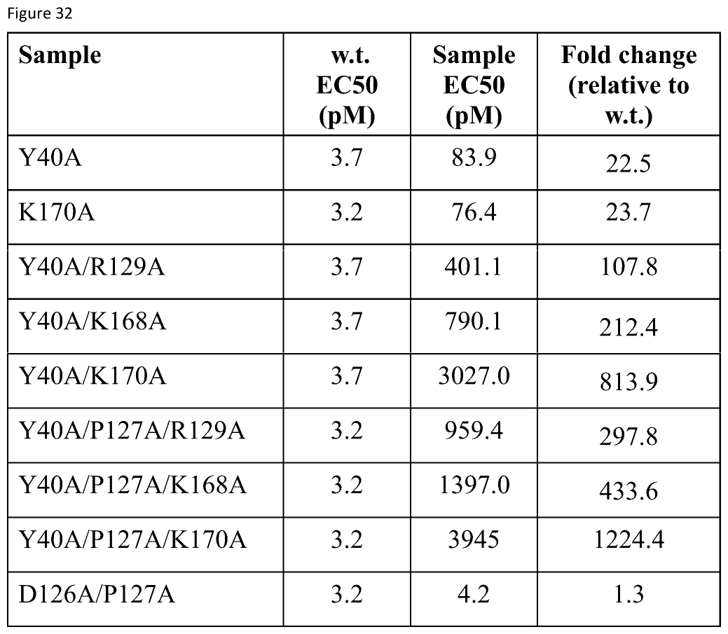

The activity of the additional single mutants, double mutants, and triple mutants was also investigated in a primary T cell IFNγ release assay, as described in Example 5, alongside wild-type IL-12 Fc and IL-12 Fc Y40A mutant for comparison. The results of the T cell IFNγ release assay is shown in FIG. 31 and summarized in FIG. 32. The capacity of mutants to stimulate IFNγ release correlated with their potency in the HEK-Blue IL-12 reporter assay, whereby K170A resulted in a similar potency reduction as Y40A (a 24-fold reduction for K170A compared to a 23-fold reduction for Y40A, approximately) and each of R129A, K168A, and K170A conferred a further reduction in potency when combined with Y40A (in a double mutant) or when combined with Y40A/P127A (in a triple mutant), with a hierarchy similar to that observed in the HEK-Blue reporter assay.

### H. Example 8—Selected IL-12 Alanine Mutants in an Alternative Fc-Fusion Format

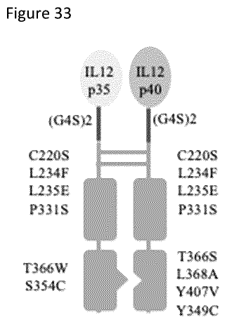

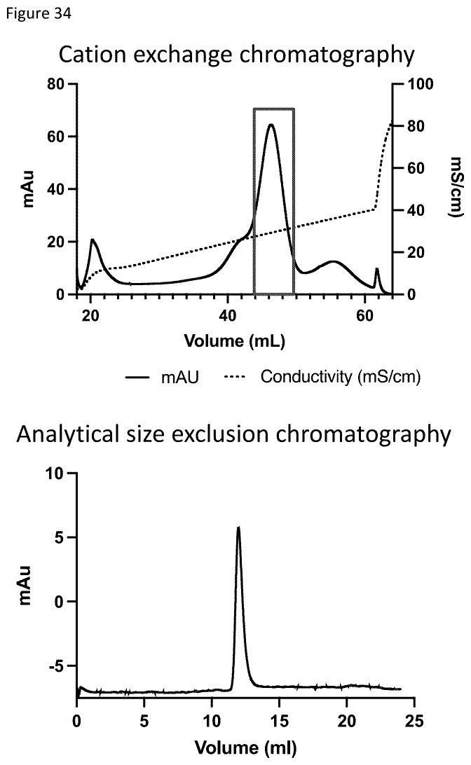

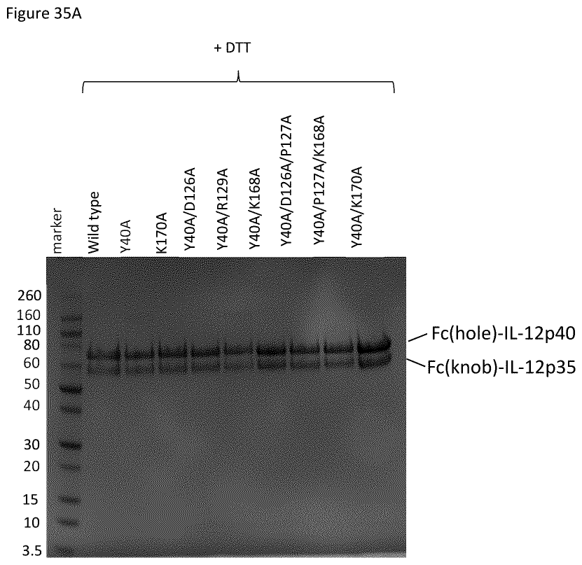

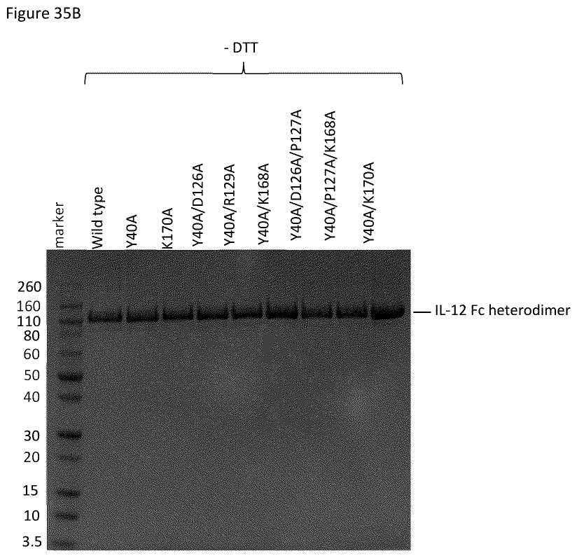

Eight IL-12 alanine mutants that spanned a range of potencies were selected for further analysis. These included: Y40A, K170A, Y40A/D126A, Y40A/R129A, Y40A/K168A, Y40A/D126A/P127A, Y40A/P127A/K168A, and Y40A/K170A, and are collectively referred to as the “alanine potency series.” Alongside wild-type IL-12, the selected IL-12 alanine mutants were expressed using an alternate Fc-fusion format whereby the IL-12 subunits were attached to the N-terminus of the Fc region via a (G4S)2 linker (see, e.g., FIG. 33). In addition to knob-in-hole mutations to promote Fc heterodimerization, other modifications to the sequences included: (i) L234F, L235E, and P331S within the Fc domains, (ii) C220S within the Fc hinge, and (iii) a C252S substitution within the IL-12p40 subunit to prevent disulfide linked aggregation. Wild-type IL-12-Fc and the “alanine potency series” were expressed by transient transfection of expi-CHO cells and purified by protein A and cation exchange chromatography. Elution fractions from cation exchange columns were pooled and subjected to analytical size exclusion chromatography using a Superdex 200 10/300 column to confirm that samples were free from high molecular weight aggregates. Representative chromatographs for exemplary wild-type IL-12 heterodimeric Fc fusion protein are shown in FIG. 34, and the final purity of wild-type IL-12-Fc and the “alanine potency series” are shown in FIG. 35.

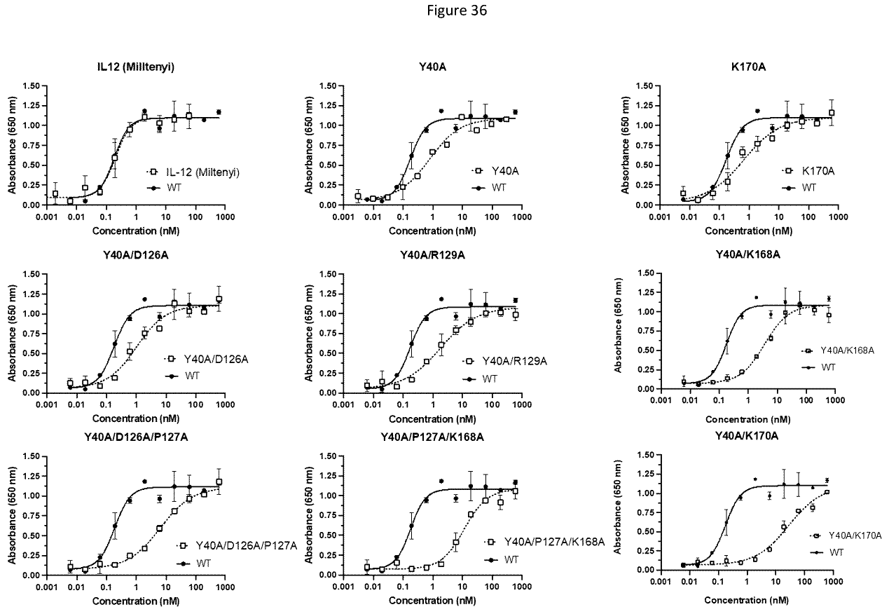

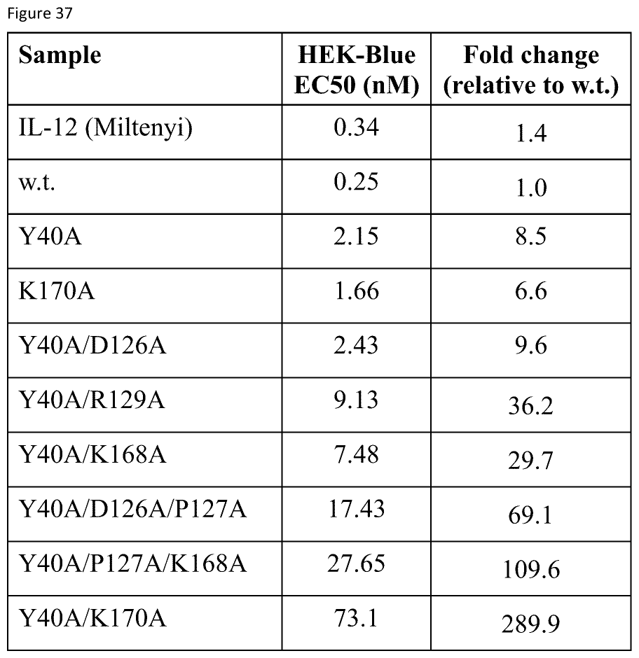

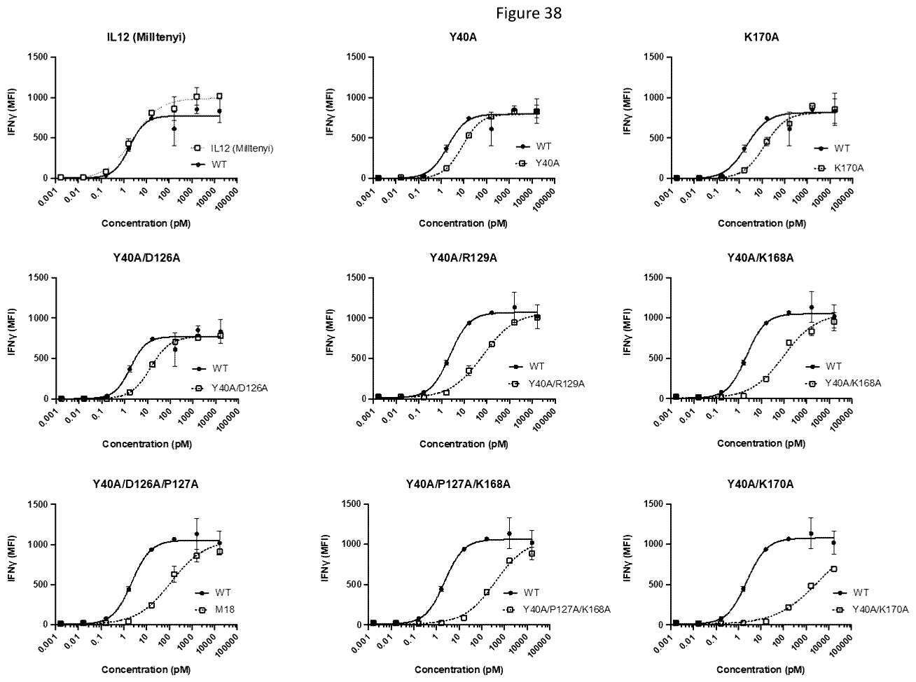

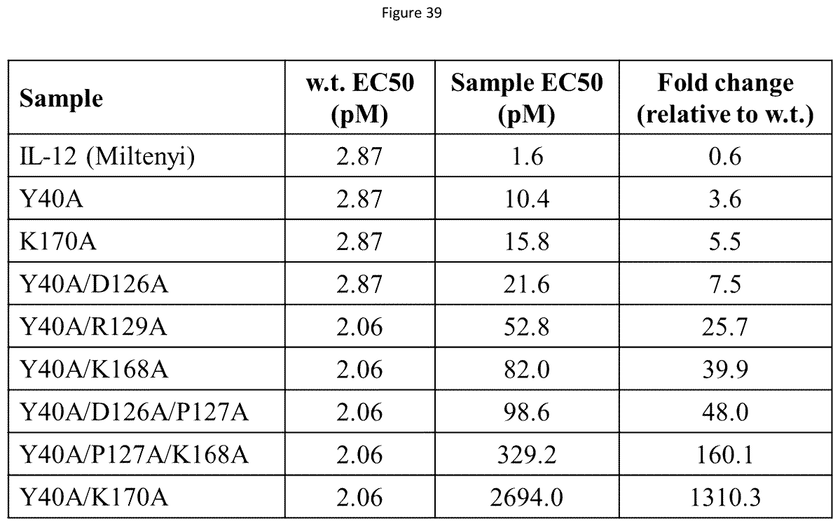

The activity of the “alanine potency series” was assessed using the IL-12 HEK reporter assay, as described in Example 4, and the primary T cell IFNγ assay, as described in Example 5, except that IFNγ levels were quantified using a Luminex system (instead of the Bio-Plex system). The results of the HEK-Blue IL-12 assay are shown in FIG. 36 and summarized in FIG. 37. The results of the T cell IFNγ assay is shown in FIG. 38 and summarized in FIG. 39. In both assays, wild-type IL-12 Fc exhibited a similar potency to the commercially sourced IL-12 (Miltenyi), indicating that IL-12 activity was not impacted by fusion of the cytokine to the N-terminus of the Fc subunit, the introduction of different mutations to silence Fc effector function, or the C252S substitution within IL-12p40. While the absolute fold change EC₅₀ values for the mutants relative to wild-type IL-12 Fc differed in magnitude to earlier experiments, the rank order of potency for the mutants followed a similar trend, whereby (i) Y40A, K170A, and Y40A/D126A resulted in a relatively smaller reduction in potency, (ii) Y40A/R129A and Y40A/K168A were associated with a relatively moderate reduction in potency, and (iii) Y40A/D126A/P127A, Y40A/P127A/K168A, and Y40A/K170A resulted in a relatively larger reduction in potency.

### I. Example 9 IL-12 Fc Alanine Mutants Modulate T Cell Phenotype In Vitro

The effect of IL-12 Fc variants exhibiting a range of reduced potencies on T cell phenotype in vitro was subsequently investigated. To expand/activate human T cells, peripheral blood mononuclear cells (PBMCs) from three independent healthy donors were cultured in RPMI 1640 media containing 10% FCS, 2 mM L-alanyl-L-glutamine dipeptide, 50 U/mL penicillin, 50 ug/mL streptomycin, 55 μM β-mercaptoethanol, 10 mM HEPES, 0.1 mM non-essential amino acids, 1 mM sodium pyruvate, 600 U/ml IL-2, and 30 ng/ml OKT3 for 2 days prior to being washed and resuspended in the same medium characterized above except that it lacked OKT3, and contained 50 U/ml IL-2 and either 1, 5, or 10 ng/ml of wild-type IL-12 Fc, or 10 ng/ml of each of individual members of the “alanine potency series.” Two days later, the T cells were stained with a surface marker panel, comprising: (i) anti-CD4 (BUV805), (ii) anti-CD8 (BUV737), (iii) anti-CD39 (PerCPCy5.5), (iv) anti-CD45RO (BV650), (v) anti-CD45RA (BUV395), (vi) anti-CD62L (APC-Cy7), (vii) anti-PD-1 (BV785), (viii) anti-CD27 (BV711), (ix) anti-Tim3 (BV421), (x) anti-CD95 (PE-Cy7), and (xi) anti-LAG-3 (AF647). Subsequently, the T cells were fixed and permeabilized using a FoxP3 Transcription Factor Kit (ThermoFisher Scientific), and then stained with an intracellular marker panel, comprising: (a) anti-TCF1 (PE), (b) anti-Ki-67 (BV605), (c) anti-FoxP3 (AF700), and (d) anti-Granzyme B (APC), and then were analyzed using a Cytek Aurora.

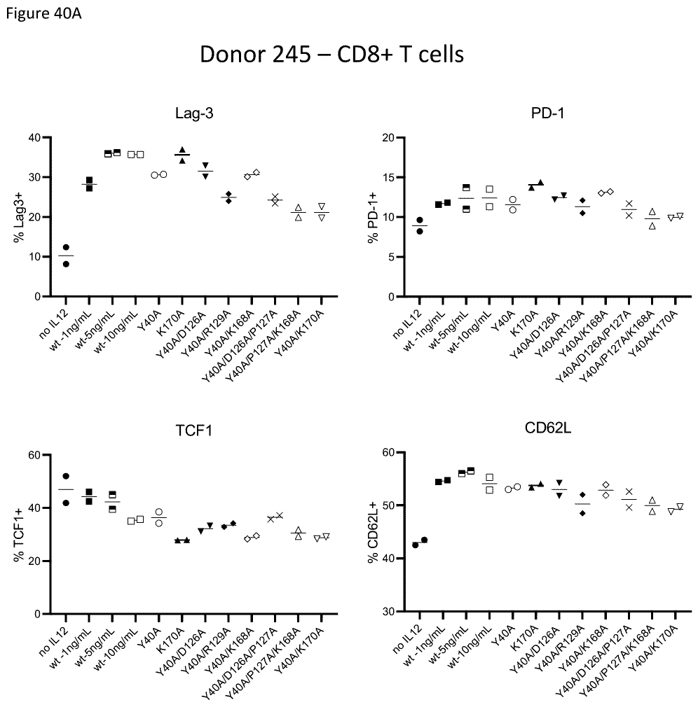

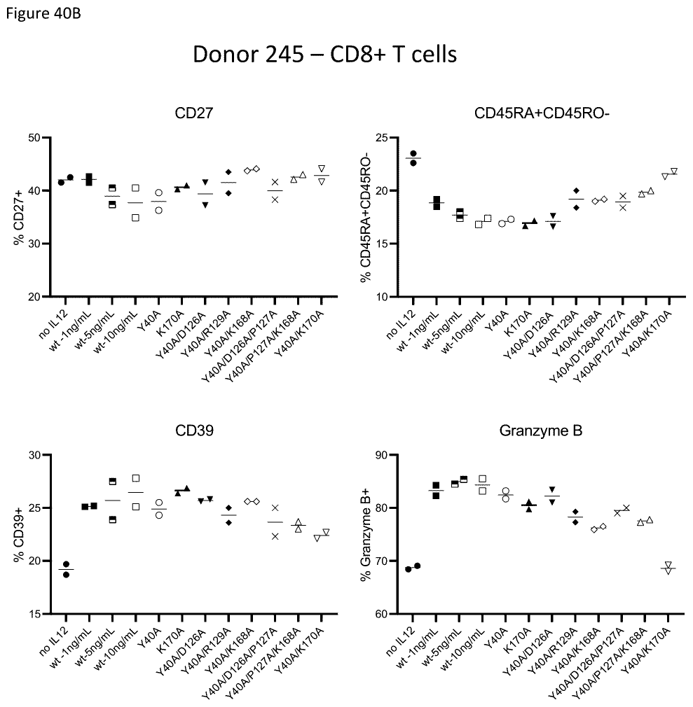

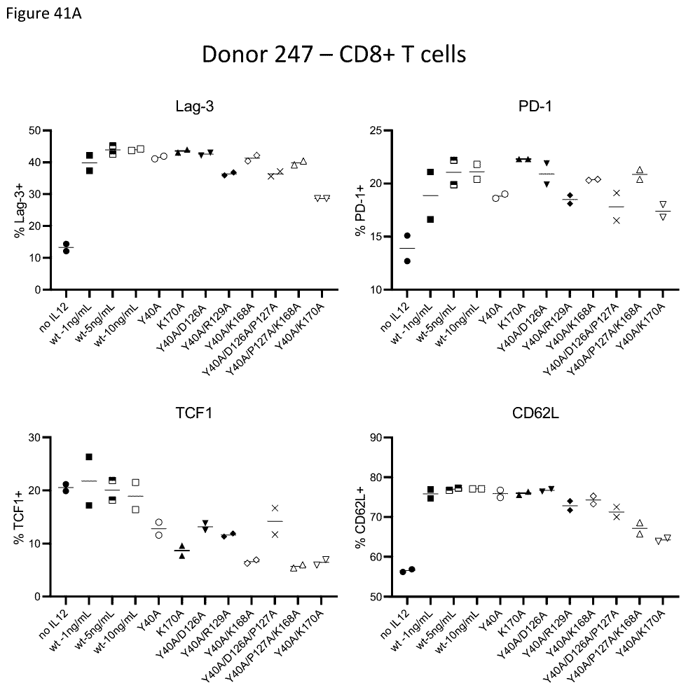

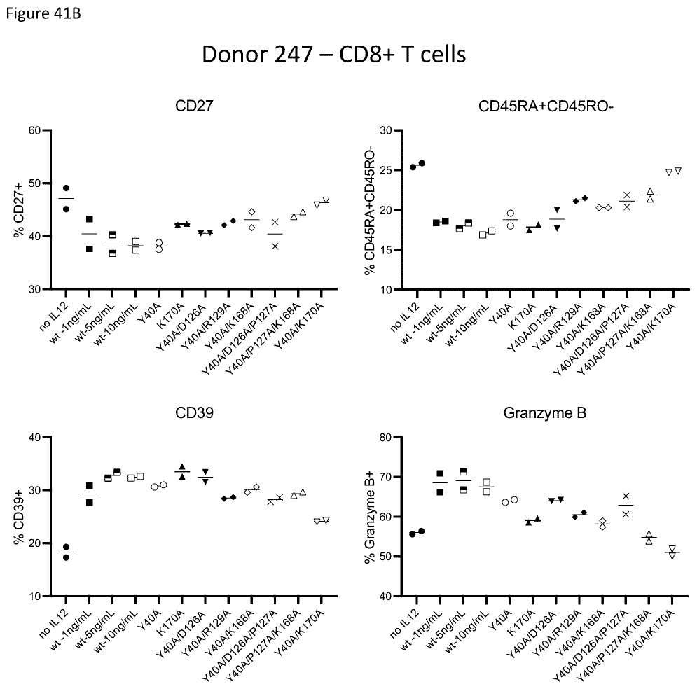

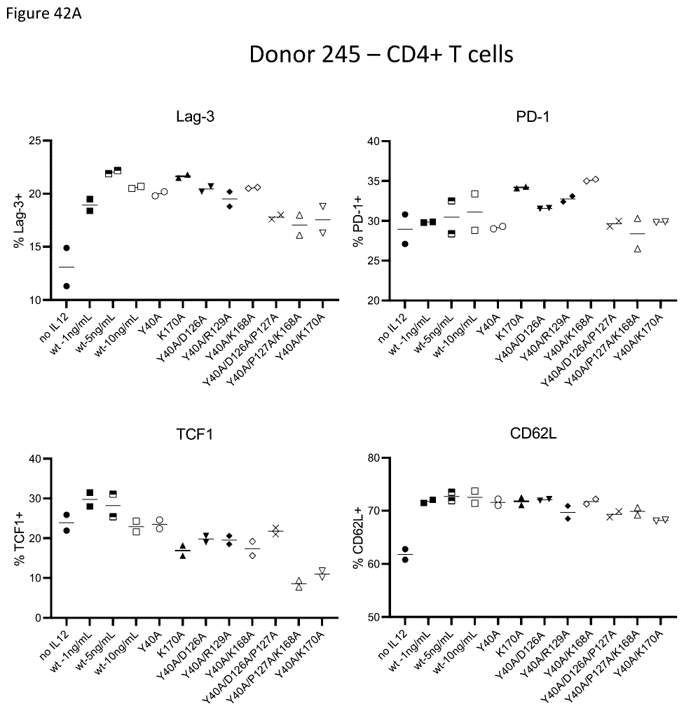

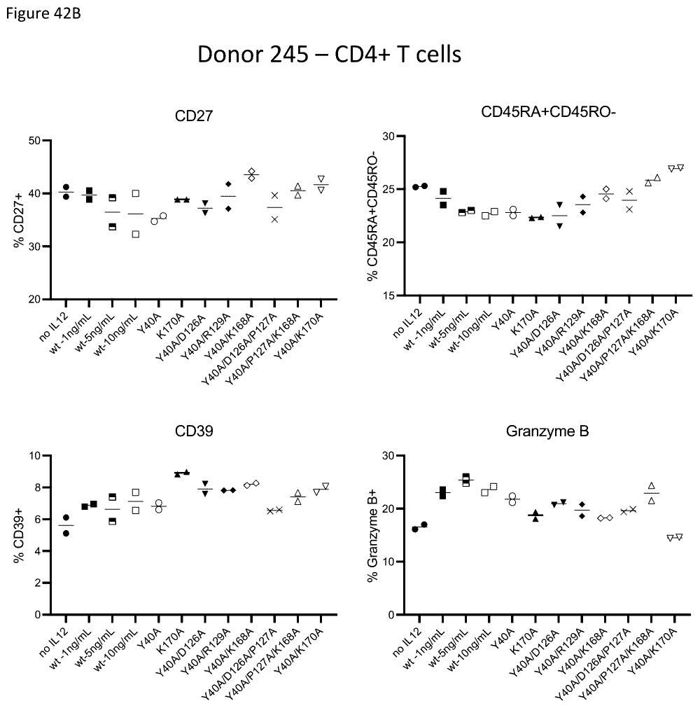

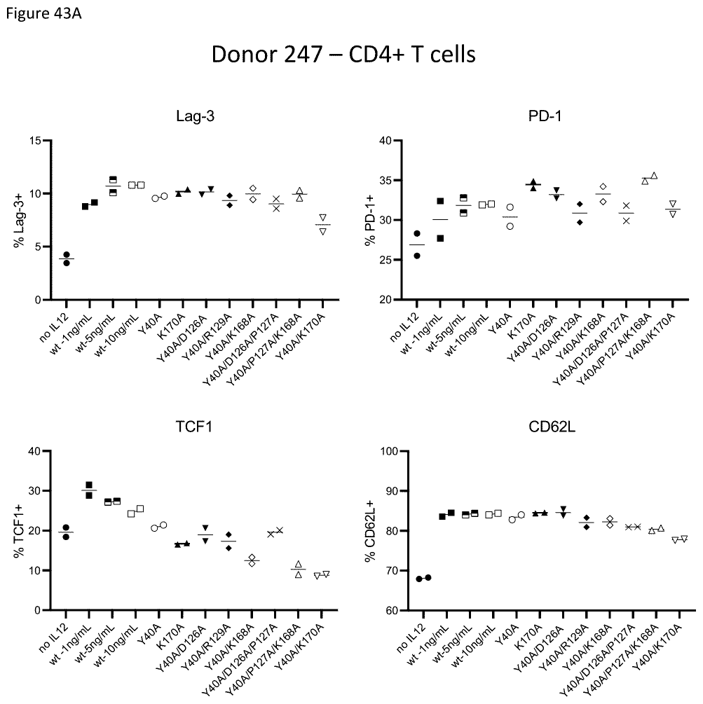

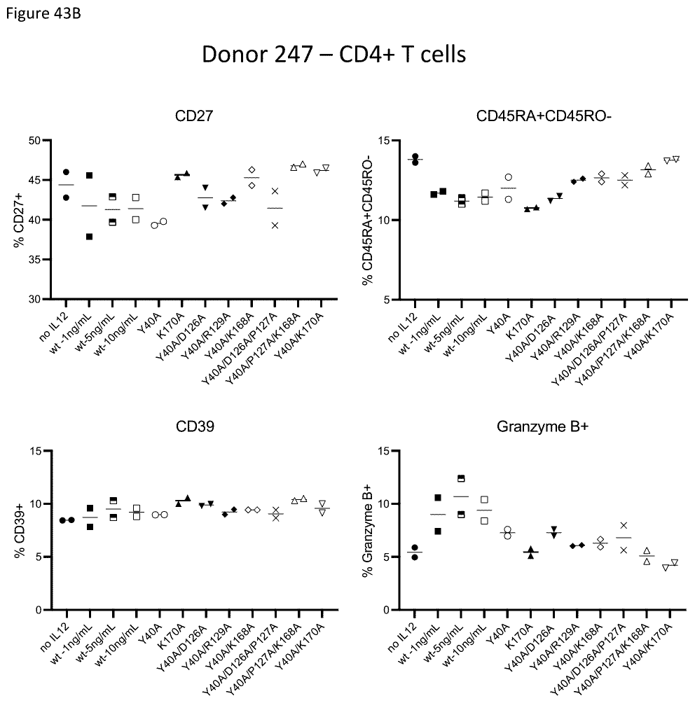

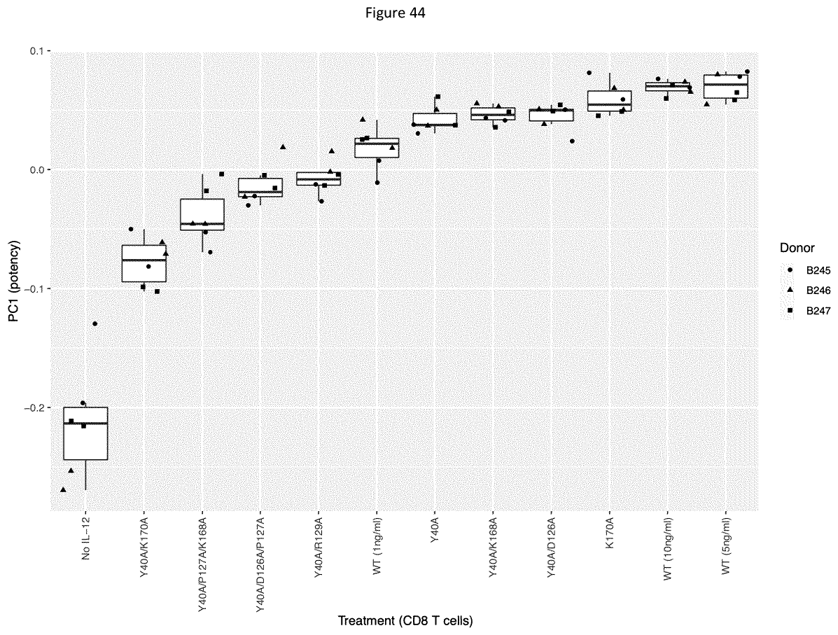

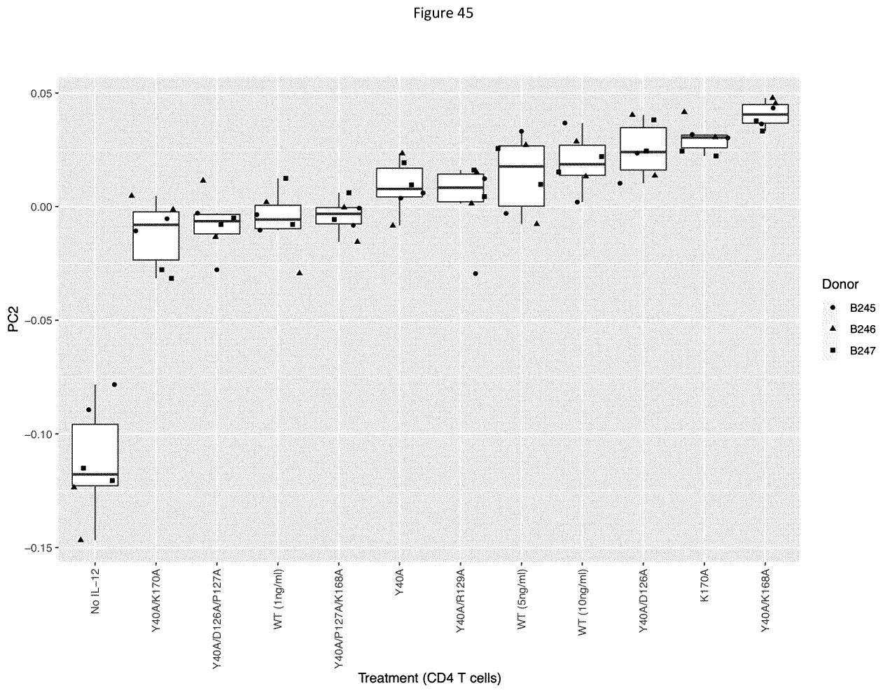

The expression of selected T cell markers on CD8+ and CD4+ T cells derived from two of the donors are depicted in FIGS. 40-43. Wild-type IL-12 Fc induced the expression of several T cell activation and exhaustion markers, including: granzyme B, LAG-3, CD39, and to a lesser extent PD-1, in both CD4+ and CD8+ cells, and was associated with a shift from naive to activated/memory phenotype as represented by a decrease in the proportion of CD45RA+CD45RO− cells. The extent to which these markers were induced varied amongst individual members of the “alanine potency series,” and directly correlated with potency whereby “higher potency” variants induced higher marker expression and “lower potency” variants induced marker expression to a lesser extent. To analyze the relationship between T cell phenotype and IL-12 potency more systematically, principal component analysis (PCA) was performed, taking into account the entire panel of phenotypic markers (described above) in both CD8+ and CD4+ T cells in 3 different PBMC donors (“B245,” “B246,” and “B247”; see, e.g., FIGS. 44 and 45). In CD8+ T cells, PC1 scores displayed strong correlation with IL-12 potency whereby wild-type IL-12 and “high potency” variants induced relatively higher scores compared to the “lower potency” variants and samples cultured in the absence of IL-12 Fc. In CD4+ T cells, PC2 correlated most strongly with IL-12 potency, although not as consistently as in CD8+ T cells.

### J. Exaample 10—IL-12 Fc Alanine Mutants Exhibit Favorable In Vivo Characteristics

The in vivo properties of the reduced potency IL-12 Fc mutants were subsequently investigated in NOD SCID gamma (NSG) mice that were intravenously injected with 10×10⁶ freshly isolated human PBMCs. Mice (n=6 per group) were either untreated, or treated intraperitoneally on day 0 (i.e., the same day as PBMC xenografting) and day 7 with either 0.1 or 0.01 mg/kg of wild-type IL-12 Fc or the following IL-12 Fc reduced potency mutants: (i) Y40A/D126A, (ii) Y40A/R129A, (iii) Y40A/K168A, or (iv) Y40A/P127A/K168A. Blood was sampled periodically (at time=24, 96, 120, 144, 192, 240, 288, and 576 hours) to allow for the determination of IFNγ and IL-12 Fc levels in plasma, as well as immune cell enumeration and phenotyping by flow cytometry.

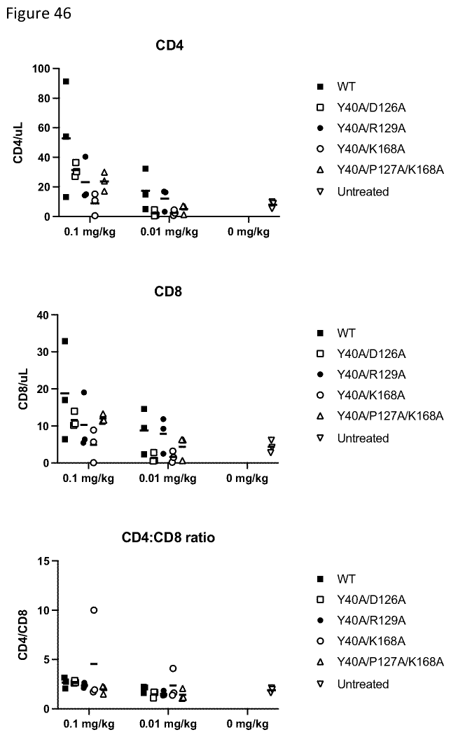

Data depicting enumeration of CD4+ and CD8+ T cells in the blood on day 12 are shown in FIG. 46. In animals receiving a 0.1 mg/kg dose of IL-12 (WT or IL-12 Fc reduced potency mutant), the highest absolute number of CD4+ and CD8+ cells were observed in animals injected with wild-type IL-12 Fc, whereas reduced numbers of CD4+ and CD8+ cells (but more than in untreated animals) were evident in mice injected with each of the reduced potency IL-12 Fc variants. Although the wild-type IL-12 Fc did result in a clear expansion of T cells at 0.01 mg/kg, differences in CD4+ and CD8+ cell numbers between untreated and the reduced potency IL-12 Fc variants were less apparent at this lower dosage. In addition to absolute numbers, the impact of IL-12 Fc potency on CD4:CD8 ratio was also assessed. While dramatic differences were not observed, the data indicated a trend whereby a similar CD4:CD8 ratio to untreated mice was observed for the “lower potency” IL-12 Fc variants, such as, for example Y40A/K168A and Y40A/P127A/K168A, whereas wild-type IL-12 Fc and the “higher potency” variants, such as, for example, Y40A/D126A, exhibited an increased CD4:CD8 ratio.

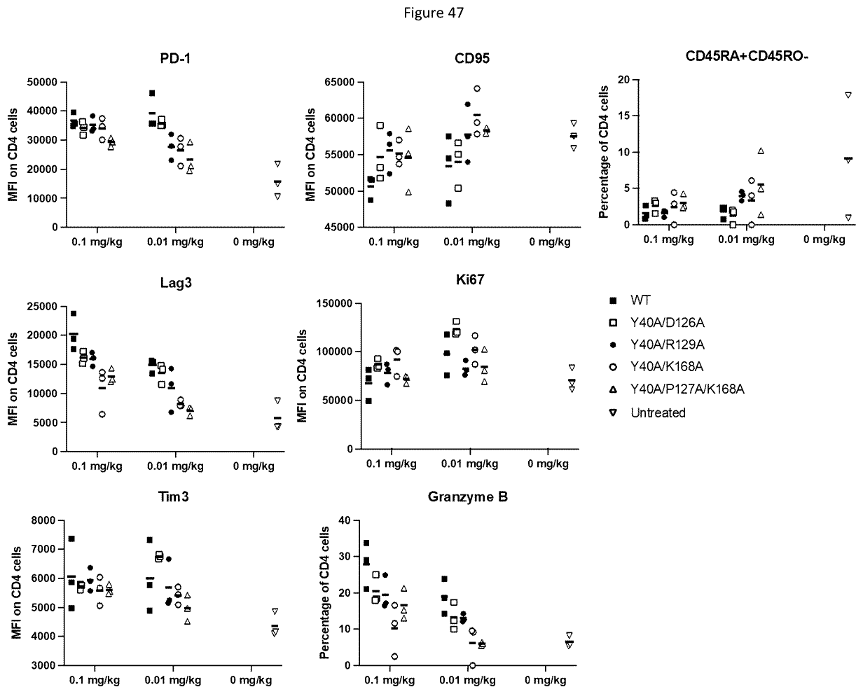

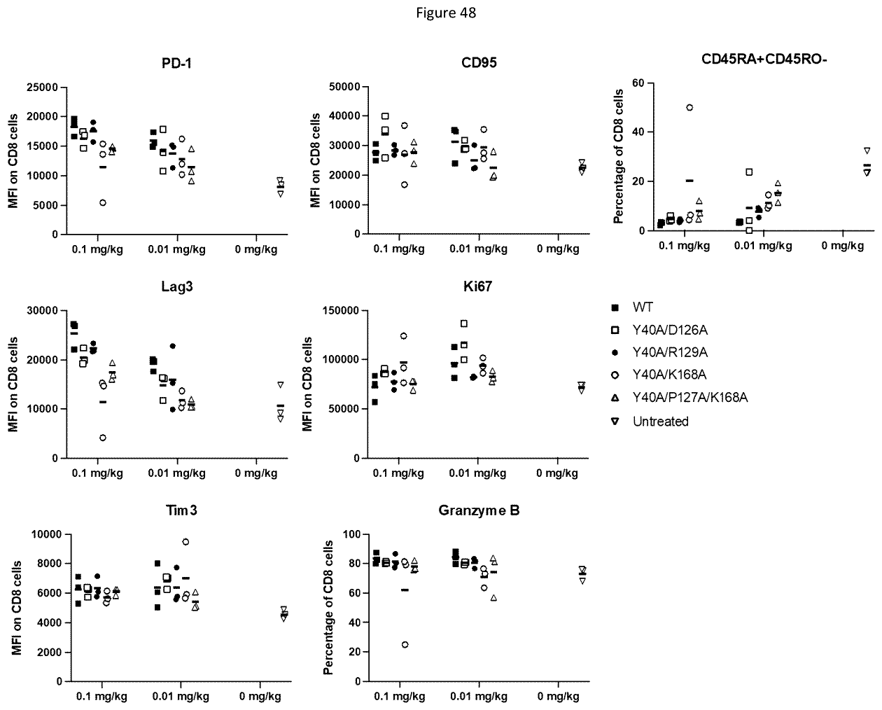

Data depicting the phenotype of CD4+ and CD8+ cells in the plasma of mice on day 12 are shown in FIGS. 47 and 48. The expression of markers associated with T cell activation (granzyme B) and exhaustion (PD-1, Lag-3, and Tim-3) all demonstrated a direct correlation with IL-12 potency, whereby “higher potency” IL-12 Fc variants, such as, for example, Y40A/D126A and Y40A/R129A, were associated with relatively higher expression (as determined by MFI or percentage positive cells) of these markers and “lower potency” IL-12 Fc variants, such as, for example, Y40A/K168A and Y40A/P127A/K168A, were associated with relatively lower expression of these markers. Similar to the results observed in the in vitro culture experiments (see, e.g., Example 9), “higher potency” IL-12 Fc variants were also associated with a shift from naive to activated/memory phenotype as determined by a decrease in the proportion of CD45RA+CD45RO− cells, which was particularly evident in the 0.01 mg/kg dose. Overall, these data indicated that “lower potency” IL-12 variants are associated with reduced T cell activation and proliferation in the blood, and thus may be useful for reducing toxicities associated with systemic IL-12 delivery.

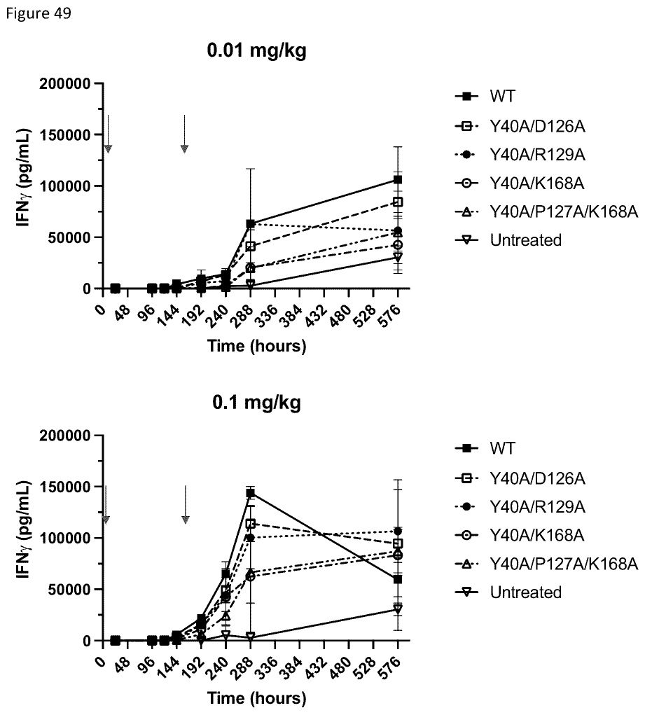

Data depicting plasma IFNγ concentrations (as determined by Luminex assay) are shown in FIG. 49. At a dose of 0.1 mg/kg, IFNγ levels in the plasma peaked at 288 hours, and then declined significantly by the next measured time point (576 hours). In contrast, mice injected with reduced potency IL-12 Fc variants exhibited a different pharmacodynamic profile, whereby IFNγ levels at 288 hours were lower than that observed for wild-type IL-12 Fc and correlated with IL-12 potency (Y40A/D126A approximately 79% of wild-type; Y40A/R129A approximately 69% of wild-type; Y40A/K168A approximately 43% of wild-type; Y40A/P127A/K168A approximately 44% of wild-type). Moreover, instead of declining, IFNγ levels in the plasma of mice treated with reduced potency IL-12 Fc variants were maintained or, in some cases, were slightly elevated at 576 hours, indicating a prolonged duration of action.

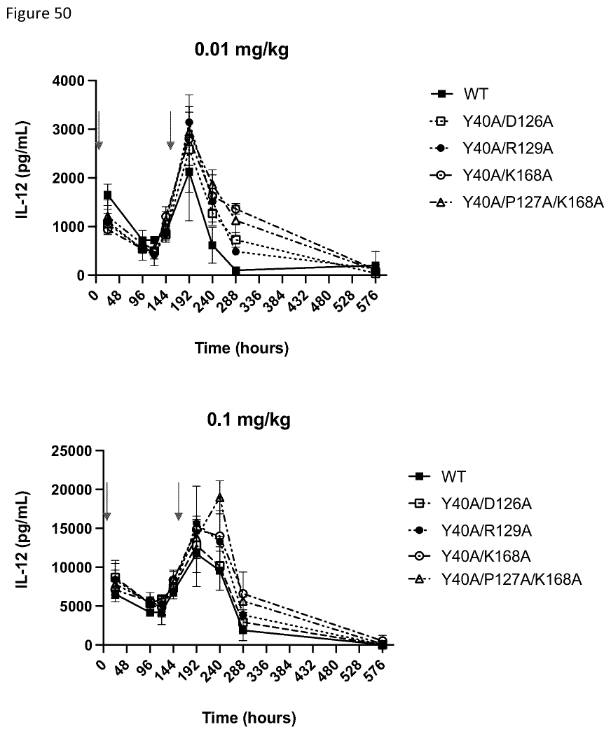

Data depicting plasma IL-12 Fc concentrations (as determined by Luminex assay detecting IL-12p40) are shown in FIG. 50. Wild-type IL-12 Fc levels rose sharply immediately following administration of the second dose on day 7 (168 hours), peaking at between 192-240 hours, but then declined sharply thereafter, presumably due to target-mediated drug disposition associated with the rapid expansion of alloreactive T cells. In contrast, the reduced potency IL-12 Fc variants exhibited a higher peak at 192-240 hours and exhibited a more gradual subsequent reduction in IL-12 Fc plasma concentration. The extent of this effect correlated with potency, whereby variants that exhibited a greater reduction in potency (Y40A/K168A and Y40A/P127A/K168A) displayed the most elevated IL-12 Fc plasma levels, and variants that exhibited a more modest reduction in potency (Y40A/D126A and Y40A/R129A) displayed intermediate IL-12 Fc plasma levels. Overall, these data demonstrate that reduced potency IL-12 Fc variants exhibit several desirable properties in vivo, including, but not limited to, reducing toxicities associated with systemic T cell activation, extended plasma half-life, and increased duration of action. These properties may ultimately enhance use for the treatment of human diseases, such as, for example, cancer.

### K. Example 11—In Vivo Anti-Tumor Activity of Reduced Potency IL-12 Fc Mutants

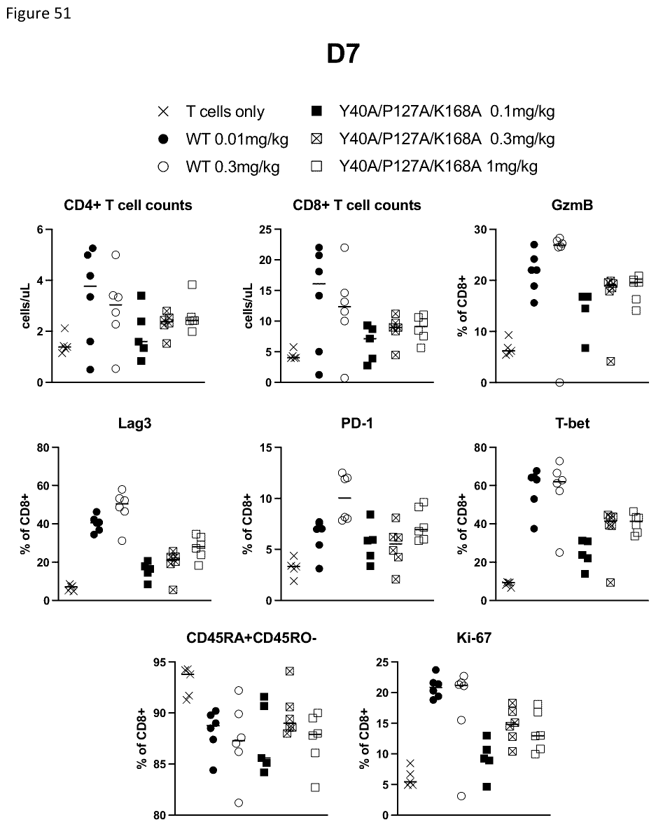

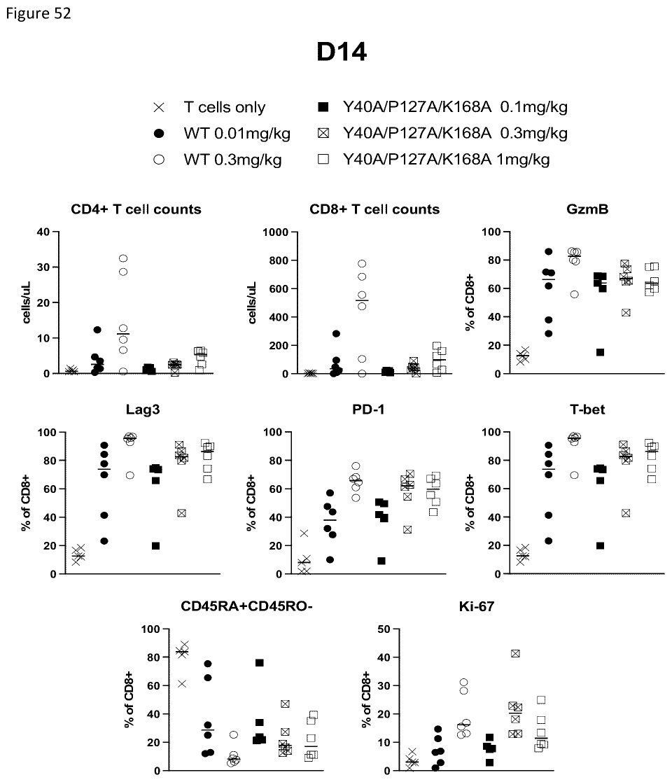

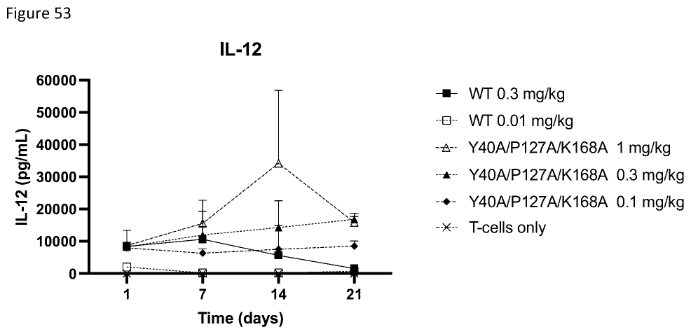

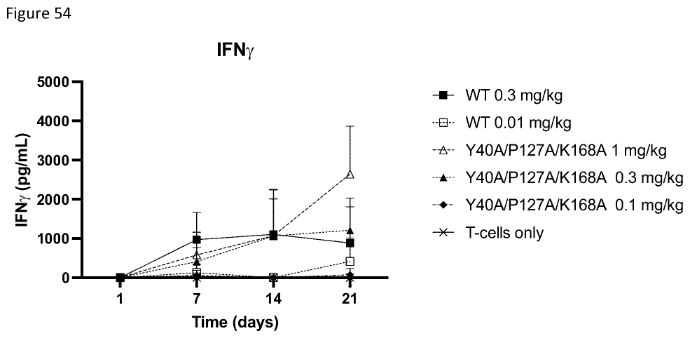

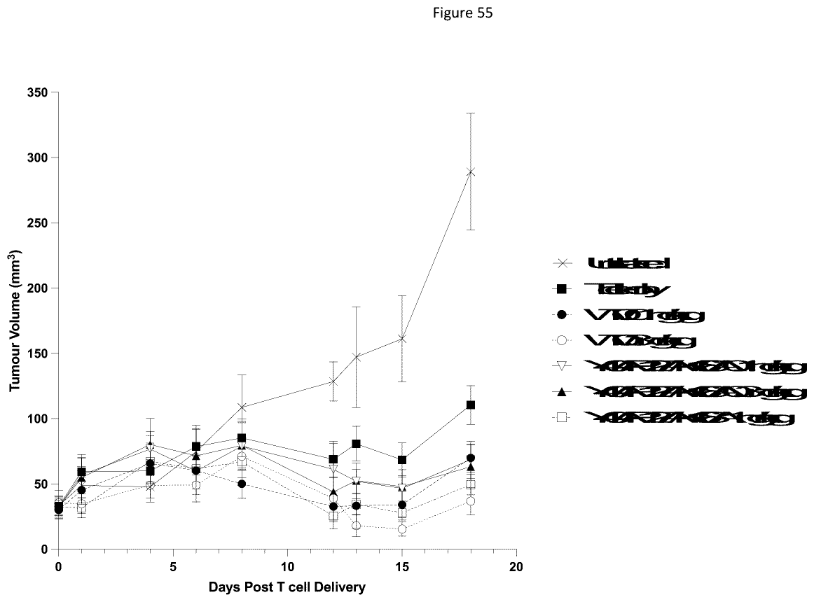

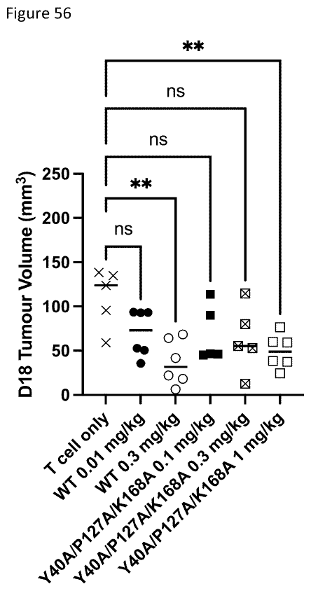

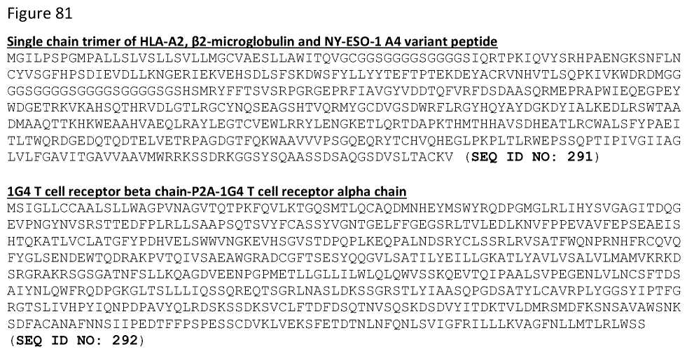

The anti-tumor activity of an exemplary reduced potency IL-12 Fc mutant (Y40A/P127A/K168A) was subsequently investigated in vivo using a human T cell adoptive transfer model. Female NSG mice (n=5-6 per treatment group) were injected subcutaneously with 2×10⁵ HCT116 human colorectal carcinoma cells that had been engineered to express a single chain trimer of HLA-A2, P2-microglobulin and a variant (A4) NY-ESO-1 peptide (SEQ ID NO: 291, as shown in FIG. 81). Fourteen days later (day 0), mice were subjected to 1 Gy total body irradiation and intravenously injected with 2.5×10⁶ PBMC-derived primary T cells that had been activated with 600 U/ml IL-2 and 30 ng/ml anti-CD3 (clone OKT3) and transduced with a high affinity variant of the HLA-A2/NY-ESO-1 restricted 1G4 T cell receptor (TCR) (SEQ ID NO: 292, as shown in FIG. 81). To prevent graft versus host disease, the expression of endogenous TCRα within T cells was ablated prior to transduction through electroporation-mediated delivery of recombinant Cas9/sgRNA ribonucleoprotein targeting the TRAC gene. In addition to adoptive T cell therapy, some mice received intraperitoneal injections of wild type IL-12 Fc (0.01 or 0.3 mg/kg) or IL-12 Fc Y40A/P127A/K168A mutant (0.1, 0.3, or 1 mg/kg) on days 0, 7, 14, and 21. Blood was sampled from the mice on day 1 and then immediately prior to each subsequent administration of IL-12 Fc to allow for analysis of immune cell phenotype and plasma IL-12 and IFNγ levels via Luminex assay, the results of which are depicted in FIGS. 51-54. Tumor growth curves are depicted in FIG. 55 and a summary of tumor sizes recorded on day 18 is provided in FIG. 56.

Overall, the data demonstrates that the reduced potency IL-12 Fc Y40A/P127A/K168A mutant was able to significantly reduce tumor burden in mice compared to the T cell-only control. In addition, the reduced potency IL-12 Fc mutant exhibited a favorable pharmacokinetic and pharmacodynamic profile relative to WT IL-12 Fc. For example, despite the weekly dosing regimen, the plasma concentration of WT IL-12 Fc decreased throughout the time course of the experiment, which was in direct contrast to the Y40A/P127A/K168A mutant, whose circulating levels were maintained up to day 21. Moreover, on day 7, the Y40A/P127A/K168A mutant resulted in lower levels of IFNγ in the plasma, and reduced the expansion and activation of peripheral T cells, as evidenced by CD4+ and CD8+ cell numbers and changes in the expression of activation (T-bet), cytotoxicity (granzyme B), proliferation (Ki-67), and maturation and exhaustion (CD45RA⁺CD45RO⁻, Lag3, PD-1) markers in CD8+ T cells relative to mice treated with WT IL-12 Fc. Although plasma IFNγ levels and CD8+ T cell activation status in mice treated with the IL-12 Fc Y40A/P127A/K168A mutant did increase at later time points, altogether these data indicate that the reduced potency IL-12 Fc variants of the invention possess an improved toxicity profile and could be dosed at less frequent intervals than WT IL-12 Fc. These characteristics would be favorable for the treatment of human disease in a clinical setting.

### L. Example 12—Additional (Non-Alanine) IL-12 Fc Mutants

While alanine mutations at positions 40, 126, 127, 129, 168, and 170 of IL-12 (p35) presumably reduce its potency via the loss of molecular interactions with IL-12Rβ2, the introduction of other (non-alanine) amino acids at these same positions could also potentially impact IL-12 potency to a greater or lesser extent. For example, the introduction of large or bulky residues into IL-12p35 could sterically impede interaction with IL-12Rβ2, whereas charged side chains could contribute attractive or repulsive forces. However, in the absence of a high-resolution structure of the IL-12/IL-12Rβ2 complex, the functional effect of IL-12p35 substitutions are difficult to predict. Accordingly, a series of IL-12 Fc mutants in which positions 40, 126, 127, 129, 168, and 170 of the p35 subunit domain were individually mutated to each of the 19 possible amino acids (collectively termed “non-alanine mutants”) were generated (in addition to the alanine mutants). Because the effect of alanine substitutions at D126, P127, R129, and K168 were relatively modest, mutations at these positions were incorporated into a p35 subunit domain that further comprised a Y40A substitution. IL-12 Fc “non-alanine mutants”’ were introduced into the same IL-12 Fc configuration as described in Example 8 with the exception that additional mutations were introduced into the IL-12p35 subunit domain (C74S) and IL-12p40 subunit domain (C177S) to remove the inter-chain disulfide bond between the subunits, as these modifications were observed to improve yield without affecting activity or stability of IL-12.

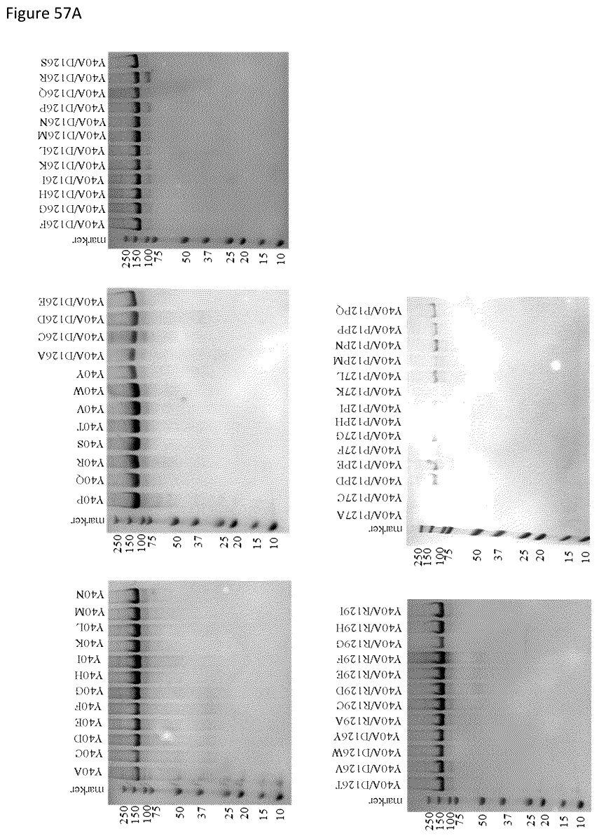

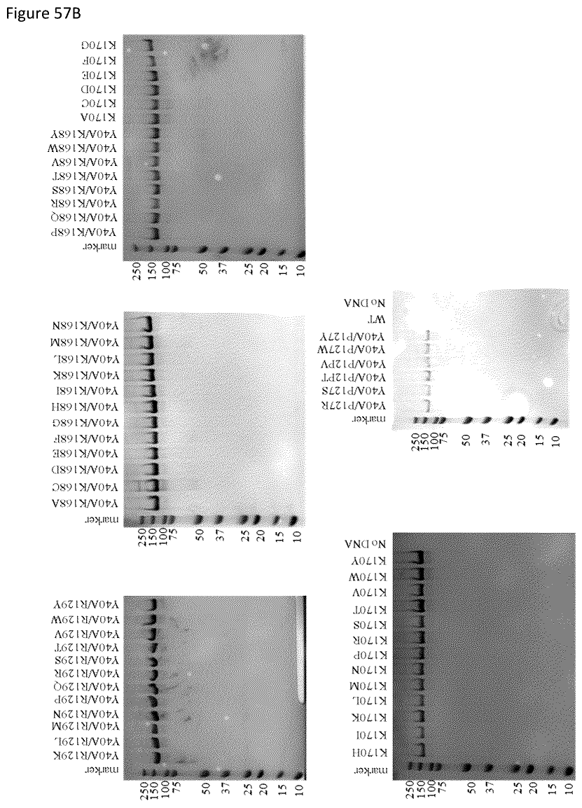

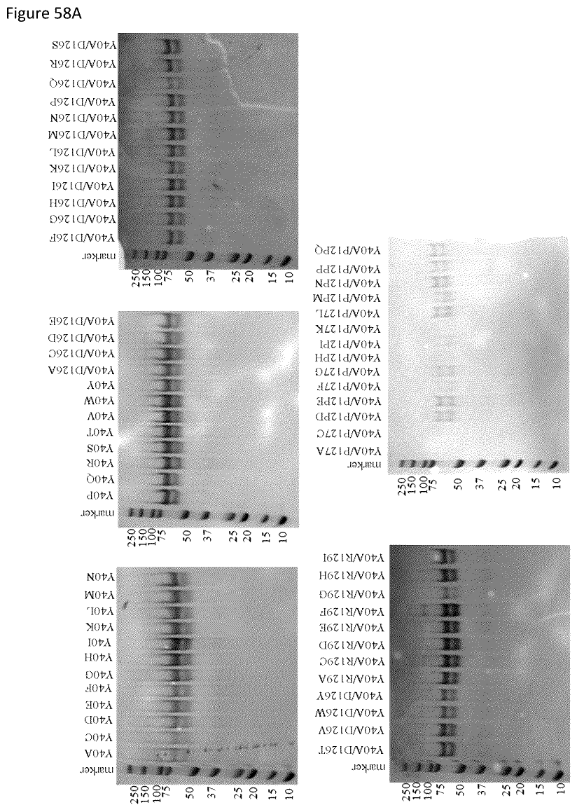

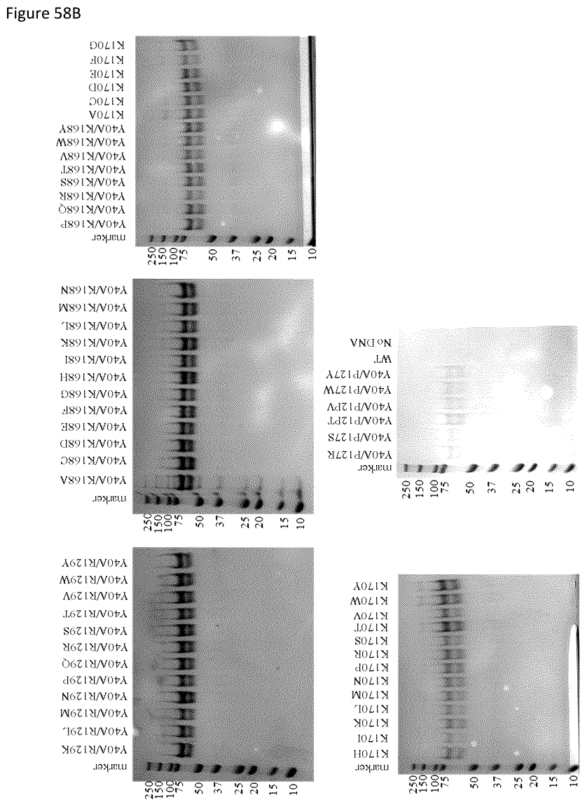

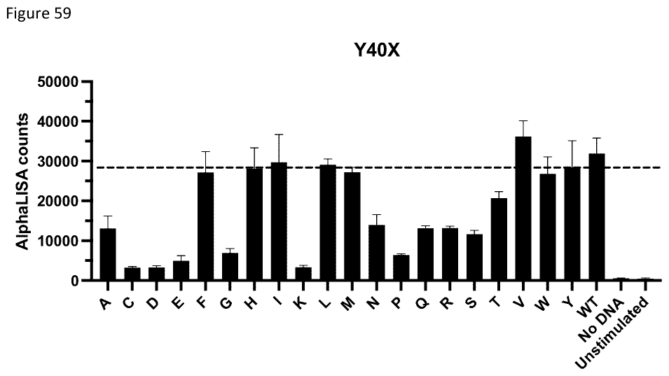

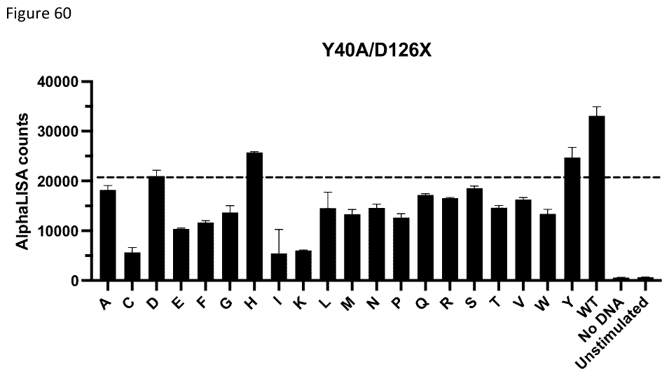

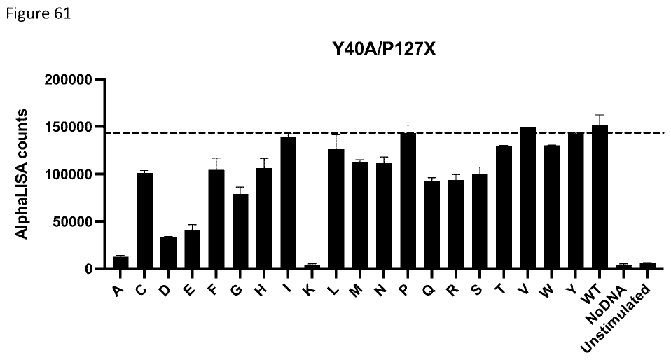

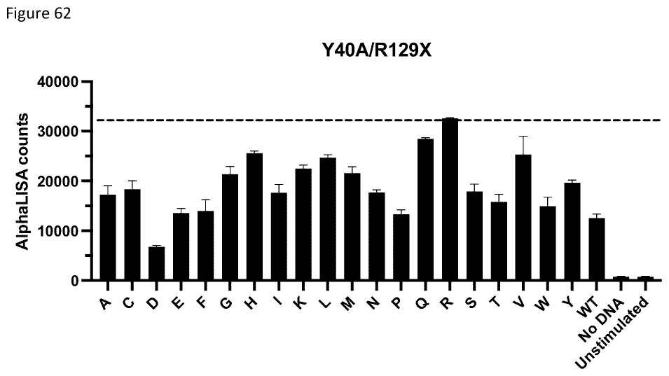

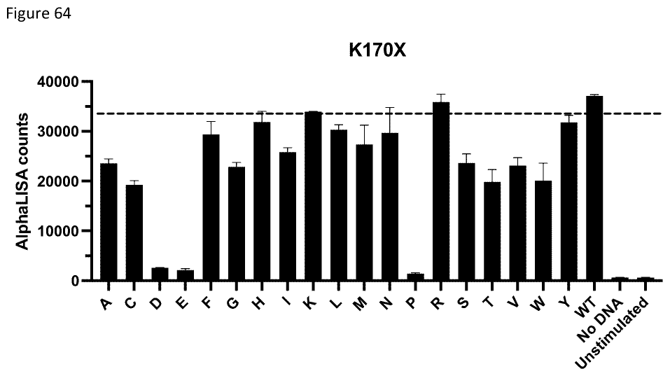

IL-12 Fc “non-alanine mutants” were expressed by transient transfection of 2.5 mL expi-CHO cell cultures, purified in batch mode using protein A sepharose resin, and quantified by AlphaLISA human IgG Fc detection kit. The integrity of each of the non-alanine mutants was also visually verified using SDS-PAGE (see, e.g., FIGS. 57 and 58). To provide a measure of potency, a single concentration (20 pM for Y40X and K170X, and 80 pM for Y40A/D126X, Y40A/R129X, and Y40A/K168X, where “X” indicates any amino acid (with the exception of K170Q)) of each purified IL-12 Fc “non-alanine mutant” was incubated with activated primary T cells, as described in Example 5, and IFNγ secretion was detected using AlphaLISA. Purified wild-type IL-12 Fc, as well as an unstimulated control, and a “No DNA” sample that represented conditioned expi-CHO media that had been subjected to the same purification procedure as the “non-alanine mutants” were included as additional controls. Plots of AlphaLISA counts (as a readout of IFNγ production) are depicted in FIGS. 59-64. Overall, distinct “non-alanine mutants” were observed to exhibit a range of different apparent activities. For position 40, amino acid substitutions that reduced activity compared to Y40Y were: (i) Ala (A; alanine), (ii) Cys (C; cysteine), (iii) Asp (D; aspartic acid), (iv) Glu (E; glutamic acid), (v) Gly (G; glycine), (vi) Lys (K; lysine), (vii) Asn (N; asparagine), (viii) Pro (P; proline), (ix) Gln (Q; glutamine), (x) Arg (R; arginine), (xi) Ser (S; serine), and (xii) Thr (T; threonine). For position 126, amino acids that reduced activity compared to Y40A/D126D were: (i) Ala, (ii) Cys, (iii) Glu, (iv) Phe (F; phenylalanine), (v) Gly, (vi) Ile (I; isoleucine), (vii) Lys, (viii) Leu (L; leucine), (ix) Met (M; methionine), (x) Asn, (xi) Pro, (xii) Gln, (xiii) Arg, (xiv) Ser, (xv) Thr, (xvi) Val (V; valine), and (xvii) Trp (W; tryptophan). For position 127, amino acids that reduced activity compared to Y40A/P127P were: (i) Ala, (ii) Cys, (iii) Asp, (iv) Glu, (v) Phe, (vi) Gly, (vii) His (H; histidine), (viii) Lys, (iv) Met, (x) Asn, (xi) Gln, (xii) Arg, and (xiii) Ser. For position 129, amino acids that reduced activity compared to Y40A/R129R were: (i) Ala, (ii) Cys, (iii) Asp, (iv) Glu, (v) Phe, (vi) Gly, (vii) His, (viii) Ile, (ix) Lys, (x) Leu, (xi) Met, (xii) Asn, (xiii) Pro, (xiv) Gln, (xv) Ser, (xvi) Thr, (xvii) Val, (xviii) Trp, and (xix) Tyr (Y). For position 168, amino acids that reduced activity compared to Y40A/K168K were: (i) Ala, (ii) Cys, (iii) Asp, (iv) Glu, (v) Phe, (vi) Gly, (vii) His, (viii) Ile, (ix) Leu, (x) Met, (xi) Asn, (xii) Pro, (xiii) Gln, (xiv) Ser, (xv) Thr, (xvi) Trp, and (xvii) Tyr. For position 170, amino acids that reduced activity compared to K170K were: (i) Ala, (ii) Cys, (iii) Asp, (iv) Glu, (v) Phe, (vi) Gly, (vii) Ile, (viii) Leu, (ix) Met, (x) Asn, (xi) Pro, (xii) Ser, (xiii) Thr, (xiv) Val, and (xv) Trp.

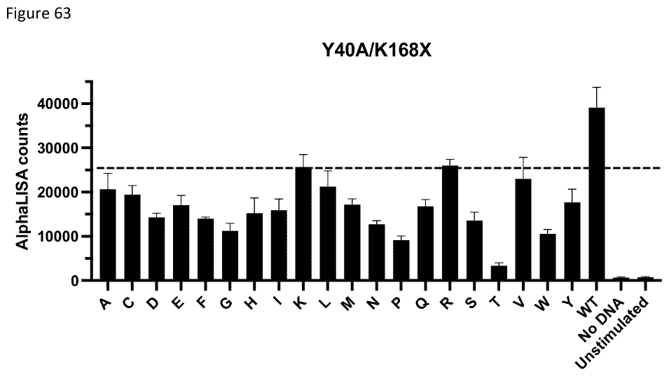

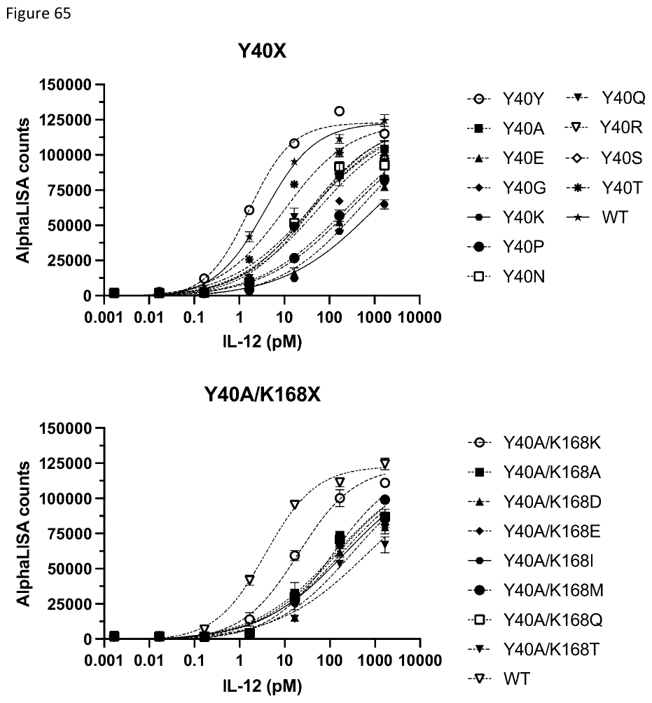

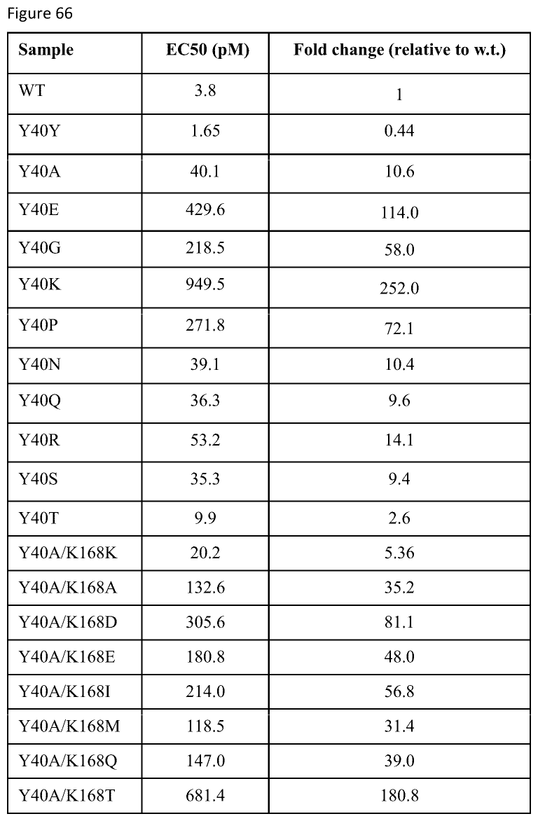

To further confirm the potency of the non-alanine mutants, IFNγ production from T cells exposed to several concentrations of selected IL-12 Fc non-alanine mutants (including Y40X single mutants and Y40A/K168X double mutants (where X indicates a variable amino acid)) was assessed. IFNγ production was measured via human IFNγ AlphaLISA Detection Kit. Plots of AlphaLISA counts are depicted in FIG. 65 and a summary of calculated EC50 values are provided in FIG. 66. Overall, each of the mutants tested exhibited weaker potency than WT IL-12 Fc, and the fold change in EC₅₀ values directly correlated with the magnitude of potency reduction observed when mutants were tested at a single concentration (data depicted in FIGS. 59 and 63), validating the initial approach to screening the potency of the non-alanine mutants.

### M. Example 13—Thermal Stability of IL-12 Fc Mutants

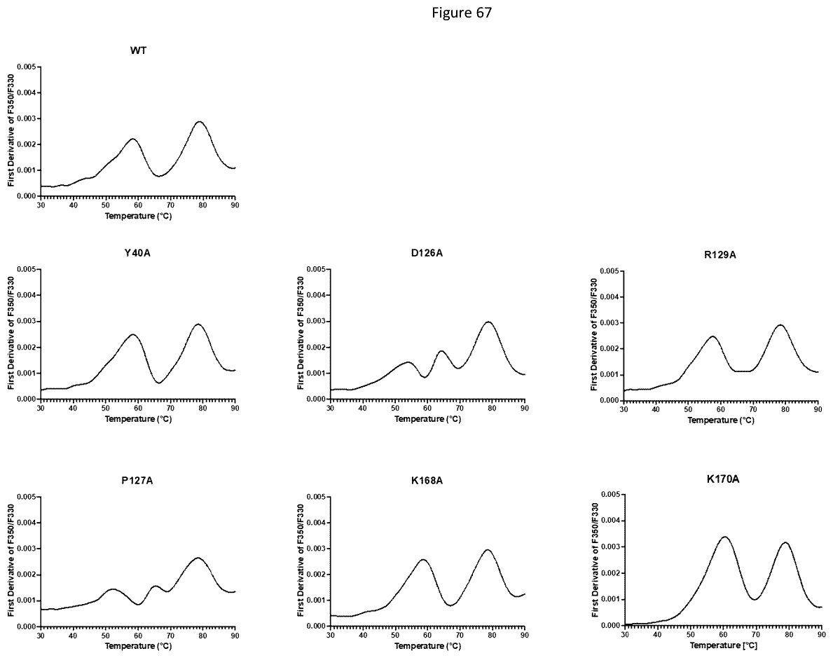

To investigate whether alanine substitutions at positions 40, 126, 127, 129, 168, or 170 impacted the stability of the IL-12p35 subunit, differential scanning fluorimetry (DSF) was performed to measure thermally induced protein unfolding using a NanoTemper Prometheus nano DSF. Wild type IL-12 Fc and IL-12 Fc harboring individual alanine mutations at either position 40, 126, 127, 129, 168, or 170 were heated to 110° C. at a rate of 1° C./min at a concentration of 0.2-0.4 mg/mL in a buffer comprising 20 mM sodium phosphate pH 7.5 containing 150 mM NaCl. Data depicting the first derivative of the F350:F330 ratio as a function of temperature is depicted in FIG. 67. Wild type IL-12 Fc was observed to undergo two distinct thermal unfolding events, with the first (melting temperature (T_{M}) approximately 60° C.) likely corresponding to the IL-12 subunit and the second (T_{M} approximately 80° C.) likely corresponding to the Fc subunit. While several of the alanine single mutants (including Y40A, R129A, K168A, and K170A) exhibited a thermal unfolding profile similar to wild type IL-12 Fe, introduction of D126A or P127A altered the stability of the IL-12 heterodimer, which was now observed to undergo two distinct thermal unfolding events with melting temperatures of approximately 55° C. (IL-12p35 subunit) and 64° C. (IL-12p40 subunit). Thus, alanine substitutions at positions D126 or P127 reduce the thermal stability of the IL-12p35 subunit, whereas alanine substitutions at positions Y40, R129, K168, and K170 of IL-12p35 do not adversely impact the stability of IL-12.

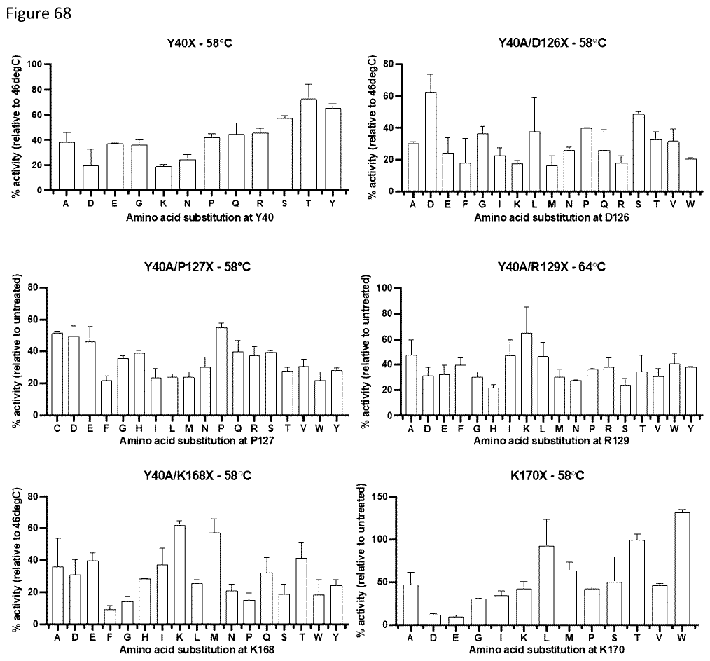

The thermal stability of selected non-alanine IL-12 mutants produced in example 12 was subsequently assessed using a functional readout. Here, each test article was heat treated at either 46, 52, 58, 64, 70, or 76° C. for 15 minutes and the capacity of each treated sample to stimulate IFNγ production by activated primary T cells was measured. Following baseline subtraction using the PBS control sample, AlphaLISA counts (as a readout of IFNγ) for each test article/treatment condition were plotted as a percentage relative to their value at 46° C. or in the untreated sample. Data for selected temperature steps are shown in FIG. 68. Pre-treatment with increasing temperature resulted in a reduction in T cell IFNγ production across all samples.

However, the rate at which IFNγ levels declined with increasing temperature varied dramatically amongst the various mutants that were examined. Among the samples tested, those that were considered thermostable were: Y40A, Y40E, Y40G, Y40P, Y40Q, Y40R, Y40S, Y40T, D126G, D126L, D126P, D126S, D126T, D126V, P127C, P127D, P127E, R129A, R129D, R129E, R129F, R129I, R129K, R129L, R129P, R129Q, R129T, R129W, R129Y, K168A, K168D, K168E, K168I, K168M, K168Q, K168T, K170A, K170L, K170M, K170P, K170S, K170T, and K170W.

### N. Example 14—Thermostable IL-12 Fc Variants

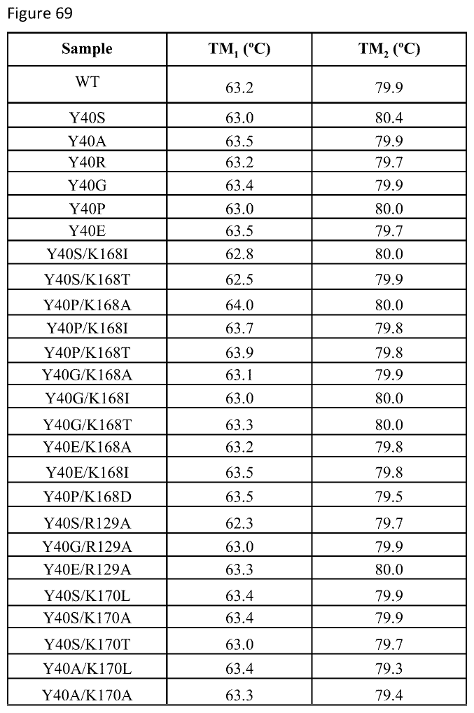

Following the experiments described in example 13, twenty-five distinct IL-12 single or double mutants that were (1) hypothesized to span a range of potencies and (2) anticipated to be thermally stable were selected for further in-depth characterization. These are collectively referred to as the “thermostable mutant series.” The “thermostable mutant series” were expressed and purified as described in example 8 and subjected to DSF analysis, the results of which are summarized in FIG. 69. Each member of the “thermostable mutant series” exhibited a thermal unfolding profile that was essentially identical to wild type IL-12 Fe, consisting of a first unfolding event (TM₁) occurring at approximately 63° C. and a second unfolding event (TM₂) occurring at approximately 80° C. These data corroborated the findings demonstrated in example 13 with respect to thermal stability and indicated that combining two individual thermostable mutants to produce a double mutant does not adversely impact the thermostability of IL-12.

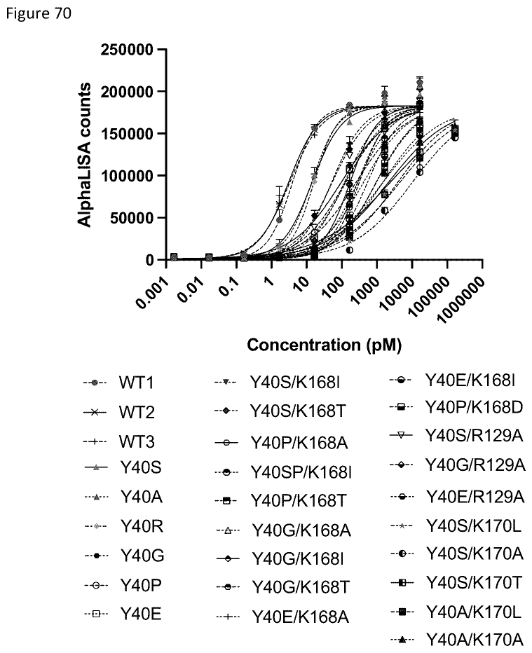

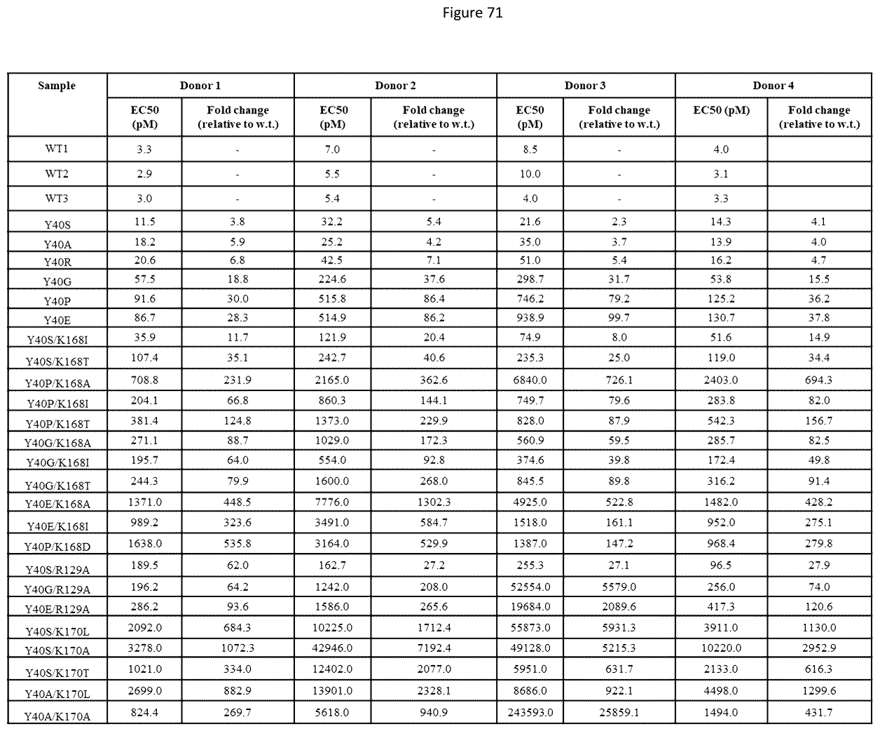

Next, the potency of individual members of the “thermostable mutant series” was assessed using the primary T cell IFNγ assay. A representative example of results from a single donor is shown in FIG. 70 and data from four independent donors are summarized in FIG. 71. All members of the “thermostable mutant series” exhibited reduced potency relative to wild type IL-12 Fc, with a range spanning from approximately 3-fold lower than wild type IL-12 Fc (for Y40S), to approximately 4,100-fold lower than wild type IL-12 Fc (for Y40S/K170A).

### O. Example 15—IL-12 Fc Mutants Enhance T Cell Killing o Cancer Cells In Vitro

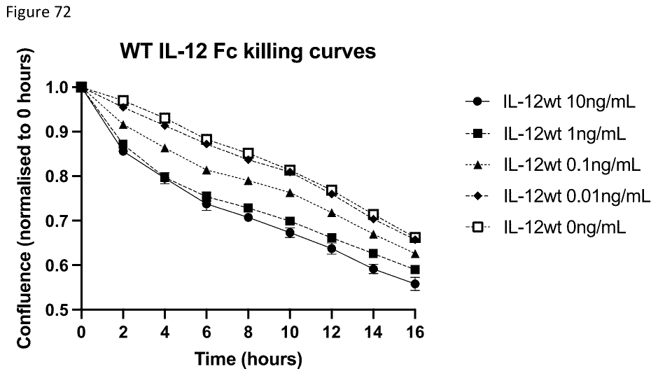

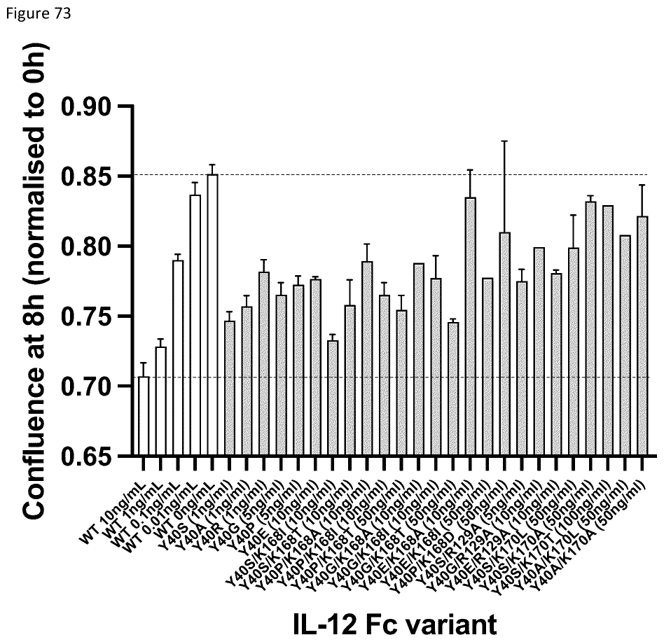

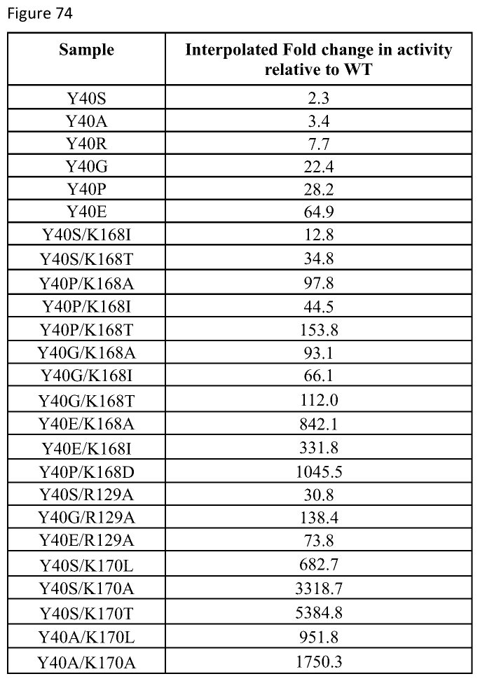

To further investigate the functional characteristics of select IL-12 Fc mutants, an in vitro T cell killing assay was performed. Here, 1G4 transduced, T cell receptor alpha chain constant region (TRAC) knockout primary T cells were pre-treated overnight with 50 U/mL of IL-2 and either 0.01-10 ng/mL of wild type IL-12 Fc or each of the “thermostable mutant series” at a single defined concentration that was selected according to their expected potency. The following day, T cells were resuspended in medium containing 50 U/mL IL-2 and added to wells containing 12,500 HCT-116 cells that expressed the HLA-A2 NY-ESO-1 A4 variant single chain trimer and green fluorescent protein (GFP) adhered overnight at an effector:target (E:T) ratio of 2.5:1. Cell mixtures were placed into an Incucyte and the confluence of live HCT-116 cells was monitored by quantifying GFP intensity over a 16h period. Killing curves for the titration of wild type IL-12 Fc are shown in FIG. 72 and the quantification of GFP confluence for all tested samples at the 8h timepoint is represented in FIG. 73. Overall, pre-incubation with wild type IL-12 Fc resulted in a dose-dependent increase in T cell killing that was evident as early as 2h following co-culture and persisted throughout the 16h time frame of the experiment. To quantify the activity of the IL-12 Fc mutants, data for the known concentrations of wild type IL-12 Fc at the 8h time point was fitted to a 4-parameter logistic curve in GraphPad Prism. Using this standard curve, an interpolated concentration could then be calculated for each of the IL-12 Fc variants (using their observed confluency at the 8h time point). The fold reduction in potency of each IL-12 Fc variant (depicted in FIG. 74) was then calculated by dividing the known concentration of the IL-12 Fc variant by the interpolated concentration derived from the standard curve.

### P. Example 16—Additional Thermostable IL-12 Fc Mutants

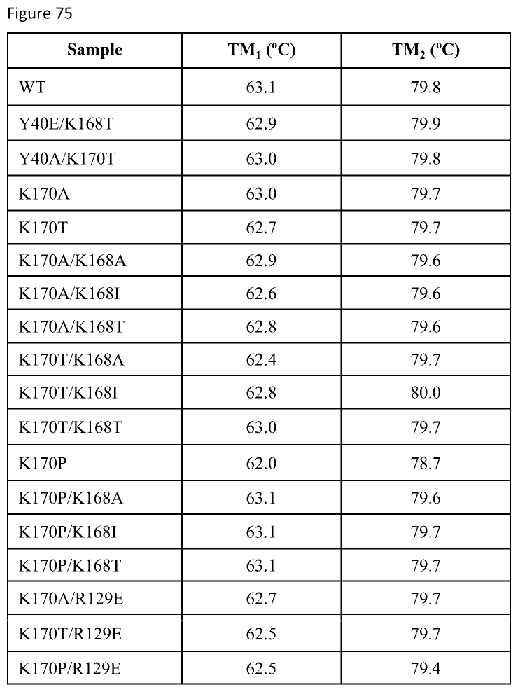

An additional set of IL-12 Fc mutants that were anticipated to be thermally stable (based on the data outlined in example 13) were subsequently designed. The additional IL-12 Fc mutants were expressed and purified as described in example 8 and subjected to DSF analysis, the results of which are summarized in FIG. 75. Each member of the IL-12 Fc mutants tested exhibited a thermal unfolding profile that was essentially identical to wild type IL-12 Fc, consisting of a first unfolding event (TM₁) occurring at approximately 63° C. and a second unfolding event (TM₂) occurring at approximately 80° C. Accordingly, these mutants were deemed to be thermally stable.

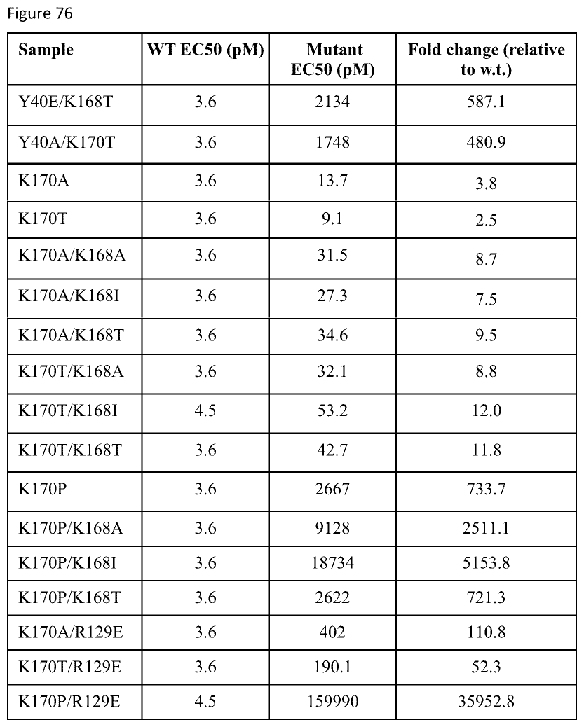

We also assessed the potency of the additional IL-12 Fc thermostable mutants using the primary T cell IFNγ assay, the data for which is summarized in FIG. 76. Each of the additional IL-12 Fc thermostable mutants exhibited reduced potency relative to wild type IL-12 Fc, with a range spanning from approximately 2.5-fold lower than wild type IL-12 Fc (for K170T), to approximately 35,953-fold lower than wild type IL-12 Fc (for K170P/R129E).

### Q. Example 17—Low Potency IL-12Fc Mutants in a p40-Fc(Knob)/p35-Fc(hole) Configuration

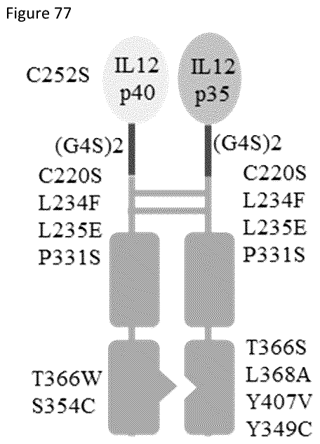

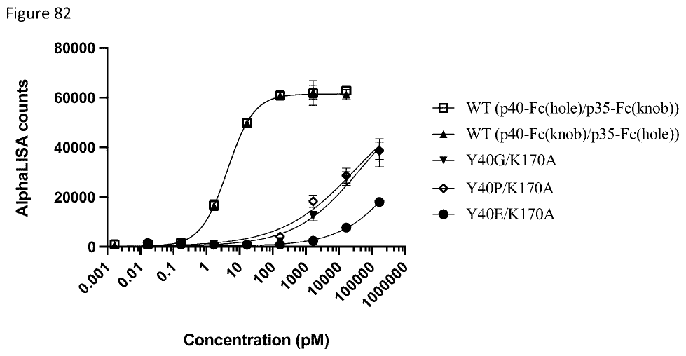

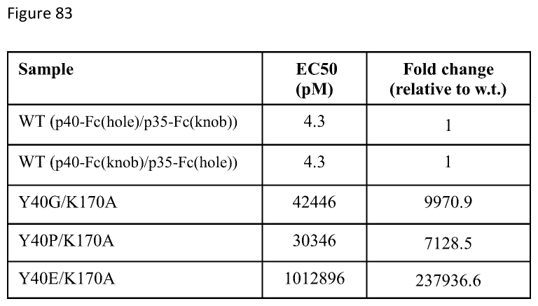

Subsequently, an additional group of IL-12p35 mutants (Y40G/K170A, Y40P/K170A, Y40E/K170A) in an alternative configuration was generated, whereby the p40 subunit was attached to the Fc chain containing knob mutations, and the p35 subunit was attached to the Fc chain containing the hole mutations (FIG. 77). As a control, wild type IL-12 Fc in the same p40-Fc(knob)/p35-Fc(hole) configuration was produced and the potency of this molecule to that of wild type IL-12 Fc in the p40-Fc(hole)/p35-Fc(knob) configuration was compared. Both of the wild type IL-12 Fc configurations tested (p40-Fc(knob)/p35-Fc(hole) and p40-Fc(hole)/p35-Fc(knob)) were observed to possess identical activity (EC₅₀ of 4.3 pM) in the T cell IFNγ assay (FIGS. 82 and 83). In addition, each of the IL-12p35 double mutants (Y40G/K170A, Y40P/K170A and Y40E/K170A) reduced the potency of the of IL-12 Fc within the context of the p40-Fc(knob)/p35-Fc(hole) configuration (FIGS. 82 and 83). Accordingly, three additional low potency IL-12 Fc mutants (with a range spanning approximately 7,128-fold to 237,937-fold weaker activity compared to wild type IL-12 Fc) were discovered and it was demonstrated that altering the IL-12 Fc configuration (p40-Fc(knob)/p35-Fc(hole) versus p40-Fc(hole)/p35-Fc(knob)) did not impact the functional attributes of the invention.

1.-268. (canceled)

269. A heterodimeric Fc fusion protein, comprising:
a) a first fusion construct, comprising: a variant IL-12p35 subunit domain, a first linker domain, and a first Fc domain, wherein:
i) the C-terminus of the variant IL-12p35 subunit domain is covalently attached to the N-terminus of the first linker domain and the C-terminus of the first linker domain is covalently attached to the N-terminus of the first Fc domain,
ii) the first linker domain comprises SEQ ID NO: 15,
iii) the first Fc domain comprises SEQ ID NO: 13, and
iv) the variant IL-12p35 subunit domain comprises one or more amino acid substitutions selected from the group consisting of: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E;
b) a second fusion construct, comprising: an IL-12p40 subunit domain, a second linker domain, and a second Fc domain, wherein:
i) the C-terminus of the IL-12p40 subunit domain is covalently attached to the N-terminus of the second linker domain and the C-terminus of the second linker domain is covalently attached to the N-terminus of the second Fc domain,
ii) the second linker domain comprises SEQ ID NO: 15,
iii) the second Fc domain comprises SEQ ID NO: 12, and
iv) the IL-12p40 subunit domain comprises SEQ ID NO: 89.

270. The heterodimeric Fc fusion protein of claim 269, wherein the variant IL-12p35 subunit domain further comprises a C74S substitution mutation.

271. The heterodimeric Fc fusion protein according to claim 270, wherein the IL-12p40 subunit domain further comprises a C177S substitution mutation.

272. The heterodimeric Fc fusion protein according to claim 269, wherein the IL-12p40 subunit domain further comprises a C177S substitution mutation.

273. The heterodimeric Fc fusion protein according to claim 269, wherein the first Fc domain and the second Fc domain comprise modifications that (i) promote heterodimerization of the first and second Fc domains and/or (ii) silence or inhibit effector function.

274. One or more nucleic acids encoding a heterodimeric Fc fusion protein according to claim 269.

275. A method of producing a heterodimeric Fc fusion protein, the method comprising:
culturing a host cell with one or more nucleic acids or vectors under conditions whereby the heterodimeric Fc fusion protein is produced, wherein the one or more nucleic acids or vectors comprises the one or more nucleic acids of claim 274,
wherein the produced heterodimeric Fc fusion protein has an increased half-life compared to half-life of a reference IL-12, wherein the reference IL-12 comprises one or more of: a wild-type IL-12, a human wild-type IL-12, a commercially available IL-12 molecule, or an IL-12 Fc fusion protein.

276. The method of claim 275, further comprising isolating and/or purifying the produced heterodimeric Fc fusion protein.

277. A non-naturally occurring IL-12 variant, comprising:
a) a variant IL-12p35 subunit, wherein the variant IL-12p35 subunit comprises a first amino acid substitution mutation, wherein the first amino acid substitution is selected from the group consisting of: Y40A, Y40E, Y40G, Y40P, Y40R, Y40S, K170A, K170P, K170T; and
b) an IL-12p40 subunit.

278. The non-naturally occurring IL-12 variant of claim 277, wherein:
i) the first amino acid substitution mutation is Y40A;
ii) the variant IL-12p35 subunit further comprises a second substitution mutation; and
iii) the second substitution mutation is selected from the group consisting of: K168A, K168D, K168E, K168I, K168M, K168Q, K168T, K170A, K170L, and K170T.

279. The non-naturally occurring IL-12 variant of claim 277, wherein:
i) the first amino acid substitution mutation is Y40E;
ii) the variant IL-12p35 subunit further comprises a second substitution mutation; and
iii) the second substitution mutation is selected from the group consisting of: K170A, K168A, K168I, K168T, and R129A.

280. The non-naturally occurring IL-12 variant of claim 277, wherein:
i) the first amino acid substitution mutation is Y40G;
ii) the variant IL-12p35 subunit further comprises a second substitution mutation; and
iii) the second substitution mutation is selected from the group consisting of: K170A, K168A, K168I, K168T, and R129A.

281. The non-naturally occurring IL-12 variant of claim 277, wherein:
i) the first amino acid substitution mutation is Y40P;
ii) the variant IL-12p35 subunit further comprises a second substitution mutation; and
iii) the second substitution mutation is selected from the group consisting of: K170A, K168A, K168D, K168I, and K168T.

282. The non-naturally occurring IL-12 variant of claim 277, wherein:
i) the first amino acid substitution mutation is Y40S;
ii) the variant IL-12p35 subunit further comprises a second substitution mutation; and
iii) the second substitution mutation is selected from the group consisting of: K168I, K168T, K170A, K170L, K170T, and R129A.

283. The non-naturally occurring IL-12 variant of claim 277, wherein:
i) the first amino acid substitution mutation is K170A;
ii) the variant IL-12p35 subunit further comprises a second substitution mutation; and
iii) the second substitution mutation is selected from the group consisting of: K168A, K168I, K168T, and R129E.

284. The non-naturally occurring IL-12 variant of claim 277, wherein:
i) the first amino acid substitution mutation is K170P;
ii) the variant IL-12p35 subunit further comprises a second substitution mutation; and
iii) the second substitution mutation is selected from the group consisting of: K168A, K168I, K168T, and R129E.

285. The non-naturally occurring IL-12 variant of claim 277, wherein:
i) the first amino acid substitution mutation is K170T;
ii) the variant IL-12p35 subunit further comprises a second substitution mutation; and
iii) the second substitution mutation is selected from the group consisting of: K168A, K168I, K168T, and R129E.

286. The non-naturally occurring IL-12 variant of claim 277, wherein the variant IL-12p35 subunit comprises any one of SEQ ID NOs: 24, 34, 103, 104, 109, 179, 180, 183, 185, 186, 194, 196, 233, 234, 238, 240, 243, 245, and 248-278.

287. The non-naturally occurring IL-12 variant of claim 277, wherein the variant IL-12p35 subunit domain further comprises a C74S substitution mutation.

288. The non-naturally occurring IL-12 variant of claim 277, wherein the IL-12p40 subunit comprises a variant IL-12p40 subunit, wherein the variant IL-12p40 subunit comprises one or more amino acid substitutions selected from the group consisting of: C177S, C252S, and C177S/C252S.

289. The non-naturally occurring IL-12 variant of claim 277, wherein the IL-12p40 subunit comprises any one of SEQ ID NOs: 4, 88, 89, and 90.

290. The non-naturally occurring IL-12 variant of claim 277, wherein the non-naturally occurring IL-12 variant further comprises one or more of the following fused to the variant IL-12p35 subunit and/or the IL-12p40 subunit: (i) a Fc domain, wherein the Fc domain comprises one or more amino acid sequences selected from the group consisting of: SEQ ID NO: 9, SEQ ID NO: 10, SEQ ID NO: 11, SEQ ID NO: 12, and SEQ ID NO: 13, (ii) an albumin, (iii) one or more unstructured biodegradable polypeptides (“XTEN”), or (iv) a polyethylene glycol (PEG).

291. The non-naturally occurring IL-12 variant of claim 277, wherein the C-terminus of the variant IL-12p35 subunit is covalently attached to the N-terminus of the IL-12p40 subunit.

292. The non-naturally occurring IL-12 variant of claim 291, wherein the non-naturally occurring IL-12 variant further comprises a linker domain comprising an amino acid sequence selected from the group consisting of: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the variant IL-12p35 subunit is covalently attached to the N-terminus of the linker domain and the C-terminus of the linker domain is covalently attached to the N-terminus of the IL-12p40 subunit.

293. The non-naturally occurring TL-12 variant of claim 291, wherein the non-naturally occurring IL-12 variant further comprises a Fc domain, wherein the Fc domain comprises one or more amino acid sequences selected from the group consisting of: SEQ ID NO: 9, SEQ ID NO: 10, SEQ ID NO: 11, SEQ ID NO: 12, and SEQ ID NO: 13, and the Fc domain is fused to the variant IL-12p35 subunit or the IL-12p40 subunit.

294. The non-naturally occurring IL-12 variant of claim 293, wherein the non-naturally occurring IL-12 variant is fused to the Fc domain using a linker.

295. The non-naturally occurring IL-12 variant of claim 277, wherein the C-terminus of the IL-12p40 subunit is covalently attached to the N-terminus of the variant IL-12p35 subunit.

296. The non-naturally occurring IL-12 variant of claim 295, wherein the non-naturally occurring IL-12 variant further comprises a linker domain comprising an amino acid sequence selected from the group consisting of: SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23, and wherein the C-terminus of the IL-12p40 subunit is covalently attached to the N-terminus of the linker domain and the C-terminus of the linker domain is covalently attached to the N-terminus of the variant IL-12p35 subunit.

297. The non-naturally occurring IL-12 variant of claim 295, wherein the non-naturally occurring IL-12 variant further comprises a Fc domain, wherein the Fc domain comprises one or more amino acid sequences selected from the group consisting of: SEQ ID NO: 9, SEQ ID NO: 10, SEQ ID NO: 11, SEQ ID NO: 12, and SEQ ID NO: 13, and the Fc domain is fused to the variant IL-12p35 subunit or the IL-12p40 subunit.

298. The non-naturally occurring IL-12 variant of claim 297, wherein the non-naturally occurring IL-12 variant is fused to the Fc domain using a linker.

299. The non-naturally occurring IL-12 variant of claim 277, wherein the variant IL-12p35 subunit comprises one or more additional amino acid substitutions.

300. One or more nucleic acids encoding a non-naturally occurring IL-12 variant according to claim 277.

301. A method for treating a cancer in a subject, comprising:
administering to the subject a therapeutically effective amount of a non-naturally occurring IL-12 variant, wherein the non-naturally occurring IL-12 variant comprises:
a) a variant IL-12p35 subunit, wherein the variant IL-12p35 subunit comprises one or more amino acid substitutions selected from the group consisting of: Y40A, Y40A/K168A, Y40A/K168D, Y40A/K168E, Y40A/K168I, Y40A/K168M, Y40A/K168Q, Y40A/K168T, Y40A/K170A, Y40A/K170L, Y40A/K170T, Y40E, Y40E/K170A, Y40E/K168A, Y40E/K168I, Y40E/K168T, Y40E/R129A, Y40G, Y40G/K170A, Y40G/K168A, Y40G/K168I, Y40G/K168T, Y40G/R129A, Y40P, Y40P/K170A, Y40P/K168A, Y40P/K168D, Y40P/K168I, Y40P/K168T, Y40R, Y40S, Y40S/K168I, Y40S/K168T, Y40S/K170A, Y40S/K170L, Y40S/K170T, Y40S/R129A, K170A, K170A/K168A, K170A/K168I, K170A/K168T, K170A/R129E, K170P, K170P/K168A, K170P/K168I, K170P/K168T, K170P/R129E, K170T, K170T/K168A, K170T/K168I, K170T/K168T, and K170T/R129E; and
b) an IL-12p40 subunit.

302. A host cell comprising one or more nucleic acids or vectors encoding a non-naturally occurring IL-12 variant, wherein the non-naturally occurring IL-12 variant comprises:
(i) a variant IL-12p35 subunit, wherein the variant IL-12p35 subunit comprises a first amino acid substitution mutation, wherein the first amino acid substitution is selected from the group consisting of: Y40A, Y40E, Y40G, Y40P, Y40R, Y40S, K170A, K170P, K170T; and
(ii) an IL-12p40 subunit.

303. The host cell according to claim 302, wherein the variant IL-12p35 subunit comprises any one of SEQ ID NOs: 24, 34, 103, 104, 109, 179, 180, 183, 185, 186, 194, 196, 233, 234, 238, 240, 243, 245, and 248-278.

304. The host cell according to claim 302, wherein the variant IL-12p35 subunit domain further comprises a C74S substitution mutation.

305. The host cell according to claim 302, wherein the IL-12p40 subunit comprises a variant IL-12p40 subunit, wherein the variant IL-12p40 subunit comprises one or more amino acid substitutions selected from the group consisting of: C177S, C252S, and C177S/C252S.

306. The host cell according to claim 302, wherein the IL-12p40 subunit comprises any one of SEQ ID NOs: 4, 88, 89, and 90.

307. The host cell according to claim 302, wherein the non-naturally occurring IL-12 variant is expressed.

308. The host cell according to claim 307, wherein the expressed non-naturally occurring IL-12 variant is secreted.
